(19) United States
(12) Patent Application Publication
(10) Pub. No.: US 2023/0084176 A1
(43) Pub. Date: Mar. 16, 2023

(54) LIGHT-EMITTING DEVICE AND ELECTRONIC APPARATUS INCLUDING THE SAME

(71) Applicant: Samsung Display Co., Ltd., Yongin-si (KR)

(72) Inventors: Yeongrong PARK, Yongin-si (KR); Kyungsik KIM, Yongin-si (KR); Seokgyu YOON, Yongin-si (KR); Jaeyong LEE, Yongin-si (KR); Hyeonmi LEE, Yongin-si (KR); Kunwook CHO, Yongin-si (KR)

(21) Appl. No.: 17/858,953

(22) Filed: Jul. 6, 2022

(30) Foreign Application Priority Data
Jul. 13, 2021 (KR) 10-2021-0091689

(51) Int. Cl. H01L 51/00; C07D 405/12; C09K 11/06; C07D 209/88

(52) U.S. Cl. CPC H01L 51/0061; C07D 405/12; C09K 11/06; C07D 209/88; H01L 51/006; H01L 51/5056

(57) ABSTRACT

A light-emitting device and an electronic apparatus including the light-emitting device, in which the light-emitting device includes: a first electrode; a second electrode facing the first electrode; and an interlayer arranged between the first electrode and the second electrode and including an emission layer, wherein the interlayer further includes a hole transport region between the first electrode and the emission layer, the first electrode includes a metal oxide having a work function of 5.3 eV or more, and the hole transport region includes a heterocyclic compound represented by Formula 1:

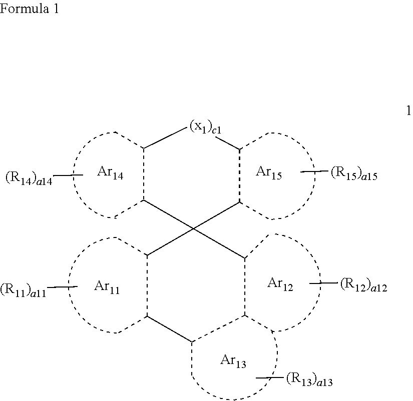

wherein the detailed description of Formula 1 is the same as described in the present specification.

### CROSS-REFERENCE TO RELATED APPLICATION

This application claims priority from and the benefit of Korean Patent Application No. 10-2021-0091689, filed on Jul. 13, 2021, which is hereby incorporated by reference for all purposes as if fully set forth herein.

### BACKGROUND

### Field

Embodiments of the invention relate generally to a light-emitting device and an electronic apparatus including the same.

### Discussion of the Background

From among light-emitting devices, organic light-emitting devices are self-emissive devices that have, as compared with devices in the art, wide viewing angles, high contrast ratios, short response times, and excellent characteristics in terms of luminance, driving voltage, and response speed.

In an organic light-emitting device, a first electrode is arranged on a substrate, and a hole transport region, an emission layer, an electron transport region, and a second electrode are sequentially stacked on the first electrode. Holes provided from the first electrode move toward the emission layer through the hole transport region, and electrons provided from the second electrode move toward the emission layer through the electron transport region. Carriers, such as holes and electrons, recombine in the emission layer to produce excitons. These excitons transition from an excited state to a ground state, thereby generating light.

The above information disclosed in this Background section is only for understanding of the background of the inventive concepts, and, therefore, it may contain information that does not constitute prior art.

### SUMMARY

One or more embodiments include a light-emitting device having excellent light efficiency, low driving voltage, and a long lifespan, and an electronic apparatus including the light-emitting device.

Additional features of the inventive concepts will be set forth in the description which follows, and in part will be apparent from the description, or may be learned by practice of the inventive concepts.

According to one or more embodiments, a light-emitting device includes a first electrode, a second electrode facing the first electrode, an interlayer arranged between the first electrode and the second electrode and including an emission layer, wherein the interlayer further includes a hole transport region arranged between the first electrode and the emission layer, the first electrode includes a metal oxide having a work function of 5.3 eV or more, and the hole transport region includes a heterocyclic compound represented by Formula 1:

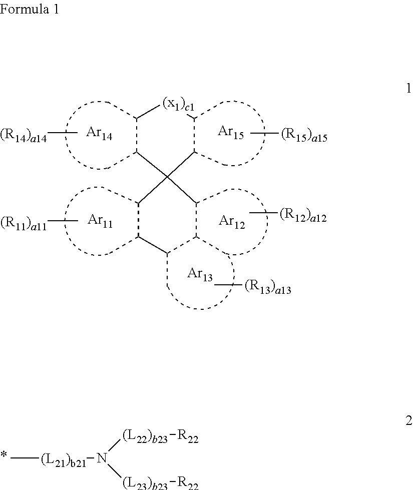

wherein, in Formula 1,
X₁ is a single bond, *—N(Z₁₁ₐ)—*′, *—B(Z₁₁ₐ)—*′, *—P(Z₁₁ₐ)—*′, *—C(Z₁₁)(Z_{11b})—*′, *—Si(Z₁₁ₐ)(Z_{11b})—*′, *—Ge(Z₁₁ₐ)(Z_{11b})—*′, *—S—*′, *—Se—*′, *—O—*, *—C(═O)—*′, *—S(═O)—*′, *—S(═O)₂—*′, *—C(Z₁₁ₐ)=*′, *═C(Z₁₁ₐ)—*′, *—C(Z₁₁ₐ)═C(Z_{11b})—*′, *—C(═S)—*′, or *—C≡C—*′,

wherein * and *′ each indicate a neighboring atom,

c1 is 0 or 1,
Ar₁₁ to Ar₁₅ are each independently a C₃-C₆₀ carbocyclic group or a C₁-C₆₀ heterocyclic group,
R₁₁ to R₁₅, Z₁₁ₐ, and Z_{11b} are each independently a group represented by Formula 2, hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₁-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂),
at least one of R₁₁ to R₁₅ is a group represented by Formula 2,
a11 to a15 are each independently an integer from 0 to 10, wherein, in Formula 2,
L₂₁ to L₂₃ are each independently a single bond, a C₃-C₆₀ carbocyclic group unsubstituted or substituted with at least one R₁₀ₐ, or a C₁-C₆₀ heterocyclic group unsubstituted or substituted with at least one R₁₀ₐ,
b21 to b23 are each independently an integer from 0 to 5,
R₂₂ and R₂₃ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂),
two or more groups selected from R₁₁(s) in the number of a11, R12(s) in the number of a12, R₁₃(s) in the number of a13, R₁₄(s) in the number of a14, R₁₅(s) in the number of a15, R₂₂, and R₂₃ are optionally linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group unsubstituted or substituted with at least one R₁₀ₐ,
R₁₀ₐ is deuterium, —F, —Cl, —Br, —I, a hydroxyl group, a cyano group, or a nitro group, a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —B(Q₁₁)(Q₁₂), —C(═O)(Q₁₁), —S(═O)₂(Q₁₁), —P(═O)(Q₁₁)(Q₁₂), or any combination thereof, a C₃-C₆₀ carbocyclic group, a C₁-C₆₀ heterocyclic group, a C₆-C₆₀ aryloxy group; or a C₆-C₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —B(Q₂₁)(Q₂₂), —C(═O)(Q₂₁), —S(═O)₂(Q₂₁), —P(═O)(Q₂₁)(Q₂₂), or any combination thereof, or —Si(Q₃₁)(Q₃₂)(Q₃₃), —B(Q₃₁)(Q₃₂), —C(═O)(Q₃₁), —S(═O)₂(Q₃₁), or —P(═O)(Q₃₁)(Q₃₂), Q₁ to Q₃, Q₁₁ to Q₁₃, Q₂₁ to Q₂₃, and Q₃₁ to Q₃₃ are each independently hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof, and * indicates a binding site to a neighboring atom.

According to one or more embodiments, an electronic apparatus includes the light-emitting device.

It is to be understood that both the foregoing general description and the following detailed description are illustrative and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate illustrative embodiments of the invention, and together with the description serve to explain the inventive concepts.

FIG. 1 shows a schematic cross-sectional view of a light-emitting device according to an embodiment that is constructed according to principles of the invention.

FIGS. 2 and 3 are each a cross-sectional view showing a light-emitting apparatus according to an embodiment.

### DETAILED DESCRIPTION

In the following description, for the purposes of explanation, numerous specific details are set forth in order to provide a thorough understanding of various embodiments or implementations of the invention. As used herein “embodiments” and “implementations” are interchangeable words that are non-limiting examples of devices or methods employing one or more of the inventive concepts disclosed herein. It is apparent, however, that various embodiments may be practiced without these specific details or with one or more equivalent arrangements. In other instances, well-known structures and devices are shown in block diagram form in order to avoid unnecessarily obscuring various embodiments. Further, various embodiments may be different, but do not have to be exclusive. For example, specific shapes, configurations, and characteristics of an embodiment may be used or implemented in another embodiment without departing from the inventive concepts.

Unless otherwise specified, the illustrated embodiments are to be understood as providing illustrative features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various embodiments are described herein with reference to sectional and/or exploded illustrations that are schematic illustrations of idealized embodiments and/or intermediate structures. As such, variations from the shapes of the illustrations as a result, for example, of manufacturing techniques and/or tolerances, are to be expected. Thus, embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

An aspect of the present disclosure provides a light-emitting device including: a first electrode; a second electrode facing the first electrode; an interlayer arranged between the first electrode and the second electrode and including an emission layer, wherein the interlayer further includes a hole transport region arranged between the first electrode and the emission layer, the first electrode includes a metal oxide having a work function of 5.3 eV or more, and the hole transport region includes a heterocyclic compound represented by Formula 1. The metal oxide and the heterocyclic compound will be described in detail below.

In an embodiment, the first electrode may further include a conductive oxide material, a metal material, a metal alloy material, or any combination thereof.

For example, the conductive oxide material may include indium tin oxide (ITO), indium zinc oxide (IZO), tin oxide (SnO₂), zinc oxide (ZnO), or any combination thereof.

For example, the metal material or the metal alloy material may include magnesium (Mg), silver (Ag), aluminum (Al), aluminum-lithium (Al—Li), calcium (Ca), magnesium-indium (Mg—In), magnesium-silver (Mg—Ag), AlNiLa, AlNd, AlNiGeLa, AlCoGeLa, or any combination thereof.

The term “AlNiLa” refers to an aluminum-nickel-lanthanum alloy, wherein, for example, an amount of nickel may be in a range of about 1 atomic % to about 3 atomic %, and an amount of lanthanum may be in a range of about 0.1 atomic % to about 0.5 atomic %.

The term “AlNd” refers to an aluminum-neodymium alloy, wherein, for example, an amount of neodymium may be in a range of about 1 atomic % to 3 about atomic %.

The term “AlNiGeLa” refers to an aluminum-nickel-germanium-lanthanum alloy, wherein, for example, an amount of nickel may be in a range of about 1 atomic % to about 3 atomic %, an amount of germanium may be in a range of about 1 atomic % to about 3 atomic %, and an amount of lanthanum may be in a range of about 0.01 atomic % to about 0.2 atomic %.

The term “AlCoGeLa” refers to an aluminum-cobalt-germanium-lanthanum alloy, wherein, for example, an amount of cobalt may be in a range of about 1 atomic % to about 3 atomic %, an amount of germanium may be in a range of about 1 atomic % to about 3 atomic %, and an amount of lanthanum may be in a range of about 0.01 atomic % to about 0.2 atomic %.

In an embodiment, in the light-emitting device, the first electrode may be an anode, the second electrode may be a cathode, and the interlayer may further include a hole transport region between the first electrode and the emission layer and an electron transport region between the emission layer and the second electrode, wherein the hole transport region may include a hole injection layer, a hole transport layer, an emission auxiliary layer, an electron blocking layer, or any combination thereof, and the electron transport region may include a hole blocking layer, an electron transport layer, an electron injection layer, or any combination thereof.

In an embodiment, the hole transport layer may include the heterocyclic compound represented by Formula 1.

In an embodiment, in the light-emitting device, the first electrode and the hole transport layer may be in direct contact with each other.

In an embodiment, in the light-emitting device, the hole transport layer and the emission layer may be in direct contact with each other.

In an embodiment, in the light-emitting device, the hole transport region may not include a p-dopant.

In an embodiment, the light-emitting device may emit red light, green light, or blue light.

Description of Metal Oxide

The first electrode of the light-emitting device may include a metal oxide having a work function of 5.3 eV or more.

In an embodiment, the metal oxide may be an oxide of tungsten (W), molybdenum (Mo), copper (Cu), nickel (Ni), vanadium (V), or any combination thereof. The any combination thereof may include, for example: an alloy of W and Mo; an alloy of Cu and W, an alloy of W and Cu; an alloy of W and Ni; an alloy of W and V; an alloy of W, Mo, and Cu; an alloy of Ni and V; and the like.

In an embodiment, the metal oxide may include WOₓ, MoOₓ, NiO_{y}, CuO_{y}, or any combination thereof (wherein x is a real number satisfying 2.5≤x≤3. 1, and y is a real number satisfying 0.5≤y≤1.0). For example, x may be a real number satisfying 2.6≤x≤3.1, 2.7≤x≤3.1, 2.8≤x≤3.1, or 2.9≤x≤3.1, but embodiments of the present disclosure are not limited thereto.

In one or more embodiments, the metal oxide may include WO₃, WO₂, WO, W₂O₃, W₂O₅, MoO₃, MoO₂, Cu₂O, CuO, Cu₂O₃, NiO, Ni₂O₃, Ni₂O, VO, VO₂, V₂O₃, V₂O₅, V₆O₁₃, or any combination thereof.

For example, the first electrode may consist of MoO₃, MoO₂, WO₃, WO₂, WO, W₂O₃, W₂O₅, Cu₂O, CuO, Cu₂O₃, NiO, Ni₂O₃, Ni₂O, VO, VO₂, V₂O₃, V₂O₅, V₆O₁₃, or any combination thereof.

Description of Heterocyclic Compound

The hole transport region of the light-emitting device may include the heterocyclic compound represented by Formula 1:

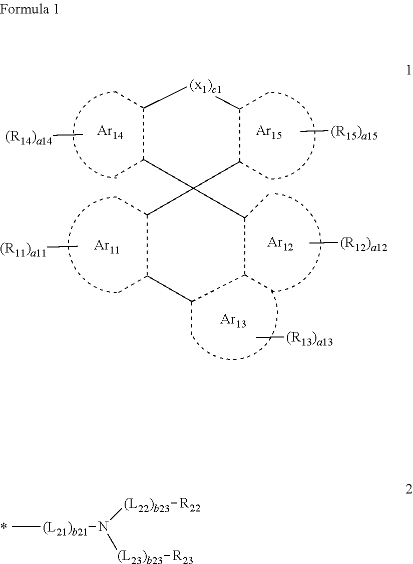

wherein, in Formula 1, X₁ may be a single bond, *—N(Z₁₁ₐ)—*′, *—B(Z₁₁ₐ)—*′, *—P(Z₁₁ₐ)—*′, *—C(Z₁₁)(Z_{11b})—*′, *—Si(Z₁₁ₐ)(Z_{11b})—*′, *—Ge(Z₁₁ₐ)(Z_{11b})—*′, *—S—*′, *—Se—*′, *—O—*′, *—C(═O)—*′, *—S(═O)—*′, *—S(═O)₂—*′, *—C(Z₁₁ₐ)=*′, *═C(Z₁₁ₐ)—*′, *—C(Z₁₁ₐ)═C(Z_{11b})—*′, *—C(═S)—*′, or *—C≡C—*′, wherein * and *′ each indicate a neighboring atom.

In an embodiment, X₁ may be a single bond, *—S—*′, or *—O—*′.

In Formula 1, c1 may be 0 or 1.

In Formula 1, Ar₁₁ to Ar₁₅ may each independently be a C₃-C₆₀ carbocyclic group or a C₁-C₆₀ heterocyclic group.

In an embodiment, Ar₁₁ to Ar₁₅ may each independently be a π electron-rich C₃-C₆₀cyclic group.

In one or more embodiments, Ar₁₁ to Ar₁₅ may each independently be a benzene group, a heptalene group, an indene group, a naphthalene group, an azulene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentaphene group, a rubicene group, a coronene group, an ovalene group, a pyrrole group, a furan group, a thiophene group, an isoindole group, an indole group, an indene group, a benzofuran group, a benzothiophene group, a benzosilole group, a naphthopyrrole group, a naphthofuran group, a naphthothiophene group, a naphthosilole group, a benzocarbazole group, a dibenzocarbazole group, a dibenzofuran group, a dibenzothiophene group, a carbazole group, a dibenzosilole group, an indenocarbazole group, an indolocarbazole group, a benzofurocarbazole group, a benzothienocarbazole group, a benzosilolocarbazole group, a triindolobenzene group, a pyrrolophenanthrene group, a furanophenanthrene group, a thienophenanthrene group, a benzonaphthofuran group, a benzonapthothiophene group, an (indolo)phenanthrene group, a (benzofurano)phenanthrene group, or a (benzothieno)phenanthrene group.

In an embodiment, Formula 1 may be represented by Formula 1-1 or 1-2:

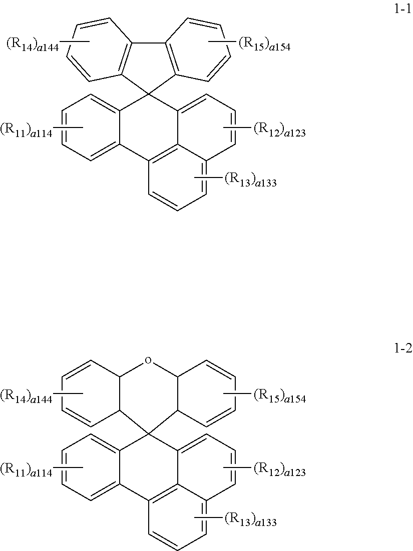

wherein, in Formulae 1-1 and 1-2,

R₁₁ to R₁₅ may respectively be the same as described herein,

a114, a144, and a154 may each independently be an integer from 0 to 4, and

a123 and a133 may each independently be an integer from 0 to 3.

In an embodiment, a group represented by

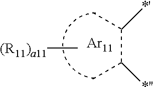

in Formula 1 may be represented by one of Formulae Ar11-1 to Ar11-5:

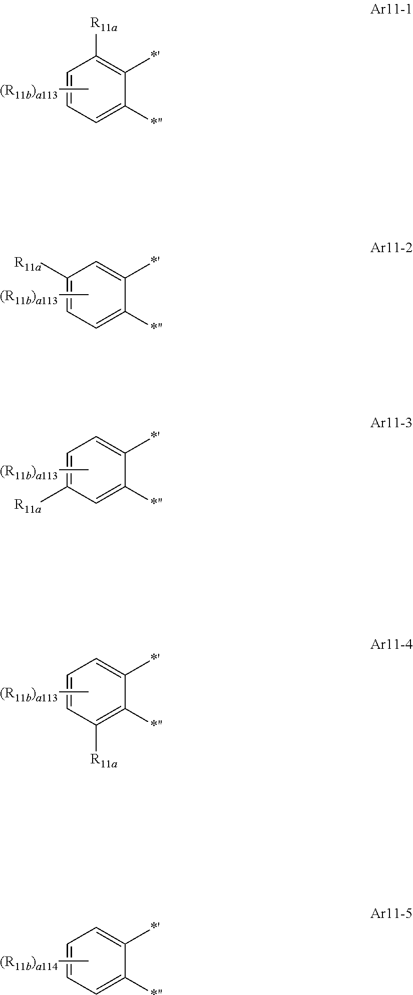

wherein, in Formulae Ar11-1 to Ar11-5,
R₁₁ₐ may be a group represented by Formula 2,
R_{11b} may be as described in connection with R₁₁, wherein R_{11b} may not be a group represented by Formula 2,
a113 may be an integer from 0 to 3,
a114 may be an integer from 0 to 4, and
′ and *″ each indicate a binding site to a neighboring atom.

In an embodiment, a group represented by

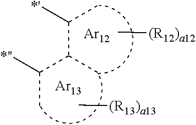

in Formula 1 may be represented by one of Formulae Ar12-1 to Ar12-7:

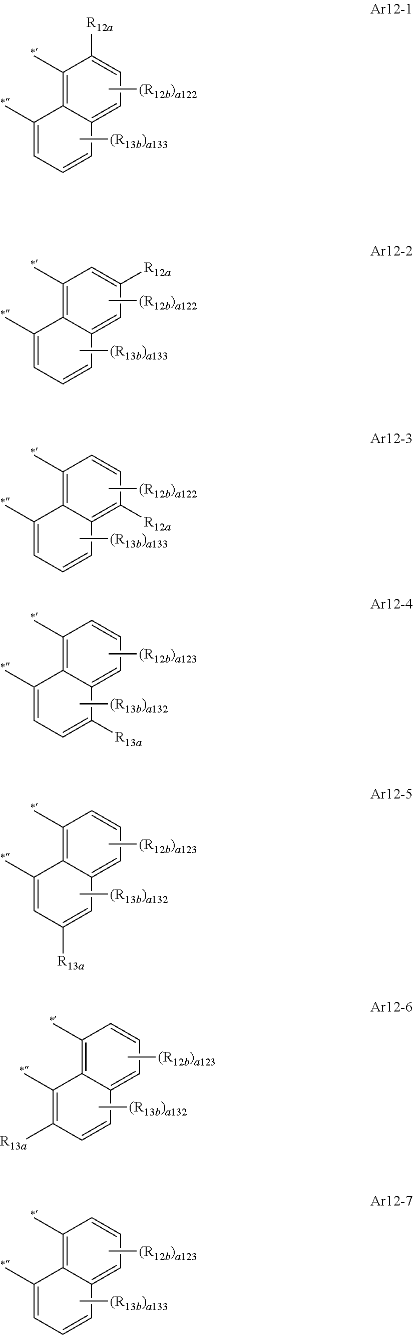

wherein, in Formulae Ar12-1 to Ar12-7,
R₁₂ₐ and R₁₃ₐ may each be a group represented by Formula 2,
R_{12b} may be the same as described in connection with R₁₂, wherein R_{12b} may not be a group represented by Formula 2,
R_{13b} may be the same as described in connection with R₁₂, wherein R_{13b} may not be a group represented by Formula 2,
a122 and a132 may each independently be an integer from 0 to 2,
a123 and a133 may each independently be an integer from 0 to 3, and
*′ and *″ each indicate a binding site to a neighboring atom.

In Formula 1, R₁₁ to R₁₅, Z₁₁ₐ, and Z_{11b} may each independently be a group represented by Formula 2, hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂),

wherein at least one of R₁₁ to R₁₅ may be a group represented by Formula 2.

In an embodiment, R₁₁ to R₁₅, Z₁₁ₐ, and Z_{11b} may each independently be: hydrogen, deuterium, —F, —Cl, —Br, —I, a hydroxyl group, a cyano group, or a nitro group; C₁-C₂₀ alkyl group, a C₂-C₂₀ alkenyl group, a C₂-C₂₀ alkynyl group, or a C₁-C₂₀ alkoxy group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a phenyl group, a naphthyl group, a pyridinyl group, a pyrimidinyl group, —Si(Q₃₁)(Q₃₂)(Q₃₃), —B(Q₃₁)(Q₃₂), or any combination thereof;

a phenyl group, a naphthyl group, a pyridinyl group, or a pyrimidinyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₂-C₂₀ alkenyl group, a C₂-C₂₀ alkynyl group, a C₁-C₂₀ alkoxy group, a phenyl group, a naphthyl group, a pyridinyl group, a pyrimidinyl group, —Si(Q₃₁)(Q₃₂)(Q₃₃), —B(Q₃₁)(Q₃₂), or any combination thereof; or

—Si(Q₁)(Q₂)(Q₃) or —B(Q₁)(Q₂), and

Q₁ to Q₃ and Q₃₁ to Q₃₃ may respectively be the same as described herein.

In Formula 1, a11 to a15 may each independently be an integer from 0 to 10. For example, a11 indicates the number of Ru(s), and may be an integer from 0 to 10. When a11 is 2 or more, two or more of Ru(s) may be identical to or different from each other. This description also applies to a12 to a15. For example, a11 to a15 may each independently be 0 or 1, but embodiments of the disclosure are not limited thereto.

In an embodiment, i) a11 may be an integer from 1 to 4, and at least one of Ru(s) in the number of a11 may be a group represented by Formula 2;

ii) a12 may be an integer from 1 to 3, and at least one of R₁₂(s) in the number of a12 may be a group represented by Formula 2; or

iii) a13 may be an integer from 1 to 3, and at least one of R13(s) in the number of a13 may be a group represented by Formula 2.

In Formula 2,

L₂₁ to L₂₃ may each independently be a single bond, a C₅-C₆₀ carbocyclic group unsubstituted or substituted with at least one R₁₀ₐ, or a C₁-C₆₀ heterocyclic group unsubstituted or substituted with at least one R₁₀ₐ.

In one or more embodiments, L21 to L23 may each independently be: a single bond; or a benzene group, a naphthalene group, an anthracene group, a phenanthrene group, a triphenylene group, a pyrene group, a chrysene group, cyclopentadiene group, a 1,2,3,4-tetrahydronaphthalene group, a thiophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-fluoren-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluoren-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isooxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group, each unsubstituted or substituted with at least one R₁₀ₐ.

In one or more embodiments, L21 to L23 may each independently be a single bond or a π electron-rich C₃-C₆₀ cyclic group unsubstituted or substituted with R₁₀ₐ.

In one or more embodiments, L₂₁ to L₂₃ may each independently be: a single bond; or a benzene group, a naphthalene group, or a carbazole group, each unsubstituted or substituted with at least one R₁₀ₐ.

In Formula 2, b21 to b23 may each independently be an integer from 0 to 5. In Formula 2, b21 indicates the number of L₂₁(s), and may be an integer from 0 to 5. When b21 is 2 or more, two or more of L₂₁(s) may be identical to or different from each other. This description also applies to b22 and b23.

In Formula 2,

R₂₂ and R₂₃ may each independently be hydrogen, deuterium, —F, —Cl, —Br, —I, a hydroxyl group, a cyano group, a nitro group, a C₁-C₆₀alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂), and

two or more groups selected from Ru(s) in the number of a11, R₁₂(s) in the number of a12, R₁₃(s) in the number of a13, R₁₄(s) in the number of a14, R₁₅(s) in the number of a15, R₂₂, and R₂₃ may optionally be linked together via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group unsubstituted or substituted with at least one R₁₀ₐ. R₁₀ₐ, Q₁ to Q₃, Q₁₁ to Q₁₃, Q₂₁ to Q₂₃, and Q₃₁ to Q₃₃ may respectively be the same as described herein.

In an embodiment, R₂₂ and R₂₃ in Formula 2 may each independently be: a group represented by Formula 3-1 or 3-2;

hydrogen, deuterium, a hydroxyl group, or a nitro group;

a C₁-C₂₀ alkyl group, a C₂-C₂₀ alkenyl group, a C₂-C₂₀ alkynyl group, or a C₁-C₂₀ alkoxy group, each substituted or unsubstituted with deuterium, —CD₃, —CD₂H, —CDH₂, a hydroxyl group, a nitro group, a phenyl group, a naphthyl group, —Si(Q₃₁)(Q₃₂)(Q₃₃), —B(Q₃₁)(Q₃₂), or any combination thereof;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a thiophenyl group, a furanyl group, an isoindolyl group, an indolyl group, a carbazolyl group, a benzofuranyl group, a benzothiophenyl group, a dibenzofuranyl group, a dibenzothiophenyl group, a benzocarbazolyl group, or a dibenzocarbazolyl group, each substituted or unsubstituted with deuterium, —CD₃, —CD₂H, —CDH₂, a hydroxyl group, a nitro group, a C₁-C₂₀ alkyl group, a C₂-C₂₀ is alkenyl group, a C₂-C₂₀ alkynyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a thiophenyl group, a furanyl group, an isoindolyl group, an indolyl group, a carbazolyl group, a benzofuranyl group, a benzothiophenyl group, a dibenzofuranyl group, a dibenzothiophenyl group, a benzocarbazolyl group, a dibenzocarbazolyl group, —Si(Q₃₁)(Q₃₂)(Q₃₃), —N(Q₃₁)(Q₃₂), —B(Q₃₁)(Q₃₂), or any combination thereof, or

—Si(Q₁)(Q₂)(Q₃) or —B(Q₁)(Q₂), and

at least one of R₂₂ and R₂₃ may be a group represented by Formula 3-1 or 3-2:

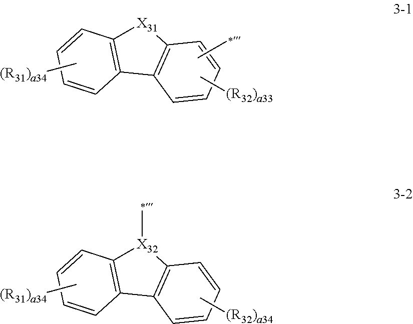

wherein, in Formulae 3-1 and 3-2,

X₃₁ may be O, S, N(Z₃₁ₐ), or C(Z₃₁ₐ)(Z_{31b}),

X₃₂ may be N or C(Z₃₂ₐ),

R₃₁, R₃₂, Z₃₁ₐ, and Z_{31b} may respectively be the same as described in connection with R₂₂,

a33 may be an integer from 0 to 3,

a34 may be an integer from 0 to 4,

two or more groups selected from R₃₁(s) in the number of a34 and R₃₂(s) in the number of a33 or a34 may optionally be linked together via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group unsubstituted or substituted with at least one R₁₀ₐ, and

*′″ indicates a binding site to R₂₂ or R₂₃.

In one or more embodiments, X₃₁ may be O, S, N(Z₃₁ₐ), or C(Z₃₁ₐ)(Z_{31b}), and X₃₂ may be N.

In one or more embodiments, X₃₁ may be N(Z₃₁ₐ), and X₃₂ may be N.

In one or more embodiments, the group represented by Formula 3-1 or 3-2 may be one of groups represented by Formulae 3-1A to 3-1G and 3-2:

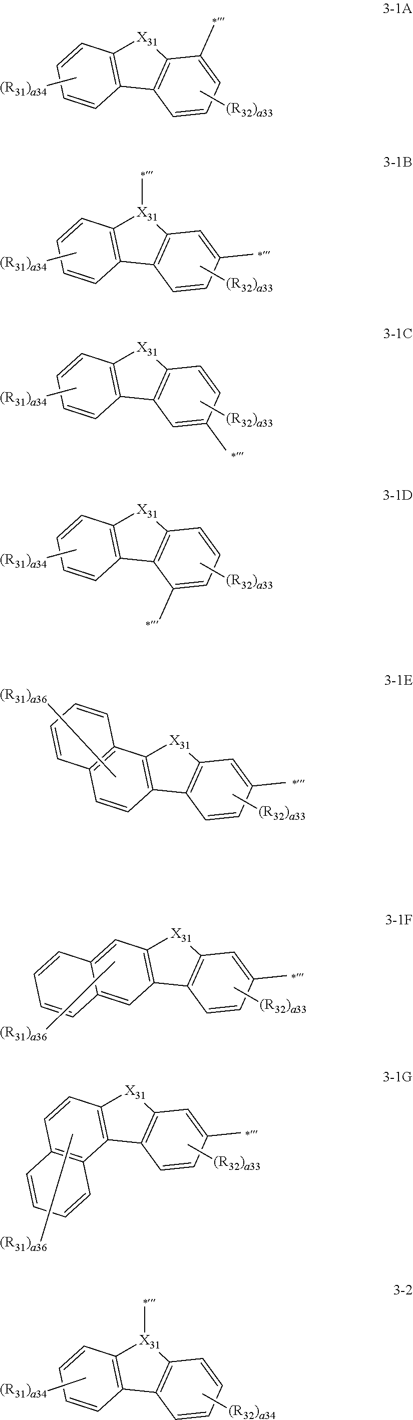

In Formulae 3-1A to 3-1G and 3-2,

X₃₁, X₃₂, R₃₁, R₃₁, R₃₂, a33, and a34 may respectively be the same as described herein,

a36 may be an integer from 0 to 6, and

*′″ indicates a binding site to R₂₂ or R₂₃.

In one or more embodiments, R₃₁, R₃₂, Z₃₁ₐ, and Z_{31b} may each independently be:

hydrogen, deuterium, a hydroxyl group, or a nitro group;

a C₁-C₂₀ alkyl group, a C₂-C₂₀ alkenyl group, a C₂-C₂₀ alkynyl group, or a C₁-C₂₀ alkoxy group, each substituted or unsubstituted with deuterium, —CD₃, —CD₂H, —CDH₂, a hydroxyl group, a nitro group, a phenyl group, a naphthyl group, —Si(Q₃₁)(Q₃₂)(Q₃₃), —B(Q₃₁)(Q₃₂), or any combination thereof;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a thiophenyl group, a furanyl group, an isoindolyl group, an indolyl group, a carbazolyl group, a benzofuranyl group, a benzothiophenyl group, a dibenzofuranyl group, a dibenzothiophenyl group, a benzocarbazolyl group, or a dibenzocarbazolyl group, each substituted or unsubstituted with deuterium, —CD₃, —CD₂H, —CDH₂, a hydroxyl group, a nitro group, a C₁-C₂₀ alkyl group, a C₂-C₂₀ alkenyl group, a C₂-C₂₀ alkynyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a thiophenyl group, a furanyl group, an isoindolyl group, an indolyl group, a carbazolyl group, a benzofuranyl group, a benzothiophenyl group, a dibenzofuranyl group, a dibenzothiophenyl group, a benzocarbazolyl group, a dibenzocarbazolyl group, —Si(Q₃₁)(Q₃₂)(Q₃₃), —N(Q₃₁)(Q₃₂), —B(Q₃₁)(Q₃₂), or any combination thereof, or

—Si(Q₁)(Q₂)(Q₃) or —B(Q₁)(Q₂), and

Q₁ to Q₃ and Q₃₁ to Q₃₃ may respectively be the same as described herein.

In an embodiment, the heterocyclic compound represented by Formula 1 may be selected from Compounds 1 to 99:

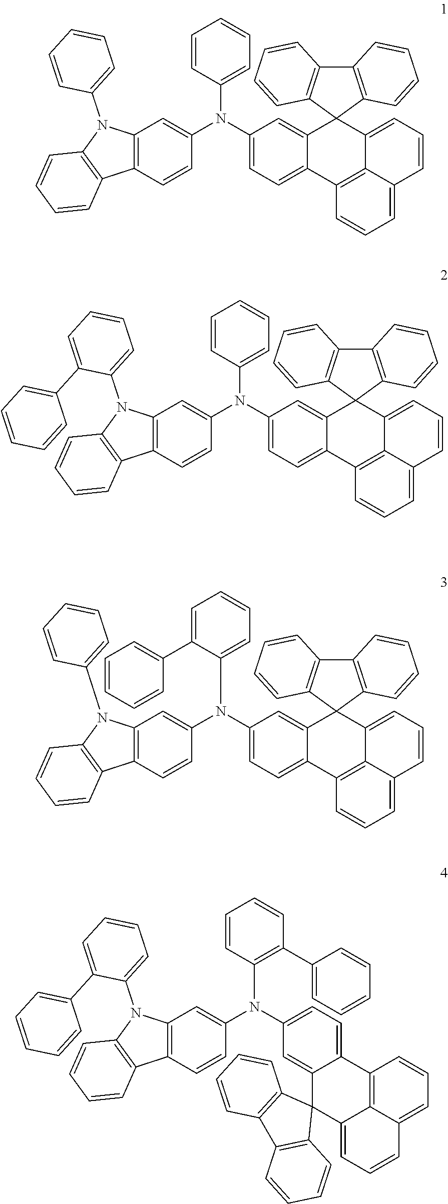 [SMILES: c1ccc(N(c2ccc3c(c2)C2(c4ccccc4-c4ccccc42)c2cccc4cccc-3c24)c2ccc3c4ccccc4n(-c4ccccc4)c3c2)cc1 ; c1ccc(-c2ccccc2-n2c3ccccc3c3ccc(N(c4ccccc4)c4ccc5c(c4)C4(c6ccccc6-c6ccccc64)c4cccc6cccc-5c46)cc32)cc1 ; c1ccc(-c2ccccc2N(c2ccc3c(c2)C2(c4ccccc4-c4ccccc42)c2cccc4cccc-3c24)c2ccc3c4ccccc4n(-c4ccccc4)c3c2)cc1 ; c1ccc(-c2ccccc2N(c2ccc3c(c2)C2(c4ccccc4-c4ccccc42)c2cccc4cccc-3c24)c2ccc3c4ccccc4n(-c4ccccc4-c4ccccc4)c3c2)cc1]

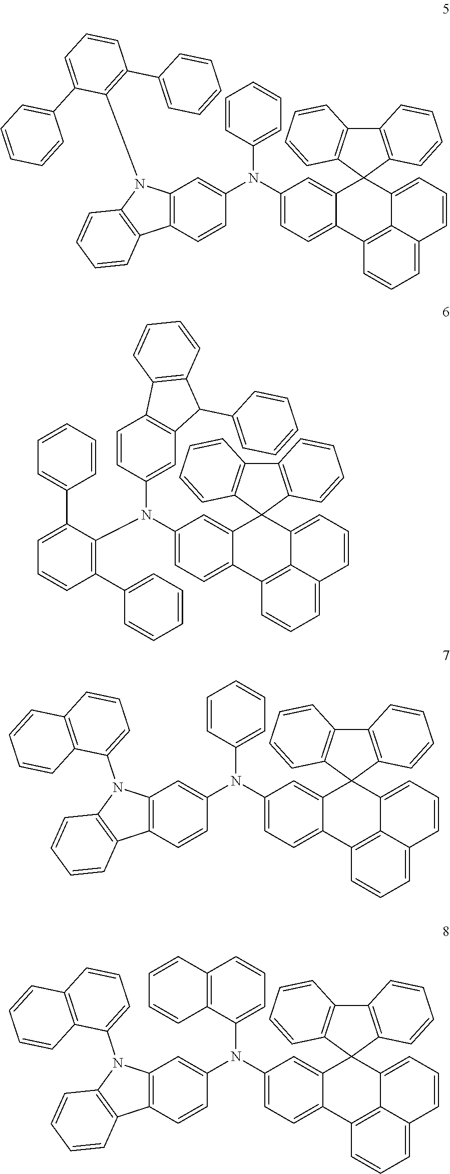 [SMILES: c1ccc(-c2cccc(-c3ccccc3)c2-n2c3ccccc3c3ccc(N(c4ccccc4)c4ccc5c(c4)C4(c6ccccc6-c6ccccc64)c4cccc6cccc-5c46)cc32)cc1 ; c1ccc(-c2cccc(-c3ccccc3)c2N(c2ccc3c(c2)C(c2ccccc2)c2ccccc2-3)c2ccc3c(c2)C2(c4ccccc4-c4ccccc42)c2cccc4cccc-3c24)cc1 ; c1ccc(N(c2ccc3c(c2)C2(c4ccccc4-c4ccccc42)c2cccc4cccc-3c24)c2ccc3c4ccccc4n(-c4cccc5ccccc45)c3c2)cc1 ; c1ccc2c(c1)-c1ccccc1C21c2cc(N(c3ccc4c5ccccc5n(-c5cccc6ccccc56)c4c3)c3cccc4ccccc34)ccc2-c2cccc3cccc1c23]

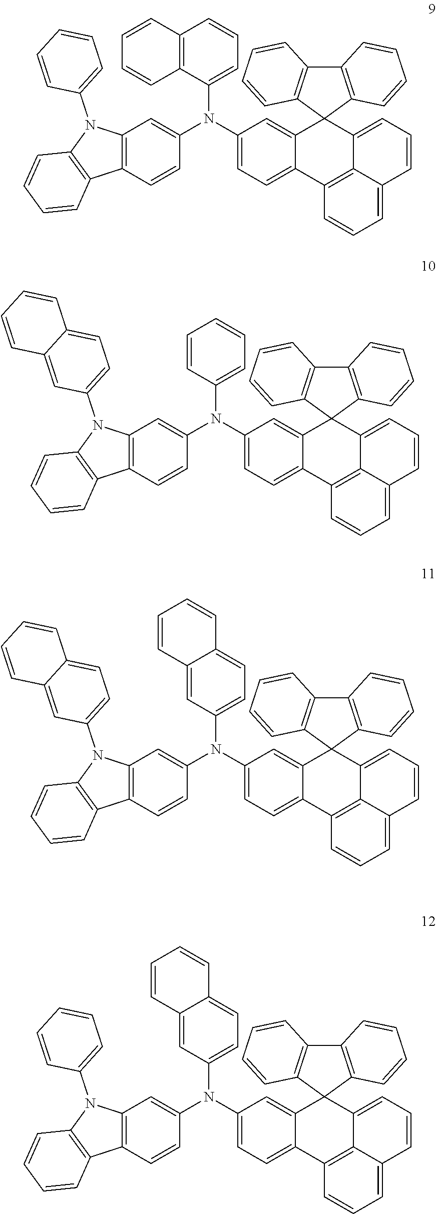 [SMILES: c1ccc(-n2c3ccccc3c3ccc(N(c4ccc5c(c4)C4(c6ccccc6-c6ccccc64)c4cccc6cccc-5c46)c4cccc5ccccc45)cc32)cc1 ; c1ccc(N(c2ccc3c(c2)C2(c4ccccc4-c4ccccc42)c2cccc4cccc-3c24)c2ccc3c4ccccc4n(-c4ccc5ccccc5c4)c3c2)cc1 ; c1ccc2c(c1)-c1ccccc1C21c2cc(N(c3ccc4ccccc4c3)c3ccc4c5ccccc5n(-c5ccc6ccccc6c5)c4c3)ccc2-c2cccc3cccc1c23 ; c1ccc(-n2c3ccccc3c3ccc(N(c4ccc5c(c4)C4(c6ccccc6-c6ccccc64)c4cccc6cccc-5c46)c4ccc5ccccc5c4)cc32)cc1]

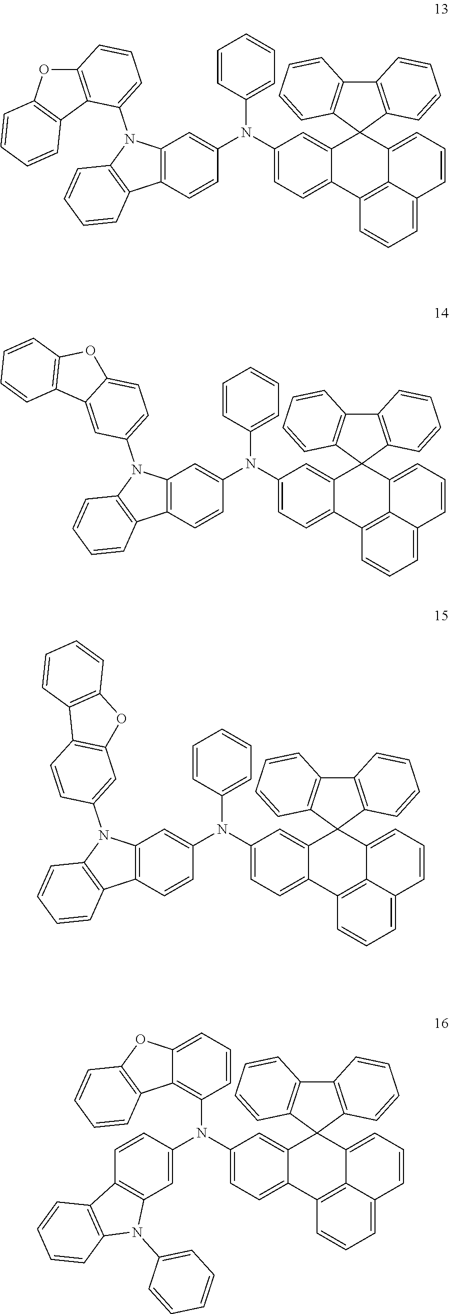 [SMILES: c1ccc(N(c2ccc3c(c2)C2(c4ccccc4-c4ccccc42)c2cccc4cccc-3c24)c2ccc3c4ccccc4n(-c4cccc5oc6ccccc6c45)c3c2)cc1 ; c1ccc(N(c2ccc3c(c2)C2(c4ccccc4-c4ccccc42)c2cccc4cccc-3c24)c2ccc3c4ccccc4n(-c4ccc5oc6ccccc6c5c4)c3c2)cc1 ; c1ccc(N(c2ccc3c(c2)C2(c4ccccc4-c4ccccc42)c2cccc4cccc-3c24)c2ccc3c4ccccc4n(-c4ccc5c(c4)oc4ccccc45)c3c2)cc1 ; c1ccc(-n2c3ccccc3c3ccc(N(c4ccc5c(c4)C4(c6ccccc6-c6ccccc64)c4cccc6cccc-5c46)c4cccc5oc6ccccc6c45)cc32)cc1]

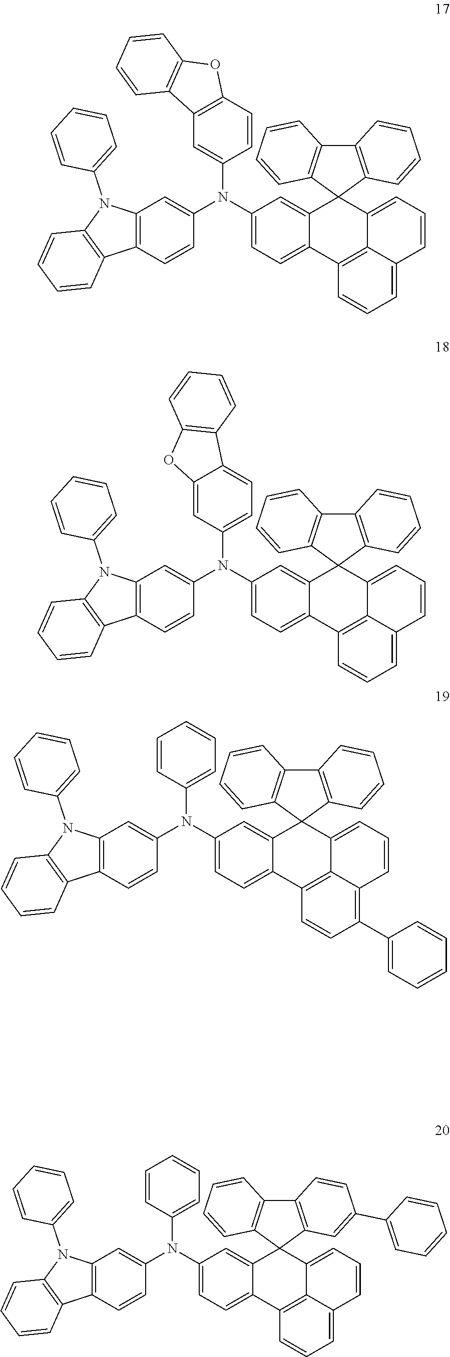 [SMILES: c1ccc(-n2c3ccccc3c3ccc(N(c4ccc5c(c4)C4(c6ccccc6-c6ccccc64)c4cccc6cccc-5c46)c4ccc5oc6ccccc6c5c4)cc32)cc1 ; c1ccc(-n2c3ccccc3c3ccc(N(c4ccc5c(c4)C4(c6ccccc6-c6ccccc64)c4cccc6cccc-5c46)c4ccc5c(c4)oc4ccccc45)cc32)cc1 ; c1ccc(-c2ccc3c4c(cccc24)C2(c4ccccc4-c4ccccc42)c2cc(N(c4ccccc4)c4ccc5c6ccccc6n(-c6ccccc6)c5c4)ccc2-3)cc1 ; c1ccc(-c2ccc3c(c2)C2(c4ccccc4-3)c3cc(N(c4ccccc4)c4ccc5c6ccccc6n(-c6ccccc6)c5c4)ccc3-c3cccc4cccc2c34)cc1]

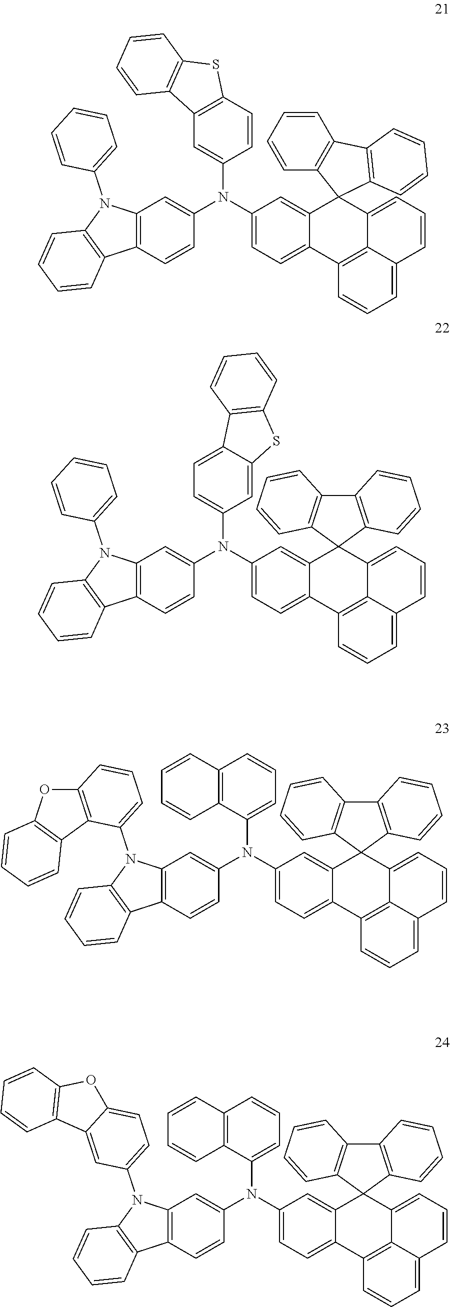 [SMILES: c1ccc(-n2c3ccccc3c3ccc(N(c4ccc5c(c4)C4(c6ccccc6-c6ccccc64)c4cccc6cccc-5c46)c4ccc5sc6ccccc6c5c4)cc32)cc1 ; c1ccc(-n2c3ccccc3c3ccc(N(c4ccc5c(c4)C4(c6ccccc6-c6ccccc64)c4cccc6cccc-5c46)c4ccc5c(c4)sc4ccccc45)cc32)cc1 ; c1ccc2c(c1)-c1ccccc1C21c2cc(N(c3ccc4c5ccccc5n(-c5cccc6oc7ccccc7c56)c4c3)c3cccc4ccccc34)ccc2-c2cccc3cccc1c23 ; c1ccc2c(c1)-c1ccccc1C21c2cc(N(c3ccc4c5ccccc5n(-c5ccc6oc7ccccc7c6c5)c4c3)c3cccc4ccccc34)ccc2-c2cccc3cccc1c23]

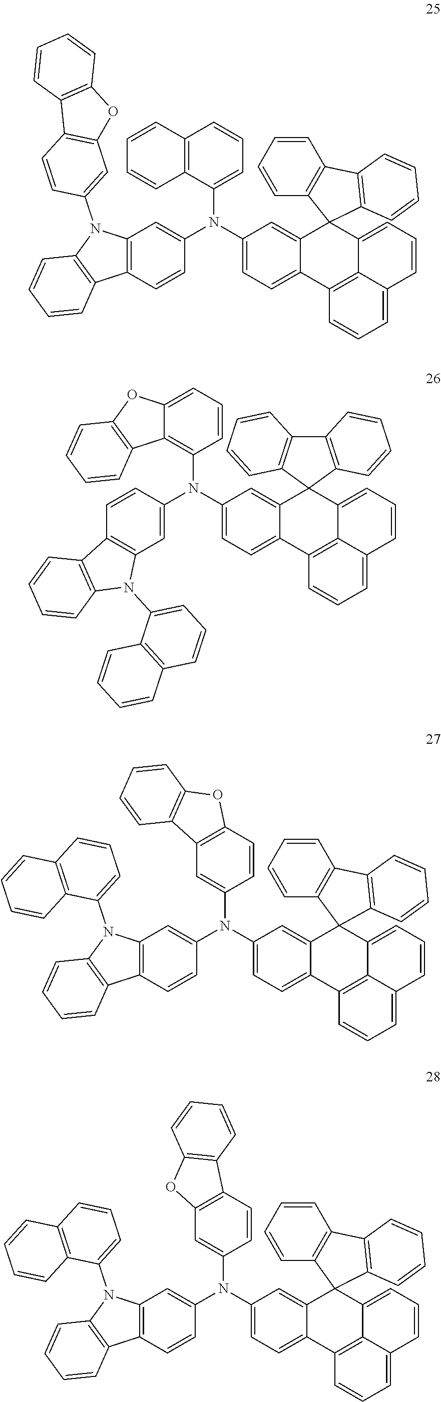 [SMILES: c1ccc2c(c1)-c1ccccc1C21c2cc(N(c3ccc4c5ccccc5n(-c5ccc6c(c5)oc5ccccc56)c4c3)c3cccc4ccccc34)ccc2-c2cccc3cccc1c23 ; c1ccc2c(c1)-c1ccccc1C21c2cc(N(c3ccc4c5ccccc5n(-c5cccc6ccccc56)c4c3)c3cccc4oc5ccccc5c34)ccc2-c2cccc3cccc1c23 ; c1ccc2c(c1)-c1ccccc1C21c2cc(N(c3ccc4oc5ccccc5c4c3)c3ccc4c5ccccc5n(-c5cccc6ccccc56)c4c3)ccc2-c2cccc3cccc1c23 ; c1ccc2c(c1)-c1ccccc1C21c2cc(N(c3ccc4c(c3)oc3ccccc34)c3ccc4c5ccccc5n(-c5cccc6ccccc56)c4c3)ccc2-c2cccc3cccc1c23]

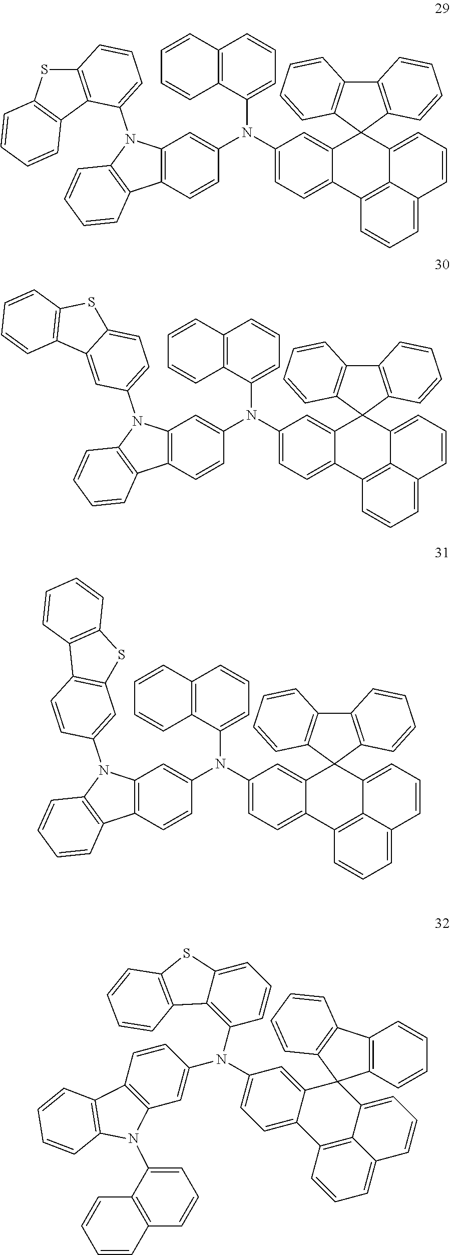 [SMILES: c1ccc2c(c1)-c1ccccc1C21c2cc(N(c3ccc4c5ccccc5n(-c5cccc6sc7ccccc7c56)c4c3)c3cccc4ccccc34)ccc2-c2cccc3cccc1c23 ; c1ccc2c(c1)-c1ccccc1C21c2cc(N(c3ccc4c5ccccc5n(-c5ccc6sc7ccccc7c6c5)c4c3)c3cccc4ccccc34)ccc2-c2cccc3cccc1c23 ; c1ccc2c(c1)-c1ccccc1C21c2cc(N(c3ccc4c5ccccc5n(-c5ccc6c(c5)sc5ccccc56)c4c3)c3cccc4ccccc34)ccc2-c2cccc3cccc1c23 ; c1ccc2c(c1)-c1ccccc1C21c2cc(N(c3ccc4c5ccccc5n(-c5cccc6ccccc56)c4c3)c3cccc4sc5ccccc5c34)ccc2-c2cccc3cccc1c23]

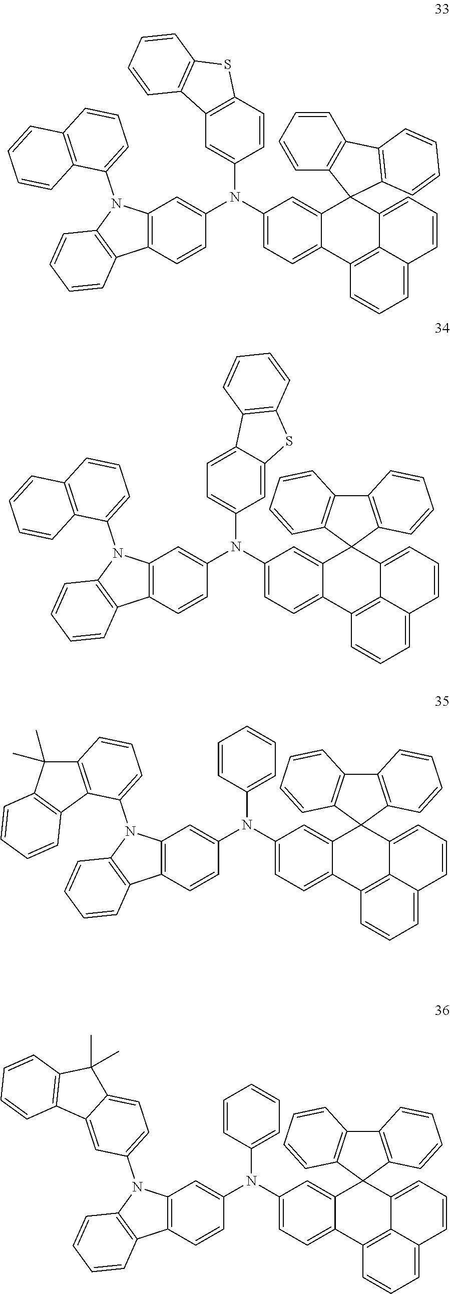 [SMILES: c1ccc2c(c1)-c1ccccc1C21c2cc(N(c3ccc4sc5ccccc5c4c3)c3ccc4c5ccccc5n(-c5cccc6ccccc56)c4c3)ccc2-c2cccc3cccc1c23 ; c1ccc2c(c1)-c1ccccc1C21c2cc(N(c3ccc4c(c3)sc3ccccc34)c3ccc4c5ccccc5n(-c5cccc6ccccc56)c4c3)ccc2-c2cccc3cccc1c23 ; CC1(C)c2ccccc2-c2c(-n3c4ccccc4c4ccc(N(c5ccccc5)c5ccc6c(c5)C5(c7ccccc7-c7ccccc75)c5cccc7cccc-6c57)cc43)cccc21 ; CC1(C)c2ccccc2-c2cc(-n3c4ccccc4c4ccc(N(c5ccccc5)c5ccc6c(c5)C5(c7ccccc7-c7ccccc75)c5cccc7cccc-6c57)cc43)ccc21]

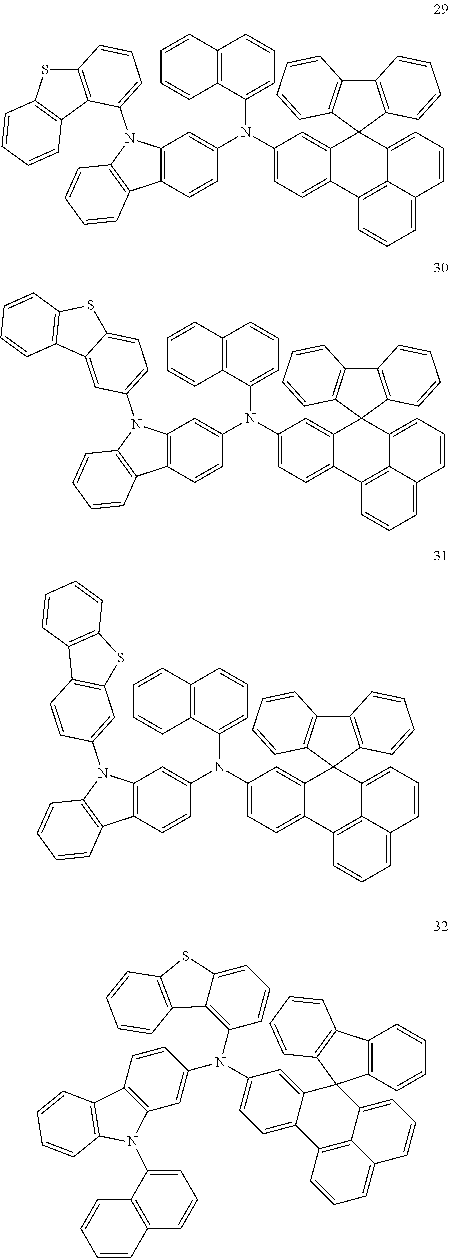 [SMILES: c1ccc2c(c1)-c1ccccc1C21c2cc(N(c3ccc4c5ccccc5n(-c5cccc6sc7ccccc7c56)c4c3)c3cccc4ccccc34)ccc2-c2cccc3cccc1c23 ; c1ccc2c(c1)-c1ccccc1C21c2cc(N(c3ccc4c5ccccc5n(-c5ccc6sc7ccccc7c6c5)c4c3)c3cccc4ccccc34)ccc2-c2cccc3cccc1c23 ; c1ccc2c(c1)-c1ccccc1C21c2cc(N(c3ccc4c5ccccc5n(-c5ccc6c(c5)sc5ccccc56)c4c3)c3cccc4ccccc34)ccc2-c2cccc3cccc1c23 ; c1ccc2c(c1)-c1ccccc1C21c2cc(N(c3ccc4c5ccccc5n(-c5cccc6ccccc56)c4c3)c3cccc4sc5ccccc5c34)ccc2-c2cccc3cccc1c23]

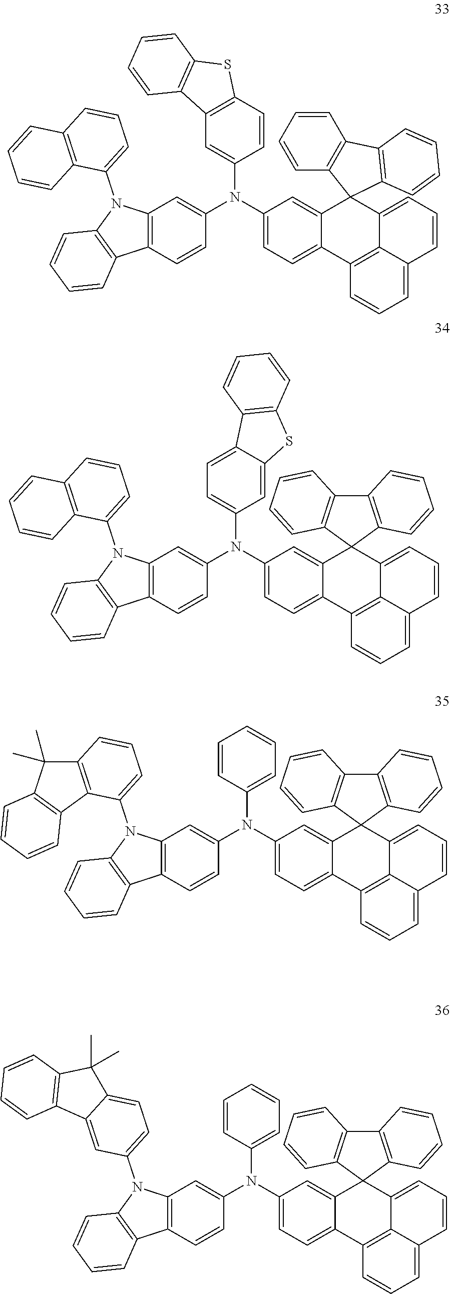 [SMILES: c1ccc2c(c1)-c1ccccc1C21c2cc(N(c3ccc4sc5ccccc5c4c3)c3ccc4c5ccccc5n(-c5cccc6ccccc56)c4c3)ccc2-c2cccc3cccc1c23 ; c1ccc2c(c1)-c1ccccc1C21c2cc(N(c3ccc4c(c3)sc3ccccc34)c3ccc4c5ccccc5n(-c5cccc6ccccc56)c4c3)ccc2-c2cccc3cccc1c23 ; CC1(C)c2ccccc2-c2c(-n3c4ccccc4c4ccc(N(c5ccccc5)c5ccc6c(c5)C5(c7ccccc7-c7ccccc75)c5cccc7cccc-6c57)cc43)cccc21 ; CC1(C)c2ccccc2-c2cc(-n3c4ccccc4c4ccc(N(c5ccccc5)c5ccc6c(c5)C5(c7ccccc7-c7ccccc75)c5cccc7cccc-6c57)cc43)ccc21]

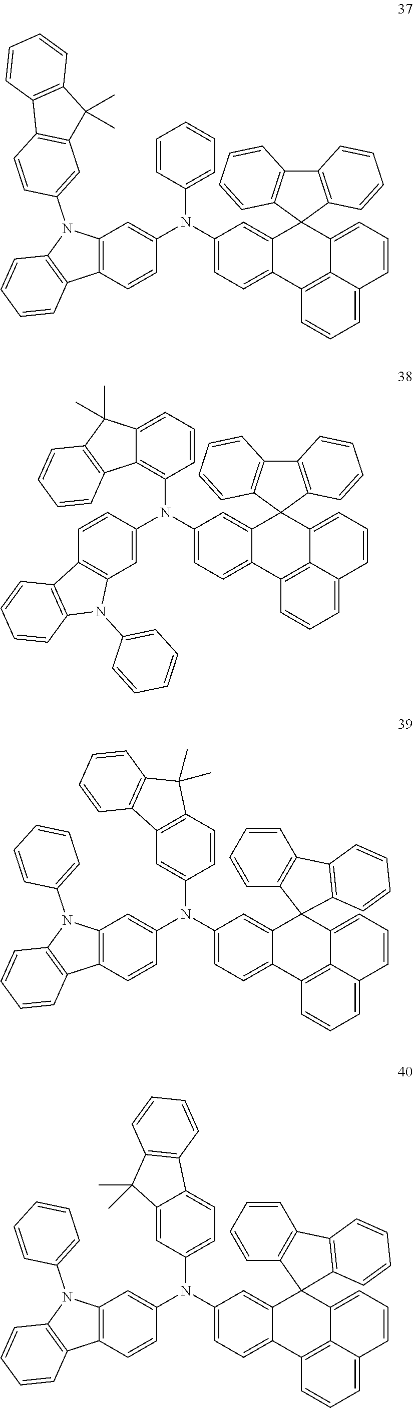 [SMILES: CC1(C)c2ccccc2-c2ccc(-n3c4ccccc4c4ccc(N(c5ccccc5)c5ccc6c(c5)C5(c7ccccc7-c7ccccc75)c5cccc7cccc-6c57)cc43)cc21 ; CC1(C)c2ccccc2-c2c(N(c3ccc4c(c3)C3(c5ccccc5-c5ccccc53)c3cccc5cccc-4c35)c3ccc4c5ccccc5n(-c5ccccc5)c4c3)cccc21 ; CC1(C)c2ccccc2-c2cc(N(c3ccc4c(c3)C3(c5ccccc5-c5ccccc53)c3cccc5cccc-4c35)c3ccc4c5ccccc5n(-c5ccccc5)c4c3)ccc21 ; CC1(C)c2ccccc2-c2ccc(N(c3ccc4c(c3)C3(c5ccccc5-c5ccccc53)c3cccc5cccc-4c35)c3ccc4c5ccccc5n(-c5ccccc5)c4c3)cc21]

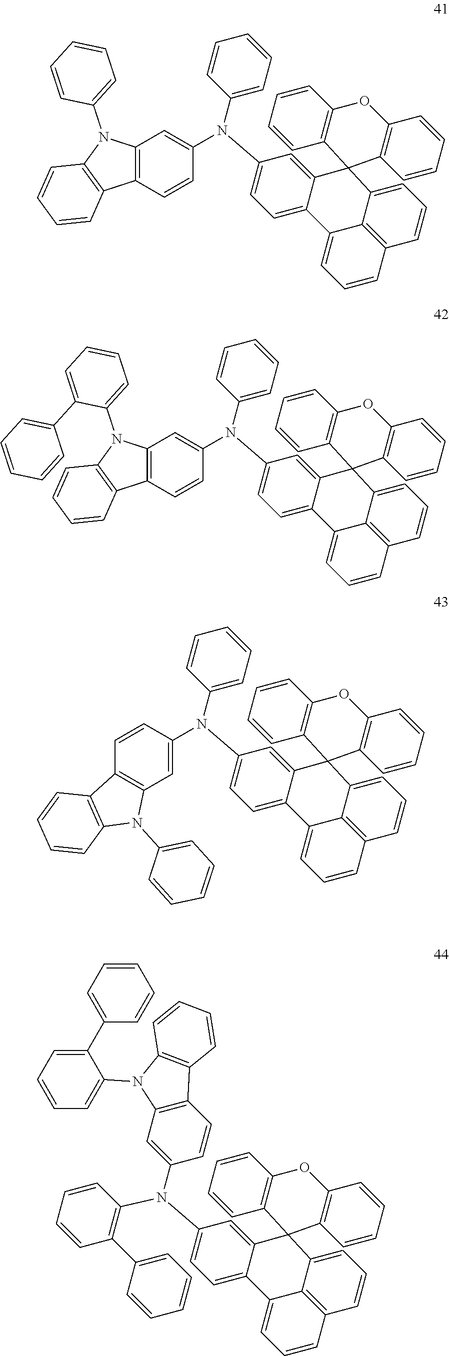 [SMILES: c1ccc(N(c2ccc3c(c2)C2(c4ccccc4Oc4ccccc42)c2cccc4cccc-3c24)c2ccc3c4ccccc4n(-c4ccccc4)c3c2)cc1 ; c1ccc(-c2ccccc2-n2c3ccccc3c3ccc(N(c4ccccc4)c4ccc5c(c4)C4(c6ccccc6Oc6ccccc64)c4cccc6cccc-5c46)cc32)cc1 ; c1ccc(N(c2ccc3c(c2)C2(c4ccccc4Oc4ccccc42)c2cccc4cccc-3c24)c2ccc3c4ccccc4n(-c4ccccc4)c3c2)cc1 ; c1ccc(-c2ccccc2N(c2ccc3c(c2)C2(c4ccccc4Oc4ccccc42)c2cccc4cccc-3c24)c2ccc3c4ccccc4n(-c4ccccc4-c4ccccc4)c3c2)cc1]

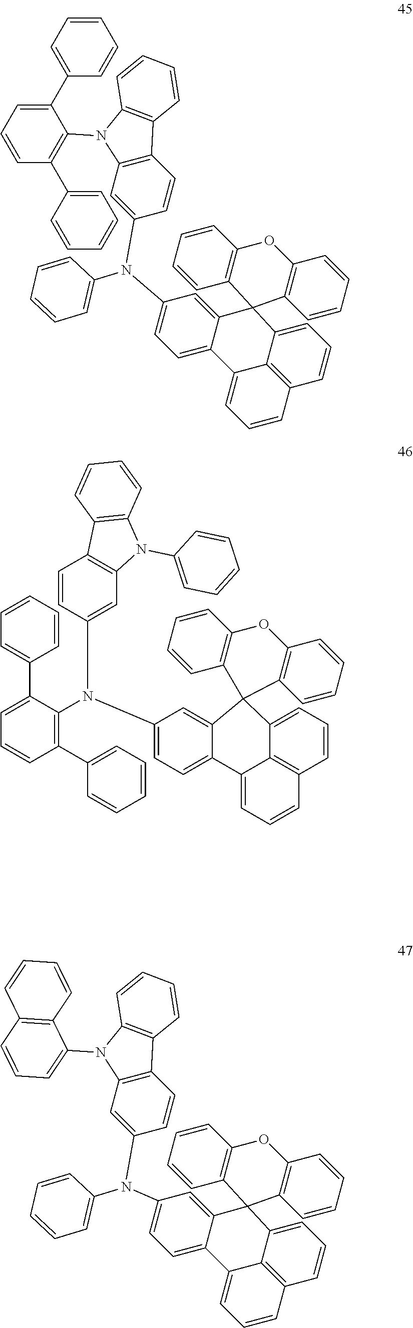 [SMILES: c1ccc(-c2cccc(-c3ccccc3)c2-n2c3ccccc3c3ccc(N(c4ccccc4)c4ccc5c(c4)C4(c6ccccc6Oc6ccccc64)c4cccc6cccc-5c46)cc32)cc1 ; c1ccc(-c2cccc(-c3ccccc3)c2N(c2ccc3c(c2)C2(c4ccccc4Oc4ccccc42)c2cccc4cccc-3c24)c2ccc3c4ccccc4n(-c4ccccc4)c3c2)cc1 ; c1ccc(N(c2ccc3c(c2)C2(c4ccccc4Oc4ccccc42)c2cccc4cccc-3c24)c2ccc3c4ccccc4n(-c4cccc5ccccc45)c3c2)cc1]

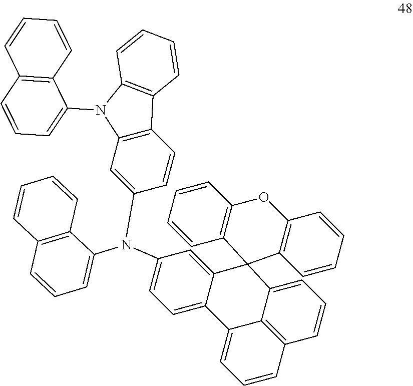 [SMILES: c1ccc2c(c1)Oc1ccccc1C21c2cc(N(c3ccc4c5ccccc5n(-c5cccc6ccccc56)c4c3)c3cccc4ccccc34)ccc2-c2cccc3cccc1c23]

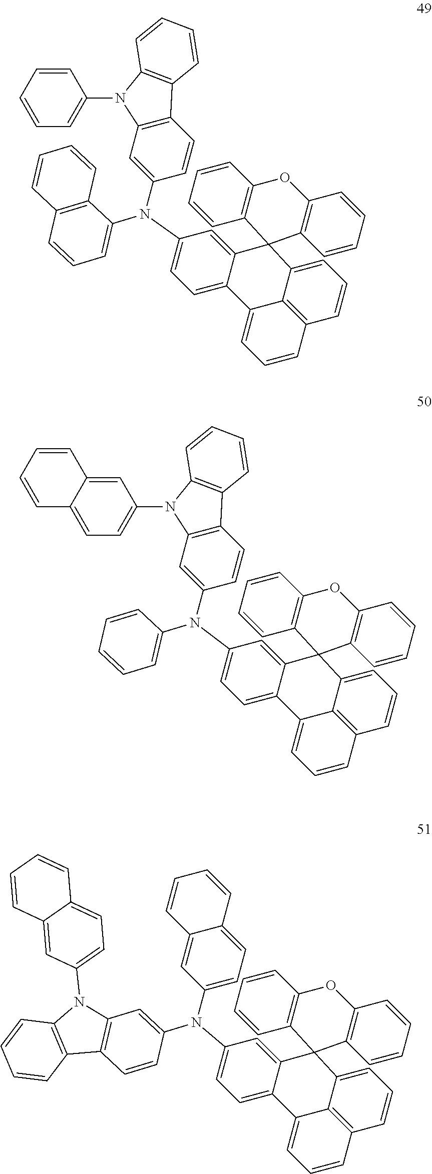 [SMILES: c1ccc(-n2c3ccccc3c3ccc(N(c4ccc5c(c4)C4(c6ccccc6Oc6ccccc64)c4cccc6cccc-5c46)c4cccc5ccccc45)cc32)cc1 ; c1ccc(N(c2ccc3c(c2)C2(c4ccccc4Oc4ccccc42)c2cccc4cccc-3c24)c2ccc3c4ccccc4n(-c4ccc5ccccc5c4)c3c2)cc1 ; c1ccc2c(c1)Oc1ccccc1C21c2cc(N(c3ccc4ccccc4c3)c3ccc4c5ccccc5n(-c5ccc6ccccc6c5)c4c3)ccc2-c2cccc3cccc1c23]

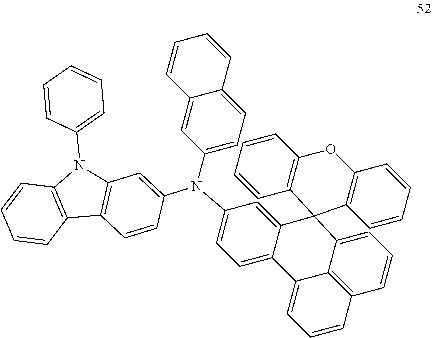 [SMILES: c1ccc(-n2c3ccccc3c3ccc(N(c4ccc5c(c4)C4(c6ccccc6Oc6ccccc64)c4cccc6cccc-5c46)c4ccc5ccccc5c4)cc32)cc1]

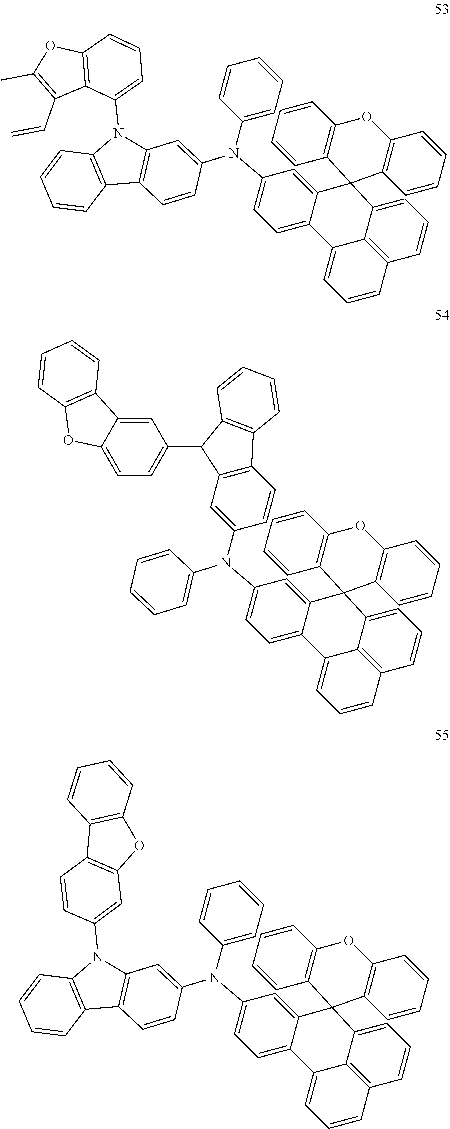 [SMILES: C=Cc1c(C)oc2cccc(-n3c4ccccc4c4ccc(N(c5ccccc5)c5ccc6c(c5)C5(c7ccccc7Oc7ccccc75)c5cccc7cccc-6c57)cc43)c12 ; c1ccc(N(c2ccc3c(c2)C(c2ccc4oc5ccccc5c4c2)c2ccccc2-3)c2ccc3c(c2)C2(c4ccccc4Oc4ccccc42)c2cccc4cccc-3c24)cc1 ; c1ccc(N(c2ccc3c(c2)C2(c4ccccc4Oc4ccccc42)c2cccc4cccc-3c24)c2ccc3c4ccccc4n(-c4ccc5c(c4)oc4ccccc45)c3c2)cc1]

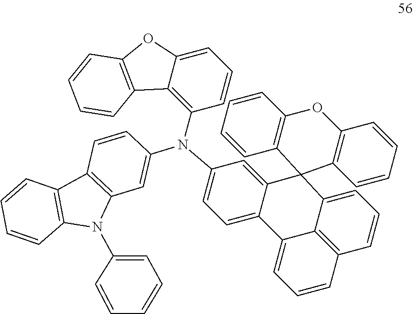 [SMILES: c1ccc(-n2c3ccccc3c3ccc(N(c4ccc5c(c4)C4(c6ccccc6Oc6ccccc64)c4cccc6cccc-5c46)c4cccc5oc6ccccc6c45)cc32)cc1]

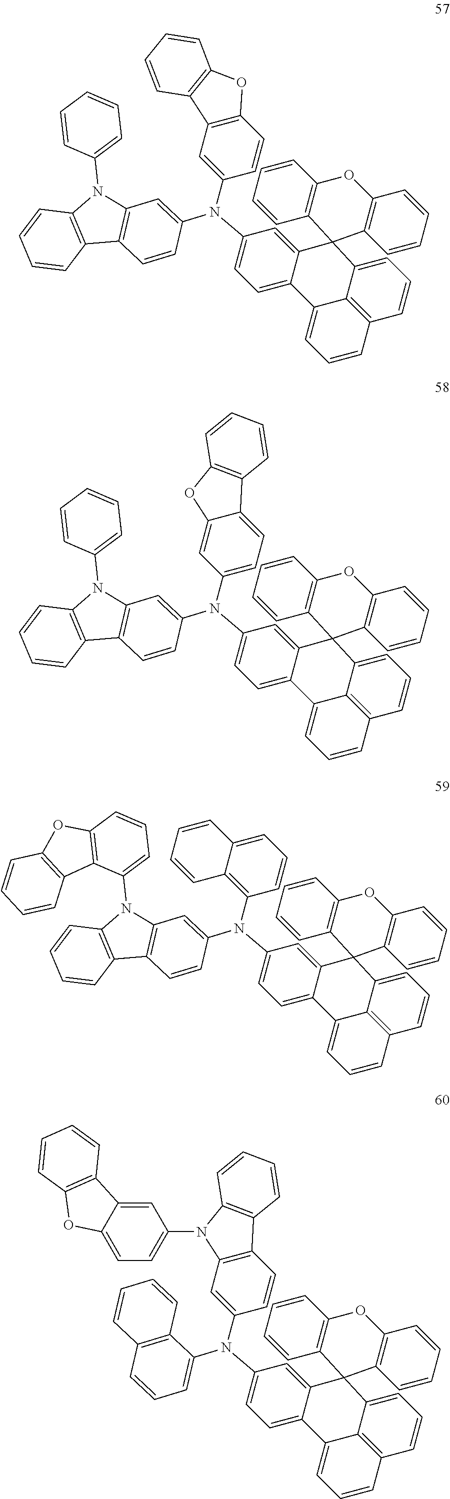 [SMILES: c1ccc(-n2c3ccccc3c3ccc(N(c4ccc5c(c4)C4(c6ccccc6Oc6ccccc64)c4cccc6cccc-5c46)c4ccc5oc6ccccc6c5c4)cc32)cc1 ; c1ccc(-n2c3ccccc3c3ccc(N(c4ccc5c(c4)C4(c6ccccc6Oc6ccccc64)c4cccc6cccc-5c46)c4ccc5c(c4)oc4ccccc45)cc32)cc1 ; c1ccc2c(c1)Oc1ccccc1C21c2cc(N(c3ccc4c5ccccc5n(-c5cccc6oc7ccccc7c56)c4c3)c3cccc4ccccc34)ccc2-c2cccc3cccc1c23 ; c1ccc2c(c1)Oc1ccccc1C21c2cc(N(c3ccc4c5ccccc5n(-c5ccc6oc7ccccc7c6c5)c4c3)c3cccc4ccccc34)ccc2-c2cccc3cccc1c23]

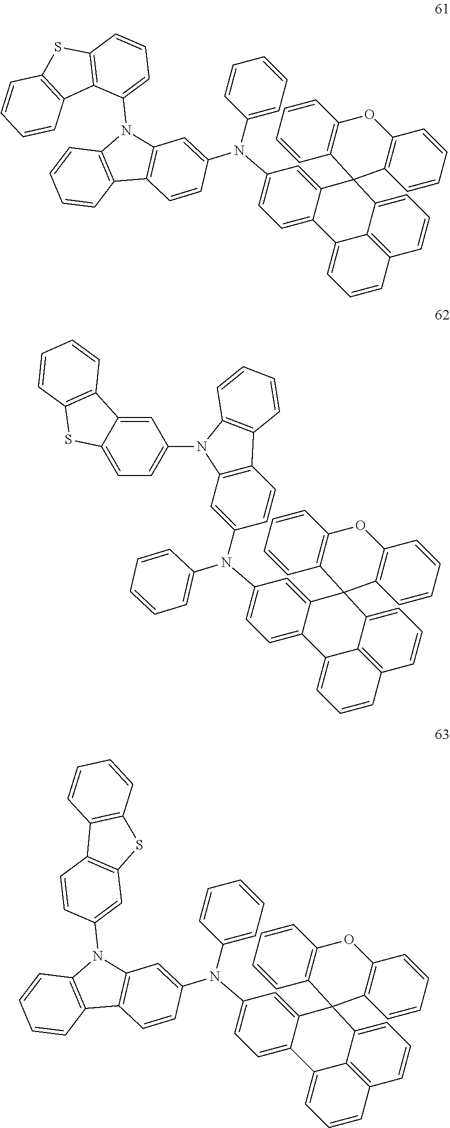 [SMILES: c1ccc(N(c2ccc3c(c2)C2(c4ccccc4Oc4ccccc42)c2cccc4cccc-3c24)c2ccc3c4ccccc4n(-c4cccc5sc6ccccc6c45)c3c2)cc1 ; c1ccc(N(c2ccc3c(c2)C2(c4ccccc4Oc4ccccc42)c2cccc4cccc-3c24)c2ccc3c4ccccc4n(-c4ccc5sc6ccccc6c5c4)c3c2)cc1 ; c1ccc(N(c2ccc3c(c2)C2(c4ccccc4Oc4ccccc42)c2cccc4cccc-3c24)c2ccc3c4ccccc4n(-c4ccc5c(c4)sc4ccccc45)c3c2)cc1]

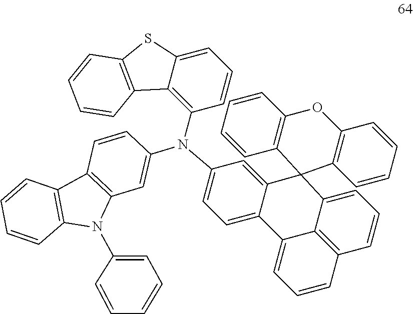 [SMILES: c1ccc(-n2c3ccccc3c3ccc(N(c4ccc5c(c4)C4(c6ccccc6Oc6ccccc64)c4cccc6cccc-5c46)c4cccc5sc6ccccc6c45)cc32)cc1]

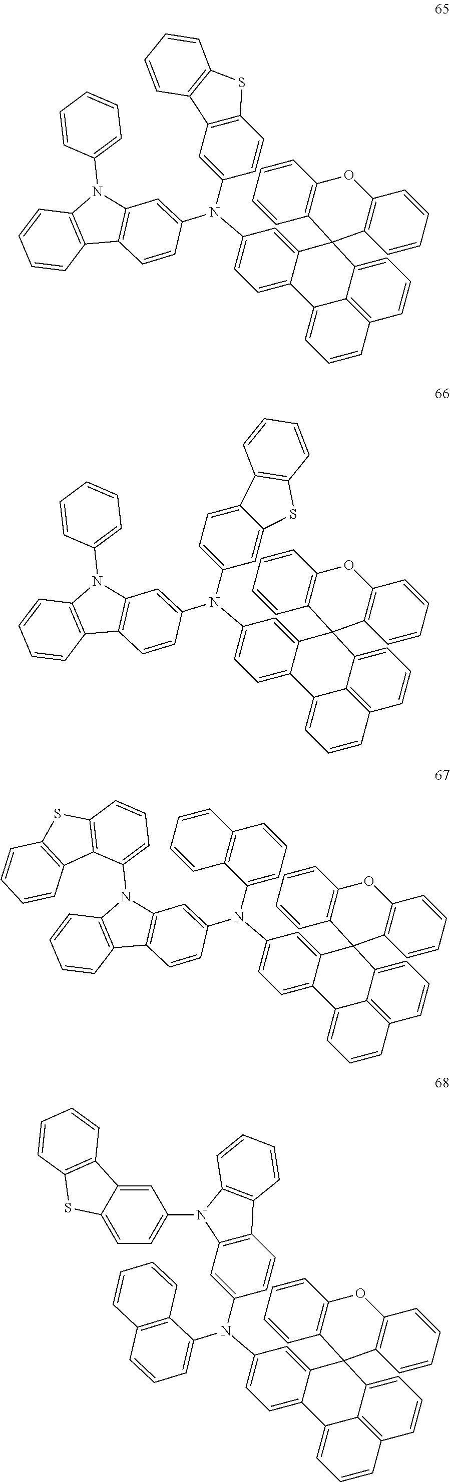 [SMILES: c1ccc(-n2c3ccccc3c3ccc(N(c4ccc5c(c4)C4(c6ccccc6Oc6ccccc64)c4cccc6cccc-5c46)c4ccc5sc6ccccc6c5c4)cc32)cc1 ; c1ccc(-n2c3ccccc3c3ccc(N(c4ccc5c(c4)C4(c6ccccc6Oc6ccccc64)c4cccc6cccc-5c46)c4ccc5c(c4)sc4ccccc45)cc32)cc1 ; c1ccc2c(c1)Oc1ccccc1C21c2cc(N(c3ccc4c5ccccc5n(-c5cccc6sc7ccccc7c56)c4c3)c3cccc4ccccc34)ccc2-c2cccc3cccc1c23 ; c1ccc2c(c1)Oc1ccccc1C21c2cc(N(c3ccc4c5ccccc5n(-c5ccc6sc7ccccc7c6c5)c4c3)c3cccc4ccccc34)ccc2-c2cccc3cccc1c23]

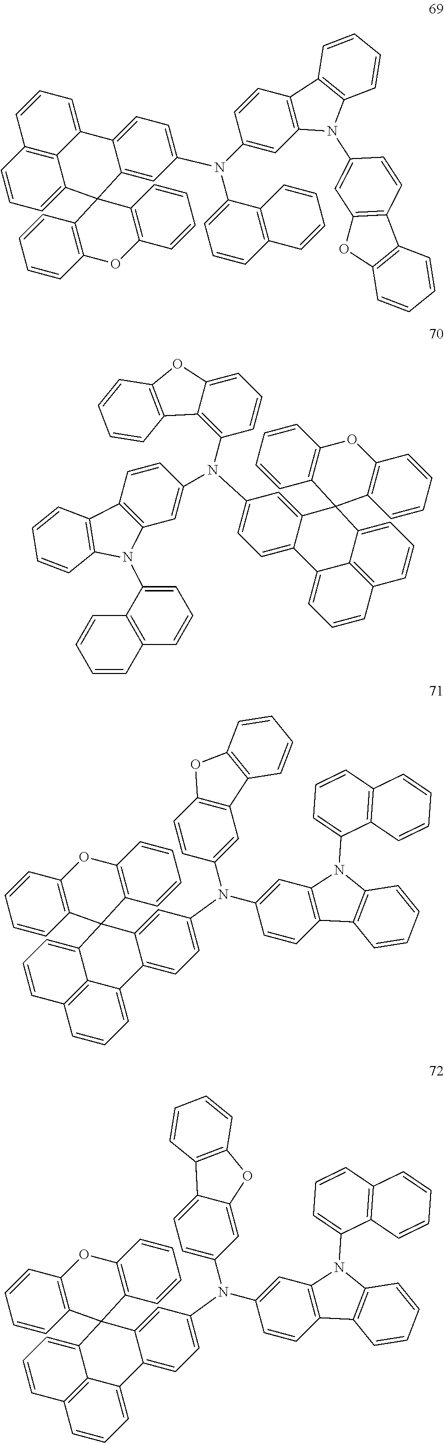 [SMILES: c1ccc2c(c1)Oc1ccccc1C21c2cc(N(c3ccc4c5ccccc5n(-c5ccc6c(c5)oc5ccccc56)c4c3)c3cccc4ccccc34)ccc2-c2cccc3cccc1c23 ; c1ccc2c(c1)Oc1ccccc1C21c2cc(N(c3ccc4c5ccccc5n(-c5cccc6ccccc56)c4c3)c3cccc4oc5ccccc5c34)ccc2-c2cccc3cccc1c23 ; c1ccc2c(c1)Oc1ccccc1C21c2cc(N(c3ccc4oc5ccccc5c4c3)c3ccc4c5ccccc5n(-c5cccc6ccccc56)c4c3)ccc2-c2cccc3cccc1c23 ; c1ccc2c(c1)Oc1ccccc1C21c2cc(N(c3ccc4c(c3)oc3ccccc34)c3ccc4c5ccccc5n(-c5cccc6ccccc56)c4c3)ccc2-c2cccc3cccc1c23]

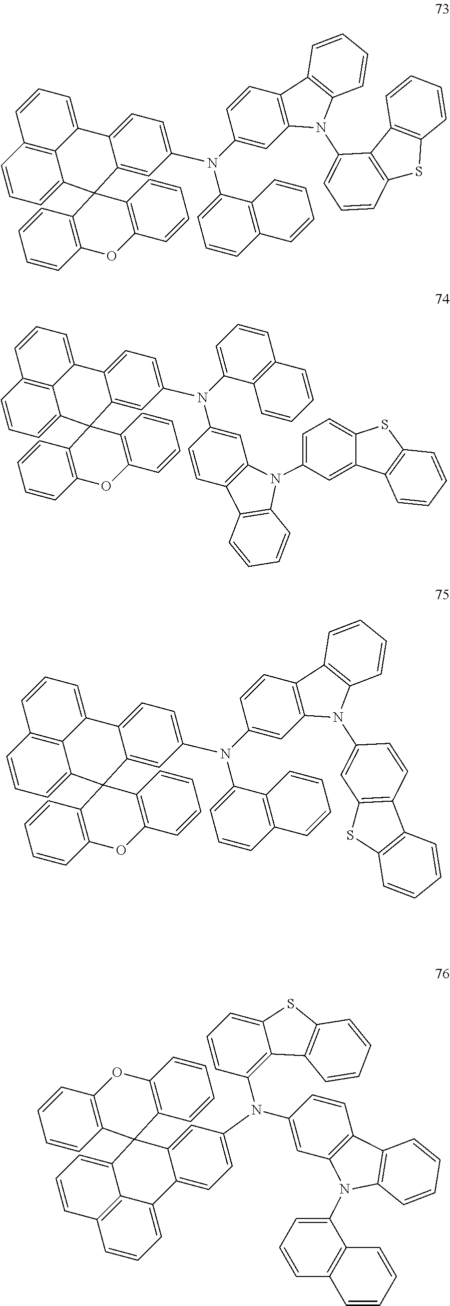 [SMILES: c1ccc2c(c1)Oc1ccccc1C21c2cc(N(c3ccc4c5ccccc5n(-c5cccc6sc7ccccc7c56)c4c3)c3cccc4ccccc34)ccc2-c2cccc3cccc1c23 ; c1ccc2c(c1)Oc1ccccc1C21c2cc(N(c3ccc4c5ccccc5n(-c5ccc6sc7ccccc7c6c5)c4c3)c3cccc4ccccc34)ccc2-c2cccc3cccc1c23 ; c1ccc2c(c1)Oc1ccccc1C21c2cc(N(c3ccc4c5ccccc5n(-c5ccc6c(c5)sc5ccccc56)c4c3)c3cccc4ccccc34)ccc2-c2cccc3cccc1c23 ; c1ccc2c(c1)Oc1ccccc1C21c2cc(N(c3ccc4c5ccccc5n(-c5cccc6ccccc56)c4c3)c3cccc4sc5ccccc5c34)ccc2-c2cccc3cccc1c23]

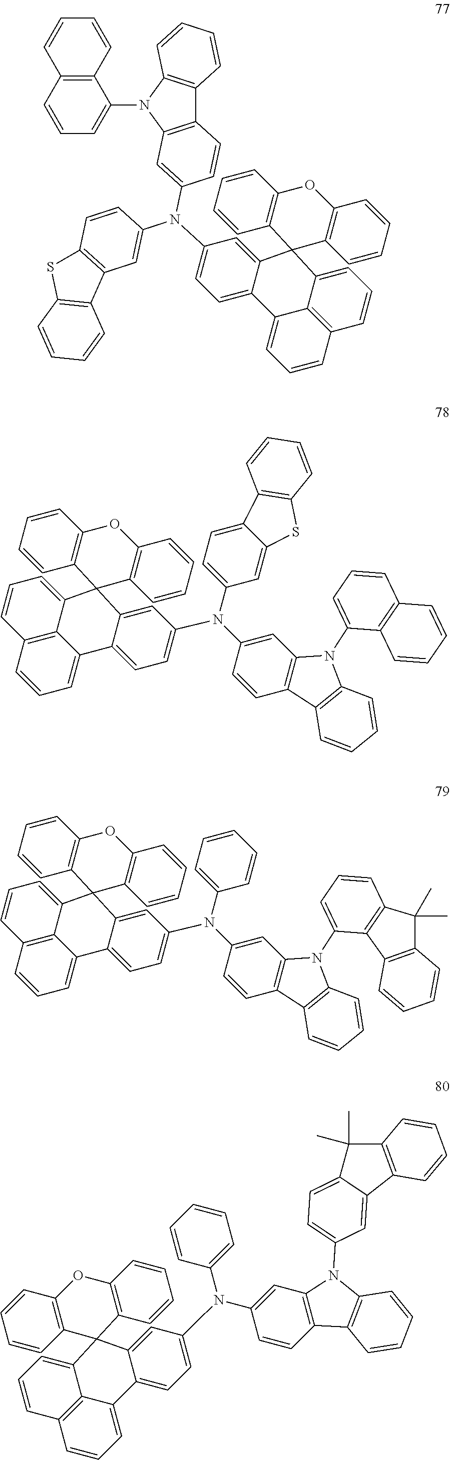 [SMILES: c1ccc2c(c1)Oc1ccccc1C21c2cc(N(c3ccc4sc5ccccc5c4c3)c3ccc4c5ccccc5n(-c5cccc6ccccc56)c4c3)ccc2-c2cccc3cccc1c23 ; c1ccc2c(c1)Oc1ccccc1C21c2cc(N(c3ccc4c(c3)sc3ccccc34)c3ccc4c5ccccc5n(-c5cccc6ccccc56)c4c3)ccc2-c2cccc3cccc1c23 ; CC1(C)c2ccccc2-c2c(-n3c4ccccc4c4ccc(N(c5ccccc5)c5ccc6c(c5)C5(c7ccccc7Oc7ccccc75)c5cccc7cccc-6c57)cc43)cccc21 ; CC1(C)c2ccccc2-c2cc(-n3c4ccccc4c4ccc(N(c5ccccc5)c5ccc6c(c5)C5(c7ccccc7Oc7ccccc75)c5cccc7cccc-6c57)cc43)ccc21]

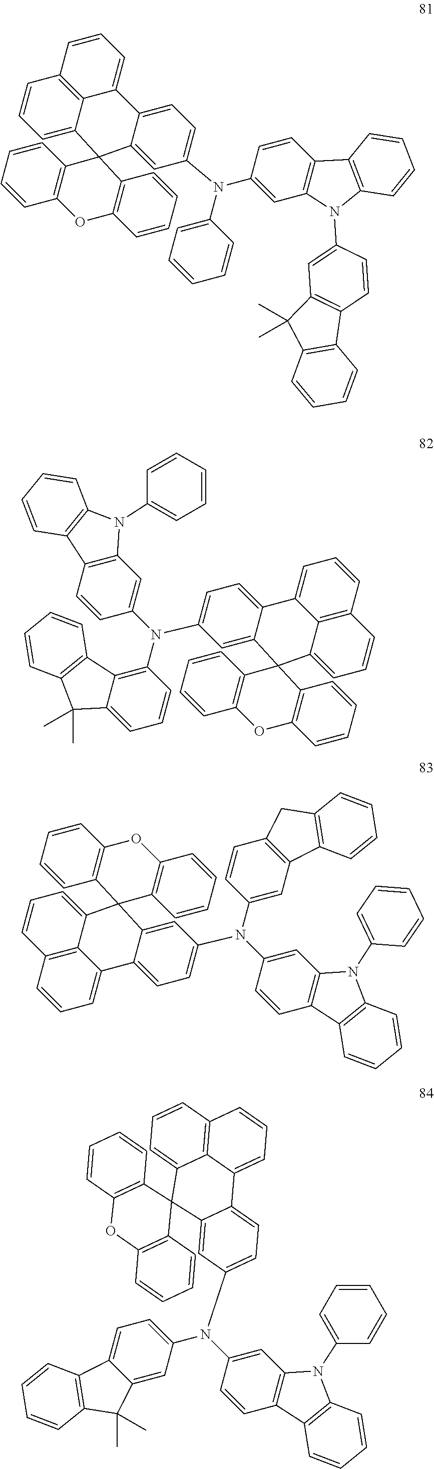 [SMILES: CC1(C)c2ccccc2-c2ccc(-n3c4ccccc4c4ccc(N(c5ccccc5)c5ccc6c(c5)C5(c7ccccc7Oc7ccccc75)c5cccc7cccc-6c57)cc43)cc21 ; CC1(C)c2ccccc2-c2c(N(c3ccc4c(c3)C3(c5ccccc5Oc5ccccc53)c3cccc5cccc-4c35)c3ccc4c5ccccc5n(-c5ccccc5)c4c3)cccc21 ; c1ccc(-n2c3ccccc3c3ccc(N(c4ccc5c(c4)-c4ccccc4C5)c4ccc5c(c4)C4(c6ccccc6Oc6ccccc64)c4cccc6cccc-5c46)cc32)cc1 ; CC1(C)c2ccccc2-c2ccc(N(c3ccc4c(c3)C3(c5ccccc5Oc5ccccc53)c3cccc5cccc-4c35)c3ccc4c5ccccc5n(-c5ccccc5)c4c3)cc21]

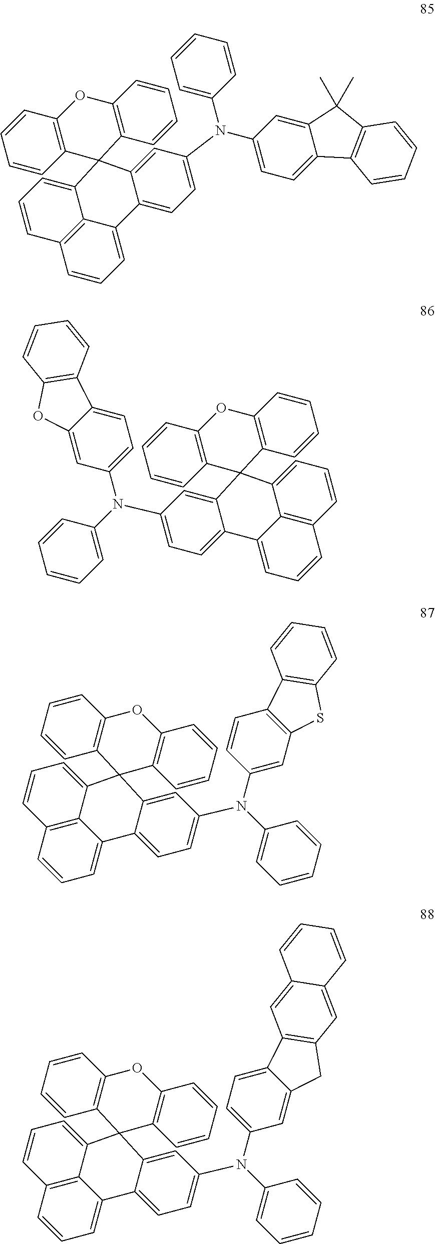

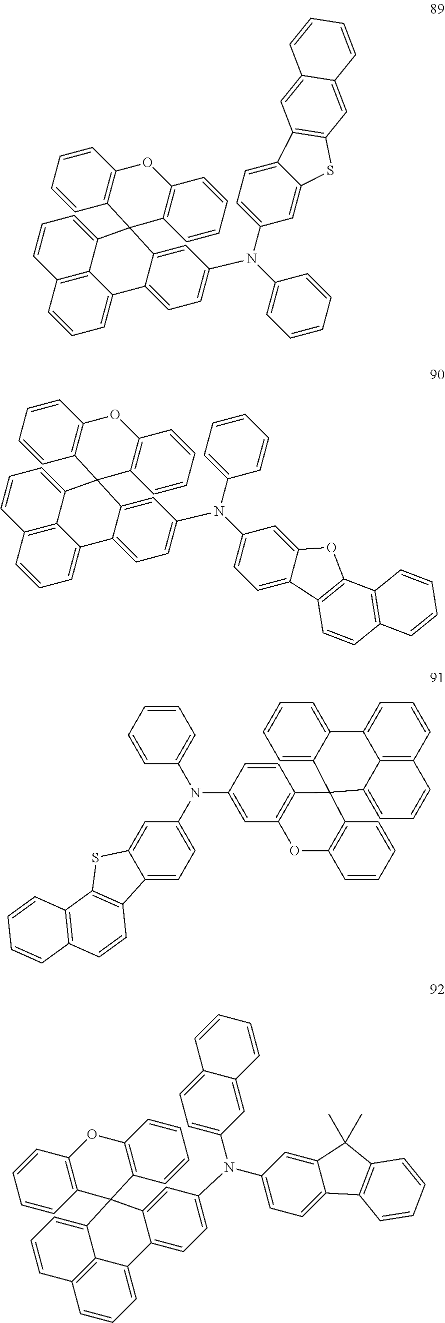 [SMILES: c1ccc(N(c2ccc3c(c2)C2(c4ccccc4Oc4ccccc42)c2cccc4cccc-3c24)c2ccc3c(c2)sc2cc4ccccc4cc23)cc1 ; c1ccc(N(c2ccc3c(c2)C2(c4ccccc4Oc4ccccc42)c2cccc4cccc-3c24)c2ccc3c(c2)oc2c4ccccc4ccc32)cc1 ; c1ccc(N(c2ccc3c(c2)Oc2ccccc2C32c3ccccc3-c3cccc4cccc2c34)c2ccc3c(c2)sc2c4ccccc4ccc32)cc1 ; CC1(C)c2ccccc2-c2ccc(N(c3ccc4c(c3)C3(c5ccccc5Oc5ccccc53)c3cccc5cccc-4c35)c3ccc4ccccc4c3)cc21]

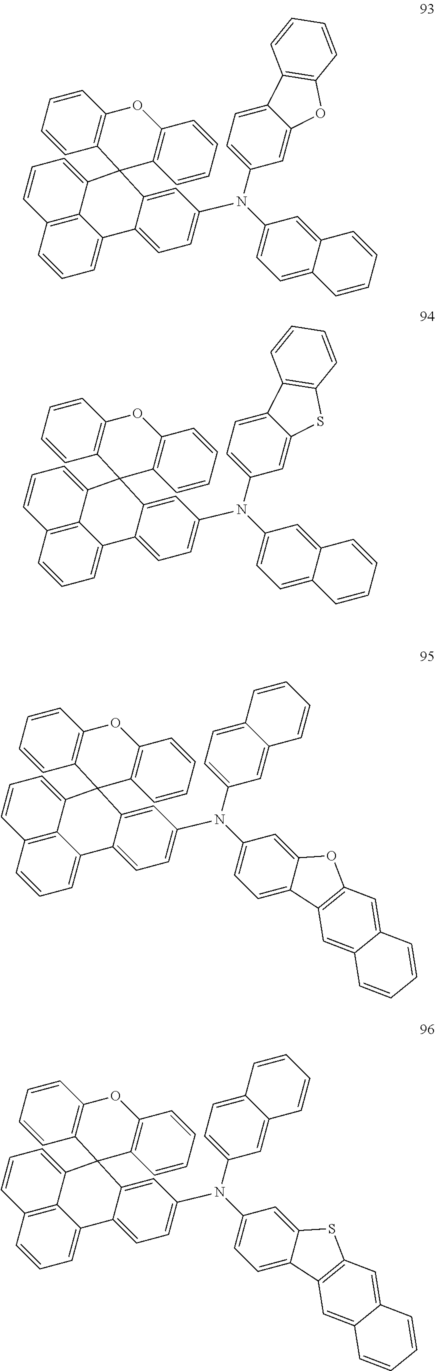

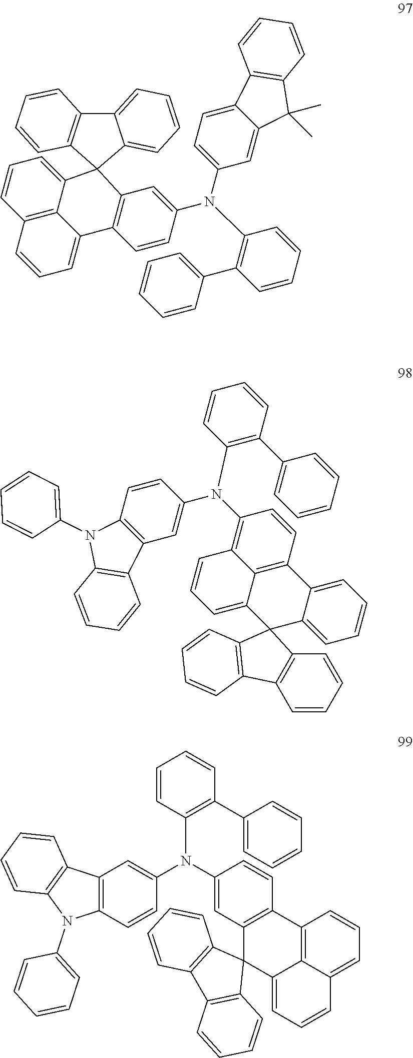 [SMILES: CC1(C)c2ccccc2-c2ccc(N(c3ccc4c(c3)C3(c5ccccc5-c5ccccc53)c3cccc5cccc-4c35)c3ccccc3-c3ccccc3)cc21 ; c1ccc(-c2ccccc2N(c2ccc3c(c2)c2ccccc2n3-c2ccccc2)c2ccc3c4c(cccc24)C2(c4ccccc4-c4ccccc42)c2ccccc2-3)cc1 ; c1ccc(-c2ccccc2N(c2ccc3c(c2)C2(c4ccccc4-c4ccccc42)c2cccc4cccc-3c24)c2ccc3c(c2)c2ccccc2n3-c2ccccc2)cc1]

Since the first electrode of the light-emitting device includes a metal oxide having a work function of 5.3 eV or more, injection of holes may be smooth, such that holes may be efficiently transferred to the emission layer. In addition, since the hole transport region includes the heterocyclic compound represented by Formula 1, an energy barrier may be lowered to improve hole injection and transport characteristics. Accordingly, the light-emitting device may have high efficiency, a low driving voltage, and a long lifespan, and thus may be used for manufacturing a high-quality electronic device.

Synthesis methods of the heterocyclic compound represented by Formula 1 may be recognizable by one of ordinary skill in the art by referring to Synthesis Examples and/or Examples provided below.

In an embodiment, the light-emitting device may include a capping layer located outside the first electrode or outside the second electrode.

In an embodiment, the light-emitting device may further include at least one of a first capping layer located outside the first electrode and a second capping layer located outside the second electrode, and at least one of the first capping layer and the second capping layer may include the heterocyclic compound represented by Formula 1. More details for the first capping layer and/or second capping layer are the same as described in the present specification.

In an embodiment, the light-emitting device may include:

the first capping layer located outside the first electrode and including the heterocyclic compound represented by Formula 1;

the second capping layer located outside the second electrode and including the heterocyclic compound represented by Formula 1; or

the first capping layer and the second capping layer.

The wording “(hole transport region and/or capping layer) includes the heterocyclic compound represented by Formula 1” as used herein may be understood as “(hole transport region and/or capping layer) may include one kind of heterocyclic compound represented by Formula 1 or two different kinds of heterocyclic compounds that are each represented by Formula 1”.

In an embodiment, the hole transport region and/or the capping layer may include Compound 1 only as the heterocyclic compound. In this case, Compound 1 may be present in the hole transport layer of the light-emitting device. In one or more embodiments, the hole transport layer and/or the capping layer may include Compounds 1 and 2 as the heterocyclic compounds. In this regard, Compound 1 and Compound 2 may all exist in an identical layer (for example, Compound 1 and Compound 2 may all exist in a hole transport layer), or different layers (for example, Compound 1 may exist in an electron transport layer and Compound 2 may exist in a buffer layer).

The term “interlayer” as used herein refers to a single layer and/or all of a plurality of layers between the first electrode and the second electrode of the light-emitting device.

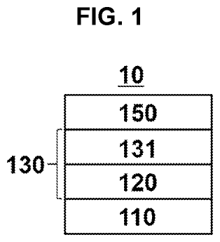

Another aspect of the embodiments described herein provides an electronic apparatus including the light-emitting device. The electronic apparatus may further include a thin-film transistor. For example, the electronic apparatus may further include a thin-film transistor including a source electrode and a drain electrode, wherein the first electrode of the light-emitting device may be electrically connected to the source electrode or the drain electrode. In an embodiment, the electronic apparatus may further include a color filter, a color conversion layer, a touch screen layer, a polarizing layer, or any combination thereof. More details for the electronic apparatus are as described herein.

[Description of FIG. 1]

FIG. 1 is a schematic cross-sectional view of a light-emitting device 10 according to an embodiment that is constructed according to principles of the invention. The light-emitting device 10 includes a first electrode 110, an interlayer 130, a hole transport region 120, an emission layer 131, and a second electrode 150.

Hereinafter, the structure of the light-emitting device 10 according to an embodiment and a method of manufacturing the light-emitting device 10 will be described with reference to FIG. 1.

[First Electrode 110]

The first electrode of the light-emitting device may include a metal oxide having a work function of 5.3 eV or more.

The first electrode may further include the conductive oxide material, the metal material, or the metal alloy material.

In FIG. 1, a substrate may be additionally arranged under the first electrode 110 or above the second electrode 150. In an embodiment, the substrate may be a glass substrate or a plastic substrate. In one or more embodiments, the substrate may be a flexible substrate, and for example, may include plastics with excellent heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing or sputtering a material for forming the first electrode 110 on the substrate. When the first electrode 110 is an anode, a material for forming the first electrode 110 may be a high-work function material that facilitates injection of holes.

The first electrode 110 may be a reflective electrode, a semi-transmissive electrode, or a transmissive electrode. In an embodiment, when the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In one or more embodiments, when the first electrode 110 is a semi-transmissive electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a single-layered structure consisting of a single layer or a multi-layered structure including a plurality of layers. For example, the first electrode 110 may have a three-layered structure of ITO/Ag/ITO.

[Interlayer 130]

The interlayer 130 is on the first electrode 110. The interlayer 130 may include an emission layer 131.

The interlayer 130 may further include a hole transport region 120 arranged between the first electrode 110 and the emission layer 131 and an electron transport region arranged between the emission layer 131 and the second electrode 150.

In an embodiment, the interlayer 130 may further include, in addition to various organic materials, a metal-containing compound such as an organometallic compound, an inorganic material such as a quantum dot, or the like.

In one or more embodiments, the interlayer 130 may further include: i) two or more emitting units sequentially stacked between the first electrode 110 and the second electrode 150; and ii) a charge generation layer between the two or more emitting units. When the interlayer 130 includes the two or more emitting units and the charge generation layer, the light-emitting device 10 may be a tandem light-emitting device.

[Hole Transport Region 120 in Interlayer 130]

The hole transport region 120 may have i) a single-layered structure consisting of a single layer consisting of a single material, ii) a single-layered structure consisting of a single layer consisting of a plurality of different materials, or iii) a multi-layered structure including a plurality of layers including different materials.

The hole transport region 120 may include a hole injection layer, a hole transport layer, an emission auxiliary layer, an electron blocking layer, or any combination thereof.

For example, the hole transport region 120 may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, wherein layers of each structure are sequentially stacked from the first electrode 110.

The hole transport region 120 may include the heterocyclic compound represented by Formula 1.

In an embodiment, the hole transport layer may include the heterocyclic compound represented by Formula 1.

The hole transport region 120 may further include a compound represented by Formula 201, a compound represented by Formula 202, or any combination thereof:

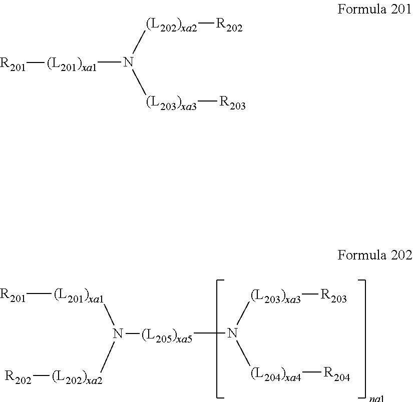

wherein, in Formulae 201 and 202,

L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,

xa1 to xa4 may each independently be an integer from 0 to 5,

xa5 may be an integer from 1 to 10,

R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group (for example, a carbazole group or the like) unsubstituted or substituted with at least one R₁₀ₐ (for example, Compound HT16),

R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group unsubstituted or substituted with at least one R₁₀ₐ, and

na1 may be an integer from 1 to 4.

For example, each of Formulae 201 and 202 may include at least one of groups represented by Formulae CY201 to CY217:

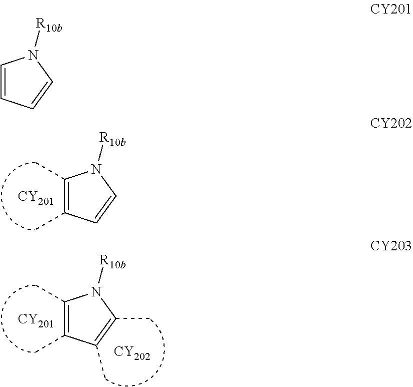

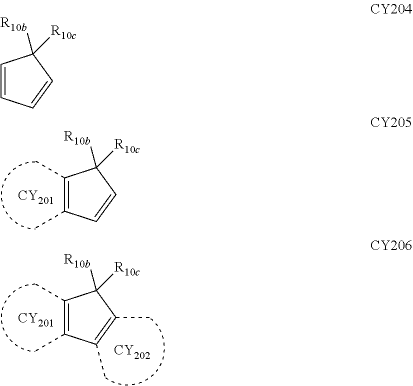

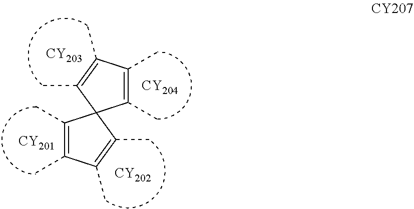

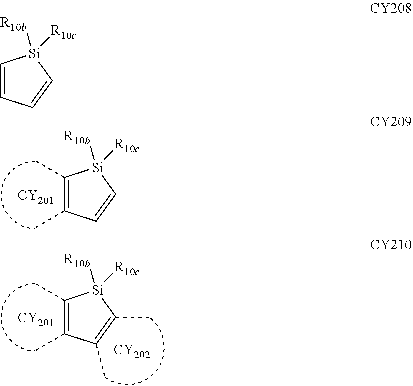

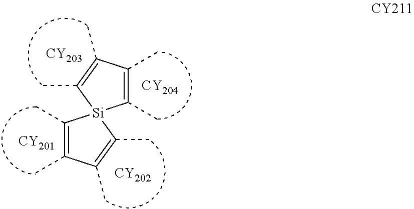

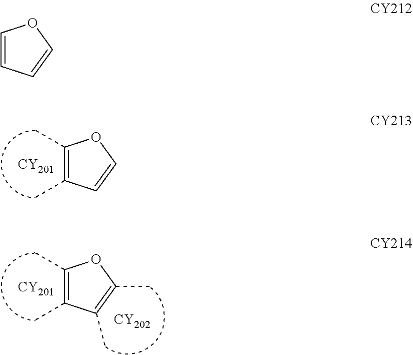

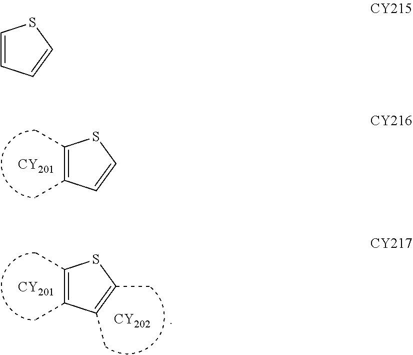

In Formulae CY201 to CY217, R_{10b} and R_{10c}, may respectively be the same as described in connection with R₁₀ₐ, ring CY201 to ring CY204 may each independently be a C₃-C₂₀ carbocyclic group or a C₁-C₂₀ heterocyclic group, and at least one hydrogen in each of Formulae CY201 to CY217 may be unsubstituted or substituted with R₁₀ₐ.

In an embodiment, in Formulae CY201 to CY217, ring CY201 to ring CY204 may each independently be a benzene group, a naphthalene group, a phenanthrene group, or an anthracene group.

In one or more embodiments, each of Formulae 201 and 202 may include at least one of groups represented by Formulae CY201 to CY203.

In one or more embodiments, Formula 201 may include at least one of groups represented by Formulae CY201 to CY203 and at least one of groups represented by Formulae CY204 to CY217.

In one or more embodiments, in Formula 201, xa1 may be 1, R₂₀₁ may be a group represented by one of Formulae CY201 to CY203, xa2 may be 0, and R₂₀₂ may be a group represented by one of Formulae CY204 to CY207.

In one or more embodiments, each of Formulae 201 and 202 may not include a group represented by one of Formulae CY201 to CY203.

In one or more embodiments, each of Formulae 201 and 202 may not include a group represented by one of Formulae CY201 to CY203, and may include at least one of groups represented by Formulae CY204 to CY217.

In one or more embodiments, each of Formulae 201 and 202 may not include a group represented by one of Formulae CY201 to CY217.

For example, the hole transport region may include one of Compounds HT1 to HT46, m-MTDATA, TDATA, 2-TNATA, NPB(NPD), β-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or any combination thereof:

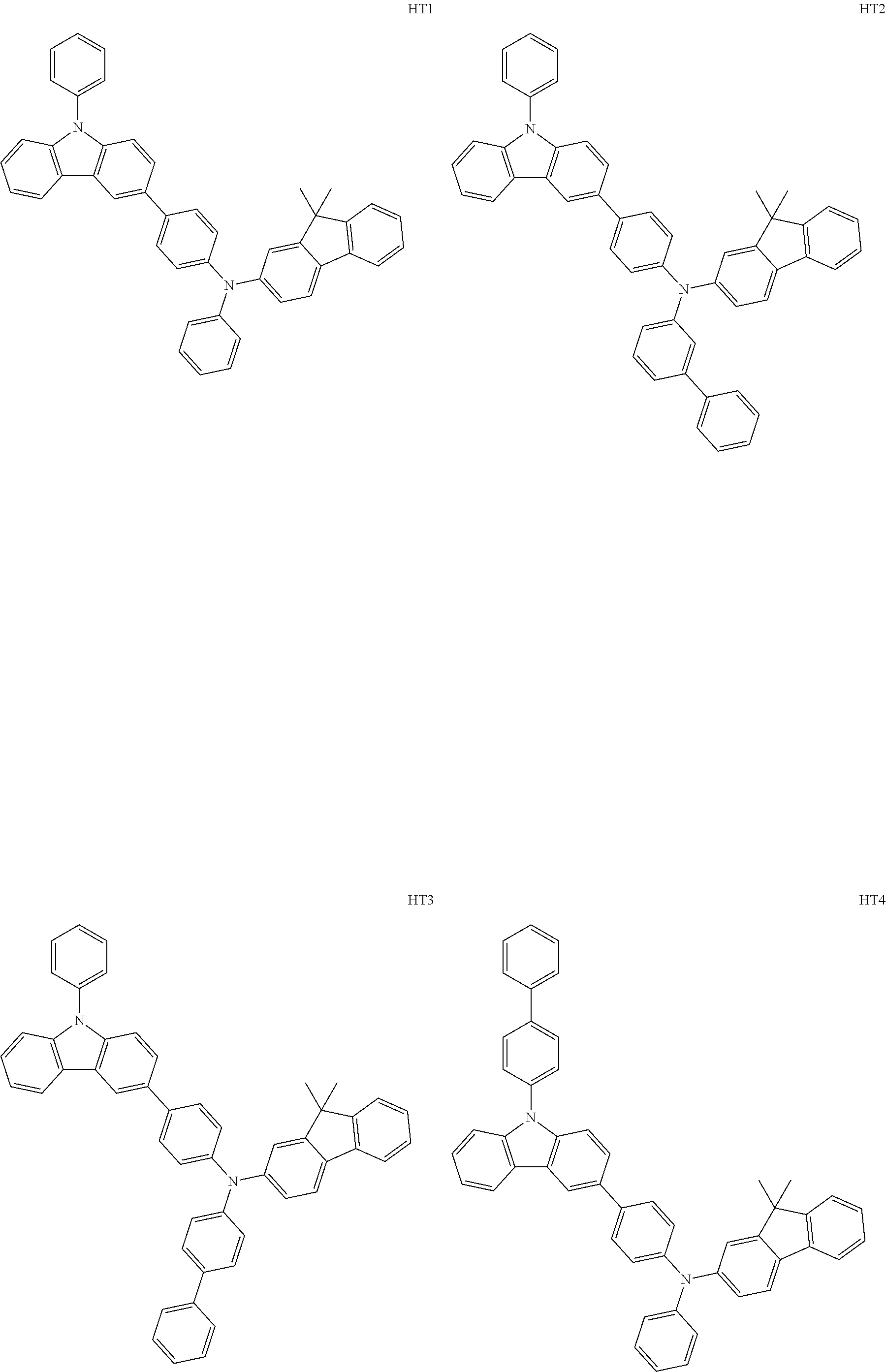

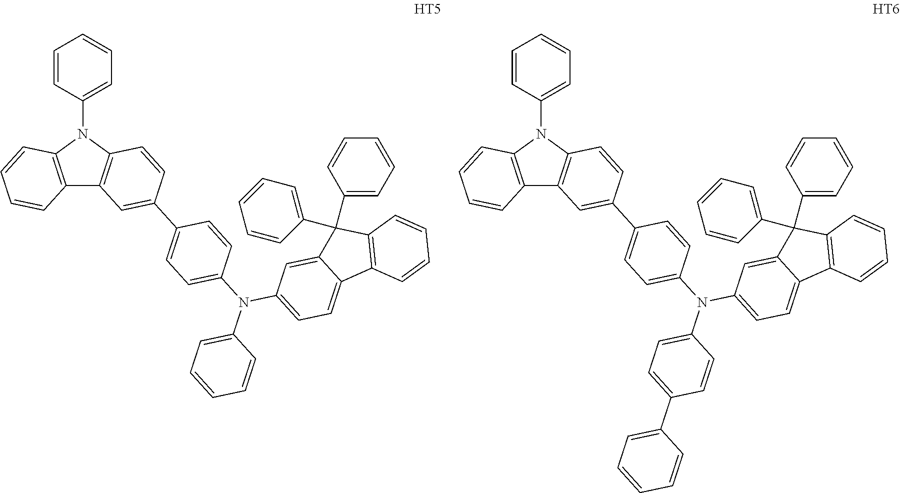 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

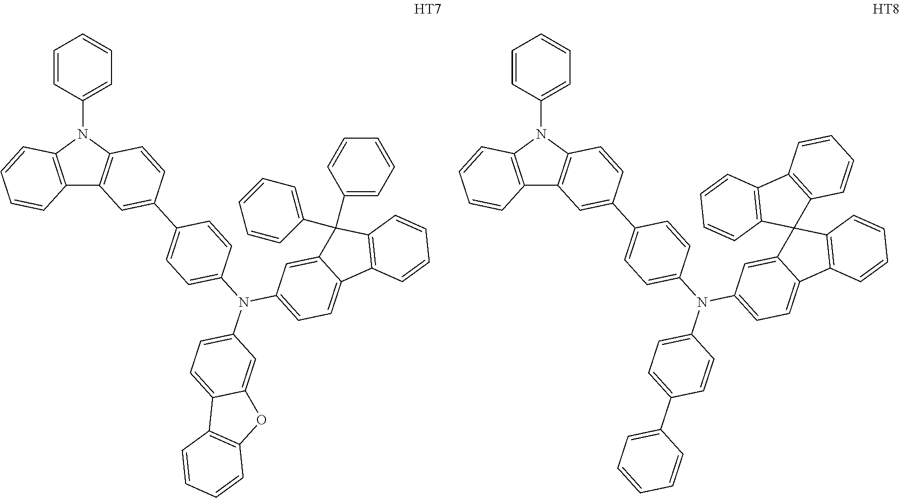 [SMILES: c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

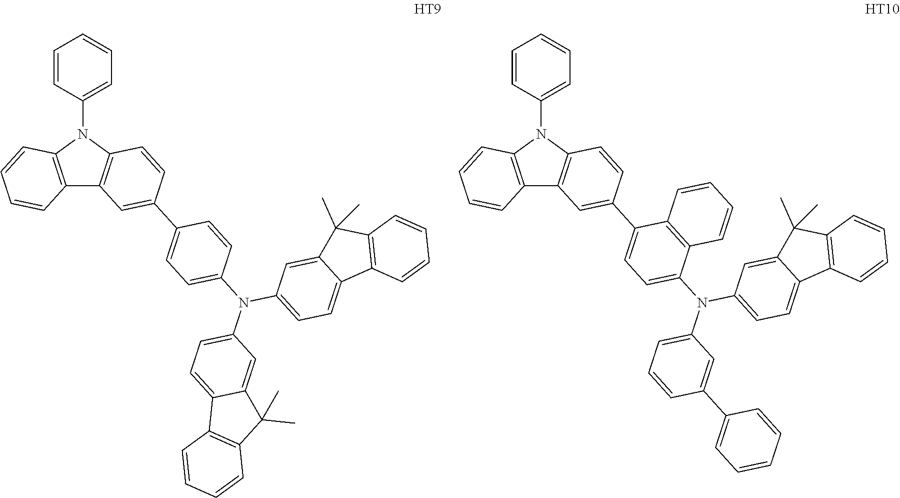 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21]

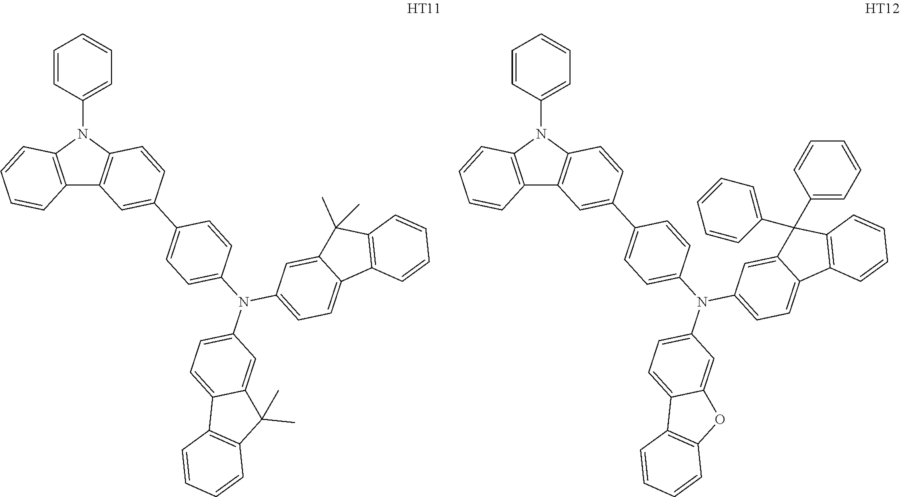 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1]

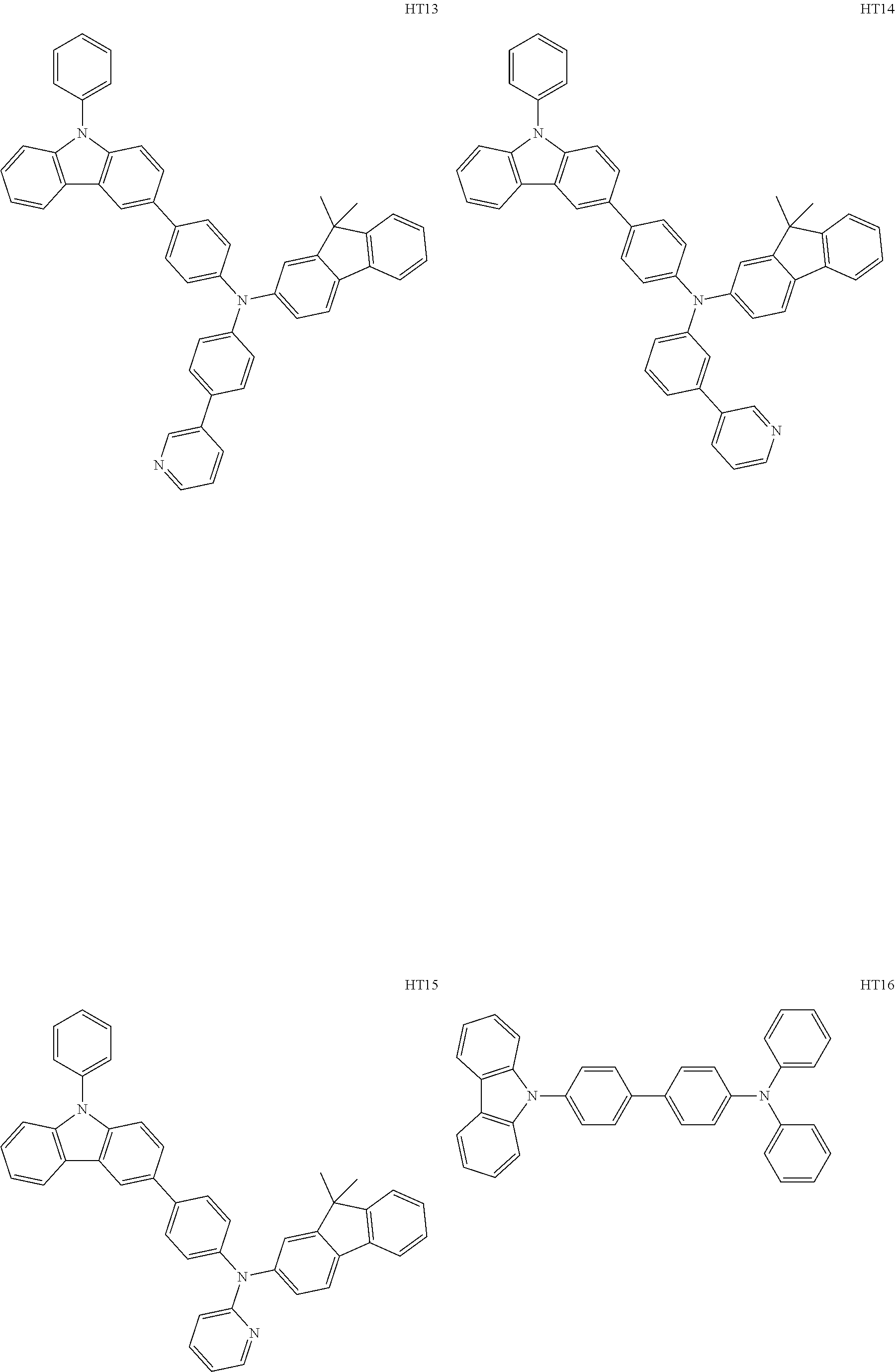 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1]

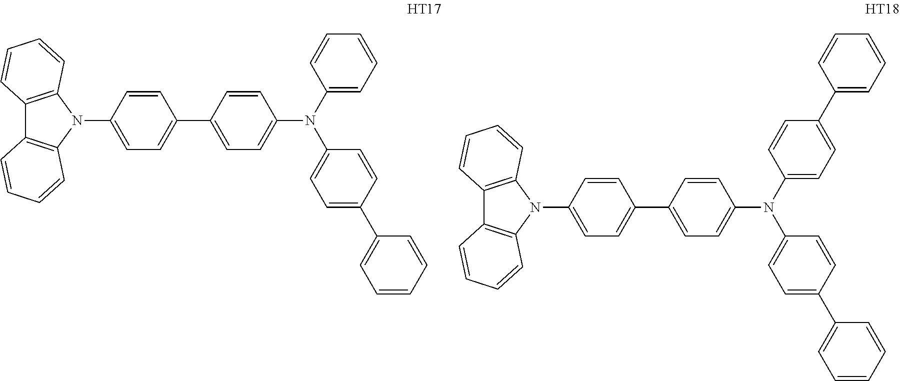

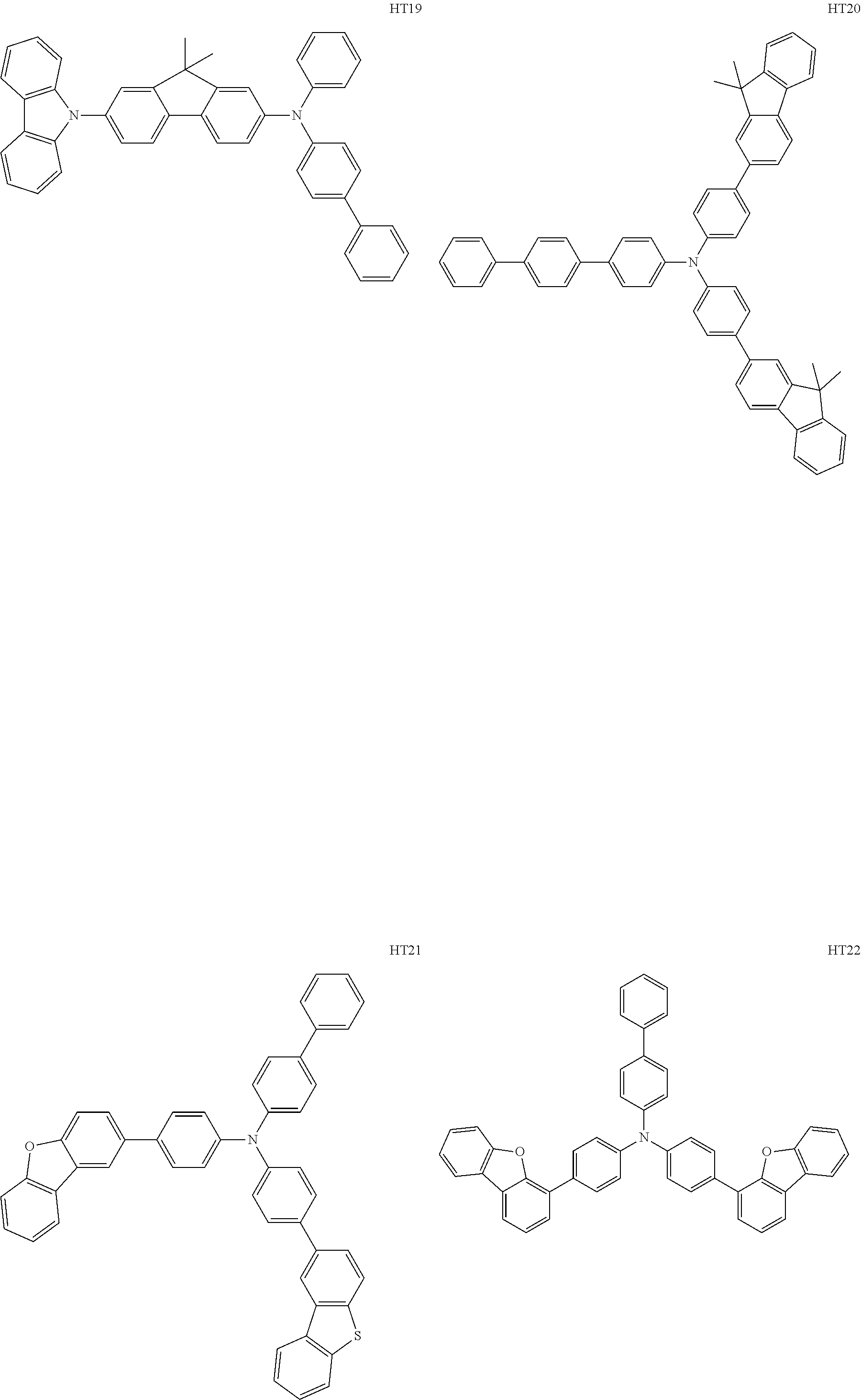

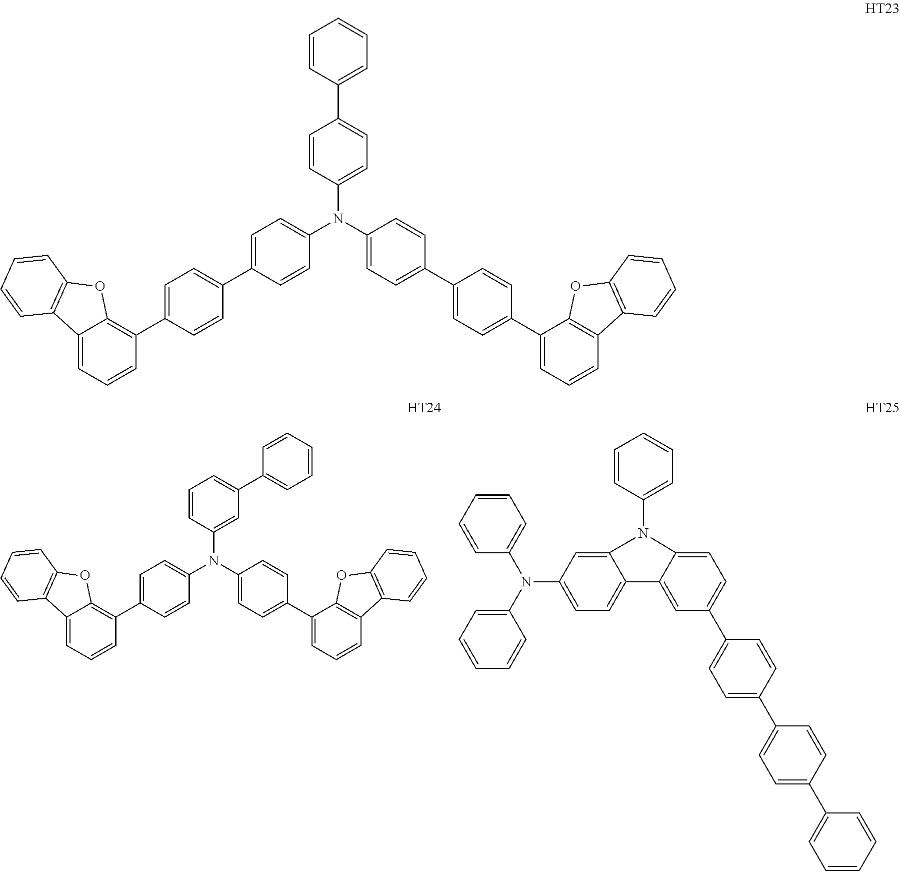 [SMILES: c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1 ; c1ccc(-c2cccc(N(c3ccc(-c4cccc5c4oc4ccccc45)cc3)c3ccc(-c4cccc5c4oc4ccccc45)cc3)c2)cc1 ; c1ccc(-c2ccc(-c3ccc(-c4ccc5c(c4)c4ccc(N(c6ccccc6)c6ccccc6)cc4n5-c4ccccc4)cc3)cc2)cc1]

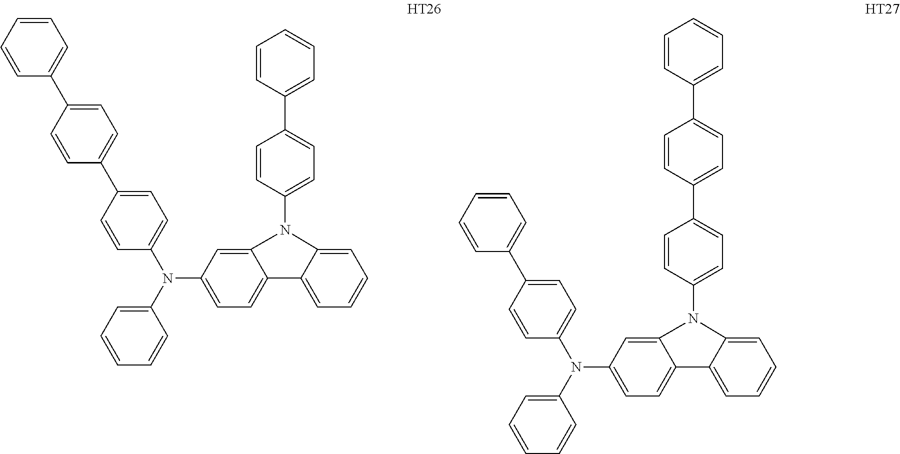 [SMILES: c1ccc(-c2ccc(-c3ccc(N(c4ccccc4)c4ccc5c6ccccc6n(-c6ccc(-c7ccccc7)cc6)c5c4)cc3)cc2)cc1 ; c1ccc(-c2ccc(-c3ccc(-n4c5ccccc5c5ccc(N(c6ccccc6)c6ccc(-c7ccccc7)cc6)cc54)cc3)cc2)cc1]

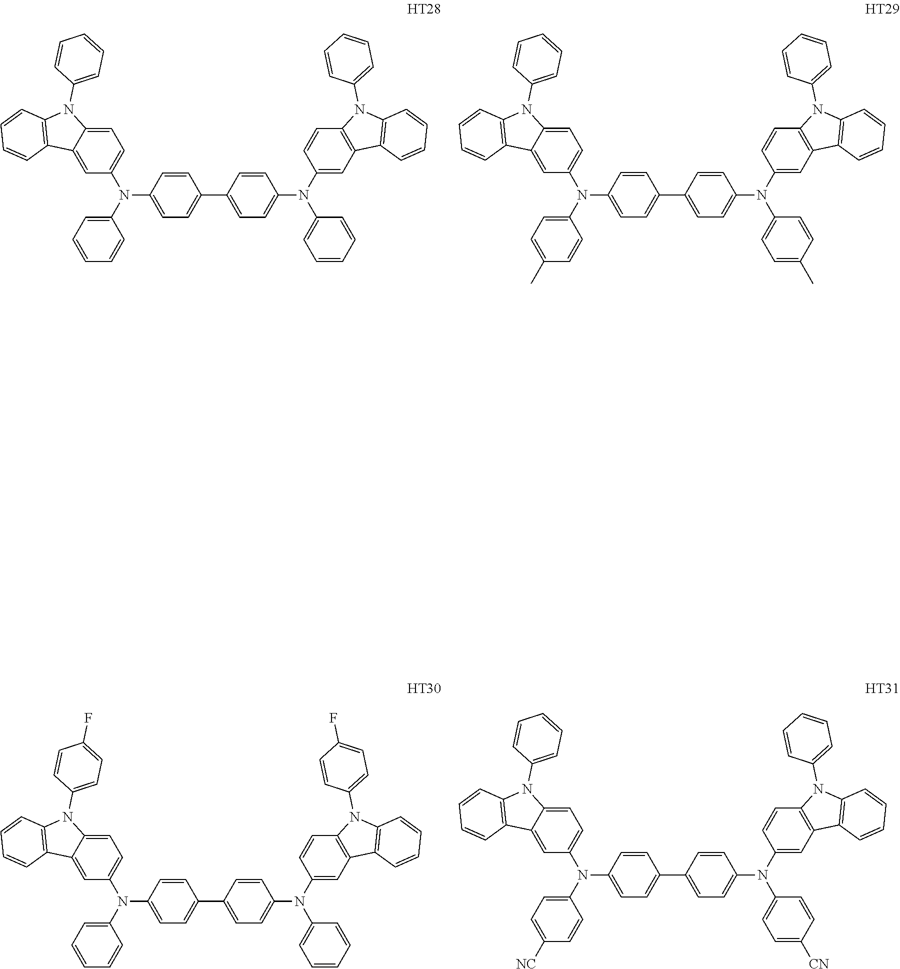

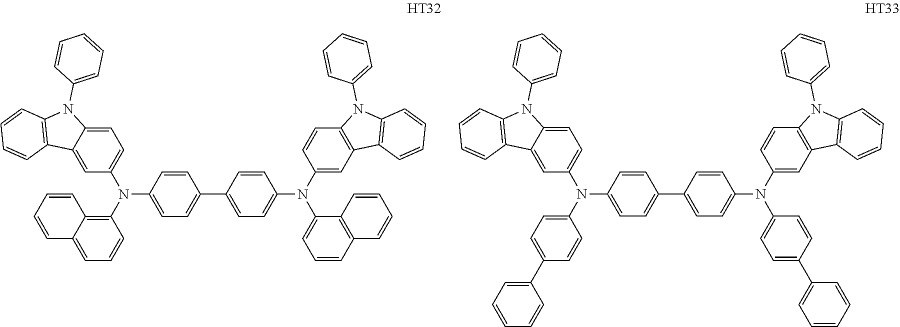

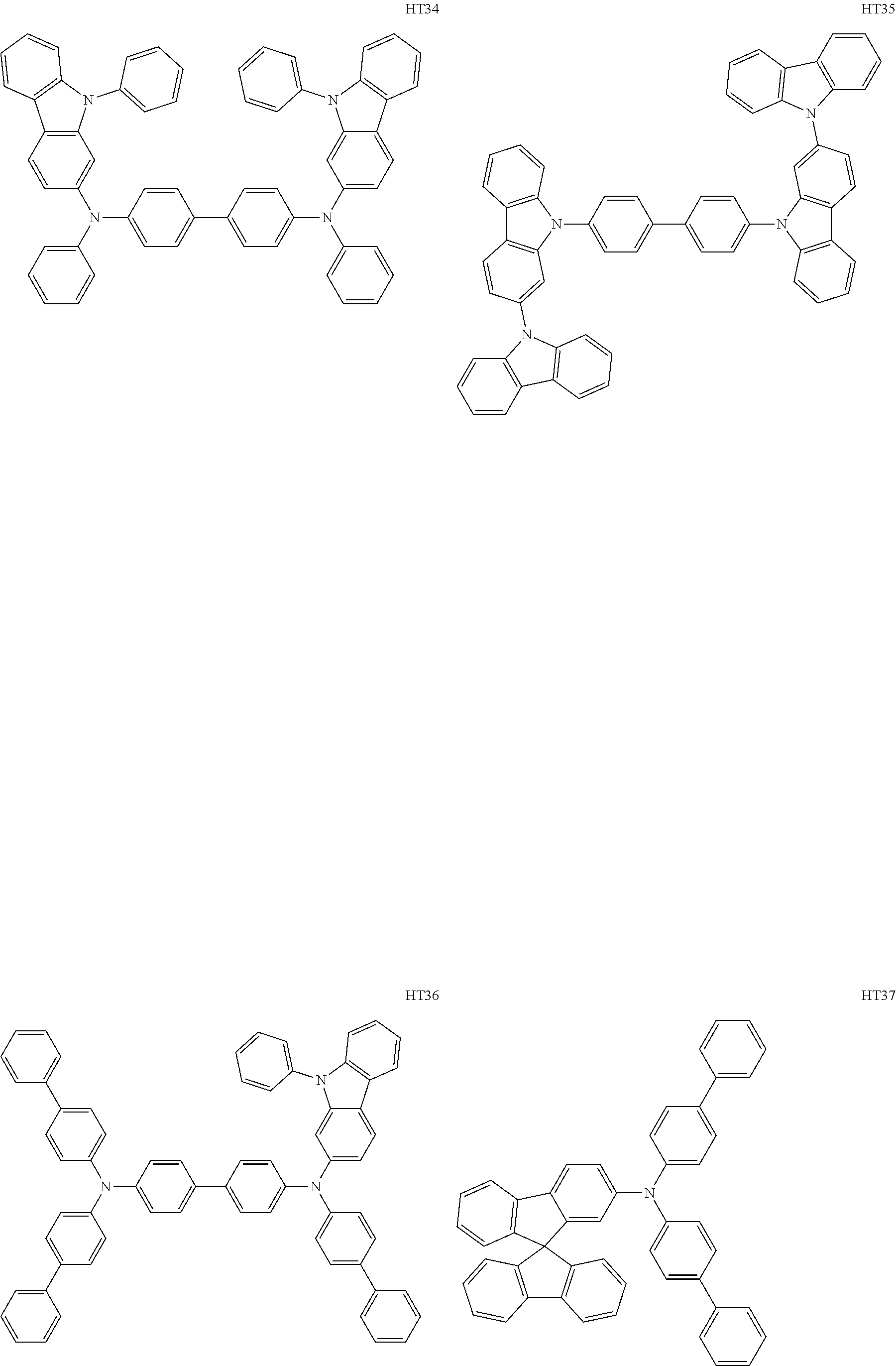 [SMILES: c1ccc(N(c2ccc(-c3ccc(N(c4ccccc4)c4ccc5c6ccccc6n(-c6ccccc6)c5c4)cc3)cc2)c2ccc3c4ccccc4n(-c4ccccc4)c3c2)cc1 ; c1ccc2c(c1)c1ccccc1n2-c1ccc2c3ccccc3n(-c3ccc(-c4ccc(-n5c6ccccc6c6ccc(-n7c8ccccc8c8ccccc87)cc65)cc4)cc3)c2c1 ; c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1]

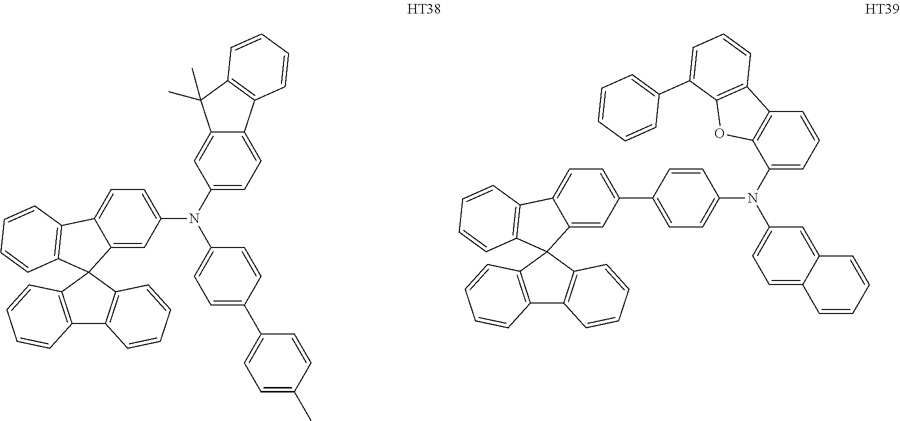 [SMILES: Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

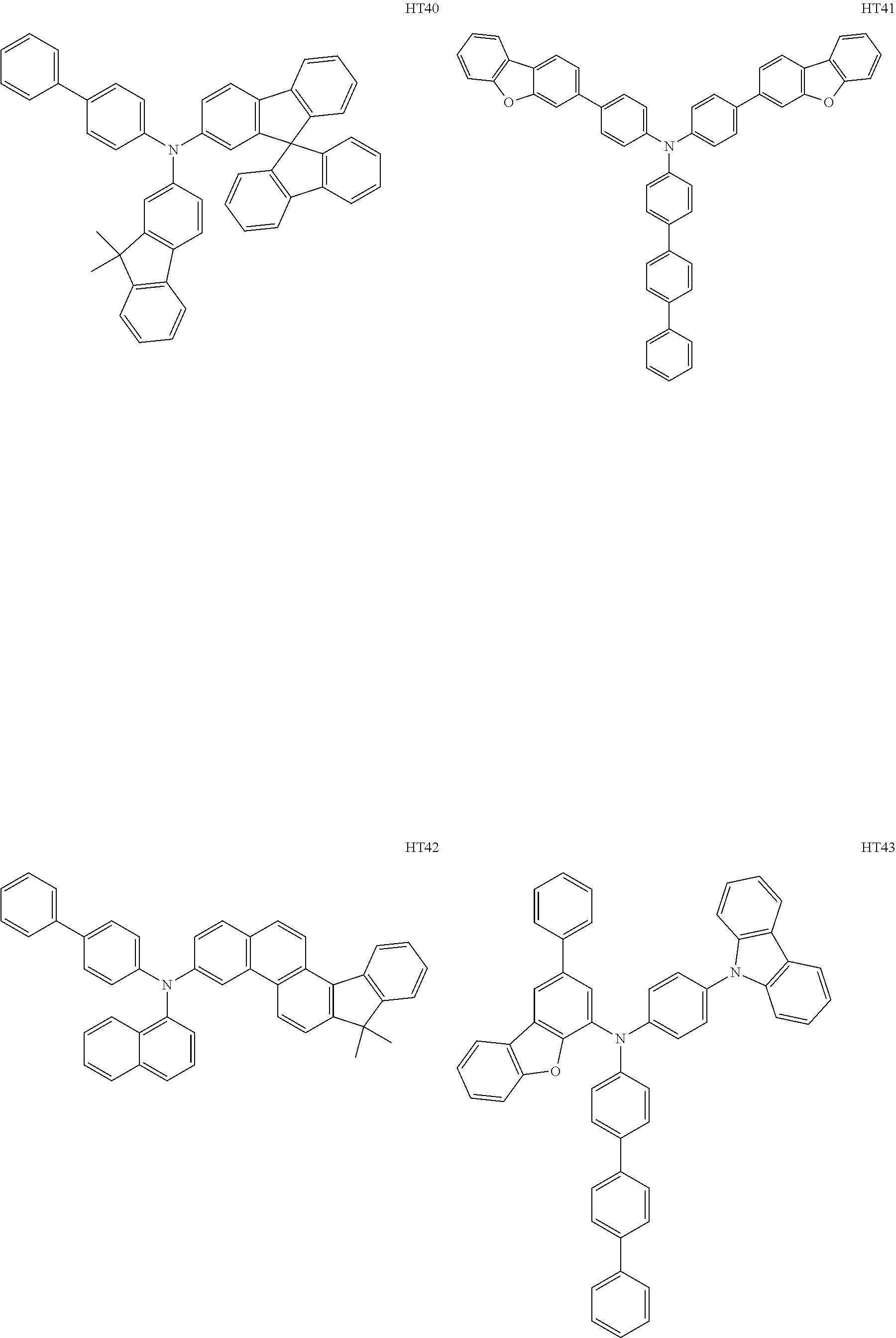 [SMILES: CC1(C)c2ccccc2-c2ccc(N(c3ccc(-c4ccccc4)cc3)c3ccc4c(c3)C3(c5ccccc5-c5ccccc53)c3ccccc3-4)cc21 ; c1ccc(-c2ccc(-c3ccc(N(c4ccc(-c5ccc6c(c5)oc5ccccc56)cc4)c4ccc(-c5ccc6c(c5)oc5ccccc56)cc4)cc3)cc2)cc1 ; CC1(C)c2ccccc2-c2c1ccc1c2ccc2ccc(N(c3ccc(-c4ccccc4)cc3)c3cccc4ccccc34)cc21 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1]

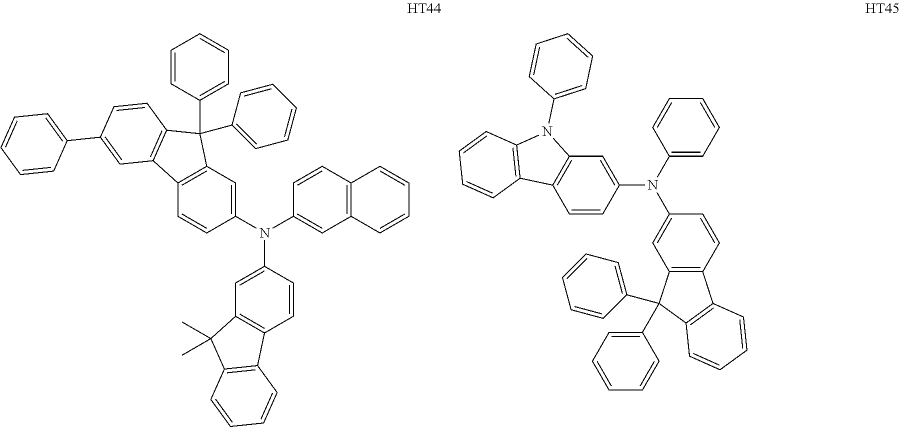 [SMILES: CC1(C)c2ccccc2-c2ccc(N(c3ccc4c(c3)C(c3ccccc3)(c3ccccc3)c3ccc(-c5ccccc5)cc3-4)c3ccc4ccccc4c3)cc21 ; c1ccc(N(c2ccc3c(c2)C(c2ccccc2)(c2ccccc2)c2ccccc2-3)c2ccc3c4ccccc4n(-c4ccccc4)c3c2)cc1]

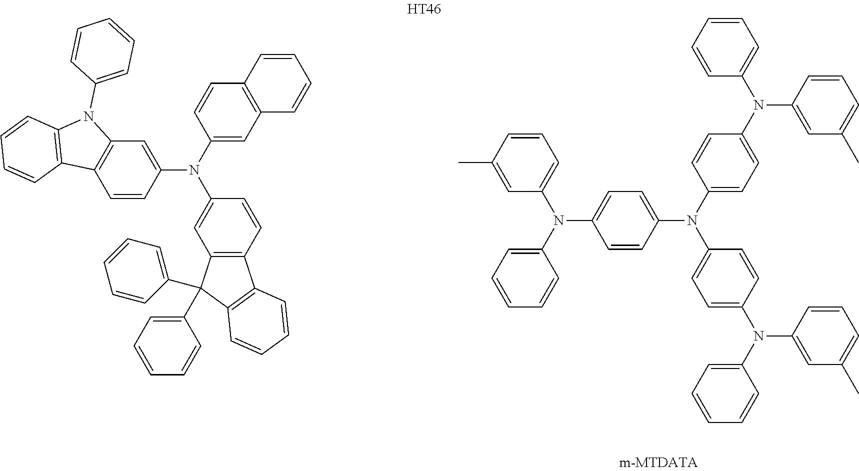

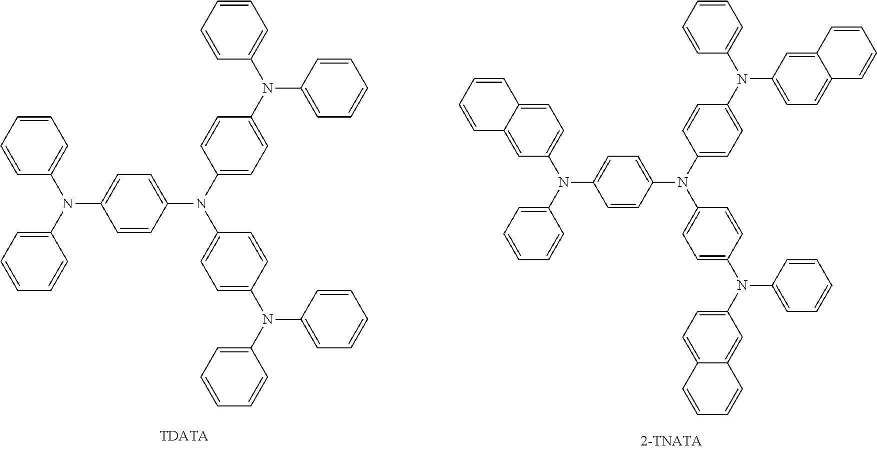 [SMILES: c1ccc(N(c2ccccc2)c2ccc(N(c3ccc(N(c4ccccc4)c4ccccc4)cc3)c3ccc(N(c4ccccc4)c4ccccc4)cc3)cc2)cc1 ; c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; "TDATA"]

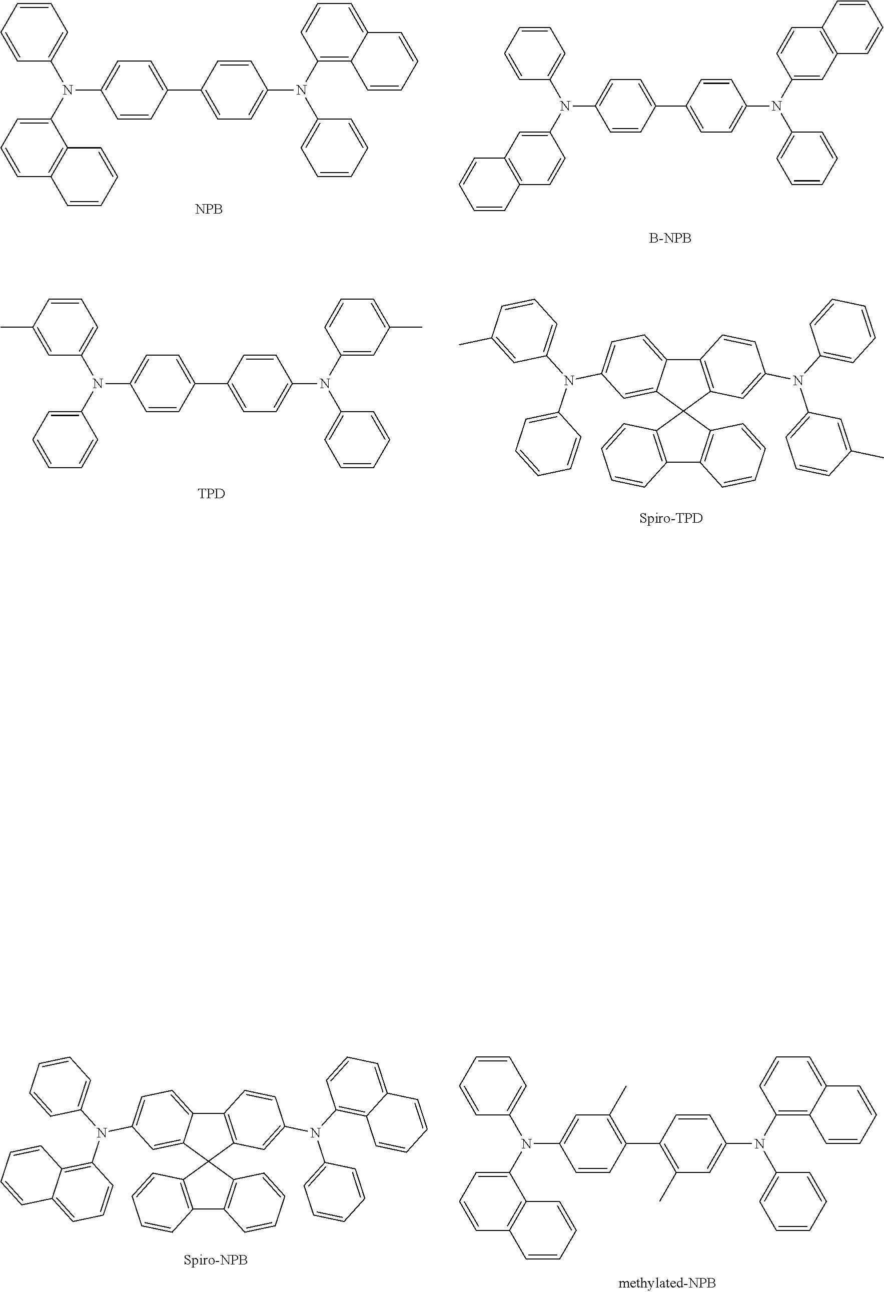

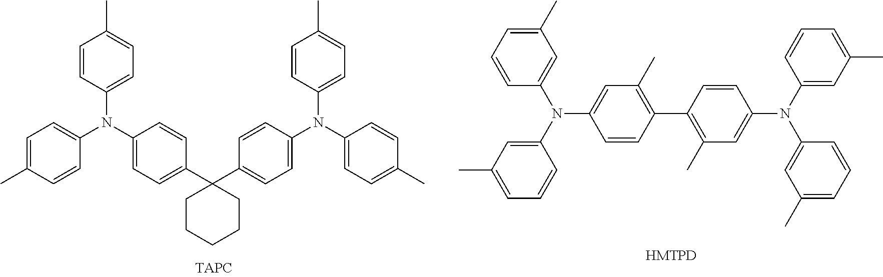

A thickness of the hole transport region may be in a range of about 50 Å to about 10,000 Å, for example, about 100 Å to about 4,000 Å. When the hole transport region includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer, and the hole transport layer are within these ranges, satisfactory hole transporting characteristics may be obtained without a substantial increase in driving voltage.

The emission auxiliary layer may increase light-emission efficiency by compensating for an optical resonance distance according to the wavelength of light emitted by the emission layer, and the electron blocking layer may block the leakage of electrons from the emission layer to the hole transport region. Materials that may be included in the hole transport region may be included in the emission auxiliary layer and the electron blocking layer.

[p-Dopant]

The hole transport region may further include, in addition to these materials, a charge-generation material for the improvement of conductive properties. The charge-generation material may be uniformly or non-uniformly dispersed in the hole transport region (for example, in the form of a single layer consisting of a charge-generation material).

The charge-generation material may be, for example, a p-dopant.

For example, the p-dopant may have a lowest unoccupied molecular orbital (LUMO) energy level of equal to or less than −3.5 eV.

In an embodiment, the p-dopant may include a quinone derivative, a cyano group-containing compound, a compound containing element EL1 and element EL2, or any combination thereof.

Examples of the quinone derivative are TCNQ, F4-TCNQ, and the like, and examples of the cyano group-containing compound are HAT-CN, a compound represented by Formula 221, and the like:

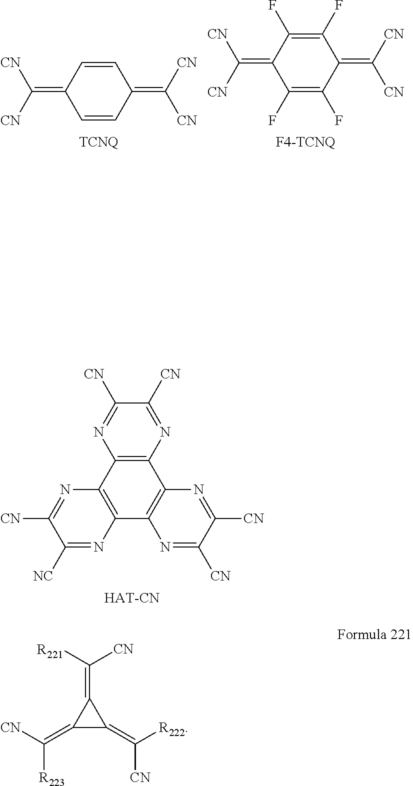

In Formula 221,

R₂₂₁ to R₂₂₃ may each independently be a C₃-C₆₀ carbocyclic group unsubstituted or substituted with at least one R₁₁ₐ or a C₁-C₆₀ heterocyclic group unsubstituted or substituted with at least one R₁₀ₐ, and

at least one of R₂₂₁ to R₂₂₃ may each independently be a C₃-C₆₀ carbocyclic group or a C₁-C₆₀ heterocyclic group, each substituted with: a cyano group; —F; —Cl; —Br; —I; a C₁-C₂₀ alkyl group substituted with a cyano group, —F, —Cl, —Br, —I, or any combination thereof, or any combination thereof.

In the compound including element EL1 and element EL2, element EL1 may be metal, metalloid, or any combination thereof, and element EL2 may be non-metal, metalloid, or any combination thereof.

Examples of the metal are an alkali metal (for example, lithium (Li), sodium (Na), potassium (K), rubidium (Rb), cesium (Cs), and the like.); alkaline earth metal (for example, beryllium (Be), magnesium (Mg), calcium (Ca), strontium (Sr), barium (Ba), and the like); transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), and the like); post-transition metal (for example, zinc (Zn), indium (In), tin (Sn), and the like); and lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), and the like).

Examples of the metalloid are silicon (Si), antimony (Sb), tellurium (Te), and the like.

Examples of the non-metal are oxygen (O), halogen (for example, F, Cl, Br, I, and the like), and the like.

Examples of the compound containing element EL1 and element EL2 are metal oxide, metal halide (for example, metal fluoride, metal chloride, metal bromide, metal iodide, and the like), metalloid halide (for example, metalloid fluoride, metalloid chloride, metalloid bromide, metalloid iodide, and the like), metal telluride, or any combination thereof.

Examples of the metal oxide mare tungsten oxide (for example, WO, W₂O₃, WO₂, WO₃, W₂O₅, and the like), vanadium oxide (for example, VO, V₂O₃, VO₂, V₂O₅, and the like), molybdenum oxide (MoO, Mo₂O₃, MoO₂, MoO₃, Mo₂O₅, and the like), rhenium oxide (for example, ReO₃, and the like), and the like.

Examples of the metal halide are alkali metal halide, alkaline earth metal halide, transition metal halide, post-transition metal halide, lanthanide metal halide, and the like.

Examples of the alkali metal halide are LiF, NaF, KF, RbF, CsF, LiCl, NaCl, KCl, RbCl, CsCl, LiBr, NaBr, KBr, RbBr, CsBr, LiI, NaI, KI, RbI, CsI, and the like.

Examples of the alkaline earth metal halide are BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, BeI₂, MgI₂, CaI₂, SrI₂, BaI₂, and the like.

Examples of the transition metal halide are titanium halide (for example, TiF₄, TiCl₄, TiBr₄, TiI₄, and the like), zirconium halide (for example, ZrF₄, ZrCl₄, ZrBr₄, ZrI₄, and the like), hafnium halide (for example, HfF₄, HfCl₄, HfBr₄, HfI₄, and the like), vanadium halide (for example, VF₃, VCl₃, VBr₃, VI₃, and the like), niobium halide (for example, NbF₃, NbCl₃, NbBr₃, NbI₃, and the like), tantalum halide (for example, TaF₃, TaCl₃, TaBr₃, TaI₃, and the like), chromium halide (for example, CrF₃, CrCl₃, CrBr₃, CrI₃, and the like), molybdenum halide (for example, MoF₃, MoCl₃, MoBr₃, MoI₃, and the like), tungsten halide (for example, WF₃, WCl₃, WBr₃, WI₃, and the like), manganese halide (for example, MnF₂, MnCl₂, MnBr₂, MnI₂, and the like), technetium halide (for example, TcF₂, TcCl₂, TcBr₂, TcI₂, and the like), rhenium halide (for example, ReF₂, ReCl₂, ReBr₂, ReI₂, and the like), iron halide (for example, FeF₂, FeCl₂, FeBr₂, FeI₂, and the like), ruthenium halide (for example, RuF₂, RuCl₂, RuBr₂, RuI₂, and the like), osmium halide (for example, OsF₂, OsCl₂, OsBr₂, OsI₂, and the like), cobalt halide (for example, CoF₂, CoCl₂, CoBr₂, CoI₂, and the like), rhodium halide (for example, RhF₂, RhCl₂, RhBr₂, RhI₂, and the like), iridium halide (for example, IrF₂, IrCl₂, IrBr₂, IrI₂, and the like), nickel halide (for example, NiF₂, NiCl₂, NiBr₂, NiI₂, and the like), palladium halide (for example, PdF₂, PdCl₂, PdBr₂, PdI₂, and the like), platinum halide (for example, PtF₂, PtCl₂, PtBr₂, PtI₂, and the like), copper halide (for example, CuF, CuCl, CuBr, CuI, and the like), silver halide (for example, AgF, AgCl, AgBr, AgI, and the like), gold halide (for example, AuF, AuCl, AuBr, AuI, and the like), and the like.

Examples of the post-transition metal halide are zinc halide (for example, ZnF₂, ZnCl₂, ZnBr₂, ZnI₂, and the like), indium halide (for example, InI₃, etc.), tin halide (for example, SnI₂, and the like), and the like.

Examples of the lanthanide metal halide are YbF, YbF₂, YbF₃, SmF₃, YbCl, YbCl₂, YbCl₃SmCl₃, YbBr, YbBr₂, YbBr₃, SmBr₃, YbI, YbI₂, YbI₃, SmI₃, and the like.

Examples of the metalloid halide are antimony halide (for example, SbCl₅ and the like) and the like.

Examples of the metal telluride are alkali metal telluride (for example, Li₂Te, Na₂Te, K₂Te, Rb₂Te, Cs₂Te, and the like), alkaline earth metal telluride (for example, BeTe, MgTe, CaTe, SrTe, BaTe, and the like), transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, and the like), post-transition metal telluride (for example, ZnTe, and the like), lanthanide metal telluride (for example, LaTe, CeTe, PrTe, NdTe, PmTe, EuTe, GdTe, TbTe, DyTe, HoTe, ErTe, TmTe, YbTe, LuTe, and the like), and the like.

[Emission Layer 131 in Interlayer 130]

When the light-emitting device 10 is a full-color light-emitting device, the emission layer may be patterned into a red emission layer, a green emission layer, and/or a blue emission layer, according to a sub-pixel. In an embodiment, the emission layer may have a stacked structure of two or more layers of a red emission layer, a green emission layer, and a blue emission layer, in which the two or more layers contact each other or are separated from each other to emit white light. In one or more embodiments, the emission layer may a structure in which two or more materials of a red light-emitting material, a green light-emitting material, and a blue light-emitting material are mixed with each other in a single layer to emit white light.

In an embodiment, the emission layer may include a host and a dopant. The dopant may include a phosphorescent dopant, a fluorescent dopant, or any combination thereof.

In the emission layer, an amount of the dopant may be in a range of about 0.01 parts by weight to about 15 parts by weight based on 100 parts by weight of the host.

In one or more embodiments, the emission layer may include quantum dots.

In one or more embodiments, the emission layer may include a delayed fluorescence material. The delayed fluorescence material may act as a host or a dopant in the emission layer.

A thickness of the emission layer 4 may be in a range of about 100 Å to about 1,000 Å, for example, about 200 Å to about 600 Å. When the thickness of the emission layer 4 is within these ranges, excellent luminescence characteristics may be obtained without a substantial increase in driving voltage.

[Host]

In an embodiment, the host may include a compound represented by Formula 301:

[Ar₃₀₁]_{xb11}-[(L₃₀₁)_{xb1}-R₃₀₁]_{xb21} Formula 301

wherein, in Formula 301,

Ar₃O₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xb11 may be 1, 2, or 3,

xb1 may be an integer from 0 to 5,

R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀1)(Q₃₀₂),

xb21 may be an integer from 1 to 5, and

Q₃₀₁ to Q₃₀₃ may respectively be the same as described in connection with Q₁.

For example, when xb11 in Formula 301 is 2 or more, two or more of Ar301(s) may be linked to each other via a single bond.

In one or more embodiments, the host may include a compound represented by Formula 301-1, a compound represented by Formula 301-2, or any combination thereof.

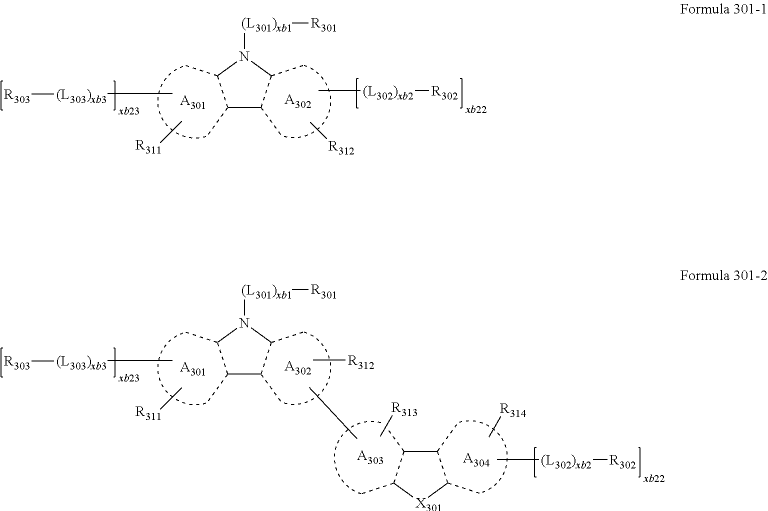

wherein, in Formulae 301-1 and 301-2,

ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

X₃₀₁ may be O, S, N-[(L₃₀₄)_{xb4}-R₃₀₄], C(R₃₀₄)(R₃₀₅), or Si(R₃₀₄)(R₃₀₅),

xb22 and xb23 may each independently be 0, 1, or 2,

L₃₀₁, xb1, and R₃₀₁ may respectively be the same as described herein,

L₃₀₂ to L₃₀₄ may each independently be the same as described in connection with L₃₀₁,

xb2 to xb4 may each independently be the same as described in connection with xb1, and

R₃₀₂ to R₃₀₅ and R₃₁₁ to R₃₁₄ may respectively be the same as described in connection with R₃₀₁.

In one or more embodiments, the host may include an alkali earth metal complex, a post-transition metal complex, or any combination thereof. For example, the host may include a Be complex (for example, Compound H55), an Mg complex, a Zn complex, or any combination thereof.

In one or more embodiments, the host may include one of Compounds H1 to H124, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di-9-carbazolylbenzene (mCP), 1,3,5-tri(carbazol-9-yl)benzene (TCP), or any combination thereof:

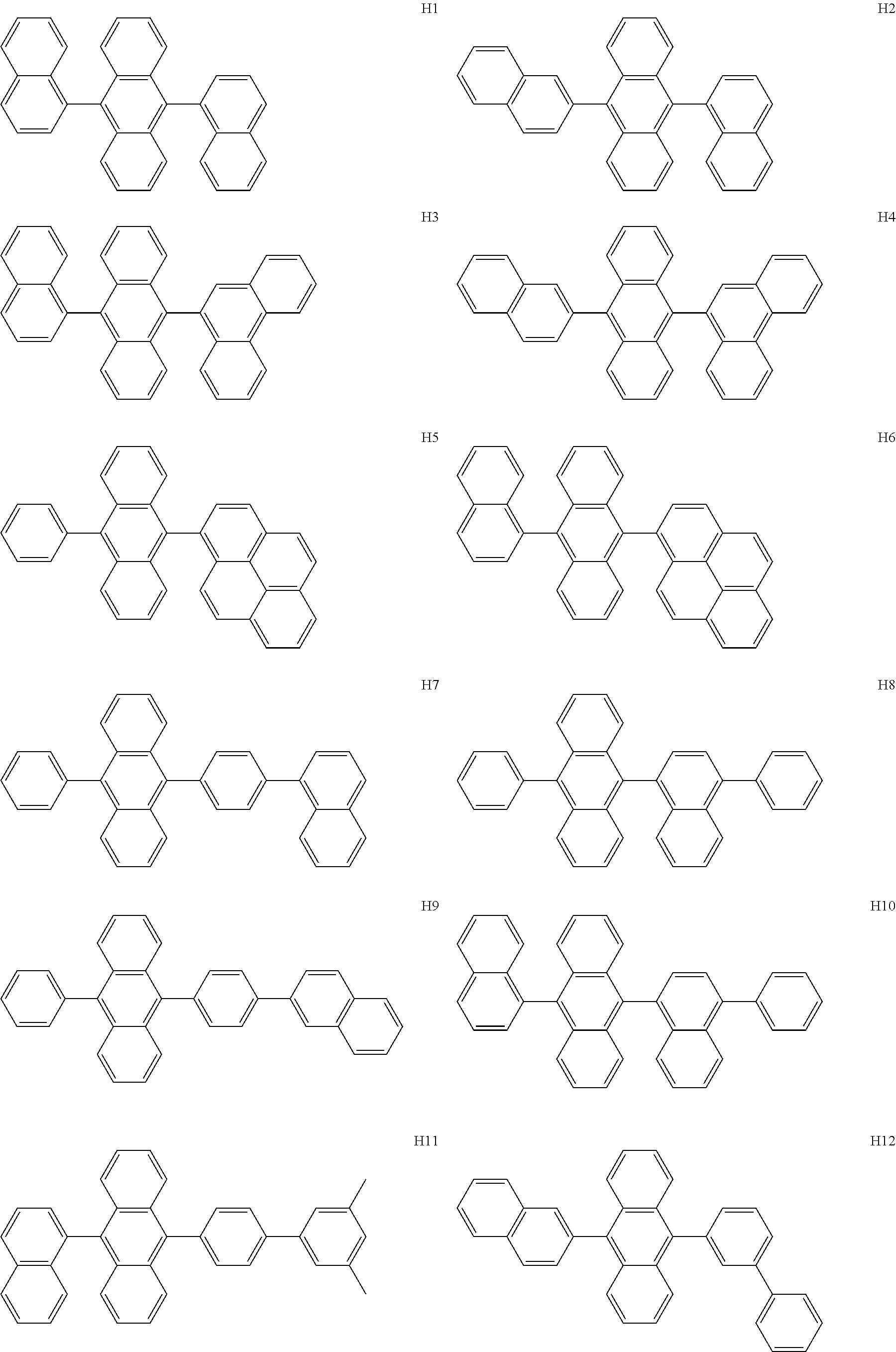

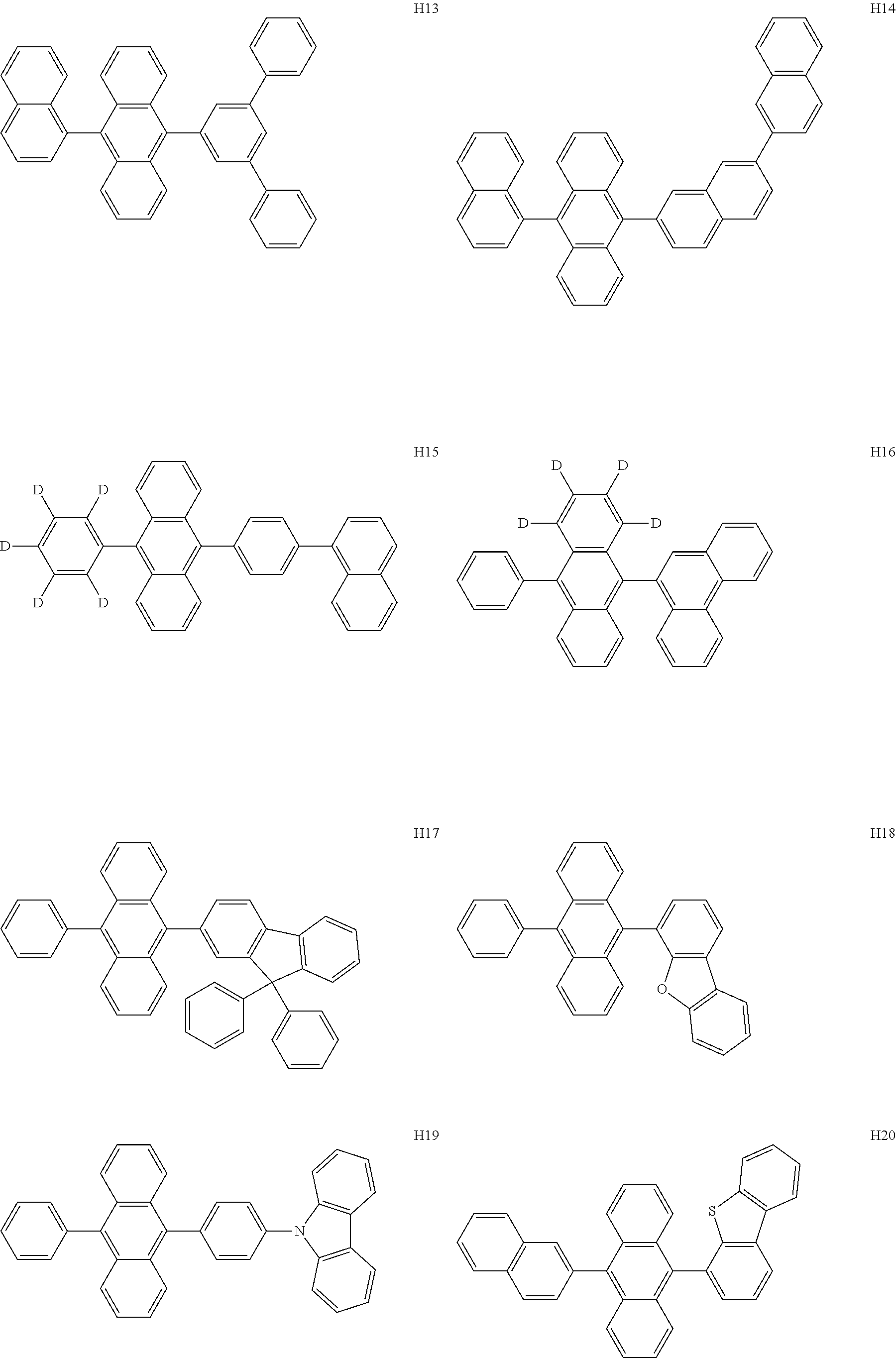

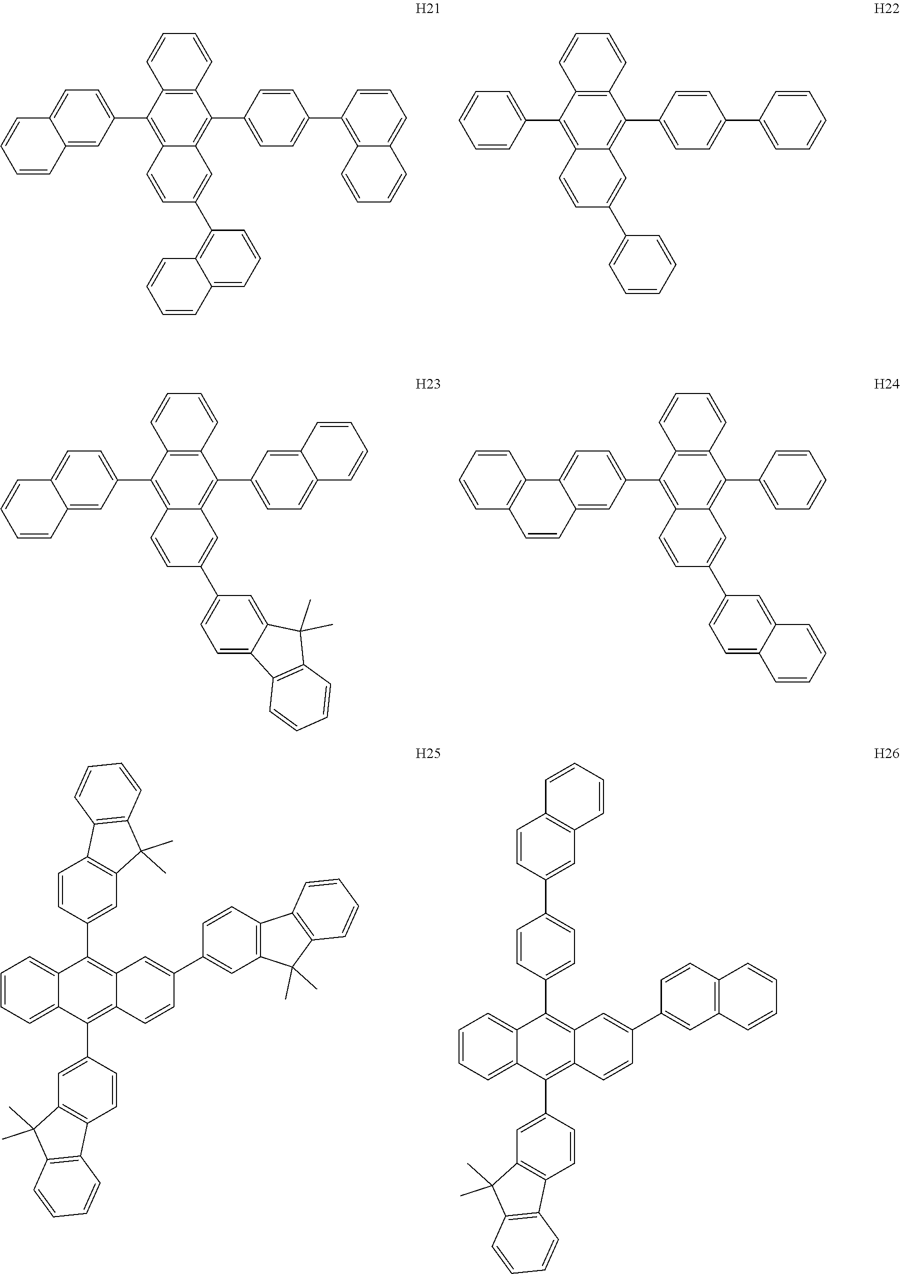

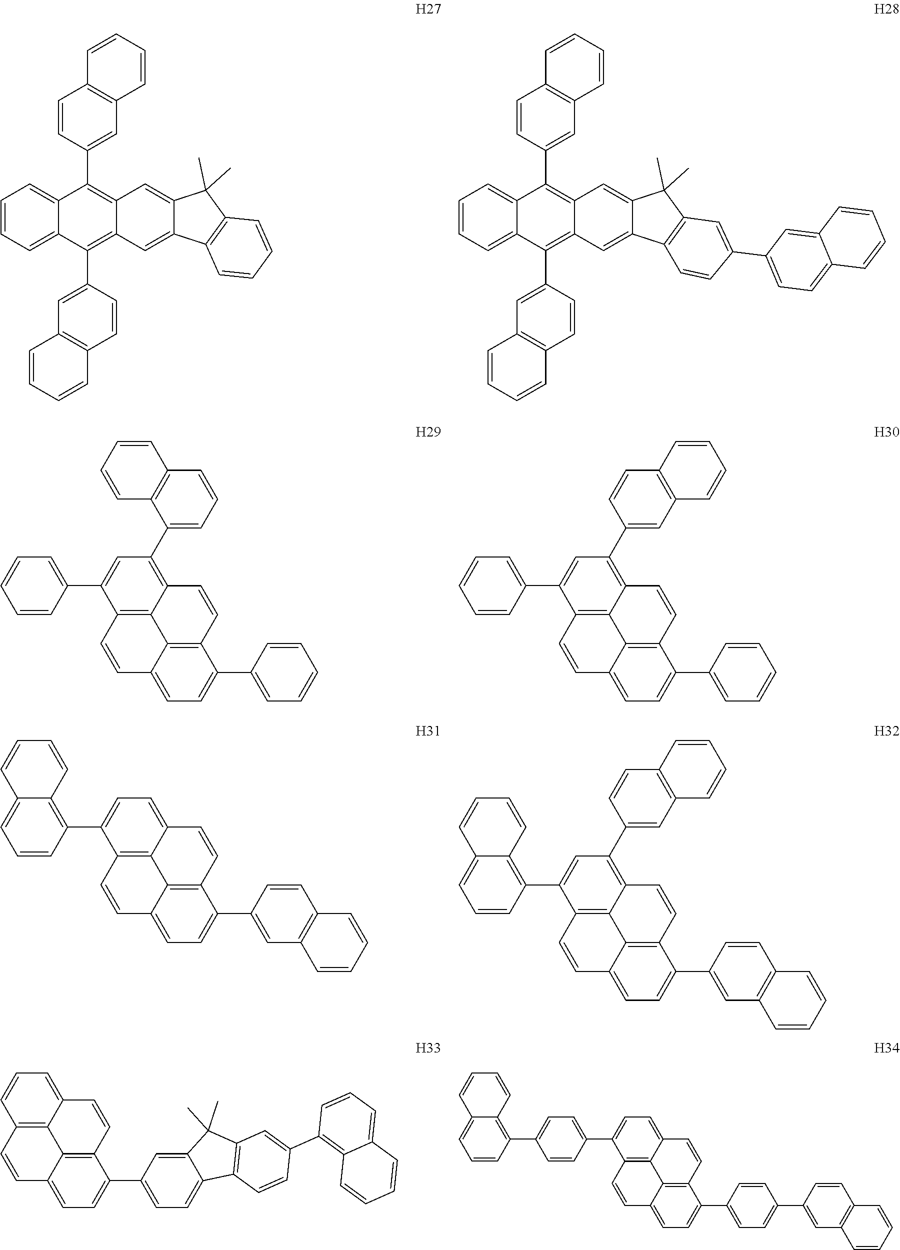

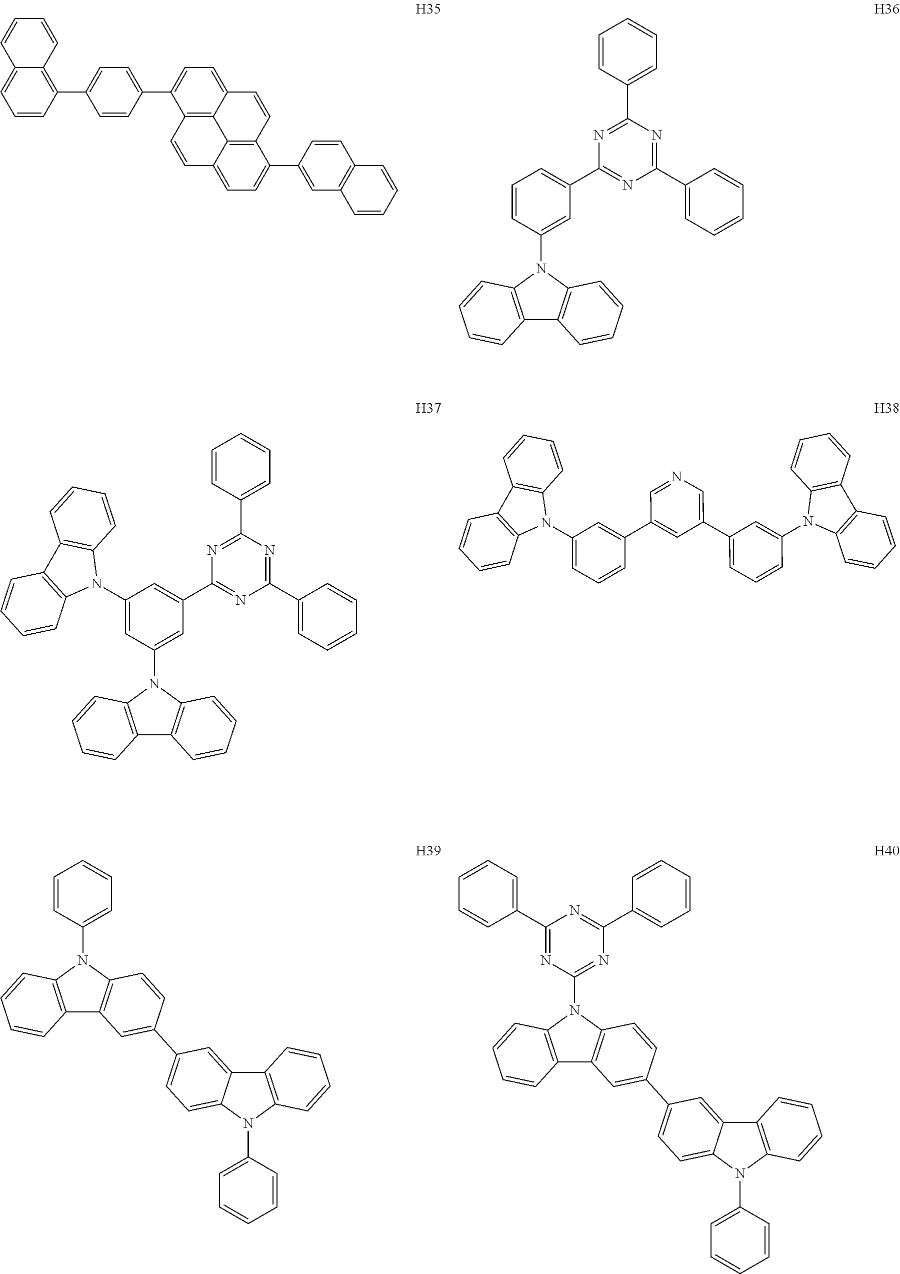

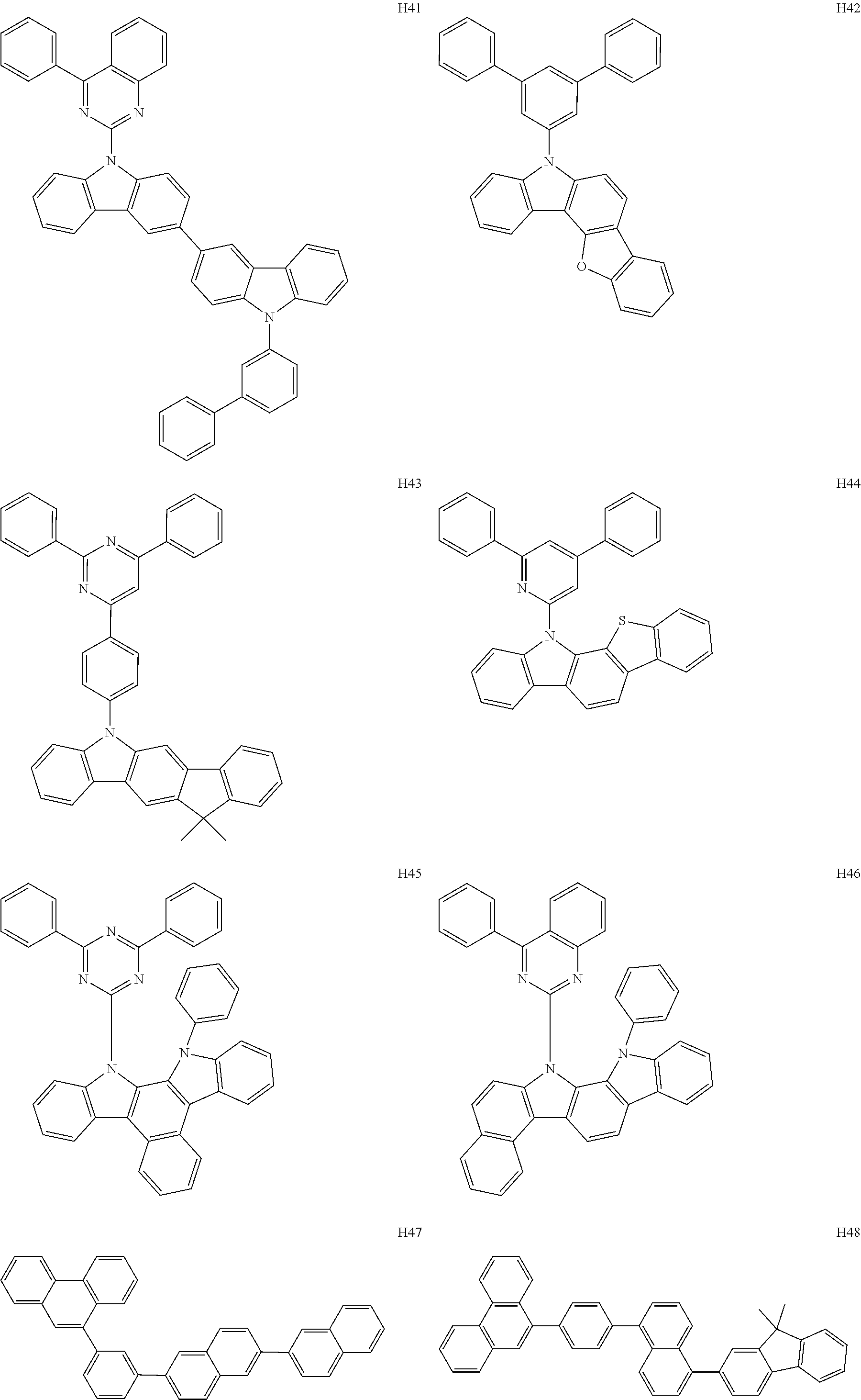

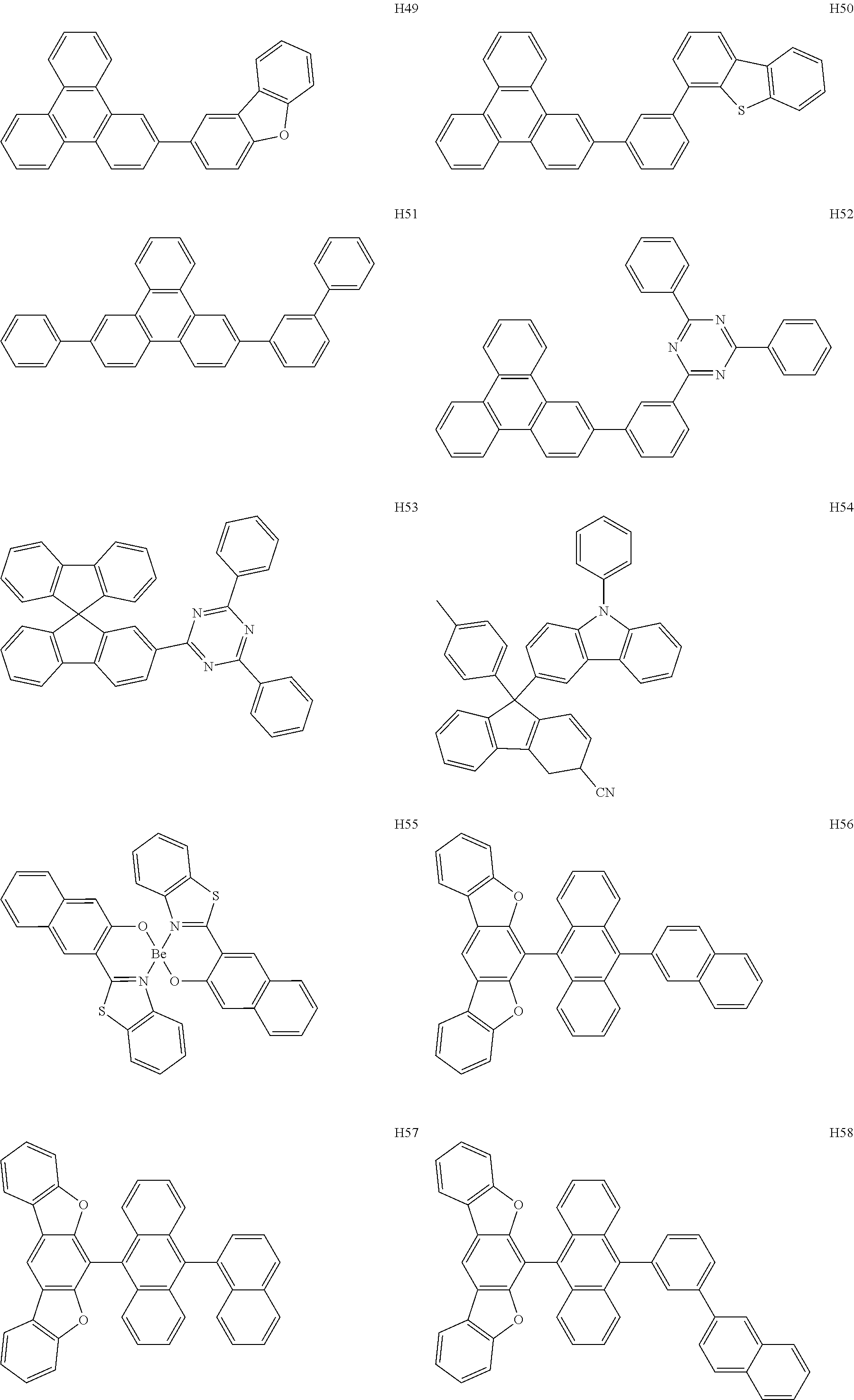

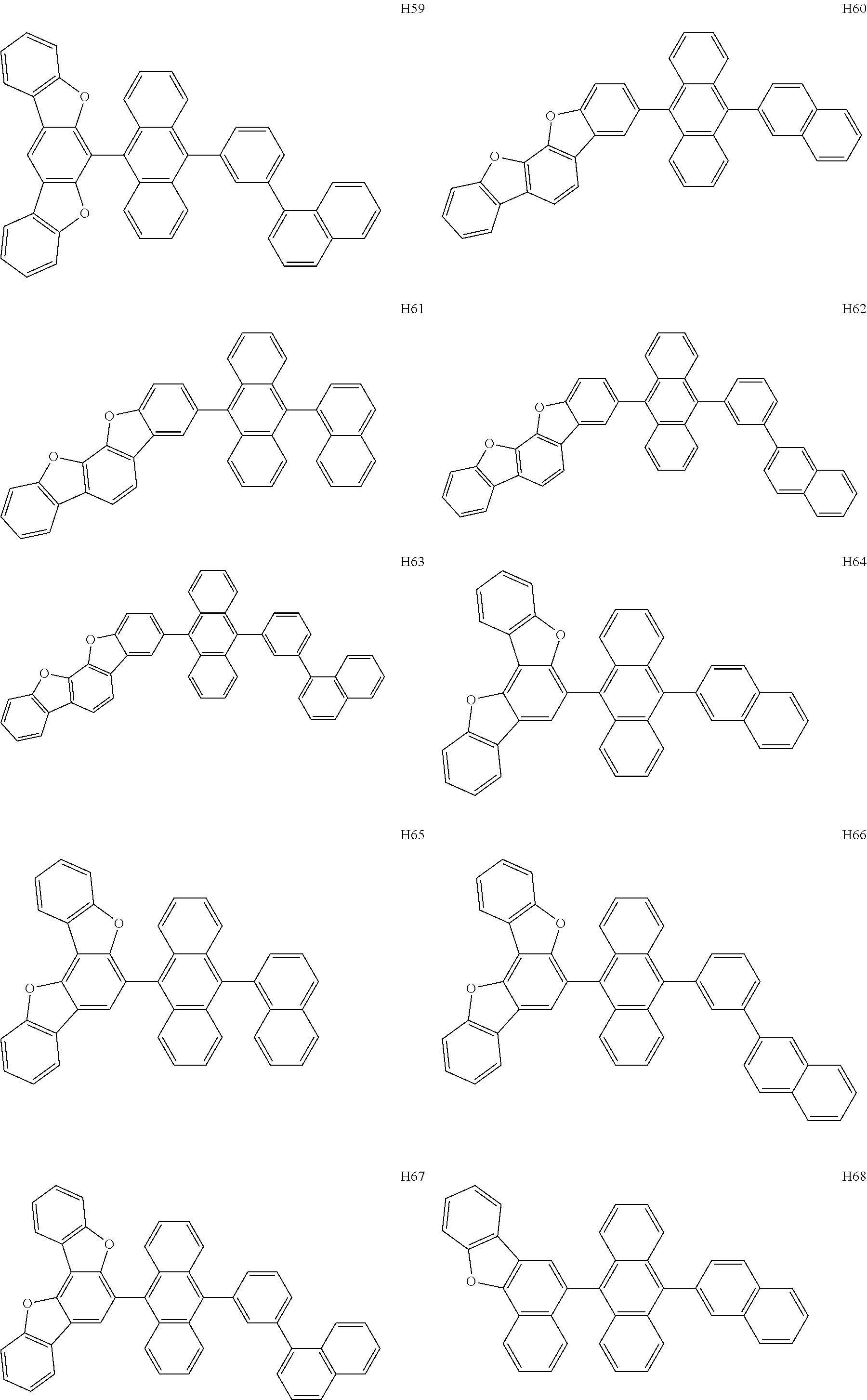

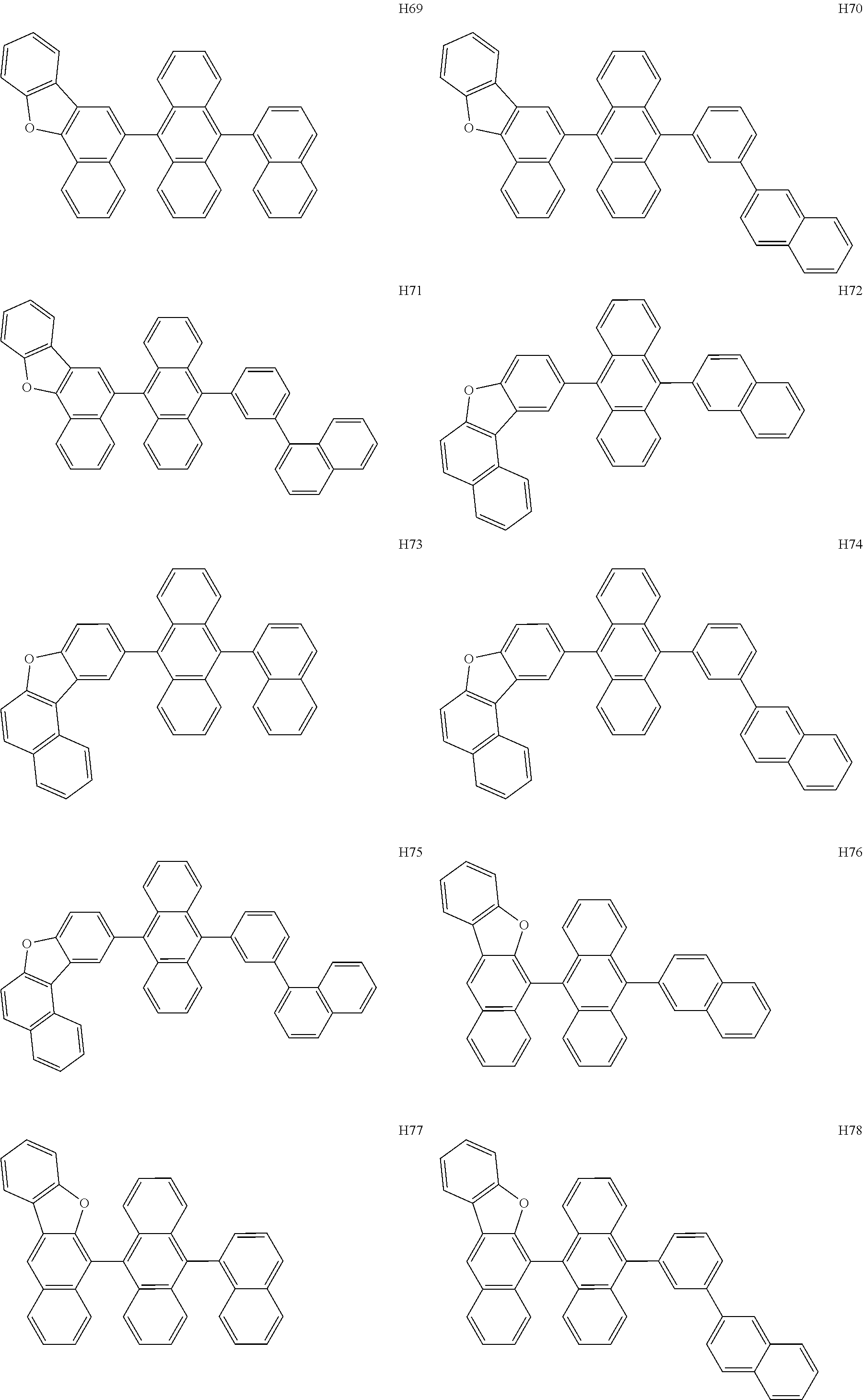

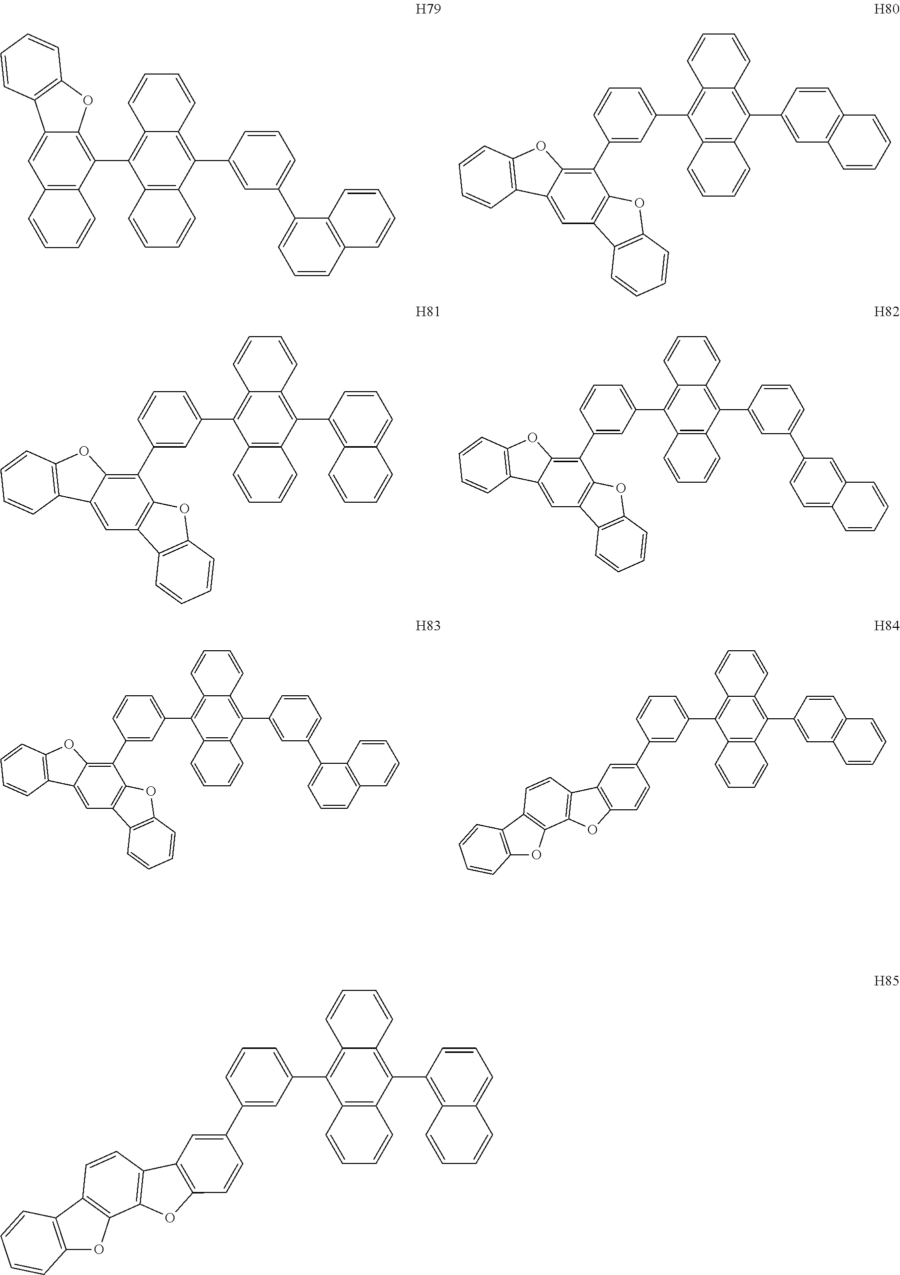

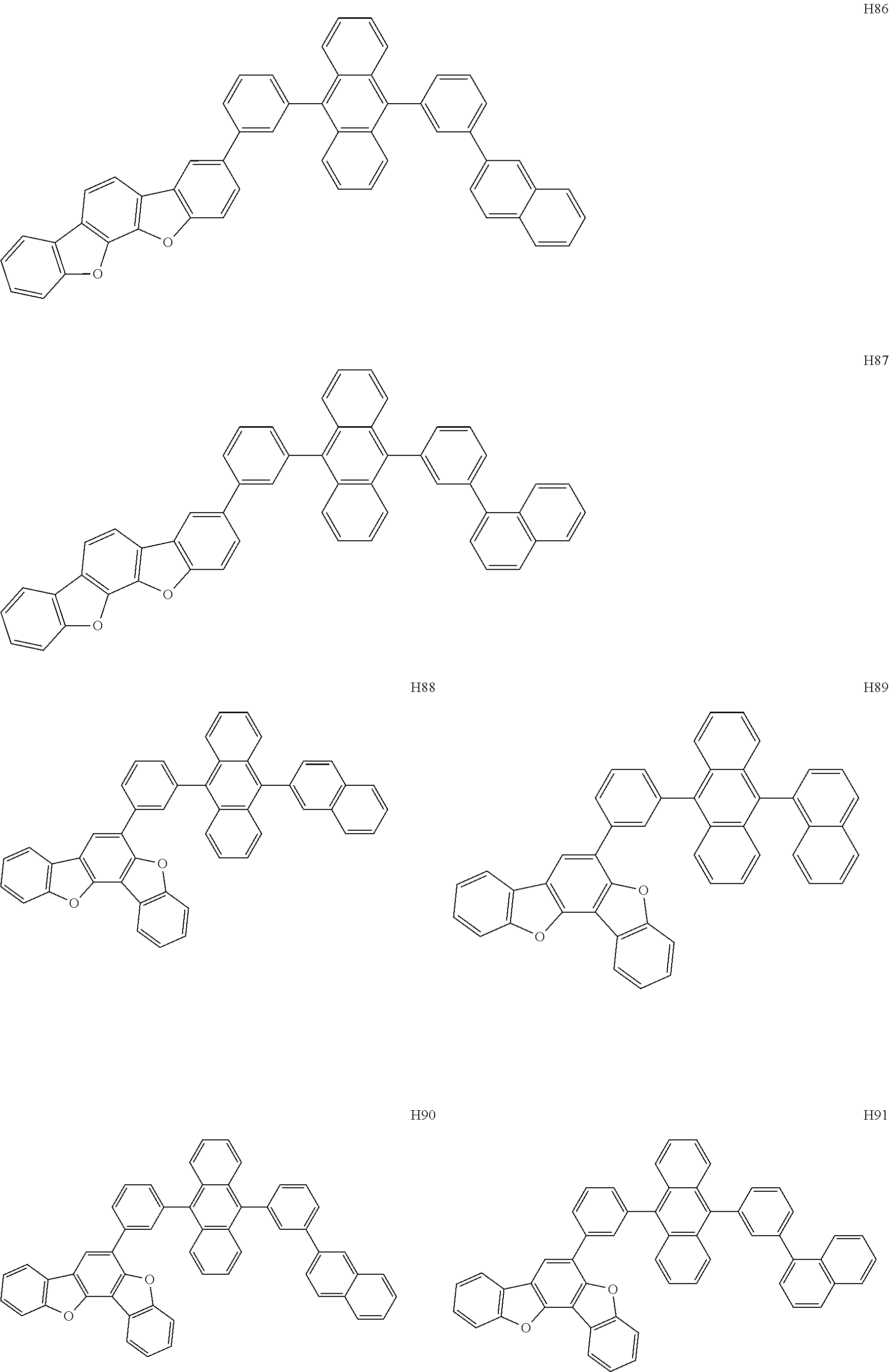

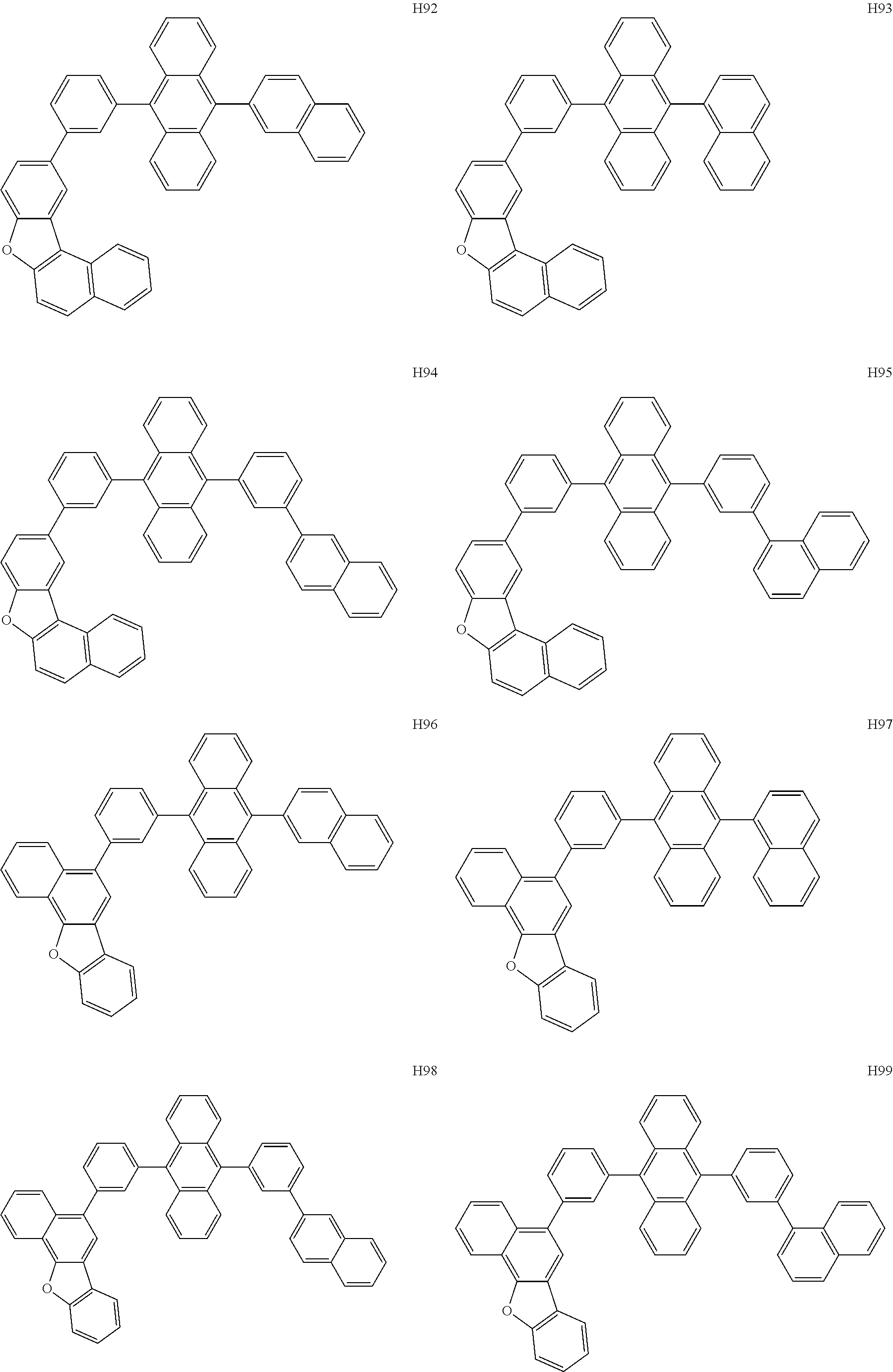

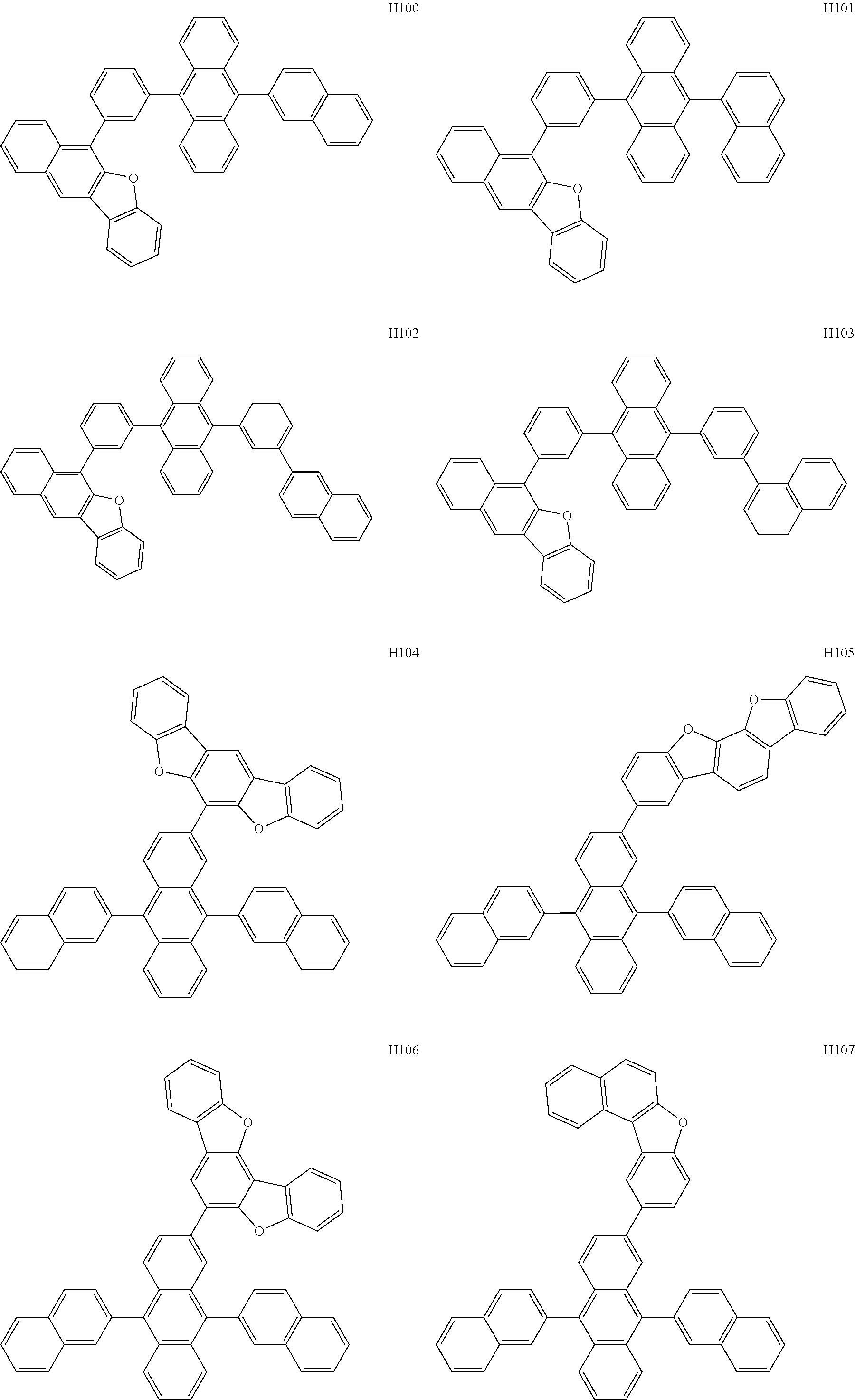

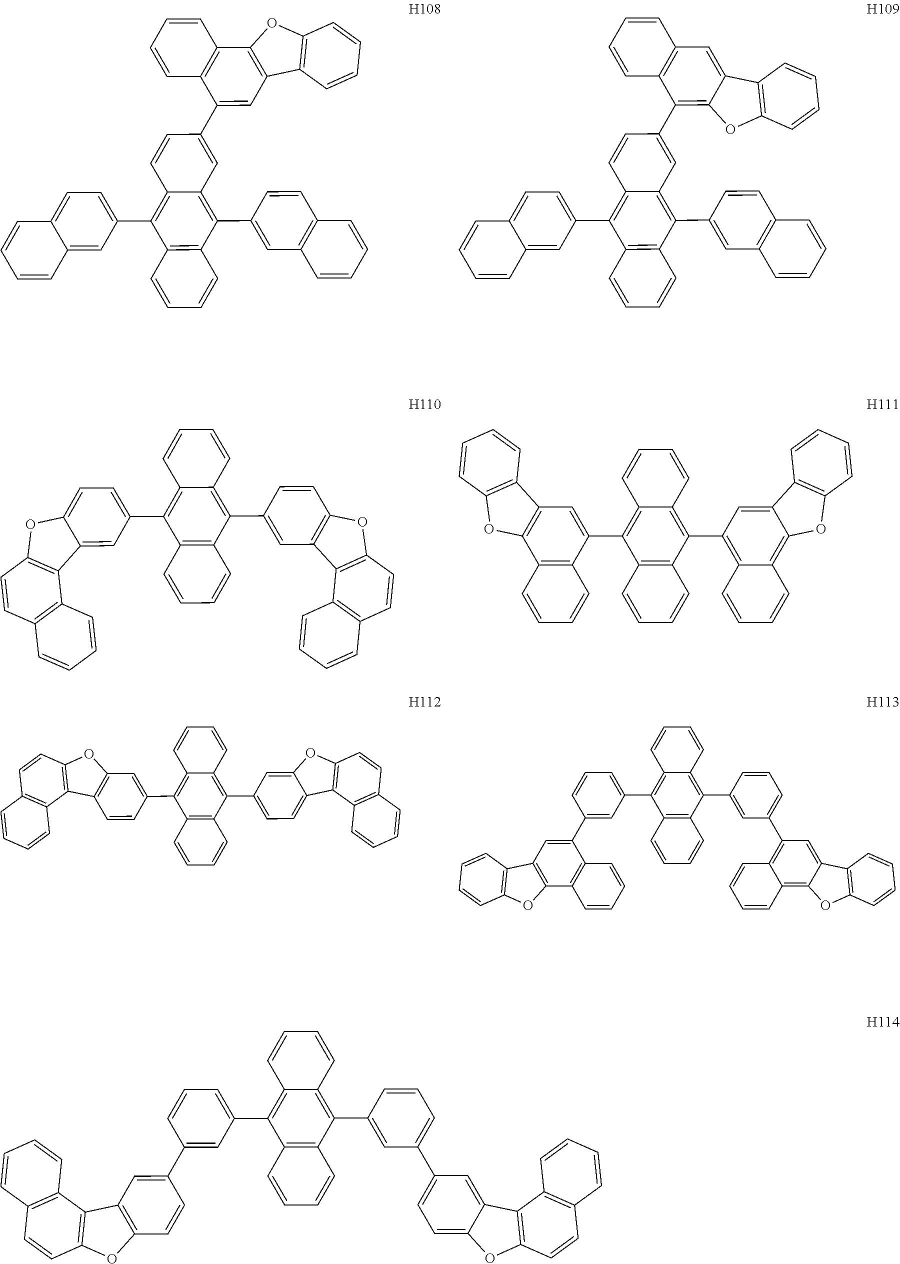

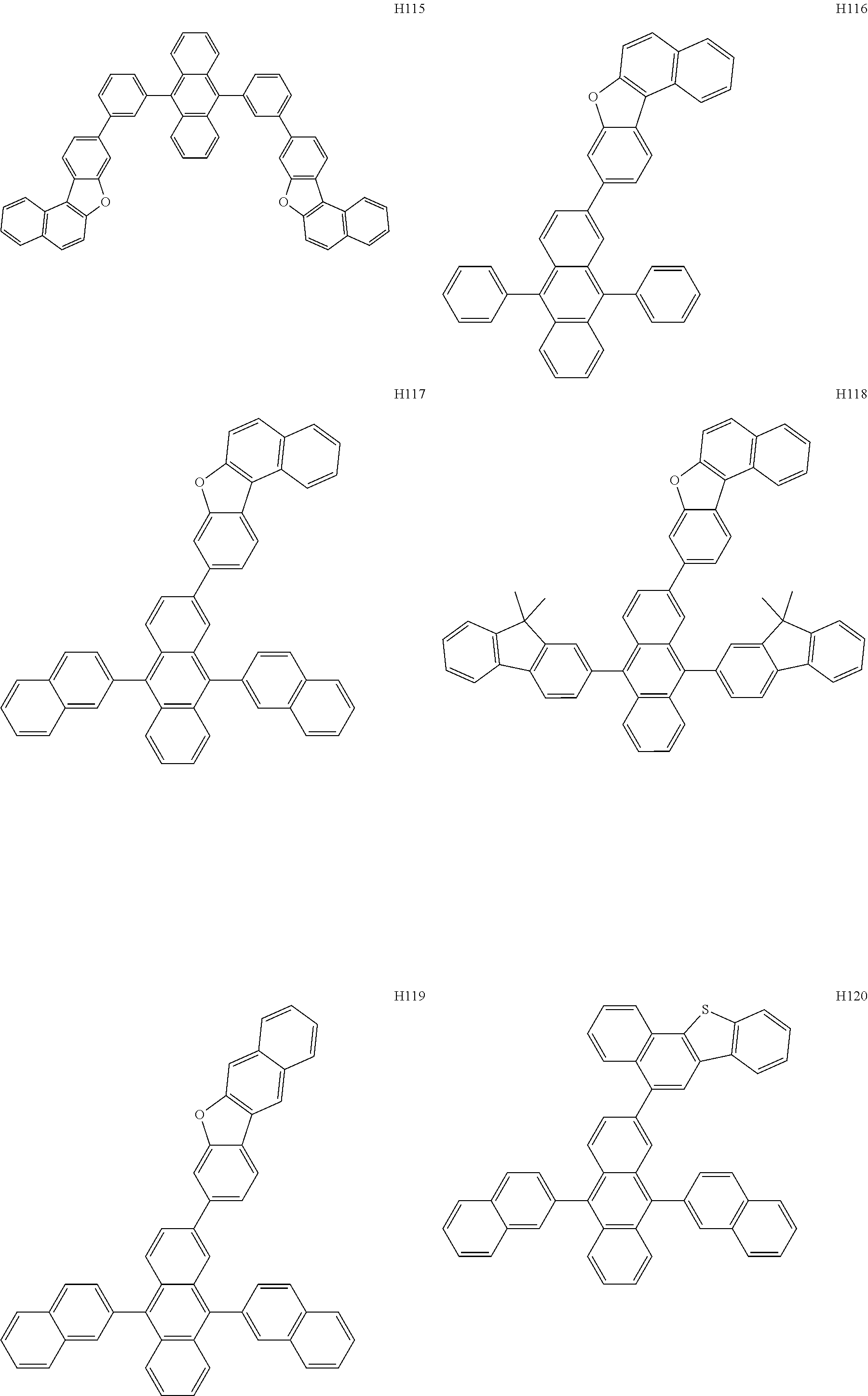

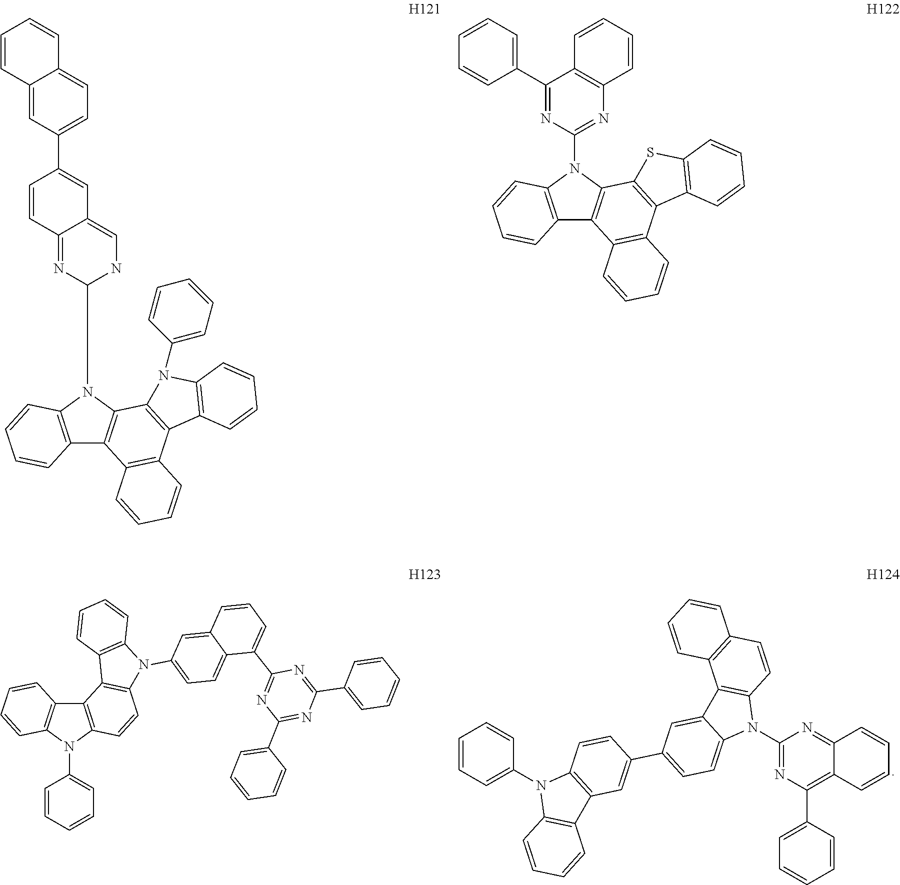

[Phosphorescent Dopant]

In an embodiment, the phosphorescent dopant may include at least one transition metal as a central metal.

In an embodiment, the phosphorescent dopant may include a monodentate ligand, a bidentate ligand, a tridentate ligand, a tetradentate ligand, a pentadentate ligand, a hexadentate ligand, or any combination thereof.

In an embodiment, the phosphorescent dopant may be electrically neutral.

In an embodiment, the phosphorescent dopant may include an organometallic compound represented by Formula 401:

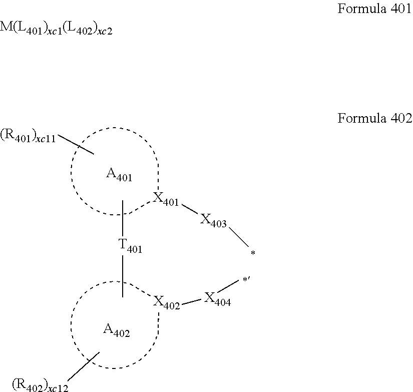

wherein, in Formulae 401 and 402,

M may be transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),

L₄₀₁ may be a ligand represented by Formula 402, and xc1 may be 1, 2, or 3, wherein, when xc1 is 2 or more, two or more of L₄₀₁(s) may be identical to or different from each other,

L₄₀₂ may be an organic ligand, and xc2 may be 0, 1, 2, 3, or 4, wherein, when xc2 is 2 or more, two or more of L₄₀₂(s) may be identical to or different from each other,

X₄₀₁ and X₄₀₂ may each independently be nitrogen or carbon,

ring A₄₀₁ and ring A₄₀₂ may each independently be a C₃-C₆₀ carbocyclic group or a C₁-C₆₀ heterocyclic group,

T₄₀₁ may be a single bond, *—O—*′, *—S—*′, *—C(═O)—*′, *—N(Q₄₁₁)—*′, *—C(Q₄₁₁)(Q₄₁₂)-*′, *—C(Q₄₁₁)=C(Q₄₁₂)-*′, *—C(Q₄₁₁)=*′, or *═C(Q₄₁₁)=*′,

X₄₀₃ and X₄₀₄ may each independently be a chemical bond (for example, a covalent bond or a coordinate bond), O, S, N(Q₄₁₃), B(Q₄₁₃), P(Q₄₁₃), C(Q₄₁₃)(Q₄₁₄), or Si(Q₄₁₃)(Q₄₁₄),

Q₄₁₁ to Q₄₁₄ may respectively be the same as described in connection with Q₁,

R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),

Q₄₀₁ to Q₄₀₃ may respectively be the same as described in connection with Q₁,

xc11 and xc12 may each independently be an integer from 0 to 10, and

* and *′ in Formula 402 each indicate a binding site to M in Formula 401.

For example, in Formula 402, i) X₄₀₁ may be nitrogen and X₄₀₂ may be carbon, or ii) each of X₄₀₁ and X₄₀₂ may be nitrogen.

When xc1 in Formula 401 is 2 or more, two ring A₄₀₁(s) in two or more of L₄₀₁(s) may optionally be linked to each other via T₄₀₂, which is a linking group, and two ring A₄₀₂(s) in two or more of L₄₀₂(s) may optionally be linked to each other via T₄₀₃, which is a linking group (see Compounds PD1 to PD4 and PD7), wherein T₄₀₂ and T₄₀₃ may respectively be the same as described in connection with T401.

In Formula 401, L₄₀₂ may be any suitable organic ligand. For example, L₄₀₂ may include a halogen group, a diketone group (for example, an acetylacetonate group), a carboxylic acid group (for example, a picolinate group), —C(═O), an isonitrile group, a —CN group, a phosphorus group (for example, a phosphine group, a phosphite group, and the like), or any combination thereof.

In one or more embodiments, the phosphorescent dopant may include, for example, one of Compounds PD1 to PD39, or any combination thereof:

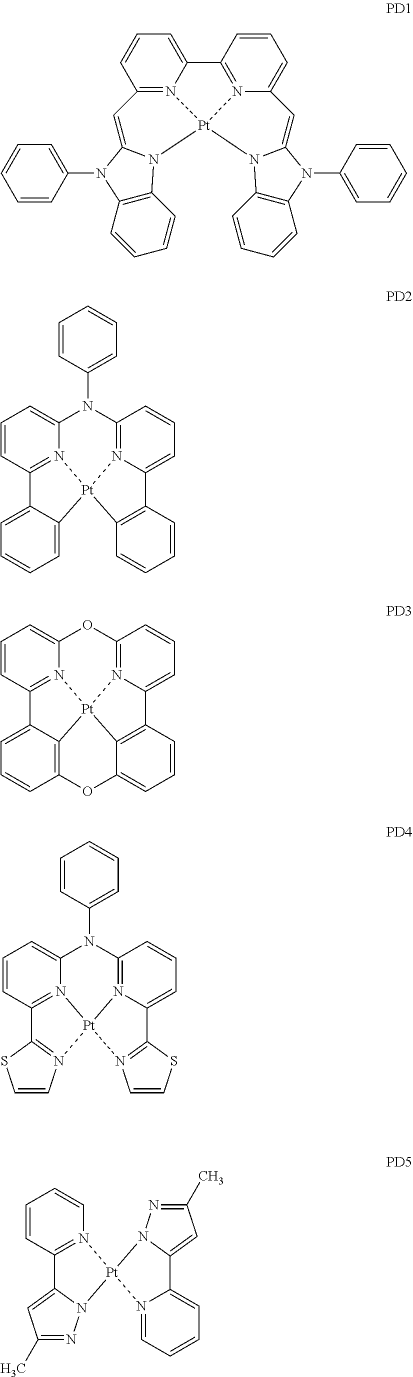

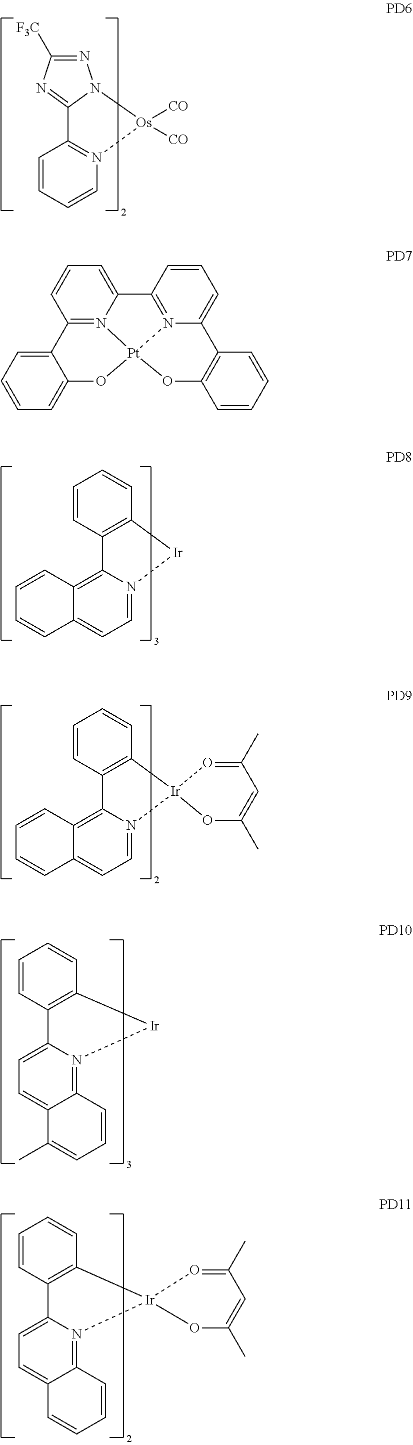

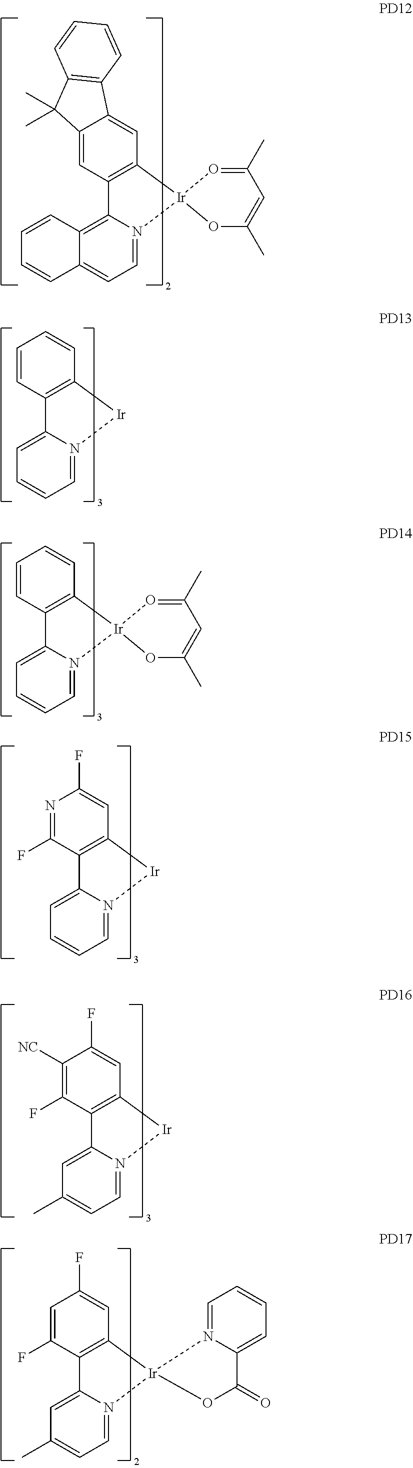

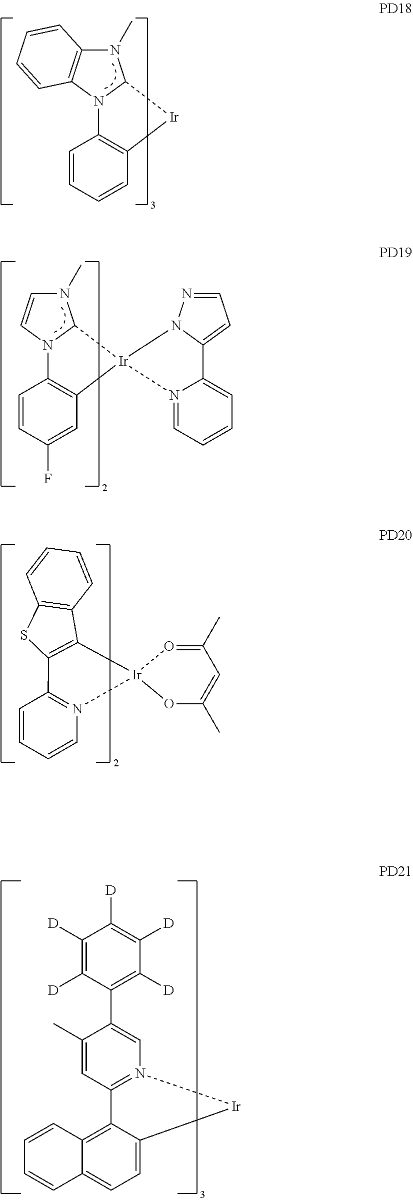

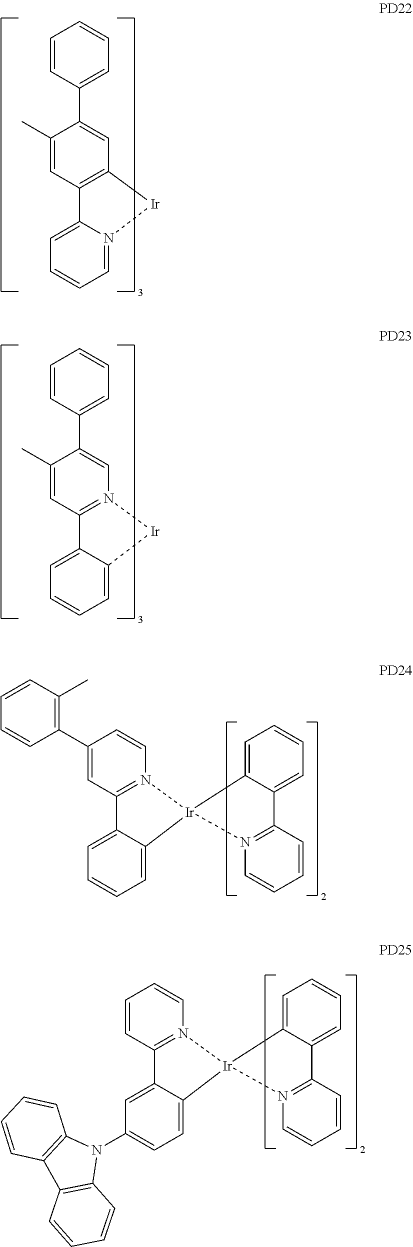

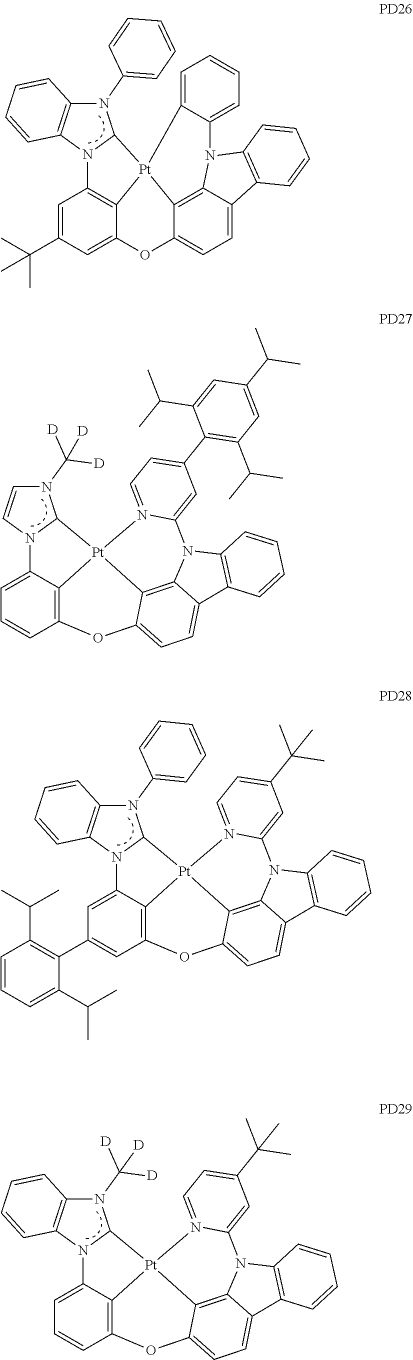

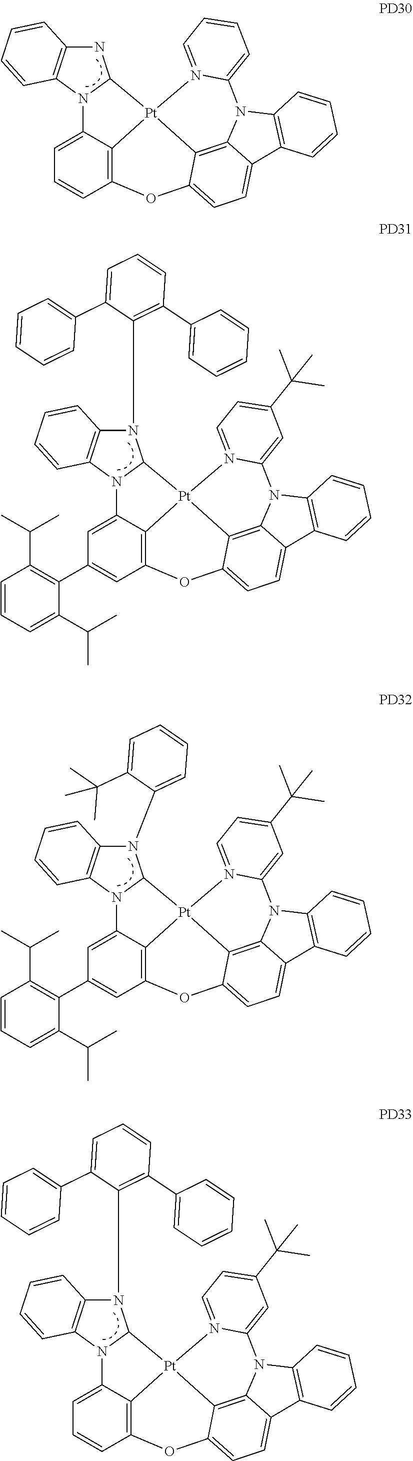

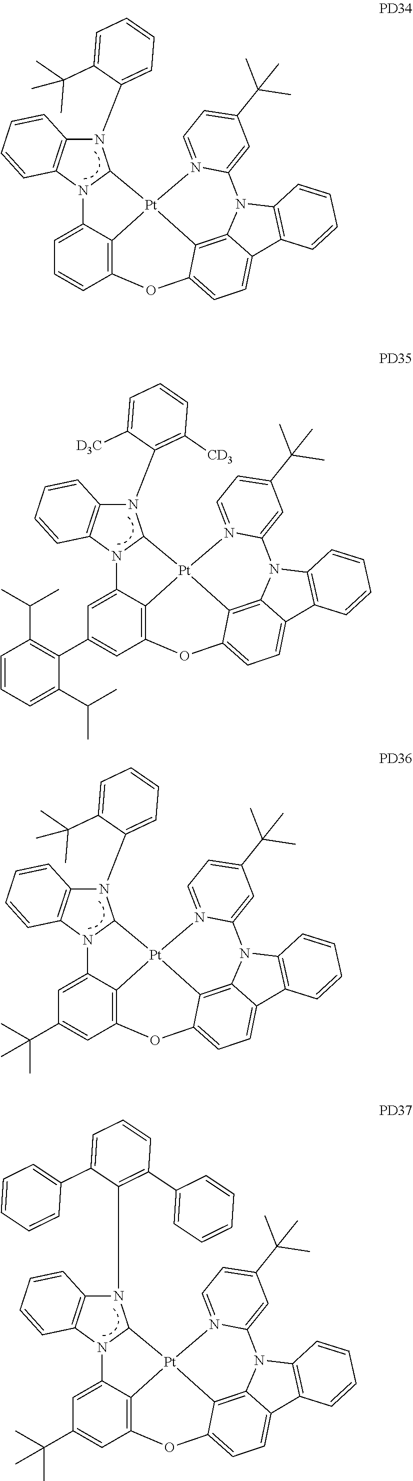

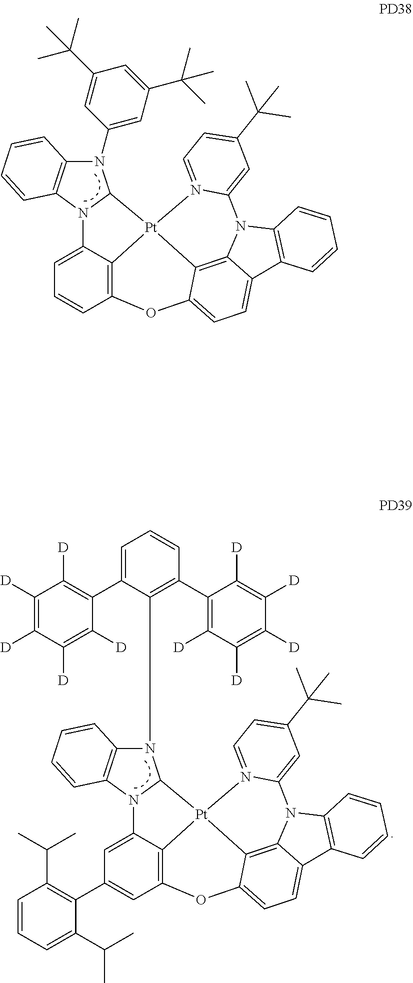

[Fluorescent Dopant]

In an embodiment, the fluorescent dopant may include an amine group-containing compound, a styryl group-containing compound, or any combination thereof.

In one or more embodiments, the fluorescent dopant may include a compound represented by Formula 501:

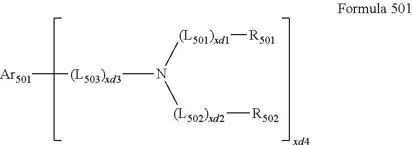

wherein, in Formula 501,

Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xd1 to xd3 may each independently be 0, 1, 2, or 3, and

xd4 may be 1, 2, 3, 4, 5, or 6.

For example, Ar₅₀₁ in Formula 501 may be a condensed cyclic group (for example, an anthracene group, a chrysene group, or a pyrene group) in which three or more monocyclic groups are condensed together.

For example, xd4 in Formula 501 may be 2.

In one or more embodiments, the fluorescent dopant may include: one of Compounds FD1 to FD36; DPVBi; DPAVBi; or any combination thereof:

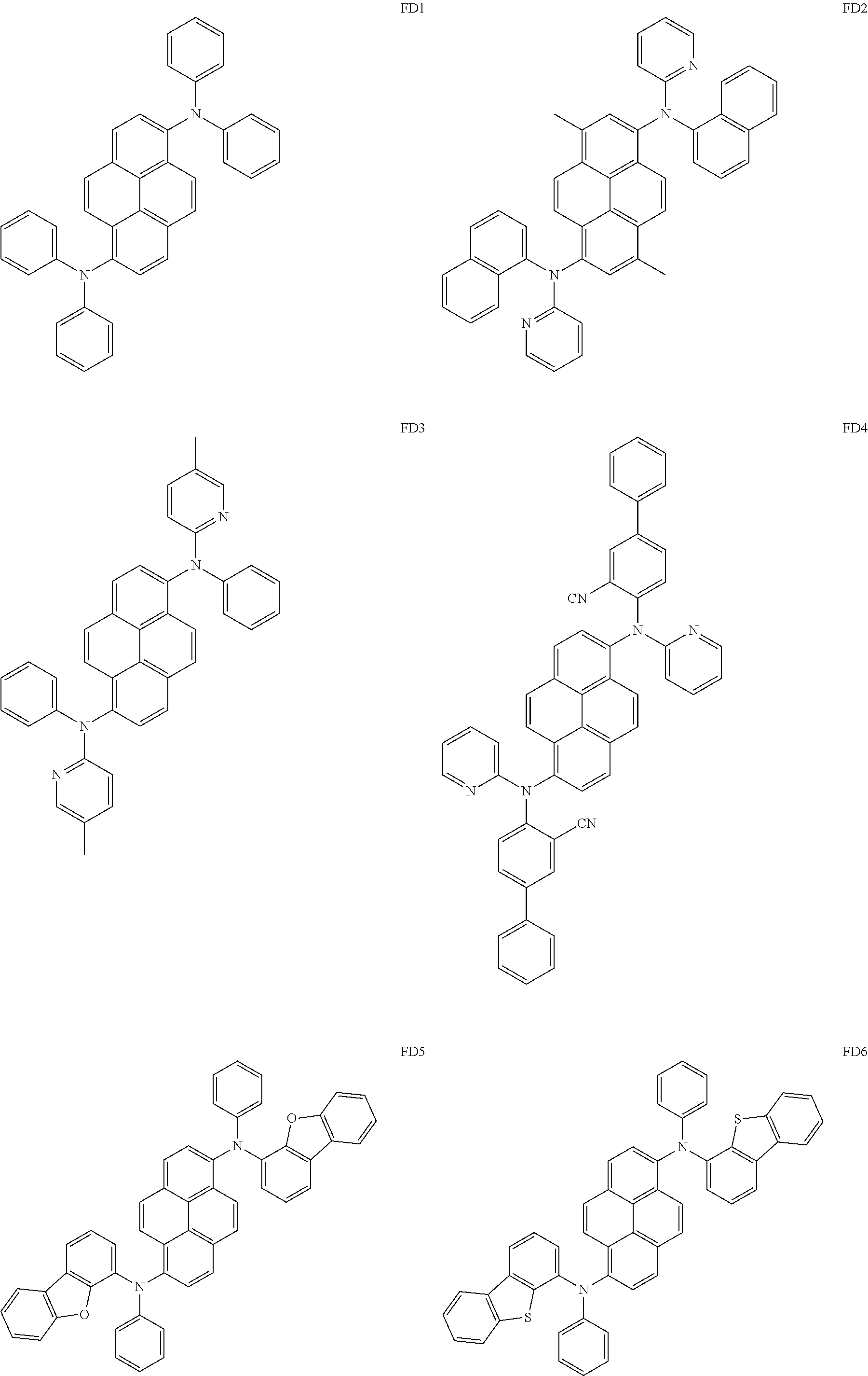

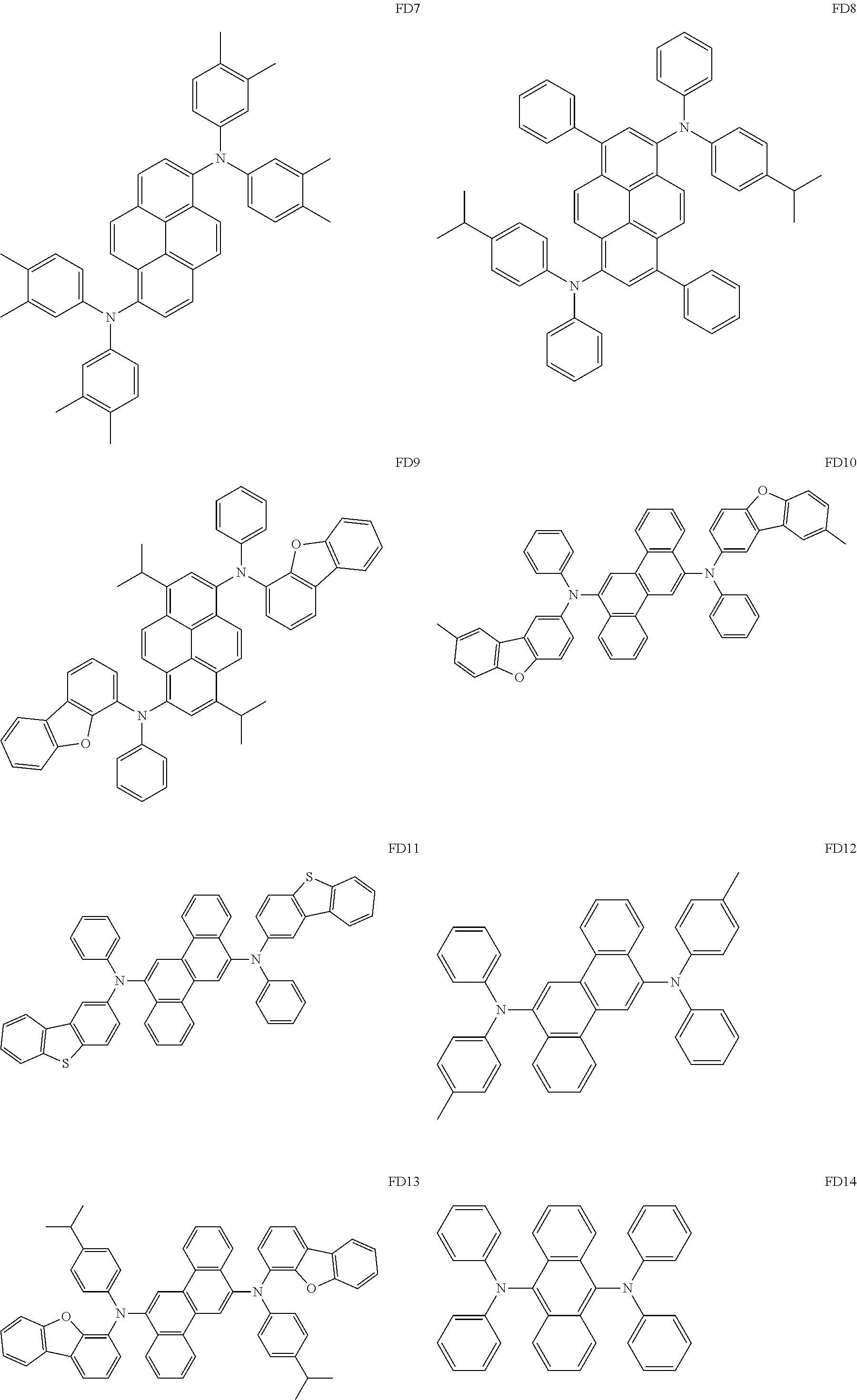

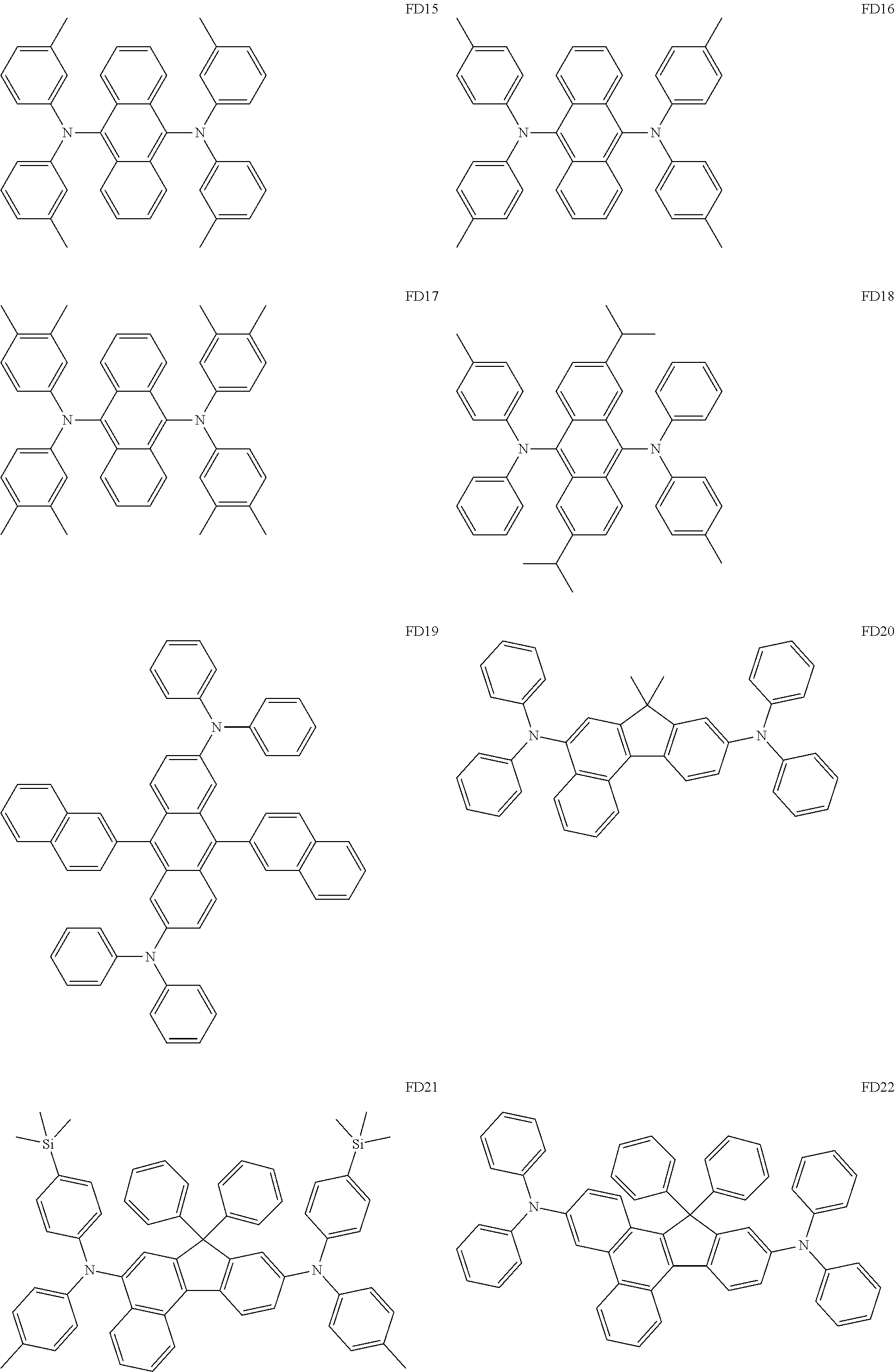

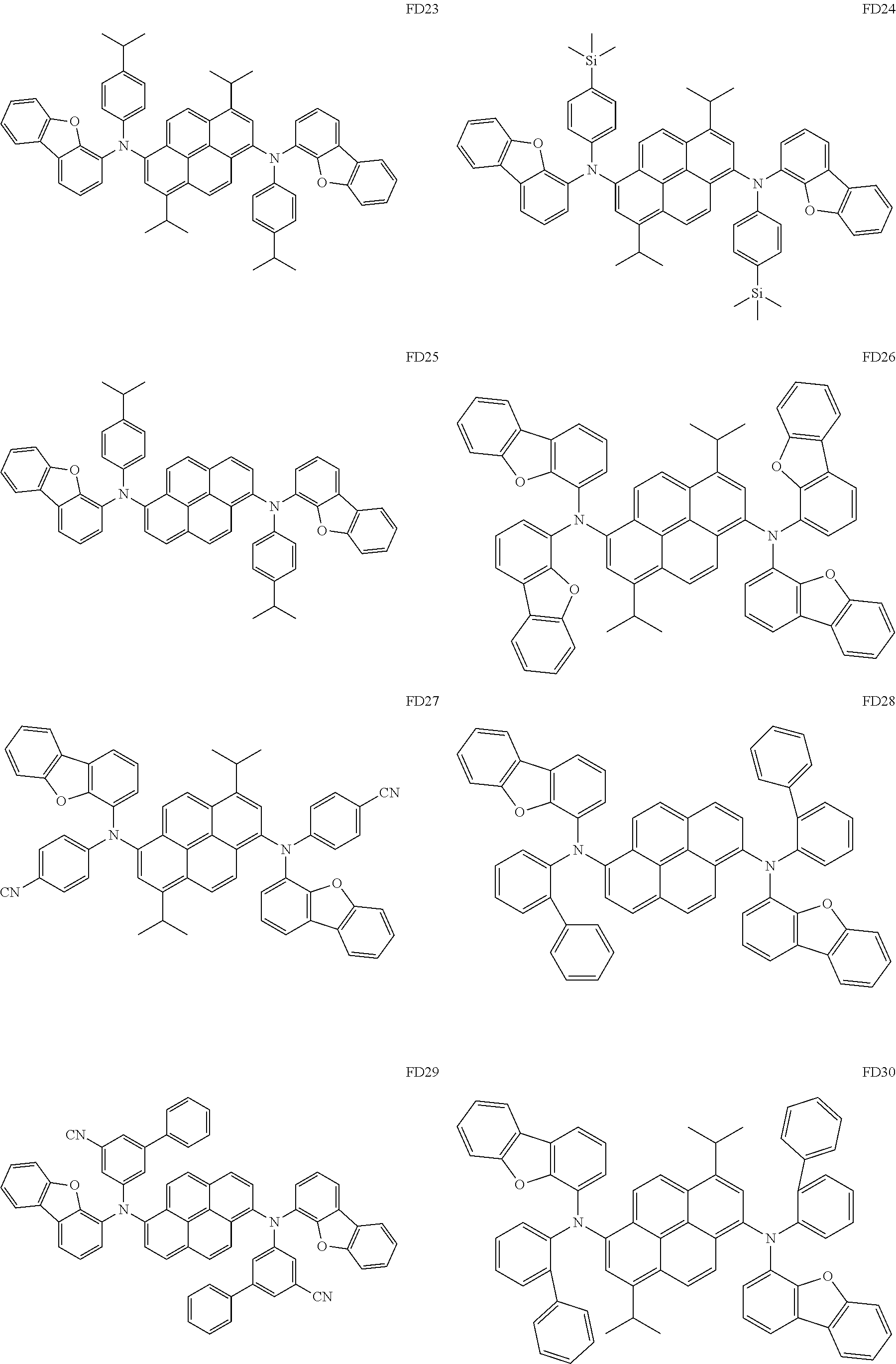

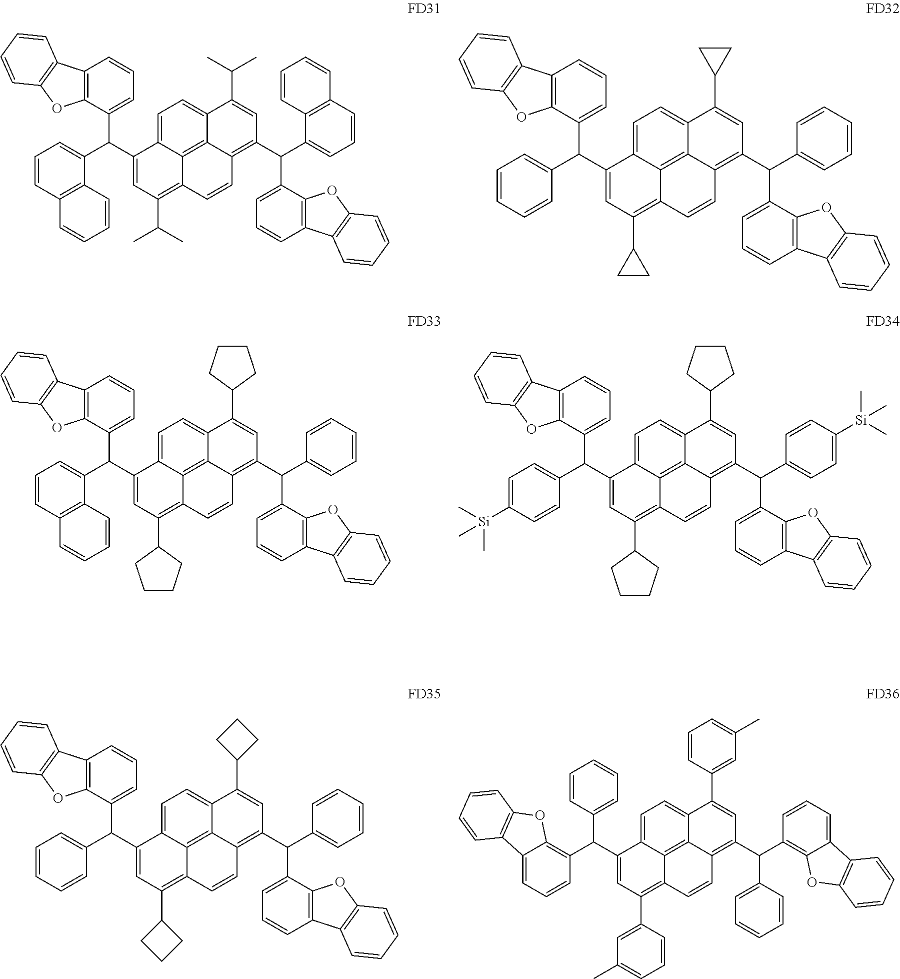

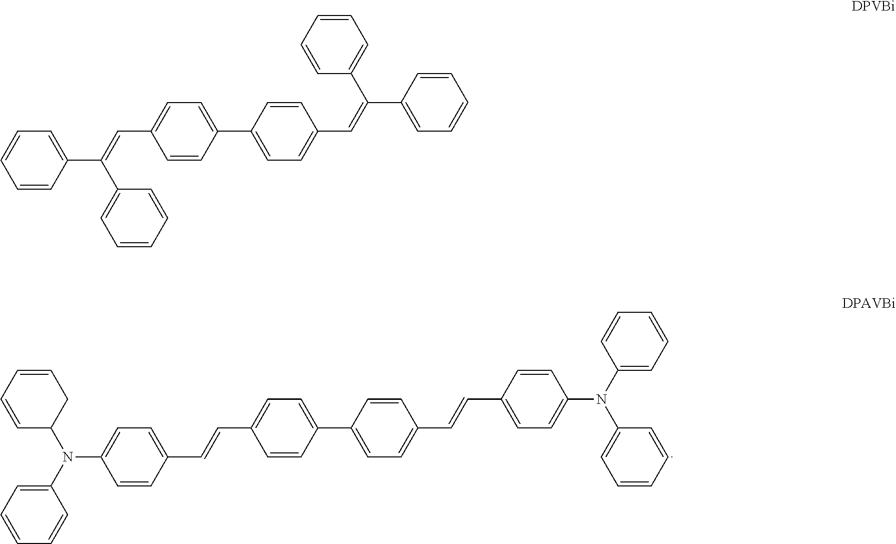

[Delayed Fluorescence Material]

The emission layer may include a delayed fluorescence material.

In the embodiments described herein, the delayed fluorescence material may be selected from compounds capable of emitting delayed fluorescence based on a delayed fluorescence emission mechanism.

The delayed fluorescence material included in the emission layer may act as a host or a dopant depending on the type of other materials included in the emission layer.

In an embodiment, a difference between a triplet energy level (eV) and a singlet energy level (eV) of the delayed fluorescence material may be about 0 eV or more and about 0.5 eV or less. When the difference between the triplet energy level (eV) and the singlet energy level (eV) of the delayed fluorescence material is within these ranges, up-conversion from the triplet state to the singlet state of the delayed fluorescence material may effectively occur, thereby improving luminescence efficiency of the light-emitting device 10.

In an embodiment, the delayed fluorescence material may include: i) a material including at least one electron donor (for example, a π electron-rich C₃-C₆₀ cyclic group and the like, such as a carbazole group) and at least one electron acceptor (for example, a sulfoxide group, a cyano group, a π electron-deficient nitrogen-containing C₁-C₆₀ cyclic group, and the like), ii) a material including a C₈-C₆₀ polycyclic group including at least two cyclic groups condensed to each other while sharing boron (B), and the like.

In one or more embodiments, the delayed fluorescence material may include at least one of Compounds DF1 to DF9:

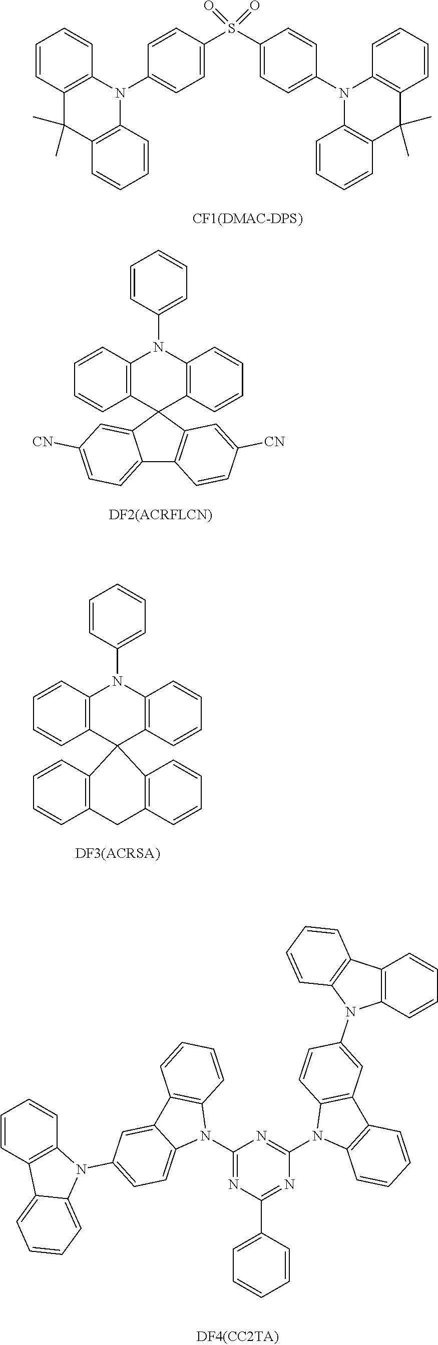

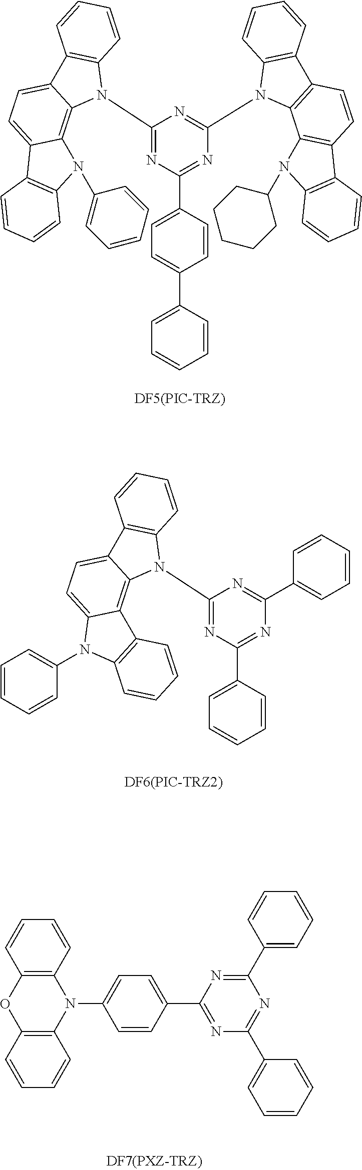 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(C7CCCCC7)c6c54)n3)cc2)cc1 ; c1ccc(-c2nc(-c3ccccc3)nc(-n3c4ccccc4c4ccc5c(c6ccccc6n5-c5ccccc5)c43)n2)cc1 ; c1ccc(-c2nc(-c3ccccc3)nc(-c3ccc(N4c5ccccc5Oc5ccccc54)cc3)n2)cc1]

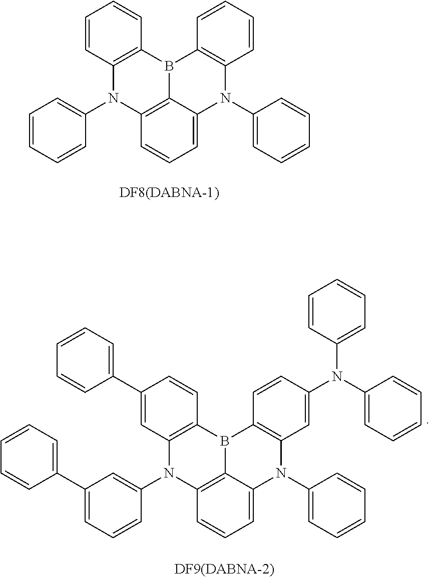 [SMILES: c1ccc(N2c3ccccc3B3c4ccccc4N(c4ccccc4)c4cccc2c43)cc1 ; c1ccc(-c2cccc(N3c4cc(-c5ccccc5)ccc4B4c5ccc(N(c6ccccc6)c6ccccc6)cc5N(c5ccccc5)c5cccc3c54)c2)cc1]

[Quantum Dot]

The emission layer may include quantum dots.

The term “quantum dot” as used herein refers to a crystal of a semiconductor compound, and may include any material capable of emitting light of various emission wavelengths according to the size of the crystal.

A diameter of the quantum dot may be, for example, in a range of about 1 nm to about 10 nm.

The quantum dots may be synthesized by a wet chemical process, a metal organic chemical vapor deposition process, a molecular beam epitaxy process, or any process similar thereto.

The wet chemical process is a method including mixing a precursor material with an organic solvent and then growing a quantum dot particle crystal. When the crystal grows, the organic solvent naturally acts as a dispersant coordinated on the surface of the quantum dot crystal and controls the growth of the crystal so that the growth of quantum dot particles can be controlled through a process which costs lower, and is easier than vapor deposition methods, such as metal organic chemical vapor deposition (MOCVD) or molecular beam epitaxy (MBE).

The quantum dot may include: a Group II-VI semiconductor compound; a Group III-V semiconductor compound; a Group III-VI semiconductor compound; a Group I-III-VI semiconductor compound; a Group IV-VI semiconductor compound; a Group IV element or compound; or any combination thereof.

Examples of the Group II-VI semiconductor compound are: a binary compound, such as CdSe, CdTe, ZnS, ZnSe, ZnTe, ZnO, HgS, HgSe, HgTe, MgSe, MgS, and the like; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and the like; a quaternary compound, such as CdZnSeS, CdZnSeTe, CdZnSTe, CdHgSeS, CdHgSeTe, CdHgSTe, HgZnSeS, HgZnSeTe, HgZnSTe, and the like; or any combination thereof.

Examples of the Group III-V semiconductor compound are: a binary compound, such as GaN, GaP, GaAs, GaSb, AlN, AlP, AlAs, AlSb, InN, InP, InAs, InSb, and the like; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, InPSb, and the like; a quaternary compound, such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and the like; or any combination thereof. The Group III-V semiconductor compound may further include a Group II element. Examples of the Group III-V semiconductor compound further including the Group II element are InZnP, InGaZnP, InAlZnP, and the like.

Examples of the Group III-VI semiconductor compound are: a binary compound, such as GaS, GaSe, Ga₂Se₃, GaTe, InS, InSe, In₂S₃, In₂Se₃, InTe, and the like; a ternary compound, such as InGaS₃, InGaSe₃, and the like; or any combination thereof.

Examples of the Group I-III-VI semiconductor compound are: a ternary compound, such as AgInS, AgInS₂, CuInS, CuInS₂, CuGaO₂, AgGaO₂, AgAlO₂, and the like; or any combination thereof.

Examples of the Group IV-VI semiconductor compound are: a binary compound, such as SnS, SnSe, SnTe, PbS, PbSe, PbTe, and the like; a ternary compound, such as SnSeS, SnSeTe, SnSTe, PbSeS, PbSeTe, PbSTe, SnPbS, SnPbSe, SnPbTe, and the like; a quaternary compound, such as SnPbSSe, SnPbSeTe, SnPbSTe, and the like; or any combination thereof.

The Group IV element or compound may include: a single element compound, such as Si, Ge, and the like; a binary compound, such as SiC, SiGe, and the like; or any combination thereof.

Each element included in a multi-element compound, such as the binary compound, the ternary compound, and the quaternary compound, may exist in a particle thereof at a uniform concentration or non-uniform concentration.

The quantum dot may have a single structure or a dual core-shell structure. In the case of the quantum dot having a single structure, a concentration of each element included in the corresponding quantum dot may be uniform. For example, a material included in the core and a material included in the shell may be different from each other.

The shell of the quantum dot may act as a protective layer that prevents chemical degeneration of the core to maintain semiconductor characteristics, and/or as a charging layer that imparts electrophoretic characteristics to the quantum dot. The shell may be a single layer or a multi-layer. The interface between the core and the shell may have a concentration gradient in which the concentration of an element existing in the shell decreases toward the center of the core.

Examples of the shell of the quantum dot are oxide of metal, metalloid, or non-metal, a semiconductor compound, or any combination thereof. Examples of the oxide of metal, metalloid, or non-metal are: a binary compound, such as SiO₂, Al₂O₃, TiO₂, ZnO, MnO, Mn₂O₃, Mn₃O₄, CuO, FeO, Fe₂O₃, Fe₃O₄, CoO, CO₃O₄, NiO, and the like; a ternary compound, such as MgAl₂O₄, CoFe₂O₄, NiFe₂O₄, CoMn₂O₄, and the like; or any combination thereof. Examples of the semiconductor compound are: as described herein, a Group II-VI semiconductor compound; a Group III-V semiconductor compound; a Group III-VI semiconductor compound; a Group I-III-VI semiconductor compound; a Group IV-VI semiconductor compound; or any combination thereof. Examples of the semiconductor compound are CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or any combination thereof.

A full width at half maximum (FWHM) of an emission wavelength spectrum of the quantum dots may be equal to or less than about 45 nm, for example, equal to or less than about 40 nm, and for example, equal to or less than about 30 nm, and within these ranges, color purity or color reproducibility may be improved. In addition, since the light emitted through the quantum dots is emitted in all directions, the wide viewing angle may be improved.

In addition, the quantum dot may be specifically, spherical, pyramidal, multi-arm, or cubic nanoparticles, nanotubes, nanowires, nanofibers, or nanoplate particles.

Since the energy band gap may be adjusted by controlling the size of the quantum dots, light having various wavelength bands may be obtained from the emission layer including the quantum dots. Accordingly, by using the quantum dots of different sizes, a light-emitting device that emits light of various wavelengths may be implemented. In detail, the size of the quantum dots may be selected to emit red light, green light, and/or blue light. In addition, the size of the quantum dots may be selected to emit white light by combination of light of various colors.

[Electron Transport Region in Interlayer 130]

The electron transport region may have i) a single-layered structure consisting of a single layer consisting of a single material, ii) a single-layered structure consisting of a single layer consisting of a plurality of different materials, or iii) a multi-layered structure including a plurality of layers including different materials.

The electron transport region may include a buffer layer, a hole blocking layer, an electron control layer, an electron transport layer, an electron injection layer, or any combination thereof.

For example, the electron transport region may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, wherein layers of each structure are sequentially stacked from the emission layer.

In an embodiment, the electron transport region (for example, the buffer layer, the hole blocking layer, the electron control layer, or the electron transport layer in the electron transport region) may include a metal-free compound including at least one π electron-deficient nitrogen-containing C₁-C₆₀ cyclic group.

In one or more embodiments, the electron transport region may include a compound represented by Formula 601:

[Ar₆₀₁]ₓₑ₁₁-[(L₆₀₁)ₓₑ₁-R₆₀₁]ₓₑ₂₁ Formula 601

wherein, in Formula 601,

Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xe11 may be 1, 2, or 3,

xe1 may be 0, 1, 2, 3, 4, or 5,

R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),

Q₆₀₁ to Q₆₀₃ may respectively be the same as described in connection with Q₁,

xe21 may be 1, 2, 3, 4, or 5, and

at least one of Ar₆₀₁, L₆₀₁, and R₆₀₁ may each independently be a π electron-deficient nitrogen-containing C₁-C₆₀ cyclic group unsubstituted or substituted with at least one R₁₀ₐ.

In an embodiment, when xe11 in Formula 601 is 2 or more, two or more of Ar₆₀₁(s) may be linked to each other via a single bond.

In an embodiment, Ar₆₀₁ in Formula 601 may be a substituted or unsubstituted anthracene group.

In one or more embodiments, the electron transport region may include a compound represented by Formula 601-1:

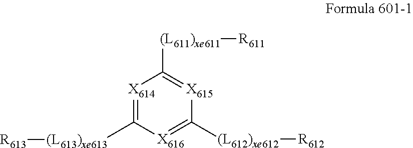

wherein, in Formula 601-1,

X₆₁₄ may be N or C(R₆₁₄), X₆₁₅ may be N or C(R₆₁₅), X₆₁₆ may be N or C(R₆₁₆), and at least one of X₆₁₄ to X₆₁₆ may be N,

L₆₁₁ to L₆₁₃ may respectively be the same as described in connection with L₆₀₁,

xe611 to xe613 may respectively be the same as described in connection with xe1,

R₆₁₁ to R₆₁₃ may respectively be the same as described in connection with R₆₀₁, and

R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

The electron transport region may include one of Compounds ET1 to ET45, 2,9-dimethyl-4,7-diphenyl-1,10-phenanthroline (BCP), 4,7-diphenyl-1,10-phenanthroline (Bphen), Alq₃, BAlq, TAZ, NTAZ, or any combination thereof:

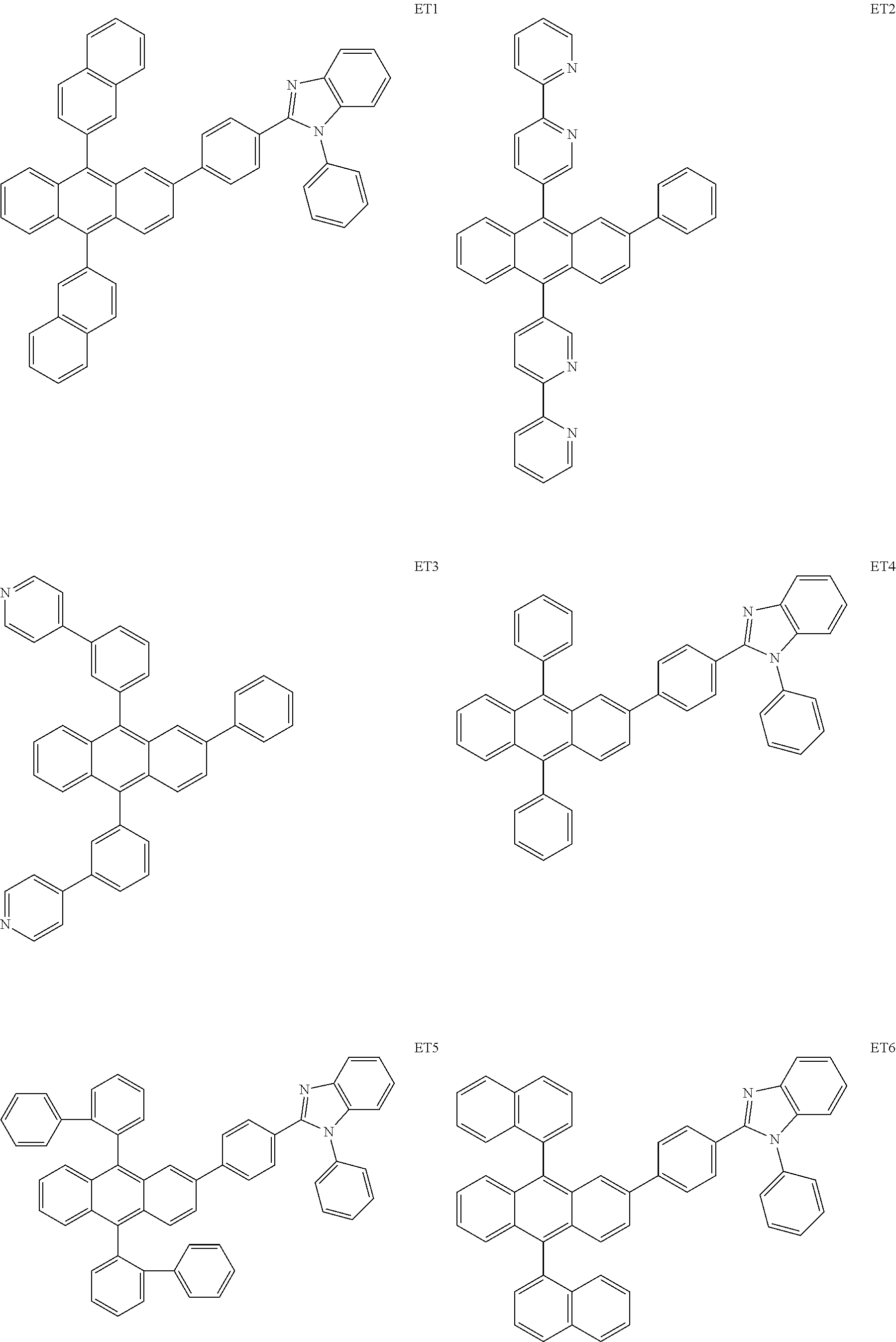

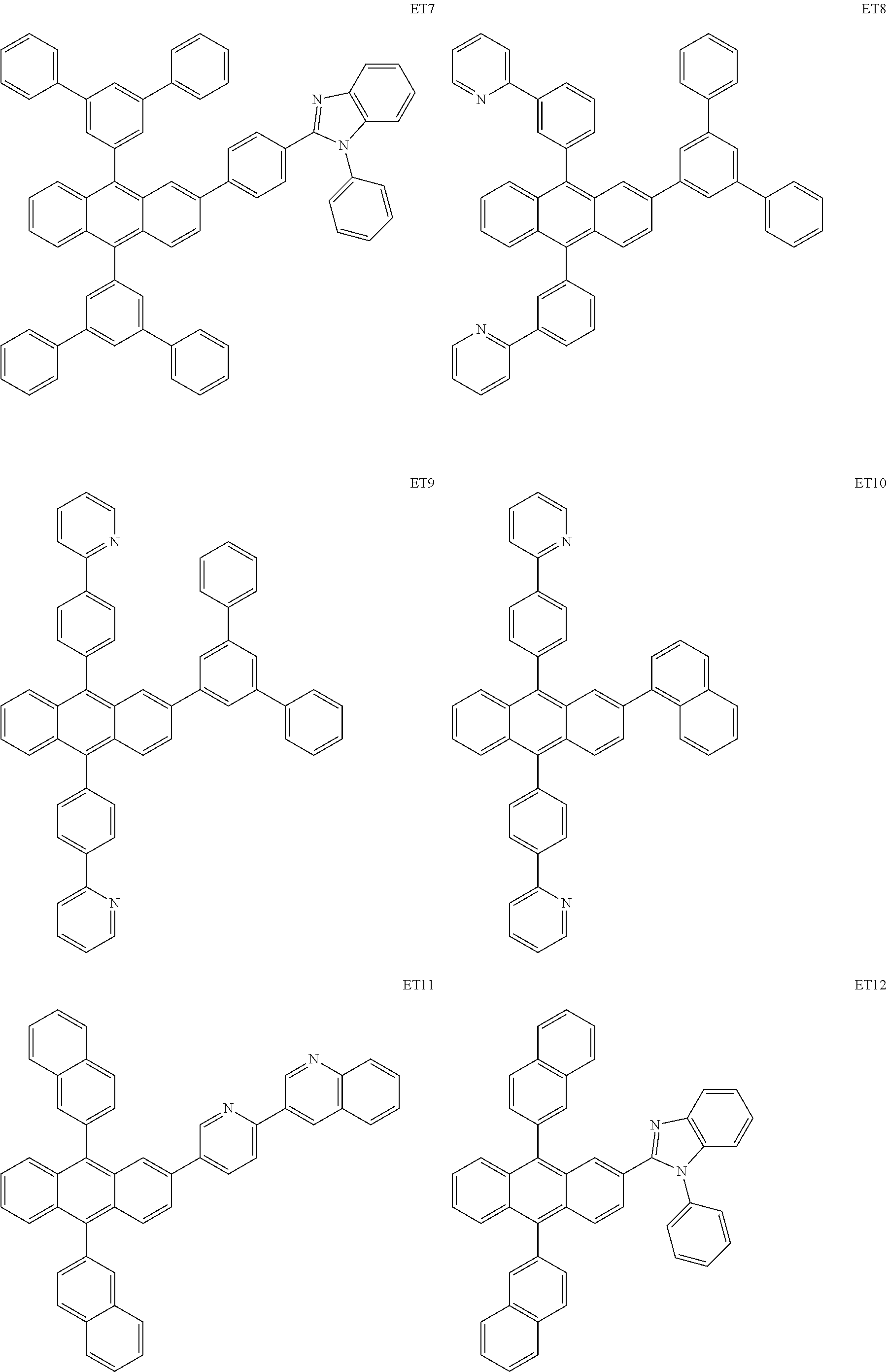

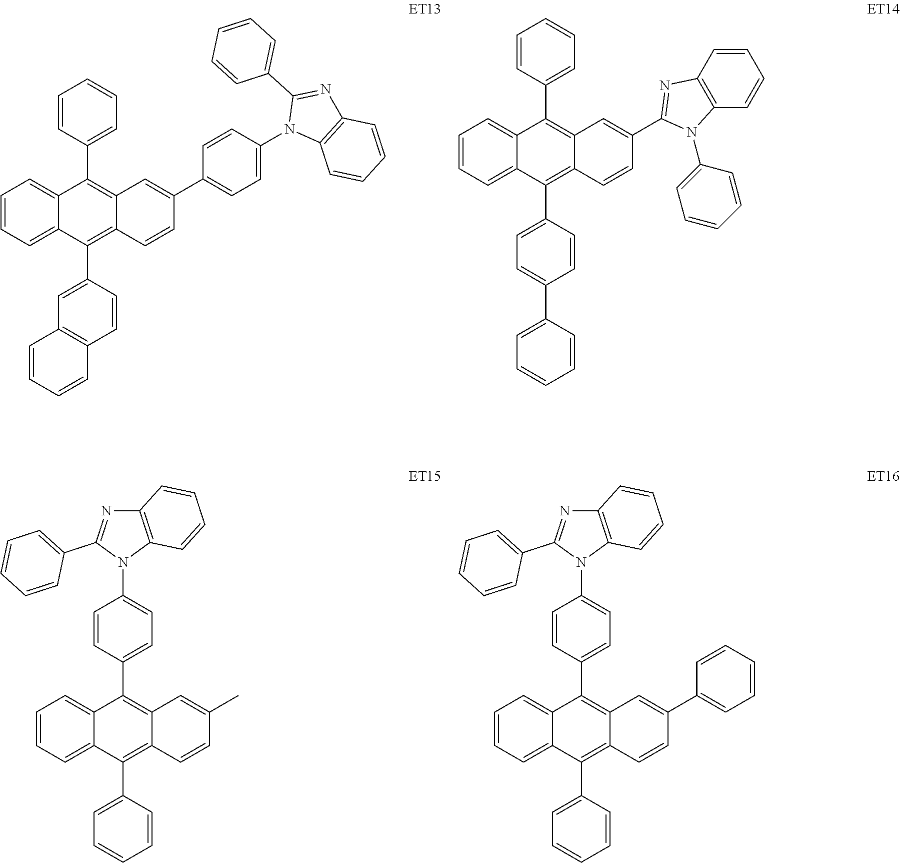 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

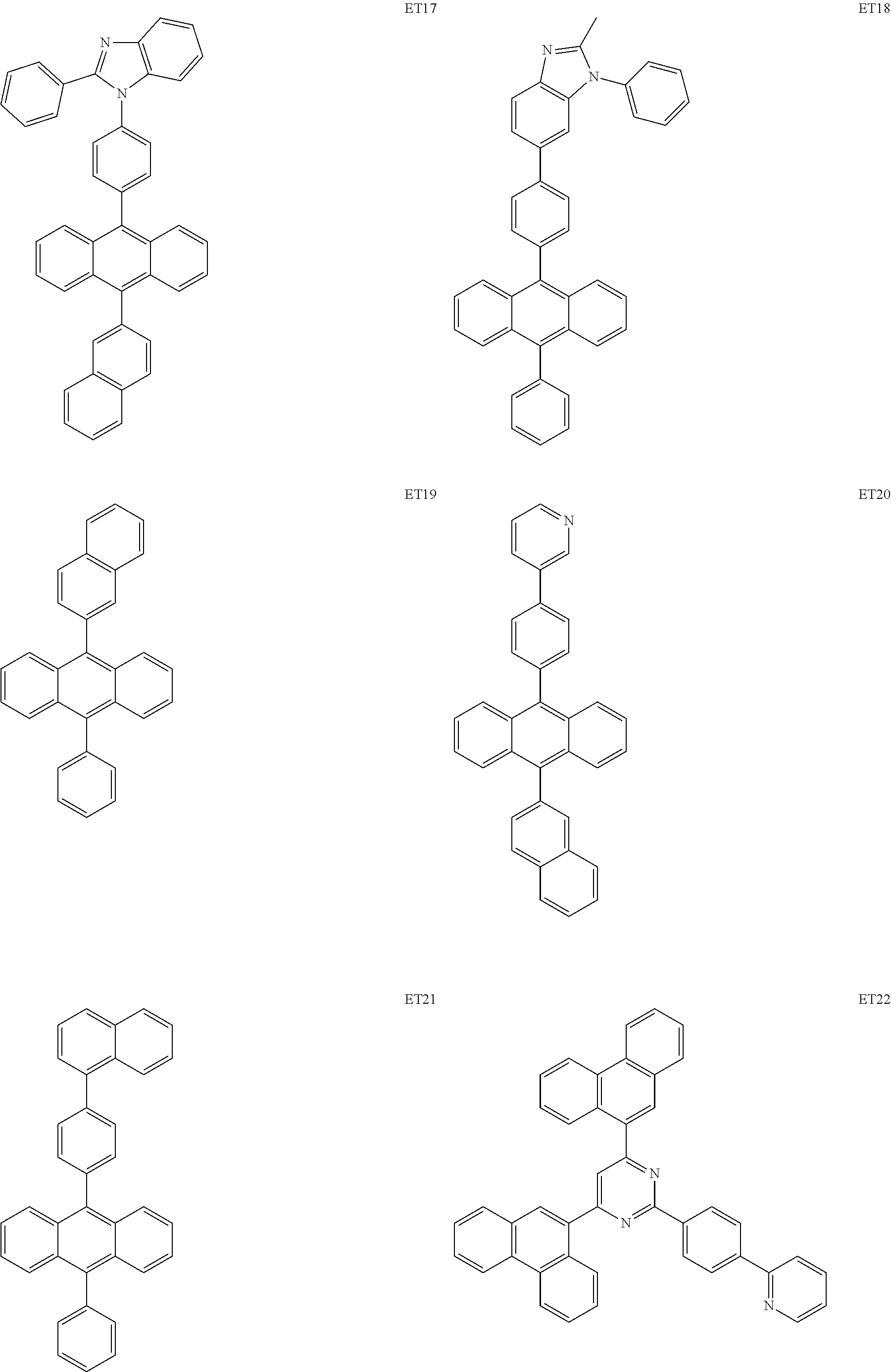

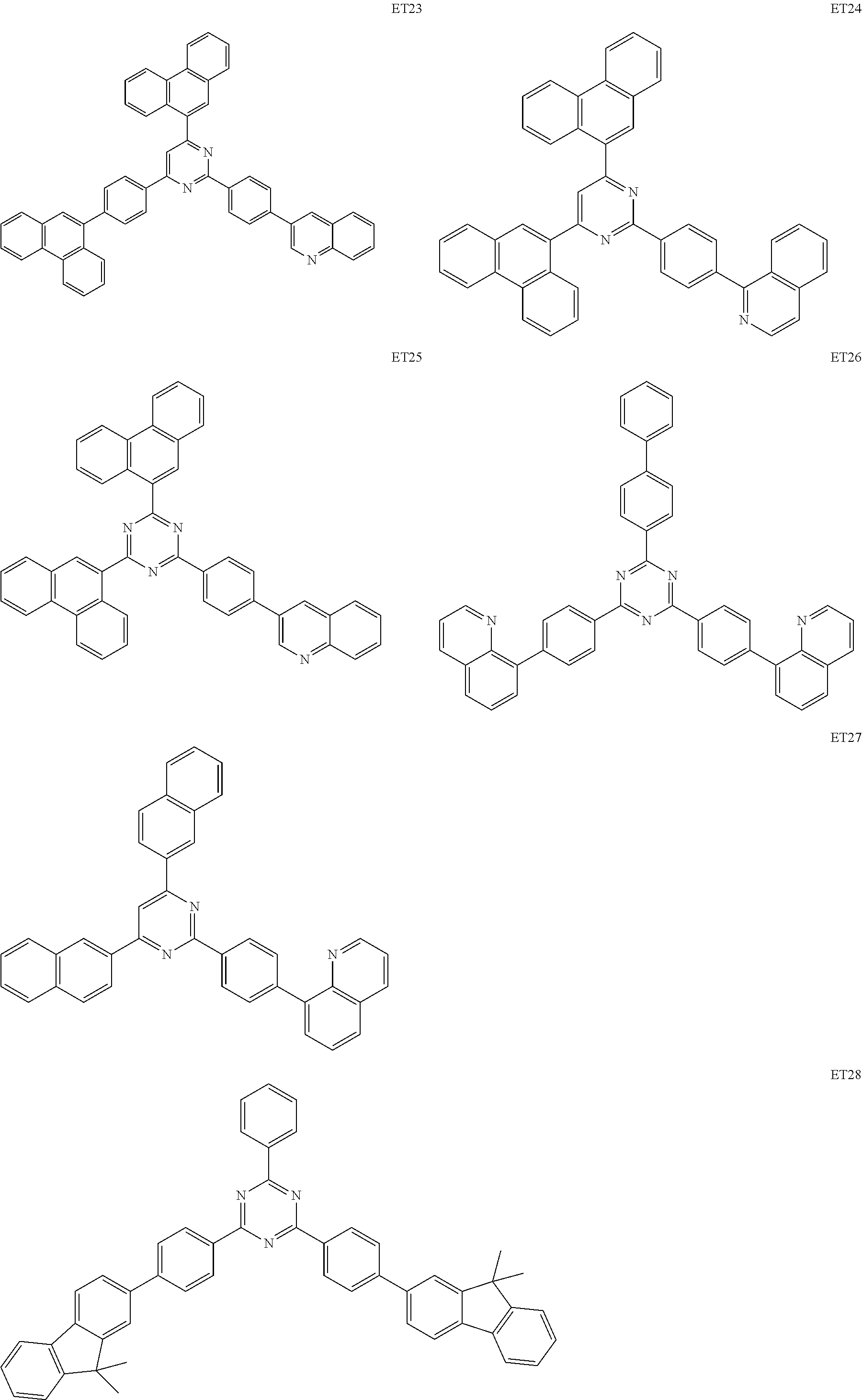

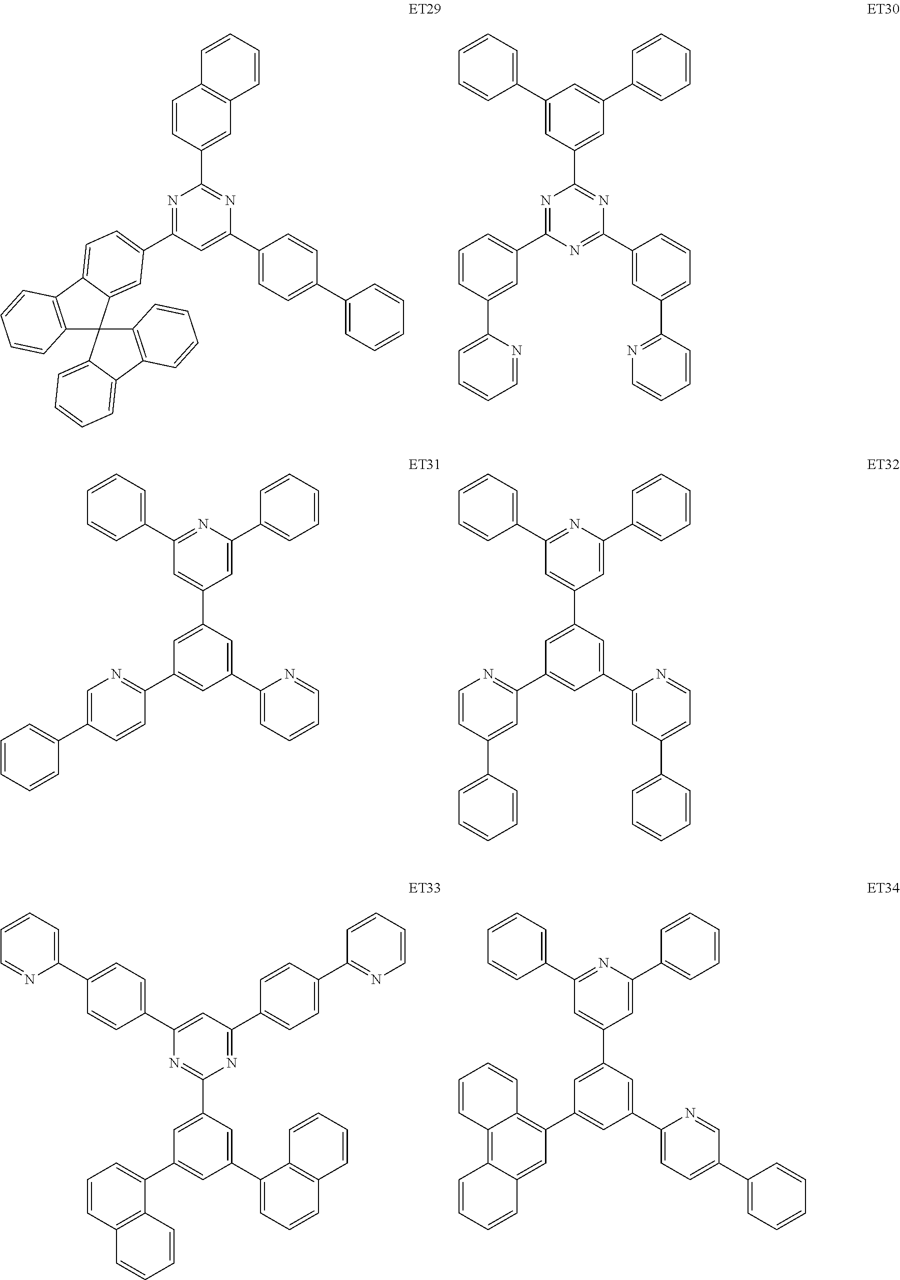

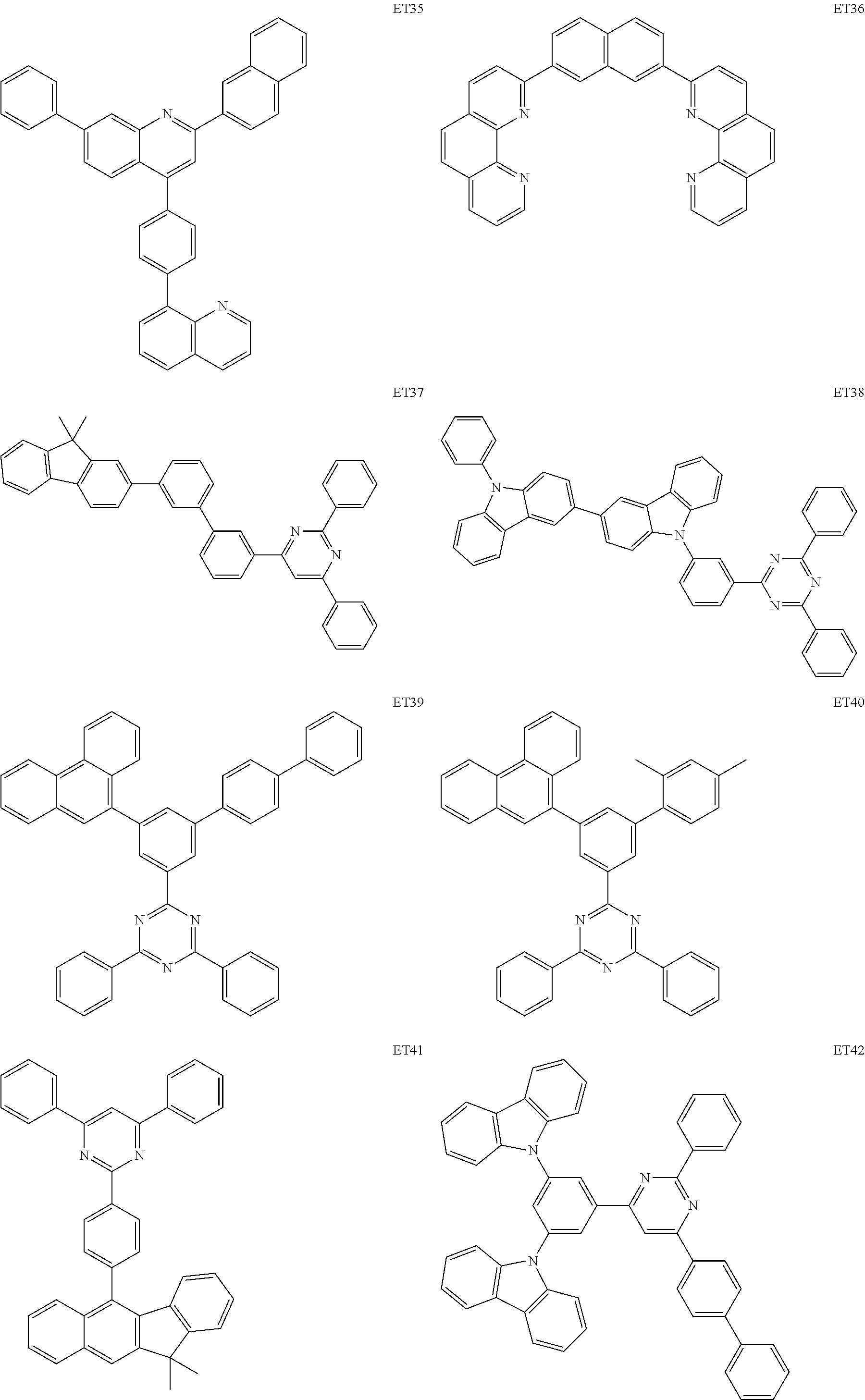

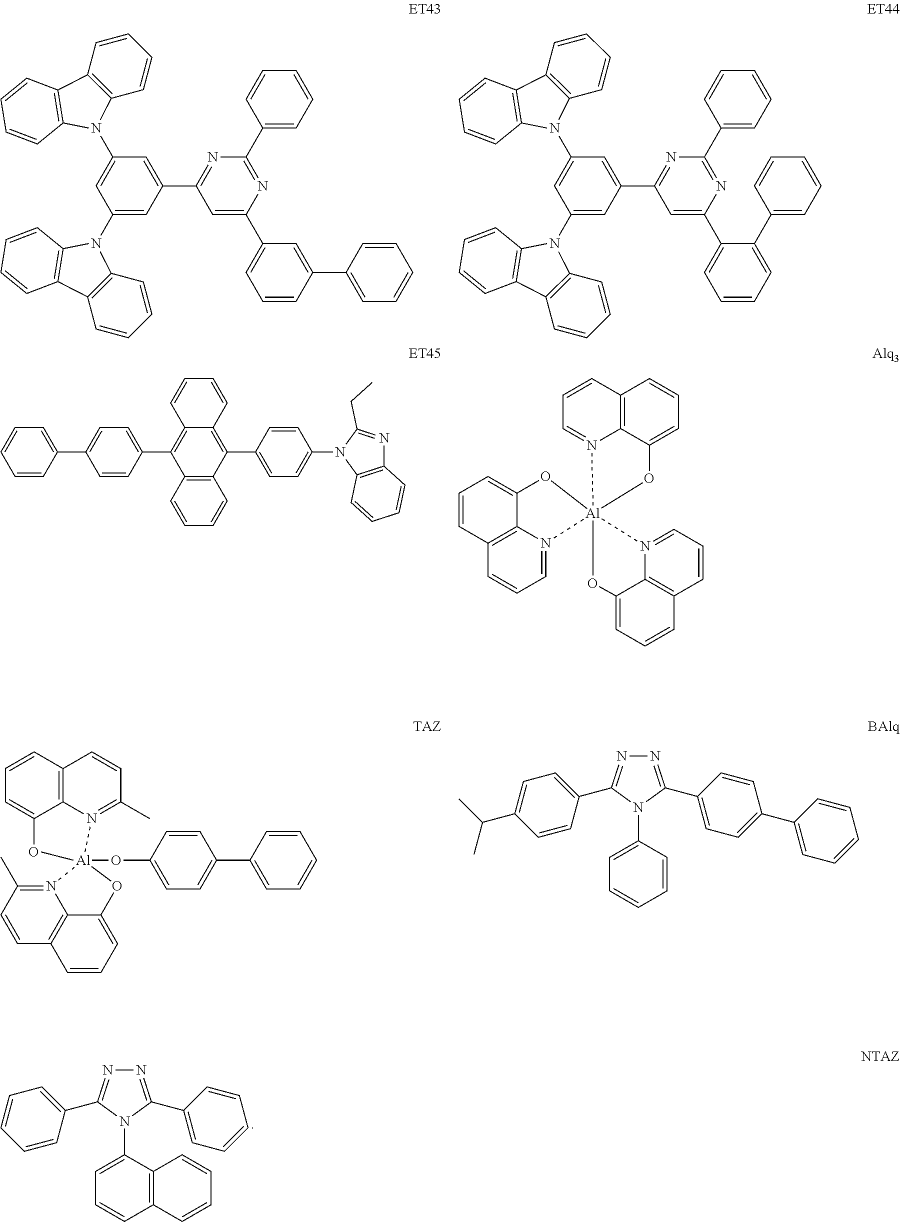

A thickness of the electron transport region may be in a range of about 100 Å to about 5,000 Å, for example, about 160 Å to about 4,000 Å. When the electron transport region includes a buffer layer, a hole blocking layer, an electron control layer, an electron transport layer, or any combination thereof, a thickness of the buffer layer, the hole blocking layer, or the electron control layer may each independently be in a range of about 20 Å to about 1,000 Å, for example, about 30 Å to about 300 Å, and a thickness of the electron transport layer may be in a range of about 100 Å to about 1,000 Å, for example, about 150 Å to about 500 Å. When the thickness of the buffer layer, the hole blocking layer, the electron control layer, the electron transport layer, and/or the electron transport region are within these ranges, satisfactory electron transporting characteristics may be obtained without a substantial increase in driving voltage.

The electron transport region (for example, the electron transport layer in the electron transport region) may further include, in addition to the materials described above, a metal-containing material.

The metal-containing material may include an alkali metal complex, an alkaline earth metal complex, or any combination thereof. The metal ion of an alkali metal complex may be a Li ion, a Na ion, a K ion, a Rb ion, or a Cs ion, and the metal ion of an alkaline earth metal complex may be a Be ion, a Mg ion, a Ca ion, a Sr ion, or a Ba ion. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

For example, the metal-containing material may include a Li complex. The Li complex may include, for example, Compound ET-D1 (LiQ) or ET-D2:

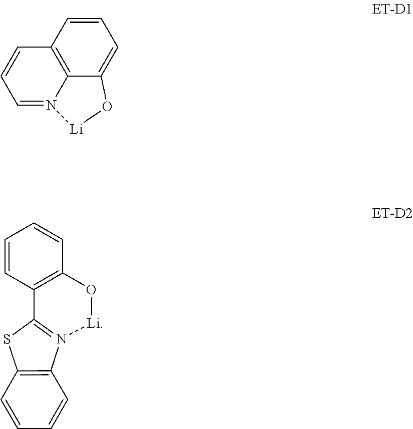

The electron transport region may include an electron injection layer that facilitates the injection of electrons from the second electrode 150. The electron injection layer may directly contact the second electrode 150.

The electron injection layer may have: i) a single-layered structure consisting of a single layer consisting of a single material, ii) a single-layered structure consisting of a single layer consisting of a plurality of different materials, or iii) a multi-layered structure including a plurality of layers including different materials.

The electron injection layer may include alkali metal, alkaline earth metal, rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may be oxides, halides (for example, fluorides, chlorides, bromides, iodides, and the like), or tellurides of the alkali metal, the alkaline earth metal, and the rare earth metal, or any combination thereof.

The alkali metal-containing compound may include alkali metal oxides, such as Li₂O, Cs₂O, K₂O, and the like, alkali metal halides, such as LiF, NaF, CsF, KF, LiI, NaI, CsI, KI, and the like, or any combination thereof. The alkaline earth metal-containing compound may include an alkaline earth metal compound, such as BaO, SrO, CaO, BaₓSr₁₋ₓO (wherein x is a real number satisfying the condition of 0≤x≤1), BaₓCa₁₋ₓO (wherein x is a real number satisfying the condition of 0≤x≤1), or the like. The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. For example, the rare earth metal-containing compound may include lanthanide metal telluride. Examples of the lanthanide metal telluride are LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, Lu₂Te₃, and the like.

The alkali metal complex, the alkaline earth-metal complex, and the rare earth metal complex may include i) one of ions of the alkali metal, the alkaline earth metal, and the rare earth metal and ii), as a ligand bonded to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

In an embodiment, the electron injection layer may consist of an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (for example, the compound represented by Formula 601).

In one or more embodiments, the electron injection layer may consist of: i) an alkali metal-containing compound (for example, an alkali metal halide); or ii) a) an alkali metal-containing compound (for example, an alkali metal halide), and b) an alkali metal, an alkaline earth metal, a rare earth metal, or any combination thereof. In one or more embodiments, the electron injection layer may be a KI:Yb co-deposited layer, an RbI:Yb co-deposited layer, and the like.

When the electron injection layer further includes an organic material, alkali metal, alkaline earth metal, rare earth metal, an alkali metal-containing compound, an alkaline earth metal-containing compound, a rare earth metal-containing compound, an alkali metal complex, an alkaline earth-metal complex, a rare earth metal complex, or any combination thereof may be homogeneously or non-homogeneously dispersed in a matrix including the organic material.

A thickness of the electron injection layer may be in a range of about 1 Å to about 100 Å, for example, about 3 Å to about 90 Å. When the thickness of the electron injection layer is within these ranges, satisfactory electron injection characteristics may be obtained without a substantial increase in driving voltage.

[Second Electrode 150]

The second electrode 150 may be on the interlayer 130 having a structure as described above. The second electrode 150 may be a cathode, which is an electron injection electrode, and a material for forming the second electrode 150 may include a metal, an alloy, an electrically conductive compound, or any combination thereof, each having a low-work function.

The material for forming the second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layered structure or a multi-layered structure including a plurality of layers.

[Capping Layer]

A first capping layer may be arranged outside the first electrode 110, and/or a second capping layer may be arranged outside the second electrode 150. In particular, the light-emitting device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

Light generated in the emission layer of the interlayer 130 of the light-emitting device 10 may be extracted toward the outside through the first electrode 110, which is a semi-transmissive electrode or a transmissive electrode, and the first capping layer, and light generated in the emission layer of the interlayer 130 of the light-emitting device 10 may be extracted toward the outside through the second electrode 150, which is a semi-transmissive electrode or a transmissive electrode, and the second capping layer.

The first capping layer and the second capping layer may increase external luminescence efficiency according to the principle of constructive interference. Accordingly, the light extraction efficiency of the light-emitting device 10 may be increased, so that the luminescence efficiency of the light-emitting device 10 may be improved.

Each of the first capping layer and the second capping layer may include a material having a refractive index of equal to or greater than 1.6 (at 589 nm).

The first capping layer and the second capping layer may each independently be an organic capping layer including an organic material, an inorganic capping layer including an inorganic material, or an organic-inorganic composite capping layer including an organic material and an inorganic material.

In an embodiment, at least one of the first capping layer and the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may each optionally be substituted with a substituent including O, N, S, Se, Si, F, Cl, Br, I, or any combination thereof. In one or more embodiments, at least one of the first capping layer and the second capping layer may each independently include an amine group-containing compound.

For example, at least one of the first capping layer and the second capping layer may each independently include a compound represented by Formula 201, a compound represented by Formula 202, or any combination thereof.

In one or more embodiments, at least one of the first capping layer and the second capping layer may each independently include one of Compounds HT28 to HT33, one of Compounds CP1 to CP6, β-NPB, or any combination thereof:

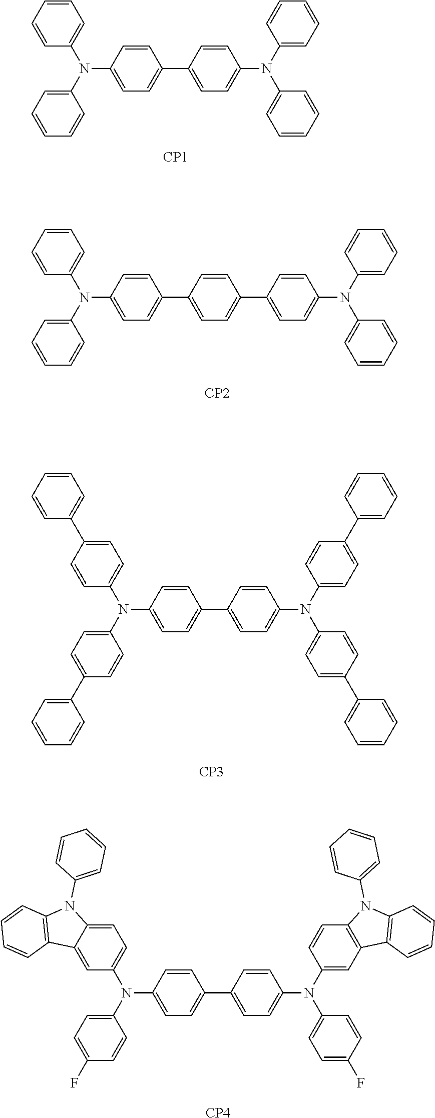

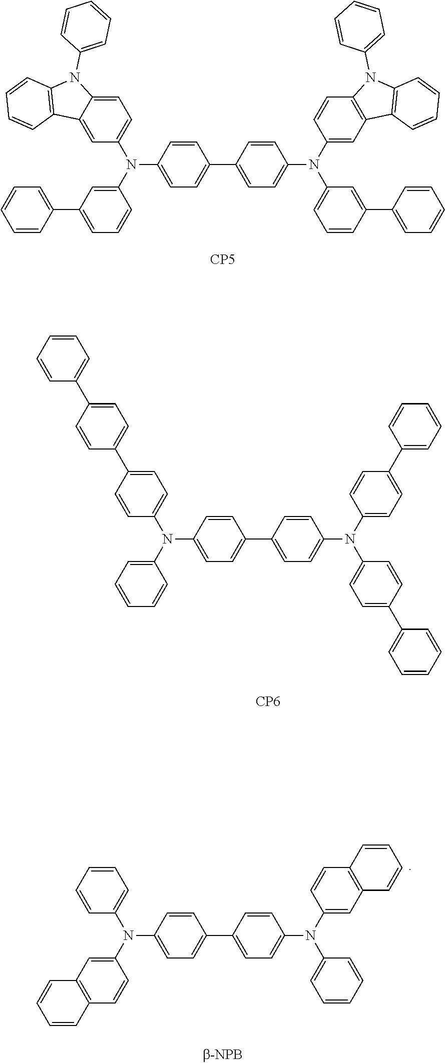

[Film]

The condensed cyclic compound represented by Formula 1 may be included in various films. Accordingly, another aspect of the present disclosure provides a film including the condensed cyclic compound represented by Formula 1. The film may be, for example, an optical member (or a light control unit) (for example, a color filter, a color conversion member, a capping layer, a light extraction efficiency enhancement layer, a selective light absorbing layer, a polarizing layer, a quantum dot-containing layer, or like), a light-blocking member (for example, a light reflective layer, a light absorbing layer, or the like), a protective member (for example, an insulating layer, a dielectric layer, or the like).

[Electronic Apparatus]

The light-emitting device may be included in various electronic apparatuses. For example, the electronic apparatus including the light-emitting device may be a light-emitting apparatus, an authentication apparatus, and the like.

The electronic apparatus (for example, a light-emitting apparatus) may further include, in addition to the light-emitting device, i) a color filter, ii) a color conversion layer, or iii) both a color filter and a color conversion layer. The color filter and/or the color conversion layer may be arranged in at least one traveling direction of light emitted from the light-emitting device. For example, the light emitted from the light-emitting device may be blue light or white light. For details on the light-emitting device, related description provided above may be referred to. In an embodiment, the color conversion layer may include quantum dots. The quantum dots may be, for example, the same as described herein.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining film may be arranged among the subpixel areas to define each of the subpixel areas.

The color filter including the plurality of the color filter areas may further include light-shielding patterns interposed between the color filter areas, and the color conversion layer including the plurality of the color conversion areas may further include light-shielding patterns interposed between the color conversion areas.

The plurality of color filter areas (or the plurality of color conversion areas) may include a first area emitting first color light, a second area emitting second color light, and/or a third area emitting third color light, wherein the first color light, the second color light, and/or the third color light may have different maximum emission wavelengths from one another. For example, the first color light may be red light, the second color light may be green light, and the third color light may be blue light. For example, the color filter areas (or the color conversion areas) may include quantum dots. In detail, the first area may include red quantum dots, the second area may include green quantum dots, and the third area may not include quantum dots. Details for the quantum dots may be the same as described herein. Each of the first region, the second region, and/or the third region may further include a scatter.

In an embodiment, the light-emitting device may emit first light, the first area may absorb the first light to emit first first-color light, the second area may absorb the first light to emit second first-color light, and the third area may absorb the first light to emit third first-color light. In this regard, the first first-color light, the second first-color light, and the third first-color light may have different maximum emission wavelengths from one another. In particular, the first light may be blue light, the first-first color light may be red light, the second-first color light may be green light, and the third-first color light may be blue light.

The electronic apparatus may further include a thin-film transistor, in addition to the light-emitting device described above. The thin-film transistor may include a source electrode, a drain electrode, and an activation layer, wherein any one of the source electrode and the drain electrode may be electrically connected to any one of the first electrode and the second electrode of the light-emitting device.

The thin-film transistor may further include a gate electrode, a gate insulating film, or the like.

The activation layer may include crystalline silicon, amorphous silicon, an organic semiconductor, an oxide semiconductor, or the like.

The electronic apparatus may further include a sealing portion for sealing the light-emitting device. The sealing portion and/or the color conversion layer may be arranged between the color filter and the light-emitting device. The sealing portion allows light from the light-emitting device to be extracted to the outside, while simultaneously preventing ambient air and moisture from penetrating into the light-emitting device. The sealing portion may be a sealing substrate including a transparent glass substrate or a plastic substrate. The sealing portion may be a thin-film encapsulation layer including at least one layer of an organic layer and/or an inorganic layer. When sealing portion is a thin-film encapsulating layer, the electronic apparatus may be flexible.

Various functional layers may be additionally arranged on the sealing portion, in addition to the color filter and/or the color conversion layer, according to the use of the electronic apparatus. Examples of the functional layers are a touch screen layer, a polarizing layer, and the like. The touch screen layer may be a pressure-sensitive touch screen layer, a capacitive touch screen layer, or an infrared touch screen layer. The authentication apparatus may be, for example, a biometric authentication apparatus that authenticates an individual by using biometric information of a living body (for example, fingertips, pupils, and the like).

The authentication apparatus may further include, in addition to the light-emitting device as described above, a biometric information collector.

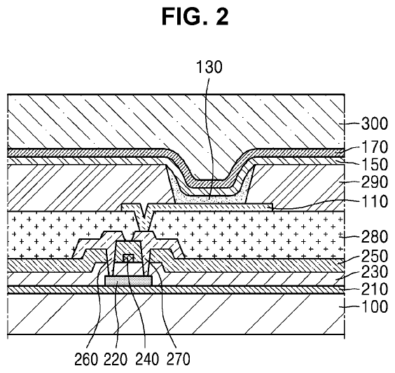

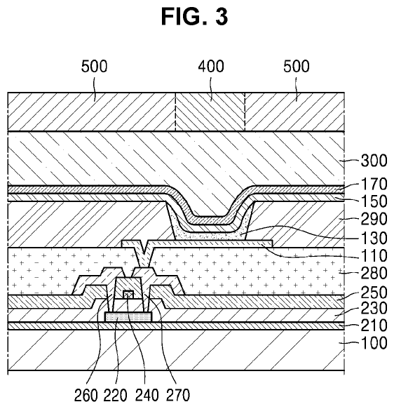

The electronic apparatus may be applied to various displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, various measuring instruments, meters (for example, meters for a vehicle, an aircraft, and a vessel), projectors, and the like.

[Description of FIGS. 2 and 3]

FIG. 2 is a cross-sectional view showing a light-emitting apparatus according to an embodiment.

The light-emitting apparatus of FIG. 2 includes a substrate 100, a thin-film transistor (TFT), a light-emitting device, and an encapsulation portion 300 that seals the light-emitting device.

The substrate 100 may be a flexible substrate, a glass substrate, or a metal substrate. A buffer layer 210 may be arranged on the substrate 100. The buffer layer 210 may prevent penetration of impurities through the substrate 100 and may provide a flat surface on the substrate 100.

The TFT may be arranged on the buffer layer 210. The TFT may include an activation layer 220, a gate electrode 240, a source electrode 260, and a drain electrode 270.

The activation layer 220 may include an inorganic semiconductor, such as silicon or polysilicon, an organic semiconductor, or an oxide semiconductor, and may include a source region, a drain region, and a channel region.

A gate insulating film 230 for insulating the activation layer 220 from the gate electrode 240 may be arranged on the activation layer 220, and the gate electrode 240 may be arranged on the gate insulating film 230.

An interlayer insulating film 250 may be arranged on the gate electrode 240. The interlayer insulating film 250 may be arranged between the gate electrode 240 and the source electrode 260 and between the gate electrode 240 and the drain electrode 270, so as to provide insulation therebetween.

The source electrode 260 and the drain electrode 270 may be arranged on the interlayer insulating film 250. The interlayer insulating film 250 and the gate insulating film 230 may be formed to expose the source region and the drain region of the activation layer 220, and the source electrode 260 and the drain electrode 270 may be arranged in contact with the exposed portions of the source region and the drain region of the activation layer 220.

The TFT may be electrically connected to the light-emitting device to drive the light-emitting device, and may be covered by a passivation layer 280. The passivation layer 280 may include an inorganic insulating film, an organic insulating film, or any combination thereof. The light-emitting device may be provided on the passivation layer 280. The light-emitting device may include the first electrode 110, the interlayer 130, and the second electrode 150.

The first electrode 110 may be arranged on the passivation layer 280. The passivation layer 280 may expose a portion of the drain electrode 270 without completely covering the drain electrode 270, and the first electrode 110 may be arranged to be connected to the exposed portion of the drain electrode 270.

A pixel defining layer 290 including an insulating material may be arranged on the first electrode 110. The pixel defining layer 290 may expose a region of the first electrode 110, and the interlayer 130 may be formed in the exposed region of the first electrode 110. The pixel defining layer 290 may be a polyimide organic film or a polyacrylic organic film. At least some layers of the interlayer 130 may extend beyond the upper portion of the pixel defining layer 290 to be arranged in the form of a common layer.

The second electrode 150 may be arranged on the interlayer 130, and a capping layer 170 may be additionally formed on the second electrode 150. The capping layer 170 may be formed to cover the second electrode 150.

The encapsulation portion 300 may be arranged on the capping layer 170. The encapsulation portion 300 may be arranged on the light-emitting device to protect the light-emitting device from moisture or oxygen. The encapsulation portion 300 may include: an inorganic film including silicon nitride (SiNx), silicon oxide (SiOx), indium tin oxide, indium zinc oxide, or any combination thereof, an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, and the like), an epoxy-based resin (for example, aliphatic glycidyl ether (AGE) and the like), or any combination thereof, or any combination of the inorganic film and the organic film.

FIG. 3 shows a cross-sectional view showing a light-emitting apparatus according to another embodiment.

The light-emitting apparatus of FIG. 3 is the same as the light-emitting apparatus of FIG. 2, except that a light-shielding pattern 500 and a functional region 400 are additionally arranged on the encapsulation portion 300. The functional region 400 may include i) a color filter area, ii) a color conversion area, or iii) a combination of a color filter area and a color conversion area. In an embodiment, a light-emitting device included in the light-emitting apparatus of FIG. 3 may be a tandem light-emitting device.

[Manufacturing Method]

Respective layers included in the hole transport region, the emission layer, and respective layers included in the electron transport region may be formed in a certain region by using one or more suitable methods selected from vacuum deposition, spin coating, casting, Langmuir-Blodgett (LB) deposition, ink-jet printing, laser-printing, laser-induced thermal imaging, and the like.

When respective layers included in the hole transport region, the emission layer, and respective layers included in the electron transport region are formed by vacuum deposition, the deposition conditions may include a deposition temperature in a range of about 100° C. to about 500° C., a vacuum degree of about 10⁻⁸ torr to about 10⁻³ torr, and a deposition speed in a range of about 0.01 Å/sec to about 100 Å/sec, depending on a material to be included in a layer to be formed and the structure of a layer to be formed.

### Definition of Terms

The term “C₃-C₆₀ carbocyclic group” as used herein refers to a cyclic group consisting of carbon only as a ring-forming atom and having three to sixty carbon atoms, and the term “C₁-C₆₀ heterocyclic group” as used herein refers to a cyclic group that has one to sixty carbon atoms and further has, in addition to carbon, a heteroatom as a ring-forming atom. Each of the C₃-C₆₀ carbocyclic group and the C₁-C₆₀ heterocyclic group may be a monocyclic group consisting of one ring or a polycyclic group consisting of two or more rings that are condensed together. For example, the C₁-C₆₀ heterocyclic group may have 3 to 61 ring-forming atoms.

The term “cyclic group” as used herein may include both the C₃-C₆₀ carbocyclic group and the C₁-C₆₀ heterocyclic group.

The term “n electron-rich C₃-C₆₀ cyclic group” as used herein refers to a cyclic group that has 3 to 60 carbon atoms and does not include *—N═*′ as a ring-forming moiety, and the term “n electron-deficient nitrogen-containing C₁-C₆₀ cyclic group” as used herein refers to a heterocyclic group that has 1 to 60 carbon atoms and includes *—N═*′ as a ring-forming moiety.

For example,

the C₃-C₆₀ carbocyclic group may be i) a T1 group or ii) a condensed cyclic group in which at least two T1 groups are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),

the C₁-C₆₀ heterocyclic group may be i) a T2 group, ii) a condensed cyclic group in which at least two T2 groups are condensed with each other, or iii) a condensed cyclic group in which at least one T2 group and at least one T1 group are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and the like),

the π electron-rich C₃-C₆₀ cyclic group may be i) a T1 group, ii) a condensed cyclic group in which at least two T1 groups are condensed with each other, iii) a T3 group, iv) a condensed cyclic group in which at least two T3 groups are condensed with each other, or v) a condensed cyclic group in which at least one T3 group and at least one T1 group are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, and the like),

the π electron-deficient nitrogen-containing C₁-C₆₀ cyclic group may be i) a T4 group, ii) a condensed cyclic group in which at least two T4 groups are condensed with each other, iii) a condensed cyclic group in which at least one T4 group and at least one T1 group are condensed with each other, iv) a condensed cyclic group in which at least one T4 group and at least one T3 group are condensed with each other, or v) a condensed cyclic group in which at least one T4 group, at least one T1 group, and at least one T3 group are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and the like),

the T1 group may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group,

the T2 group may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,

the T3 group may be a furan group, a thiophene group, a 1H-pyrrole group, a silole group, or a borole group, and

the T4 group may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “the cyclic group, the C₃-C₆₀ carbocyclic group, the C₁-C₆₀ heterocyclic group, the π electron-rich C₃-C₆₀ cyclic group, or the π electron-deficient nitrogen-containing C₁-C₆₀ cyclic group” as used herein refer to a group condensed to any cyclic group, a monovalent group, or a polyvalent group (for example, a divalent group, a trivalent group, a tetravalent group, and the like) according to the structure of a formula for which the corresponding term is used. For example, the “benzene group” may be a benzo group, a phenyl group, a phenylene group, or the like, which may be easily understood by one of ordinary skill in the art according to the structure of a formula including the “benzene group”.

Examples of the monovalent C₃-C₆₀ carbocyclic group and the monovalent C₁-C₆₀ heterocyclic group are a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group. Examples of the divalent C₃-C₆₀ carbocyclic group and the monovalent C₁-C₆₀ heterocyclic group are a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a substituted or unsubstituted divalent non-aromatic condensed heteropolycyclic group.

The term “C₁-C₆₀ alkyl group” as used herein refers to a linear or branched aliphatic hydrocarbon monovalent group that has one to sixty carbon atoms, and examples thereof are a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an iso-nonyl group, a sec-nonyl group, a tert-nonyl group, an n-decyl group, an isodecyl group, a sec-decyl group, a tert-decyl group, and the like. The term “C₁-C₆₀ alkylene group” as used herein refers to a divalent group having the same structure as the C₁-C₆₀ alkyl group.

The term “C₂-C₆₀ alkenyl group” as used herein refers to a monovalent hydrocarbon group having at least one carbon-carbon double bond in the middle or at the terminus of the C₂-C₆₀ alkyl group, and examples thereof are an ethenyl group, a propenyl group, a butenyl group, and the like. The term “C₂-C₆₀ alkenylene group” as used herein refers to a divalent group having the same structure as the C₂-C₆₀ alkenyl group.

The term “C₂-C₆₀ alkynyl group” as used herein refers to a monovalent hydrocarbon group having at least one carbon-carbon triple bond in the middle or at the terminus of the C₂-C₆₀ alkyl group, and examples thereof are an ethynyl group, a propynyl group, and the like. The term “C₂-C₆₀ alkynylene group” as used herein refers to a divalent group having the same structure as the C₂-C₆₀ alkynyl group.

The term “C₁-C₆₀ alkoxy group” as used herein refers to a monovalent group represented by —OA₁₀₁ (wherein A₁₀₁ is the C₁-C₆₀ alkyl group), and examples thereof are a methoxy group, an ethoxy group, an isopropyloxy group, and the like.

The term “C₃-C₁₀ cycloalkyl group” as used herein refers to a monovalent saturated hydrocarbon cyclic group having three to ten carbon atoms, and examples thereof are a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, a bicyclo[2.2.2]octyl group, and the like. The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent cyclic group that further includes, in addition to a carbon atom, at least one heteroatom as a ring-forming atom and has 1 to 10 carbon atoms, and examples thereof are a 1,2,3,4-oxatriazolidinyl group, a tetrahydrofuranyl group, a tetrahydrothiophenyl group, and the like. The term “C₁-C₁₀ heterocycloalkylene group” as used herein refers to a divalent group having the same structure as the C₁-C₁₀ heterocycloalkyl group.

The term “C₃-C₁₀ cycloalkenyl group” as used herein refers to a monovalent cyclic group that has 3 to 10 carbon atoms, at least one carbon-carbon double bond in the ring thereof, and no aromaticity, and examples thereof are a cyclopentenyl group, a cyclohexenyl group, a cycloheptenyl group, and the like. The term “C₃-C₁₀ cycloalkenylene group” as used herein refers to a divalent group having the same structure as the C₃-C₁₀ cycloalkenyl group.

The term “C₁-C₁₀ heterocycloalkenyl group” as used herein refers to a monovalent cyclic group that has, in addition to a carbon atom, at least one heteroatom as a ring-forming atom, 1 to 10 carbon atoms, and at least one carbon-carbon double bond in the cyclic structure thereof. Examples of the C₁-C₁₀ heterocycloalkenyl group are a 4,5-dihydro-1,2,3,4-oxatriazolyl group, a 2,3-dihydrofuranyl group, a 2,3-dihydrothiophenyl group, and the like. The term “C₁-C₁₀ heterocycloalkenylene group” as used herein refers to a divalent group having the same structure as the C₁-C₁₀ heterocycloalkenyl group.

The term “C₆-C₆₀ aryl group” as used herein refers to a monovalent group having a carbocyclic aromatic system having 6 to 60 carbon atoms, and the term “C₁-C₆₀ arylene group” as used herein refers to a divalent group having a carbocyclic aromatic system having 6 to 60 carbon atoms. Examples of the C₆-C₆₀ aryl group are a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, and the like. When the C₆-C₆₀ aryl group and the C₆-C₆₀ arylene group each include two or more rings, the rings may be condensed with each other.

The term “C₁-C₆₀ heteroaryl group” as used herein refers to a monovalent group having a heterocyclic aromatic system that has, in addition to a carbon atom, at least one heteroatom as a ring-forming atom, and 1 to 60 carbon atoms. The term “C₁-C₆₀ heteroarylene group” as used herein refers to a divalent group having a heterocyclic aromatic system that has, in addition to a carbon atom, at least one heteroatom as a ring-forming atom, and 1 to 60 carbon atoms. Examples of the C₁-C₆₀ heteroaryl group are a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, a naphthyridinyl group, and the like. When the C₁-C₆₀ heteroaryl group and the C₁-C₆₀ heteroarylene group each include two or more rings, the rings may be condensed with each other.

The term “monovalent non-aromatic condensed polycyclic group” as used herein refers to a monovalent group (for example, having 8 to 60 carbon atoms) having two or more rings condensed to each other, only carbon atoms as ring-forming atoms, and no aromaticity in its entire molecular structure. Examples of the monovalent non-aromatic condensed polycyclic group are an indenyl group, a fluorenyl group, a spiro-bifluorenyl group, a benzofluorenyl group, an indenophenanthrenyl group, and an indeno anthracenyl group. The term “divalent non-aromatic condensed polycyclic group” as used herein refers to a divalent group having the same structure as the monovalent non-aromatic condensed polycyclic group described above.

The term “monovalent non-aromatic condensed heteropolycyclic group” as used herein refers to a monovalent group (for example, having 1 to 60 carbon atoms) having two or more rings condensed with each other, at least one heteroatom other than carbon atoms, as a ring-forming atom, and no aromaticity in its entire molecular structure. Examples of the monovalent non-aromatic condensed heteropolycyclic group are a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, a benzothienodibenzothiophenyl group, and the like. The term “divalent non-aromatic condensed heteropolycyclic group” as used herein refers to a divalent group having the same structure as the monovalent non-aromatic condensed heteropolycyclic group described above.

The term “C₁-C₆₀ aryloxy group” as used herein indicates —OA₁₀₂ (wherein A₁₀₂ is the C₆-C₆₀ aryl group), and the term “C₆-C₆₀ arylthio group” as used herein indicates —SA₁₀₃ (wherein A₁₀₃ is the C₆-C₆₀ aryl group).

The term “C₇-C₆₀ arylalkyl group” as used herein refers to -A₁₀₄A₁₀₅ (wherein A₁₀₄ is a C₁-C₅₄ alkylene group and A₁₀₅ is a C₆-C₅₉ aryl group), and the term “C₂-C₆₀ heteroarylalkyl group” as used herein refers to -A₁₀₆A₁₀₇ (wherein A₁₀₆ is a C₁-C₅₉ alkylene group and A₁₀₇ is a C₁-C₅₉ heteroaryl group).

The term “R₁₀ₐ” as used herein may be:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroaryl alkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroaryl alkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof, or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

In the embodiments described herein, Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group, a C₁-C₆₀ heterocyclic group, a C₇-C₆₀ aryl alkyl group, or a C₂-C₆₀ heteroaryl alkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

The term “heteroatom” as used herein refers to any atom other than a carbon atom. Examples of the heteroatom are O, S, N, P, Si, B, Ge, Se, or any combination thereof.

The term “third-row transition metal” as used herein includes hafnium (Hf), tantalum (Ta), tungsten (W), rhenium (Re), osmium (Os), iridium (Ir), platinum (Pt), gold (Au), and the like.

“Ph” as used herein refers to a phenyl group, “Me” as used herein refers to a methyl group, “Et” as used herein refers to an ethyl group, “tert-Bu” or “Bu^{t}” as used herein refers to a tert-butyl group, and “OMe” as used herein refers to a methoxy group.

The term “biphenyl group” as used herein refers to “a phenyl group substituted with a phenyl group”. In other words, the “biphenyl group” belongs to “a substituted phenyl group having a C₆-C₆₀ aryl group as a substituent”.

The term “terphenyl group” as used herein refers to “a phenyl group substituted with a biphenyl group”. In other words, the “terphenyl group” belongs to “a substituted phenyl group having, as a substituent, a C₆-C₆₀ aryl group substituted with a C₆-C₆₀ aryl group”.

In the embodiments described herein, * and *′, unless defined otherwise, each refer to a binding site to a neighboring atom in a corresponding formula or moiety.

Hereinafter, compounds according to embodiments and light-emitting devices according to embodiments will be described in detail with reference to the following synthesis examples and examples. The wording “B was used instead of A” used in describing Synthesis Examples denotes that an identical molar equivalent of B was used in place of A.

### EXAMPLES

### Synthesis Example 1: Synthesis of Compound 41

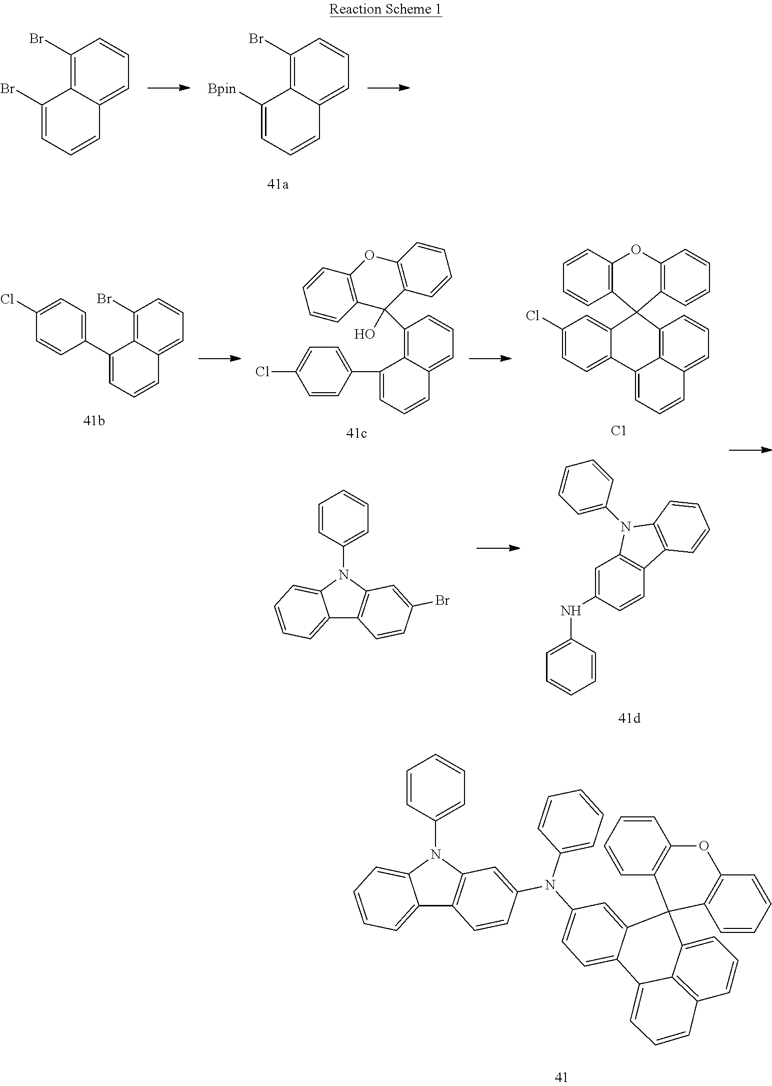

### 1-1. Synthesis of Intermediate 41a

1,8-dibromonaphthalene (1.0 eq.), bis(pinacolato)diboron (1.2 eq.), potassium acetate (4.0 eq.), and palladium acetate (0.05 eq.) were dissolved in 1,4-dioxane, and stirred for 3 hours at 80° C. under a nitrogen atmosphere to obtain a reactant. The reactant was cooled and washed using ethyl acetate and water 3 times each to obtain an organic layer. The organic layer was dried using MgSO₄ and then dried again under reduced pressure. The resultant was subjected to column chromatography to obtain Intermediate 41a. (Yield: 85%)

### 1-2. Synthesis of Intermediate 41b

Intermediate 41a (1.0 eq.), 1-bromo-4-chlorobenzene (1.2 eq.), tetrakis(triphenylphosphine)palladium (0.05 eq.), and potassium carbonate (2.0 eq.) were dissolved in a solution containing THE and H₂O at a volume ratio of 4:1, and stirred for 12 hours at 80° C. in a nitrogen atmosphere to obtain a reactant. The reactant was cooled and washed using ethyl acetate and water 3 times each to obtain an organic layer. The organic layer was dried using MgSO₄ and then dried again under reduced pressure. The resultant was subjected to column chromatography to obtain Intermediate 41b. (Yield: 66%)

### 1-3. Synthesis of Intermediate 41c

Anhydrous THE was added dropwise to Intermediate 41b (1.0 eq.), and the mixed solution was cooled down to −78° C. in a nitrogen atmosphere. n-BuLi (1.1 eq.) was slowly added dropwise to the cooled solution, and stirred for 1 hour at −78° C. Then, 9H-xanthen-9-one (1.1 eq.) was slowly added dropwise to the reaction solution, and stirred again for 3 hours at room temperature to obtain a reactant. After cooling the reactant, a washing process was performed thereon using ethyl acetate and water three times each, and an organic layer thus obtained was dried using MgSO₄ first and then dried again under reduced pressure. The resultant was subjected to column chromatography to obtain Intermediate 41c. (Yield: 82%)

### 1-4. Synthesis of Intermediate C1

Intermediate 41c (1.0 eq.) was dissolved in a solution containing acetic acid and hydrochloric acid at a volume ratio of 9:1, and stirred for 2 hours at 80° C. in a nitrogen atmosphere to obtain a reactant. The reactant was cooled and washed using ethyl acetate and water 3 times each to obtain an organic layer. The organic layer was dried using MgSO₄ and then dried again under reduced pressure. The resultant was subjected to column chromatography to obtain Intermediate C1. (Yield: 78%)

### 1-5. Synthesis of Intermediate 41d

Aniline (1.0 eq.), 2-bromo-9-phenyl-9H-carbazole (1.1 eq.), tris(dibenzylideneacetone)dipalladium(0) (0.05 eq.), tri-tert-butylphosphine (0.10 eq.), and sodium tert-butoxide (2.0 eq.) were dissolved in toluene and stirred for 2 hours at 90° C. in a nitrogen atmosphere to obtain a reactant. The reactant was cooled and washed using ethyl acetate and water 3 times each to obtain an organic layer. The organic layer was dried using MgSO₄ and then dried again under reduced pressure. The resultant was subjected to column chromatography to obtain Intermediate 41d. (Yield: 78%)

### 1-6. Synthesis of Compound 41

Intermediate C1 (1.0 eq.), Intermediate 41d (1.0 eq.), tris(dibenzylideneacetone)dipalladium(0) (0.05 eq.), tri-tert-butylphosphine (0.10 eq.), and sodium tert-butoxide (2.0 eq.) were dissolved in toluene and then stirred for 2 hours at 90° C. in a nitrogen atmosphere to obtain a reactant. The reactant was cooled and washed using ethyl acetate and water 3 times each to obtain an organic layer. The organic layer was dried using MgSO₄ and then dried again under reduced pressure. The resultant was subjected to column chromatography to obtain Compound 41. (Yield: 75%) By measuring FAB-MS, the obtained compound was identified as Compound 41 based on the molecular ion peak observed at mass number m/z of 714.27.

Synthesis methods of other compounds in addition to the compound synthesized in Synthesis Example may be easily recognized by those skilled in the art by referring to the synthesis paths and source materials.

### Example 1

To prepare an anode, ITO, Ag, and WOₓ were sequentially deposited on a glass substrate to a thickness of 150 Å, 1,000 Å, and WOₓ, respectively, by a sputtering method. The glass substrate on which ITO, Ag, and WOₓ were sputter-deposited was cut to a size of 50 mm×50 mm×0.5 mm and sonicated with isopropyl alcohol and pure water for 15 each. Then, ultraviolet light was irradiated for 30 minutes thereto and ozone was exposed thereto for cleaning. Subsequently, the resultant glass substrate was loaded onto a vacuum deposition apparatus.

Compound 1 was deposited on the anode to form a hole transport layer having a thickness of 1,200 Å.

9,10-di(2-naphthyl)anthracene (ADN), which is a known host in the art, and 4,4′-bis[2-(4-(N,N-diphenylamino)phenyl)vinyl]biphenyl (hereinafter, referred to as DPAVBi), which is a known blue phosphorescence dopant compound in the compound, were co-deposited at a weight ratio of 98:2 on the hole transport layer to form an emission layer having a thickness of 300 Å.

Spiro(benzo[de]anthracene-7,9′-fluorene) was deposited on the emission layer to form an electron transport layer having a thickness of 100 Å.

LiF was vacuum-deposited on the electron transport layer to form an electron injection layer having a thickness of 10 Å, and Mg and Ag were vacuum-deposited at a weight ratio 1:10 on the electron injection layer to form a cathode having a thickness of 100 Å, thereby completing the manufacture of an organic light-emitting device.

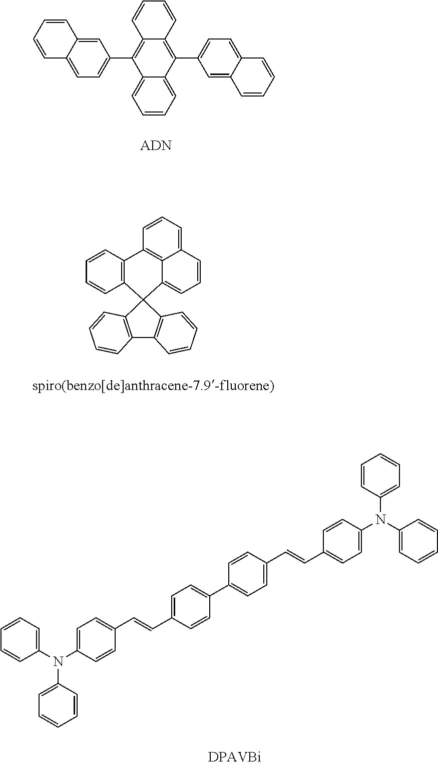

### Examples 2 to 5

Organic light-emitting devices were manufactured in the same manner as in Example 1, except that, in forming a hole transport layer, Compounds 2 to 5 were respectively used instead of Compound 1.

### Comparative Example 1

A light-emitting device was manufactured in the same manner as in Example 1, except that a glass substrate with ITO (manufactured by Corning Inc., 15 Ω/cm² (1,200 Å)) thereon was used as an anode and Compound HT1 was used instead of Compound 1 in forming a hole transport layer.

### Comparative Example 2

A light-emitting device was manufactured in the same manner as in Example 1, except that, in forming a hole transport layer, Compound HT1 was used instead of Compound 1.

### Comparative Example 3

A light-emitting device was manufactured in the same manner as in Example 1, except that a glass substrate with ITO (manufactured by Corning Inc., 15 Ω/cm² (1,200 Å)) thereon was used as an anode.

### Comparative Example 4

A light-emitting device was manufactured in the same manner as in Example 1, except that a glass substrate with ITO (manufactured by Corning Inc., 15 Ω/cm² (1,200 Å)) thereon was used as an anode and Compound 2 was used instead of Compound 1 in forming a hole transport layer.

### Comparative Example 5

A light-emitting device was manufactured in the same manner as in Example 1, except that a glass substrate with ITO (manufactured by Corning Inc., 15 Ω/cm² (1,200 Å)) thereon was used as an anode and Compound 3 was used instead of Compound 1 in forming a hole transport layer.

### Evaluation Example 1

To evaluate characteristics of the organic light-emitting devices of Examples 1 to and the light-emitting devices of Comparative Examples 11 to 5, driving voltage (V) at 1,000 cd/m², luminescence efficiency (Cd/A), and lifespan (LT₉₀) was respectively measured using Keithley MU 236 and luminance meter PR650, and results thereof are shown in Table 1. Regarding the lifespan (T₉₀), the time taken for the luminance to become 90% compared to the initial luminance of 100% was measured. In addition, the progressive driving voltage (ΔV) of each (organic) light-emitting device was measured based on the variation of driving voltage after 150 hours of the operation by using a source meter (Keithley Instrument, 2400 series) under the conditions that the temperature at the measurement was room temperature and the luminance at the measurement was MQ 420 nits. In Table 1, x refers to 2.9≤x≤3.1.

TABLE 1

| | | | | Lumi- | | |
|---|---|---|---|---|---|---|
| | | Hole- | | nes- | | |
| | | trans- | Driving | cence | | AV |
| | | porting | voltage | effici- | LT | (V, |
| | | com- | (V@10 | ency | (T₉₀, | @150 |
| | Anode | pound | mA) | (Cd/A) | hr) | hr) |
| Example 1 | ITO/Ag/WOx | 1 | 4.3 | 6.4 | 62 | 0.2 |
| Example 2 | ITO/Ag/WOx | 2 | 4.3 | 6.1 | 35 | 0.1 |
| Example 3 | ITO/Ag/WOx | 3 | 4.4 | 6.0 | 42 | 0.1 |
| Example 4 | ITO/Ag/WOx | 4 | 4.4 | 6.6 | 41 | 0.2 |
| Example 5 | ITO/Ag/WOx | 5 | 4.3 | 6.3 | 37 | 0.3 |
| Comparative | ITO | HT1 | 4.3 | 6.3 | 30 | 2.2 |
| Example 1 | | | | | | |
| Comparative | ITO/Ag/WOx | HT1 | 4.5 | 6.5 | 26 | 0.6 |
| Example 2 | | | | | | |
| Comparative | ITO | 1 | 5.5 | 6.4 | 8 | 1.8 |
| Example 3 | | | | | | |
| Comparative | ITO | 2 | 4.3 | 6.3 | 30 | 2.2 |
| Example 4 | | | | | | |
| Comparative | ITO | 3 | 5.5 | 6.3 | 30 | 0.3 |
| Example 5 | | | | | | |

| | |
|---|---|
| 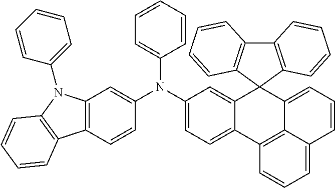 [SMILES: c1ccc(N(c2ccc3c(c2)C2(c4ccccc4-c4ccccc42)c2cccc4cccc-3c24)c2ccc3c4ccccc4n(-c4ccccc4)c3c2)cc1] | 1 |
| 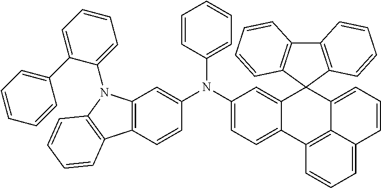 [SMILES: c1ccc(-c2ccccc2-n2c3ccccc3c3ccc(N(c4ccccc4)c4ccc5c(c4)C4(c6ccccc6-c6ccccc64)c4cccc6cccc-5c46)cc32)cc1] | 2 |
| 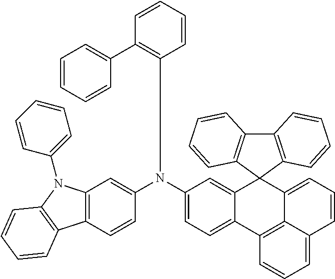 [SMILES: c1ccc(-c2ccccc2N(c2ccc3c(c2)C2(c4ccccc4-c4ccccc42)c2cccc4cccc-3c24)c2ccc3c4ccccc4n(-c4ccccc4)c3c2)cc1] | 3 |
| 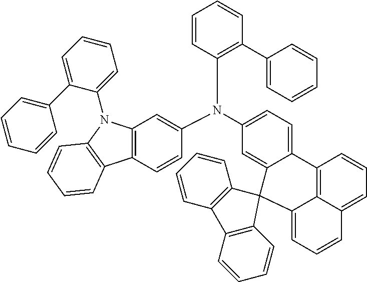 [SMILES: c1ccc(-c2ccccc2N(c2ccc3c(c2)C2(c4ccccc4-c4ccccc42)c2cccc4cccc-3c24)c2ccc3c4ccccc4n(-c4ccccc4-c4ccccc4)c3c2)cc1] | 4 |
| 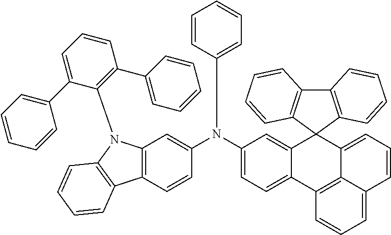 [SMILES: c1ccc(-c2cccc(-c3ccccc3)c2-n2c3ccccc3c3ccc(N(c4ccccc4)c4ccc5c(c4)C4(c6ccccc6-c6ccccc64)c4cccc6cccc-5c46)cc32)cc1] | 5 |
| 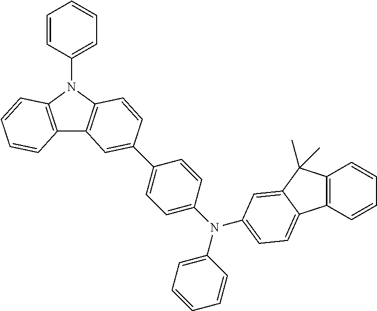 [SMILES: CC1(C)c2ccccc2-c2ccc(N(c3ccccc3)c3ccc(-c4ccc5c(c4)c4ccccc4n5-c4ccccc4)cc3)cc21] | HT1 |

Referring to Table 1, it was confirmed that the organic light-emitting devices of Examples 1 to 5 had the same or lower levels of the driving voltage and the progressive driving voltage, the same or higher levels of the luminescence efficiency, and long lifespan compared to the light-emitting devices of Comparative Examples 1 to 5.

Although certain embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

What is claimed is:

1. A light-emitting device comprising:
a first electrode;
a second electrode facing the first electrode;
an interlayer arranged between the first electrode and the second electrode and comprising an emission layer, wherein
the interlayer further comprises a hole transport region arranged between the first electrode and the emission layer,
the first electrode comprises a metal oxide having a work function of 5.3 eV or more, and
the hole transport region comprises a heterocyclic compound represented by Formula 1: 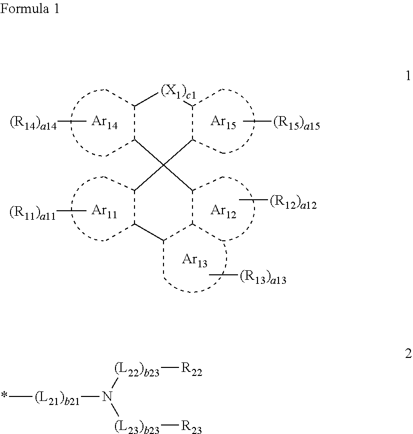
wherein, in Formula 1,
X₁ is a single bond, *—N(Z₁₁ₐ)—*′, *—B(Z₁₁ₐ)—*′, *—P(Z₁₁ₐ)—*′, *—C(Z₁₁)(Z_{11b})—*′, *—Si(Z₁₁ₐ)(Z_{11b})—*′, *—Ge(Z₁₁ₐ)(Z_{11b})—*′, *—S—*′, *—Se—*′, *—O—*′, *—C(═O)—*′, *—S(═O)—*′, *—S(═O)₂—*′, *—C(Z₁₁ₐ)=*′, *═C(Z₁₁ₐ)—*′, *—C(Z₁₁ₐ)═C(Z_{11b})—*′, *—C(═S)—*′, or *—C≡C—*′, wherein * and *′ each indicate a neighboring atom,
c1 is 0 or 1,
Ar₁₁ to Ar₁₅ are each independently a C₃-C₆₀ carbocyclic group or a C₁-C₆₀ heterocyclic group,
R₁₁ to R₁₅, Z₁₁ₐ, and Z_{11b} are each independently a group represented by Formula 2, hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂),
at least one of R₁₁ to R₁₅ is a group represented by Formula 2,
al 1 to a15 are each independently an integer from 0 to 10,
in Formula 2,
L₂₁ to L₂₃ are each independently a single bond, a C₃-C₆₀ carbocyclic group unsubstituted or substituted with at least one R₁₀ₐ, or a C₁-C₆₀ heterocyclic group unsubstituted or substituted with at least one R₁₀ₐ,
b21 to b23 are each independently an integer from 0 to 5,
R₂₂ and R₂₃ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂),
two or more groups selected from: Ru(s) in the number of al; R₁₂(s) in the number of a12; R₁₃(s) in the number of a13; R₁₄(s) in the number of a14; R₁₅(s) in the number of a15; R₂₂; and R₂₃ are optionally linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group unsubstituted or substituted with at least one R₁₀ₐ,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group; or a C₆-C₆₀ arylthio group, each unsubstituted or substituted with deuterium, —F, —Cl, —Br, —I, a hydroxyl group, a cyano group, a nitro group, a C₁-Coo alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —B(Q₂₁)(Q₂₂), —C(═O)(Q₂₁), —S(═O)₂(Q₂₁), —P(═O)(Q₂₁)(Q₂₂), or any combination thereof, or
—Si(Q₃₁)(Q₃₂)(Q₃₃), —B(Q₃₁)(Q₃₂), —C(═O)(Q₃₁), —S(═O)₂(Q₃₁), or —P(═O)(Q₃₁)(Q₃₂),
Q₁ to Q₃, Q₁₁ to Q₁₃, Q₂₁ to Q₂₃, and Q₃₁ to Q₃₃ are each independently: hydrogen;
deuterium; —F; —Cl; —Br; —I; a hydroxyl group; a cyano group; a nitro group; a C₁-C₆₀ alkyl group;
a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof, and
* indicates a binding site to a neighboring atom.

2. The light-emitting device of claim 1, wherein the first electrode further comprises a conductive oxide material, a metal material, a metal alloy material, or any combination thereof.

3. The light-emitting device of claim 1, wherein the first electrode comprises an anode,
the second electrode comprises a cathode,
the interlayer further comprises an electron transport region arranged between the emission layer and the second electrode,
the hole transport region comprises a hole injection layer, a hole transport layer, an emission auxiliary layer, an electron blocking layer, or any combination thereof, and
the electron transport region comprises a hole blocking layer, an electron transport layer, an electron injection layer, or any combination thereof.

4. The light-emitting device of claim 3, wherein the hole transport layer comprises the heterocyclic compound represented by Formula 1.

5. The light-emitting device of claim 4, wherein the first electrode and the hole transport layer are in direct contact with each other.

6. The light-emitting device of claim 4, wherein the hole transport layer and the emission layer are in direct contact with each other.

7. The light-emitting device of claim 1, wherein the hole transport region does not include a p-dopant.

8. The light-emitting device of claim 1, wherein the metal oxide is an oxide of tungsten (W), molybdenum (Mo), copper (Cu), nickel (Ni), vanadium (V), or any combination thereof.

9. The light-emitting device of claim 1, wherein X₁ is a single bond, *—S—*′, or *—O—*′.

10. The light-emitting device of claim 1, wherein Ar₁₁ to Ar15 are each independently a π electron-rich C₃-C₆₀ cyclic group.

11. The light-emitting device of claim 1, wherein a group represented by 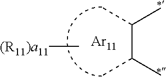
in Formula 1 is represented by one of Formulae Ar11-1 to Ar11-5: 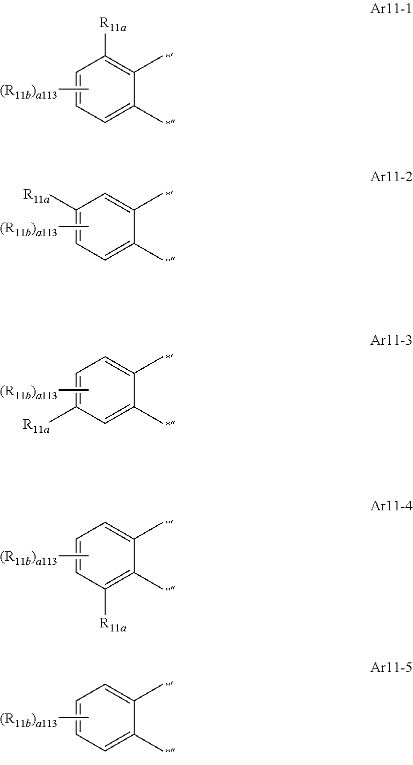
wherein, in Formulae Ar11-1 to Ar11-5,
R₁₁ₐ is a group represented by Formula 2,
R_{11b} is as described in connection with R₁₁ in claim 1, but R_{11b} is not a group represented by Formula 2,
a113 is an integer from 0 to 3,
a114 is an integer from 0 to 4, and
′ and *″ each indicate a binding site to a neighboring atom.

12. The light-emitting device of claim 1, wherein
i) a11 is an integer from 1 to 4, and at least one of R₁₁(s) in the number of a11 is a group represented by Formula 2;
ii) a12 is an integer from 1 to 3, and at least one of R₁₂(s) in the number of a12 is a group represented by Formula 2; or
iii) a13 is an integer from 1 to 3, and at least one of R₁₃(s) in the number of a13 is a group represented by Formula 2.

13. The light-emitting device of claim 1, wherein R₁₁ to R₁₅, Z₁₁ₐ, and Z_{11b} are each independently:
hydrogen, deuterium, —F, —Cl, —Br, —I, a hydroxyl group, a cyano group, or a nitro group;
a C₁-C₂₀ alkyl group, a C₂-C₂₀ alkenyl group, a C₂-C₂₀ alkynyl group, or a C₁-C₂₀ alkoxy group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a phenyl group, a naphthyl group, a pyridinyl group, a pyrimidinyl group, —Si(Q₃₁)(Q₃₂)(Q₃₃), —B(Q₃₁)(Q₃₂), or any combination thereof;
a phenyl group, a naphthyl group, a pyridinyl group, or a pyrimidinyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, a C₁-C₂₀ alkyl group, a C₂-C₂₀ alkenyl group, a C₂-C₂₀ alkynyl group, a C₁-C₂₀ alkoxy group, a phenyl group, a naphthyl group, a pyridinyl group, a pyrimidinyl group, —Si(Q₃₁)(Q₃₂)(Q₃₃), —B(Q₃₁)(Q₃₂), or any combination thereof; or
—Si(Q₁)(Q₂)(Q₃) or —B(Q₁)(Q₂), and
Q₁ to Q₃ and Q₃₁ to Q₃₃ are respectively as described in claim 1.

14. The light-emitting device of claim 1, wherein R₂₂ and R₂₃ in Formula 2 are each independently: a group represented by Formula 3-1 or 3-2;
hydrogen, deuterium, a hydroxyl group, or a nitro group;
a C₁-C₂₀ alkyl group, a C₂-C₂₀ alkenyl group, a C₂-C₂₀ alkynyl group, or a C₁-C₂₀ alkoxy group, each substituted or unsubstituted with deuterium, —CD₃, —CD₂H, —CDH₂, a hydroxyl group, a nitro group, a phenyl group, a naphthyl group, —Si(Q₃₁)(Q₃₂)(Q₃₃), —B(Q₃₁)(Q₃₂), or any combination thereof;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a thiophenyl group, a furanyl group, an isoindolyl group, an indolyl group, a carbazolyl group, a benzofuranyl group, a benzothiophenyl group, a dibenzofuranyl group, a dibenzothiophenyl group, a benzocarbazolyl group, or a dibenzocarbazolyl group, each substituted or unsubstituted with deuterium, —CD₃, —CD₂H, —CDH₂, a hydroxyl group, a nitro group, a C₁-C₂₀ alkyl group, a C₂-C₂₀ alkenyl group, a C₂-C₂₀ alkynyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a thiophenyl group, a furanyl group, an isoindolyl group, an indolyl group, a carbazolyl group, a benzofuranyl group, a benzothiophenyl group, a dibenzofuranyl group, a dibenzothiophenyl group, a benzocarbazolyl group, a dibenzocarbazolyl group, —Si(Q₃₁)(Q₃₂)(Q₃₃), —N(Q₃₁)(Q₃₂), —B(Q₃₁)(Q₃₂), or any combination thereof, or
—Si(Q₁)(Q₂)(Q₃) or —B(Q₁)(Q₂), wherein
at least one of R₂₂ and R₂₃ is a group represented by Formula 3-1 or 3-2: 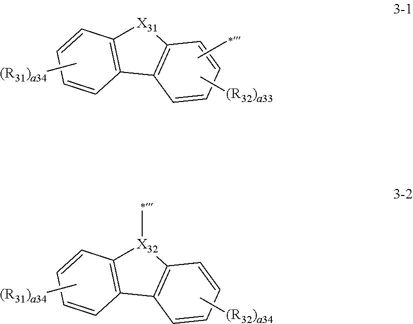
wherein, in Formulae 3-1 and 3-2,
X₃₁ is O, S, N(Z₃₁ₐ), or C(Z₃₁ₐ)(Z_{31b}),
X₃₂ is N or C(Z₃₂ₐ),
R₃₁, R₃₂, Z₃₁ₐ, and Z_{31b} are respectively as described in connection with R₂₂ in claim 1,
a33 is an integer from 0 to 3,
a34 is an integer from 0 to 4,
two or more groups selected from R₃₁(s) in the number of a34 and R₃₂(s) in the number of a33 or a34 are optionally linked together via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group unsubstituted or substituted with at least one R₁₀ₐ, and
*′″ indicates a binding site to R₂₂ or R₂₃.

15. The light-emitting device of claim 14, wherein the group represented by Formula 3-1 or 3-2 is each independently one of groups represented by Formulae 3-1A to 3-1G and 3-2: 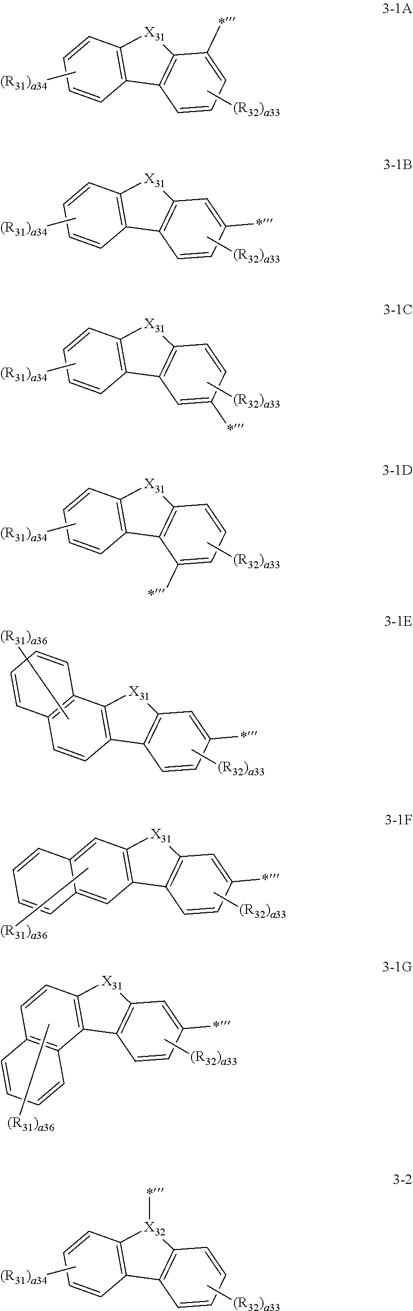
wherein, in Formulae 3-1A to 3-1G and 3-2,
X₃₁, X₃₂, R₃₁, R₃₁, R₃₂, a33, and a34 are respectively as described in claim 14,
a36 is an integer from 0 to 6, and
*′″ indicates a binding site to R₂₂ or R₂₃.

16. The light-emitting device of claim 14, wherein R₃₁, R₃₂, Z₃₁ₐ, and Z_{31b} are each independently:
hydrogen, deuterium, a hydroxyl group, or a nitro group;
a C₁-C₂₀ alkyl group, a C₂-C₂₀ alkenyl group, a C₂-C₂₀ alkynyl group, or a C₁-C₂₀ alkoxy group, each substituted or unsubstituted with deuterium, —CD₃, —CD₂H, —CDH₂, a hydroxyl group, a nitro group, a phenyl group, a naphthyl group, —Si(Q₃₁)(Q₃₂)(Q₃₃), —B(Q₃₁)(Q₃₂), or any combination thereof;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a thiophenyl group, a furanyl group, an isoindolyl group, an indolyl group, a carbazolyl group, a benzofuranyl group, a benzothiophenyl group, a dibenzofuranyl group, a dibenzothiophenyl group, a benzocarbazolyl group, or a dibenzocarbazolyl group, each substituted or unsubstituted with deuterium, —CD₃, —CD₂H, —CDH₂, a hydroxyl group, a nitro group, a C₁-C₂₀ alkyl group, a C₂-C₂₀ alkenyl group, a C₂-C₂₀ alkynyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a thiophenyl group, a furanyl group, an isoindolyl group, an indolyl group, a carbazolyl group, a benzofuranyl group, a benzothiophenyl group, a dibenzofuranyl group, a dibenzothiophenyl group, a benzocarbazolyl group, a dibenzocarbazolyl group, —Si(Q₃₁)(Q₃₂)(Q₃₃), —N(Q₃₁)(Q₃₂-B(Q₃₁)(Q₃₂), or any combination thereof, or
—Si(Q₁)(Q₂)(Q₃) or —B(Q₁)(Q₂), and
Q₁ to Q₃ and Q₃₁ to Q₃₃ are respectively as described in claim 1.

17. The light-emitting device of claim 1, wherein the heterocyclic compound represented by Formula 1 is selected from Compounds 1 to 99:

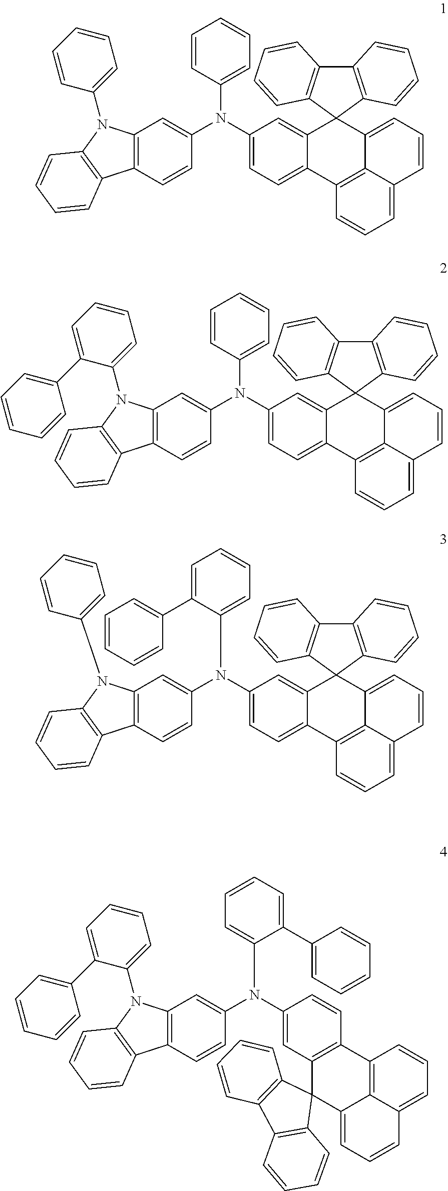 [SMILES: c1ccc(N(c2ccc3c(c2)C2(c4ccccc4-c4ccccc42)c2cccc4cccc-3c24)c2ccc3c4ccccc4n(-c4ccccc4)c3c2)cc1 ; c1ccc(-c2ccccc2-n2c3ccccc3c3ccc(N(c4ccccc4)c4ccc5c(c4)C4(c6ccccc6-c6ccccc64)c4cccc6cccc-5c46)cc32)cc1 ; c1ccc(-c2ccccc2N(c2ccc3c(c2)C2(c4ccccc4-c4ccccc42)c2cccc4cccc-3c24)c2ccc3c4ccccc4n(-c4ccccc4)c3c2)cc1 ; c1ccc(-c2ccccc2N(c2ccc3c(c2)C2(c4ccccc4-c4ccccc42)c2cccc4cccc-3c24)c2ccc3c4ccccc4n(-c4ccccc4-c4ccccc4)c3c2)cc1]

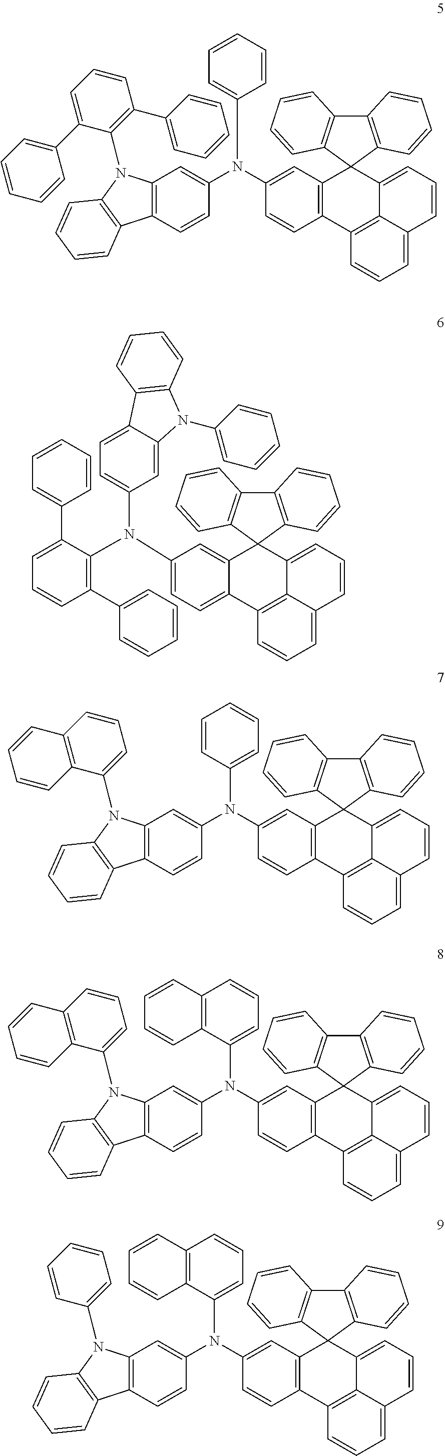

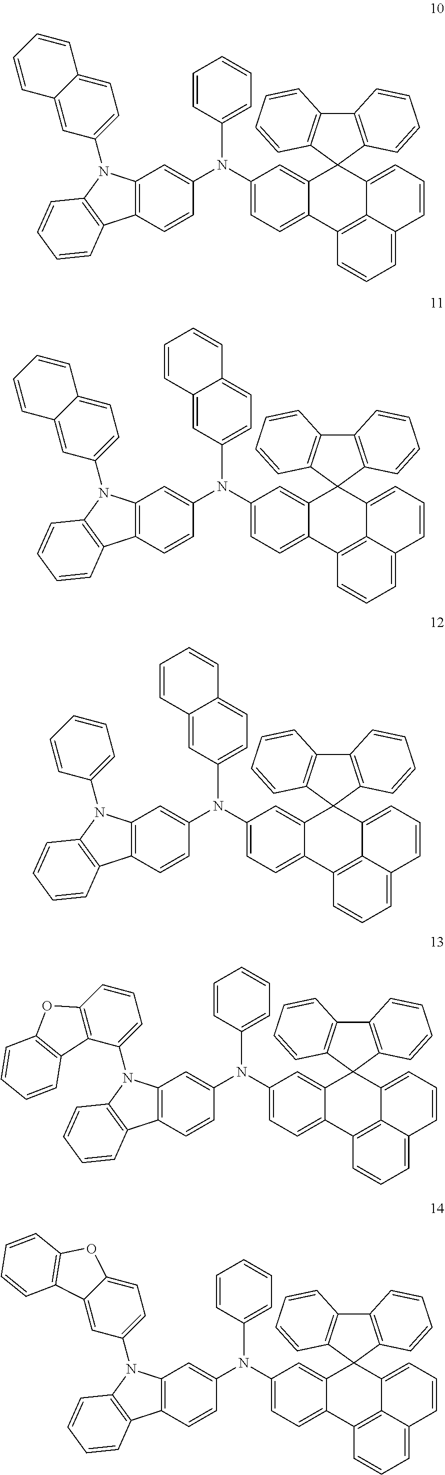

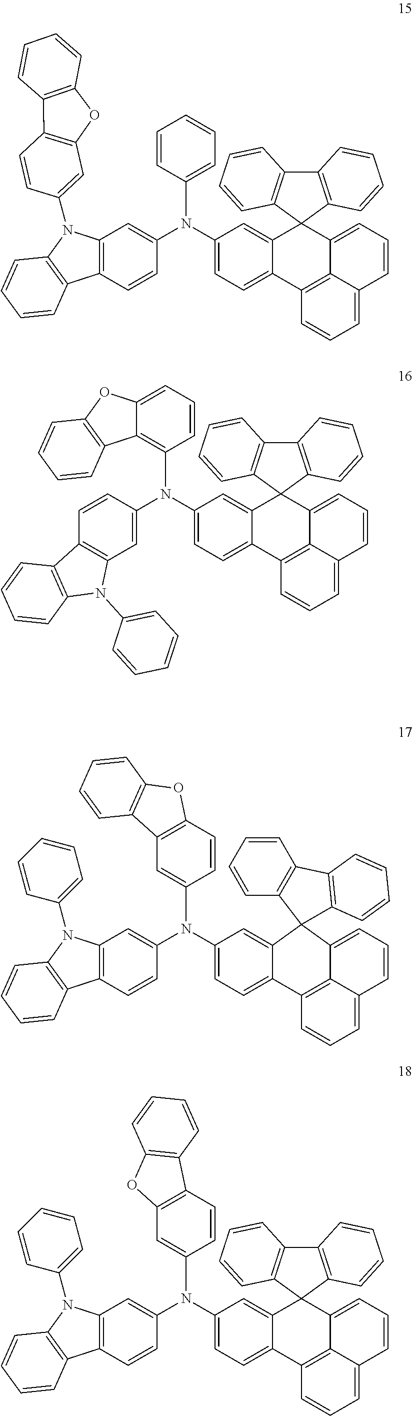 [SMILES: c1ccc(N(c2ccc3c(c2)C2(c4ccccc4-c4ccccc42)c2cccc4cccc-3c24)c2ccc3c4ccccc4n(-c4ccc5c(c4)oc4ccccc45)c3c2)cc1 ; c1ccc(-n2c3ccccc3c3ccc(N(c4ccc5c(c4)C4(c6ccccc6-c6ccccc64)c4cccc6cccc-5c46)c4cccc5oc6ccccc6c45)cc32)cc1 ; c1ccc(-n2c3ccccc3c3ccc(N(c4ccc5c(c4)C4(c6ccccc6-c6ccccc64)c4cccc6cccc-5c46)c4ccc5oc6ccccc6c5c4)cc32)cc1 ; c1ccc(-n2c3ccccc3c3ccc(N(c4ccc5c(c4)C4(c6ccccc6-c6ccccc64)c4cccc6cccc-5c46)c4ccc5c(c4)oc4ccccc45)cc32)cc1]

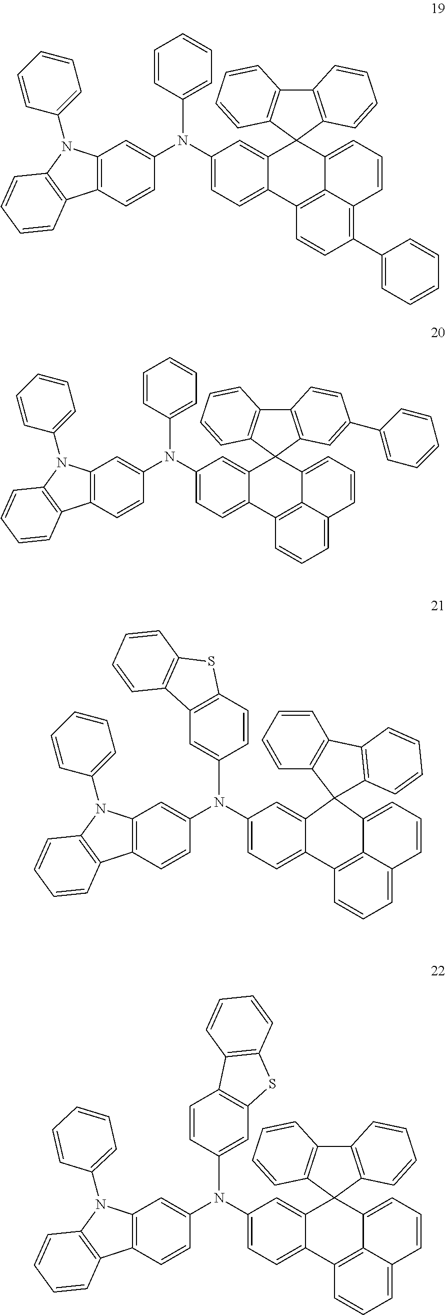 [SMILES: c1ccc(-c2ccc3c4c(cccc24)C2(c4ccccc4-c4ccccc42)c2cc(N(c4ccccc4)c4ccc5c6ccccc6n(-c6ccccc6)c5c4)ccc2-3)cc1 ; c1ccc(-c2ccc3c(c2)C2(c4ccccc4-3)c3cc(N(c4ccccc4)c4ccc5c6ccccc6n(-c6ccccc6)c5c4)ccc3-c3cccc4cccc2c34)cc1 ; c1ccc(-n2c3ccccc3c3ccc(N(c4ccc5c(c4)C4(c6ccccc6-c6ccccc64)c4cccc6cccc-5c46)c4ccc5sc6ccccc6c5c4)cc32)cc1 ; c1ccc(-n2c3ccccc3c3ccc(N(c4ccc5c(c4)C4(c6ccccc6-c6ccccc64)c4cccc6cccc-5c46)c4ccc5c(c4)sc4ccccc45)cc32)cc1]

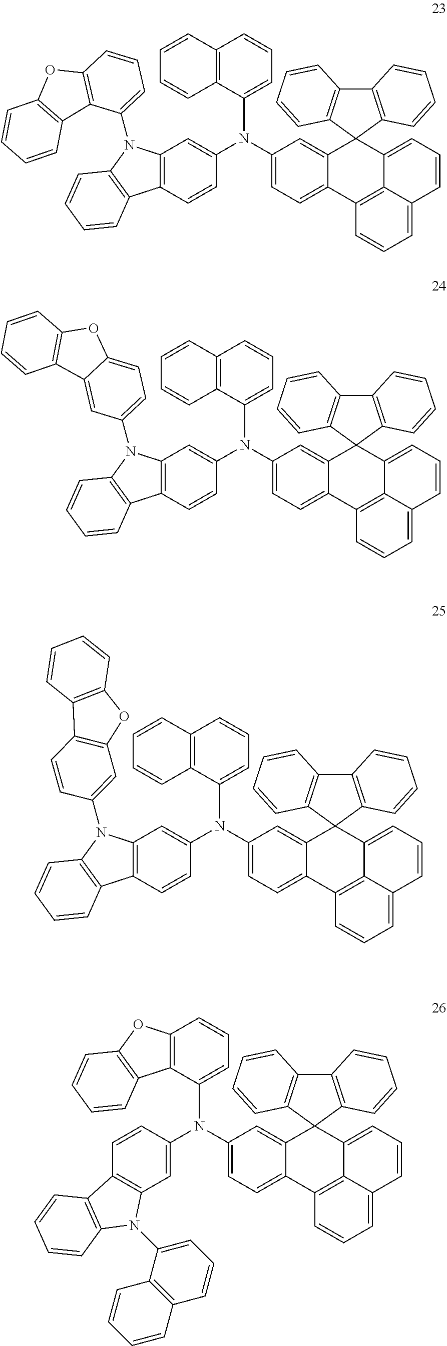 [SMILES: c1ccc2c(c1)-c1ccccc1C21c2cc(N(c3ccc4c5ccccc5n(-c5cccc6oc7ccccc7c56)c4c3)c3cccc4ccccc34)ccc2-c2cccc3cccc1c23 ; c1ccc2c(c1)-c1ccccc1C21c2cc(N(c3ccc4c5ccccc5n(-c5ccc6oc7ccccc7c6c5)c4c3)c3cccc4ccccc34)ccc2-c2cccc3cccc1c23 ; c1ccc2c(c1)-c1ccccc1C21c2cc(N(c3ccc4c5ccccc5n(-c5ccc6c(c5)oc5ccccc56)c4c3)c3cccc4ccccc34)ccc2-c2cccc3cccc1c23 ; c1ccc2c(c1)-c1ccccc1C21c2cc(N(c3ccc4c5ccccc5n(-c5cccc6ccccc56)c4c3)c3cccc4oc5ccccc5c34)ccc2-c2cccc3cccc1c23]

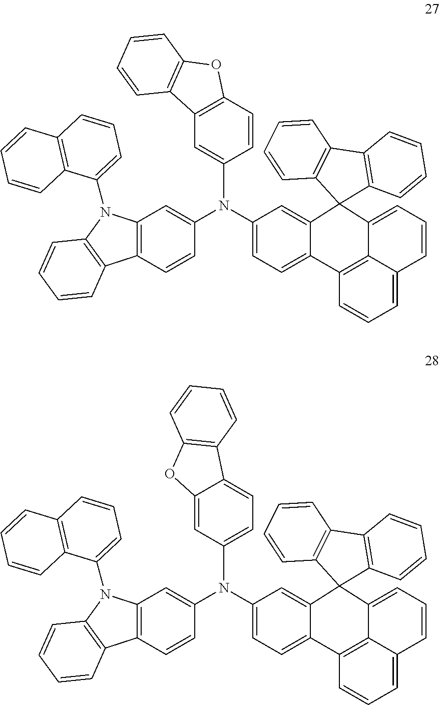 [SMILES: c1ccc2c(c1)-c1ccccc1C21c2cc(N(c3ccc4oc5ccccc5c4c3)c3ccc4c5ccccc5n(-c5cccc6ccccc56)c4c3)ccc2-c2cccc3cccc1c23 ; c1ccc2c(c1)-c1ccccc1C21c2cc(N(c3ccc4c(c3)oc3ccccc34)c3ccc4c5ccccc5n(-c5cccc6ccccc56)c4c3)ccc2-c2cccc3cccc1c23]

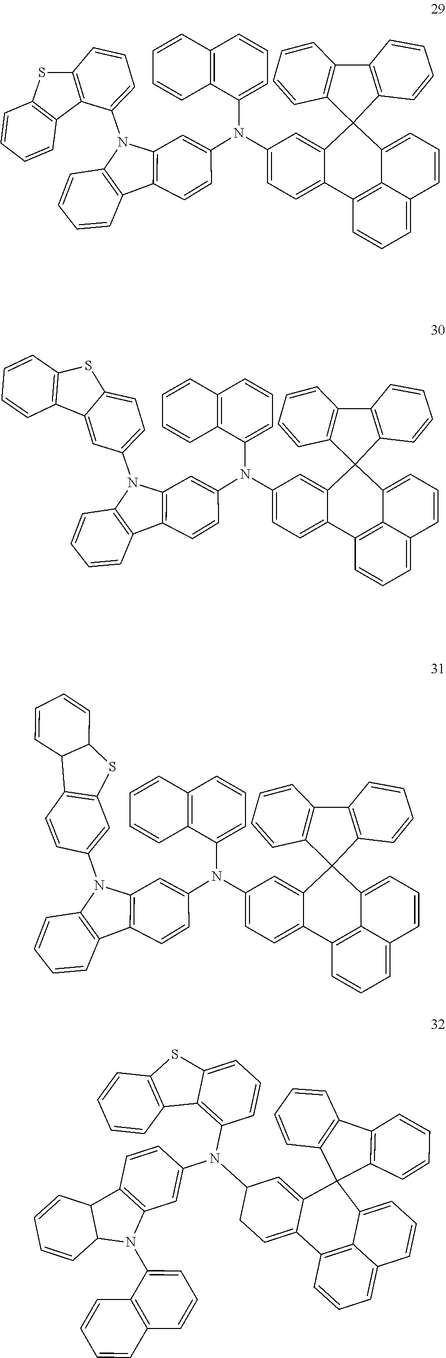 [SMILES: c1ccc2c(c1)-c1ccccc1C21c2cc(N(c3ccc4c5ccccc5n(-c5cccc6sc7ccccc7c56)c4c3)c3cccc4ccccc34)ccc2-c2cccc3cccc1c23 ; c1ccc2c(c1)-c1ccccc1C21c2cc(N(c3ccc4c5ccccc5n(-c5ccc6sc7ccccc7c6c5)c4c3)c3cccc4ccccc34)ccc2-c2cccc3cccc1c23 ; C1=CC2Sc3cc(-n4c5ccccc5c5ccc(N(c6ccc7c(c6)C6(c8ccccc8-c8ccccc86)c6cccc8cccc-7c68)c6cccc7ccccc67)cc54)ccc3C2C=C1 ; C1=CC2c3ccc(N(c4cccc5sc6ccccc6c45)C4C=C5C(=CC4)c4cccc6cccc(c46)C54c5ccccc5-c5ccccc54)cc3N(c3cccc4ccccc34)C2C=C1]

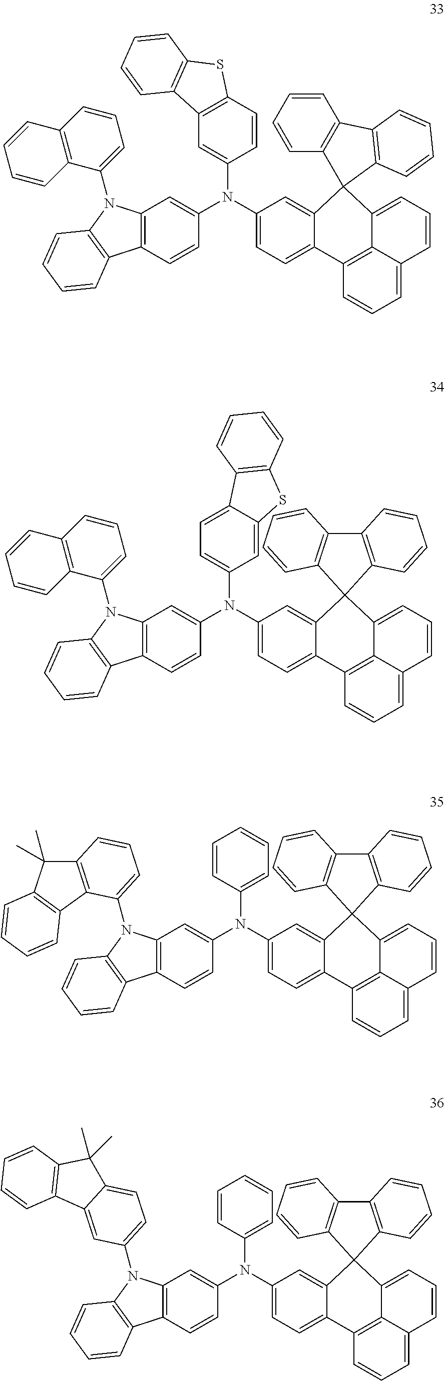 [SMILES: c1ccc2c(c1)-c1ccccc1C21c2cc(N(c3ccc4sc5ccccc5c4c3)c3ccc4c5ccccc5n(-c5cccc6ccccc56)c4c3)ccc2-c2cccc3cccc1c23 ; c1ccc2c(c1)-c1ccccc1C21c2cc(N(c3ccc4c(c3)sc3ccccc34)c3ccc4c5ccccc5n(-c5cccc6ccccc56)c4c3)ccc2-c2cccc3cccc1c23 ; CC1(C)c2ccccc2-c2c(-n3c4ccccc4c4ccc(N(c5ccccc5)c5ccc6c(c5)C5(c7ccccc7-c7ccccc75)c5cccc7cccc-6c57)cc43)cccc21 ; CC1(C)c2ccccc2-c2cc(-n3c4ccccc4c4ccc(N(c5ccccc5)c5ccc6c(c5)C5(c7ccccc7-c7ccccc75)c5cccc7cccc-6c57)cc43)ccc21]

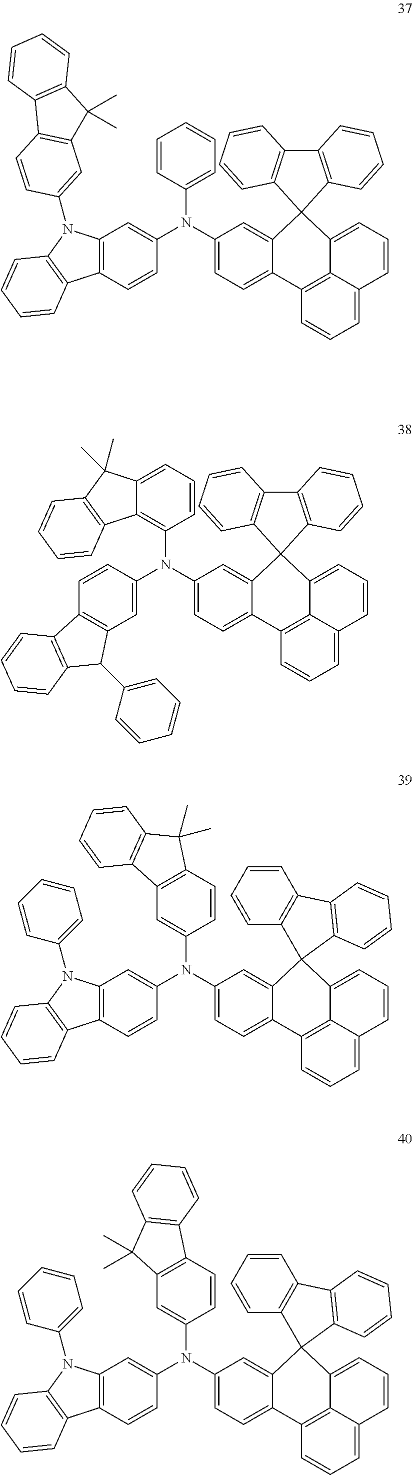 [SMILES: CC1(C)c2ccccc2-c2ccc(-n3c4ccccc4c4ccc(N(c5ccccc5)c5ccc6c(c5)C5(c7ccccc7-c7ccccc75)c5cccc7cccc-6c57)cc43)cc21 ; CC1(C)c2ccccc2-c2c(N(c3ccc4c(c3)C(c3ccccc3)c3ccccc3-4)c3ccc4c(c3)C3(c5ccccc5-c5ccccc53)c3cccc5cccc-4c35)cccc21 ; CC1(C)c2ccccc2-c2cc(N(c3ccc4c(c3)C3(c5ccccc5-c5ccccc53)c3cccc5cccc-4c35)c3ccc4c5ccccc5n(-c5ccccc5)c4c3)ccc21 ; CC1(C)c2ccccc2-c2ccc(N(c3ccc4c(c3)C3(c5ccccc5-c5ccccc53)c3cccc5cccc-4c35)c3ccc4c5ccccc5n(-c5ccccc5)c4c3)cc21]

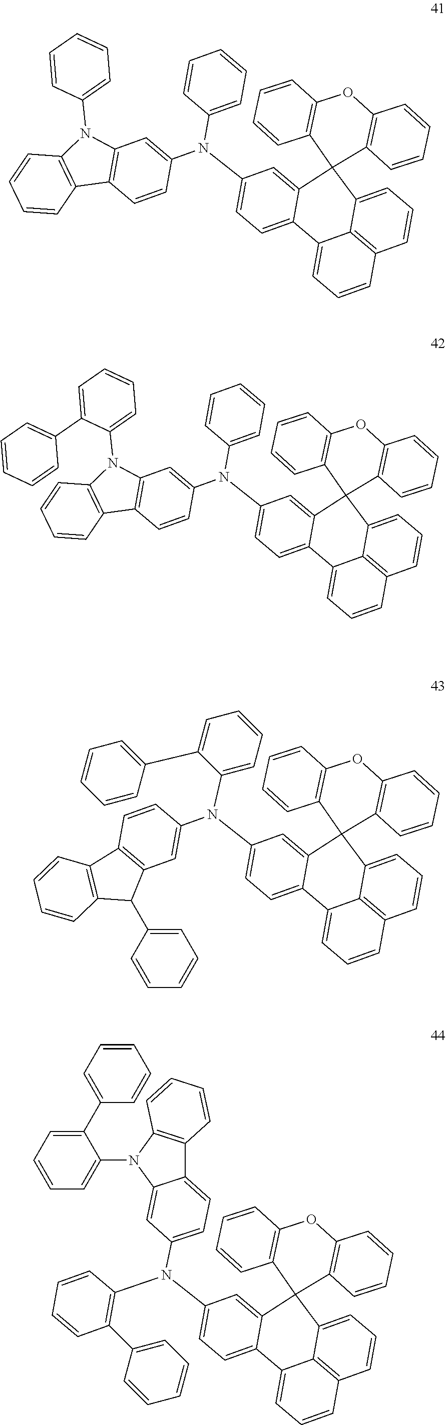 [SMILES: c1ccc(N(c2ccc3c(c2)C2(c4ccccc4Oc4ccccc42)c2cccc4cccc-3c24)c2ccc3c4ccccc4n(-c4ccccc4)c3c2)cc1 ; c1ccc(-c2ccccc2-n2c3ccccc3c3ccc(N(c4ccccc4)c4ccc5c(c4)C4(c6ccccc6Oc6ccccc64)c4cccc6cccc-5c46)cc32)cc1 ; c1ccc(-c2ccccc2N(c2ccc3c(c2)C(c2ccccc2)c2ccccc2-3)c2ccc3c(c2)C2(c4ccccc4Oc4ccccc42)c2cccc4cccc-3c24)cc1 ; c1ccc(-c2ccccc2N(c2ccc3c(c2)C2(c4ccccc4Oc4ccccc42)c2cccc4cccc-3c24)c2ccc3c4ccccc4n(-c4ccccc4-c4ccccc4)c3c2)cc1]

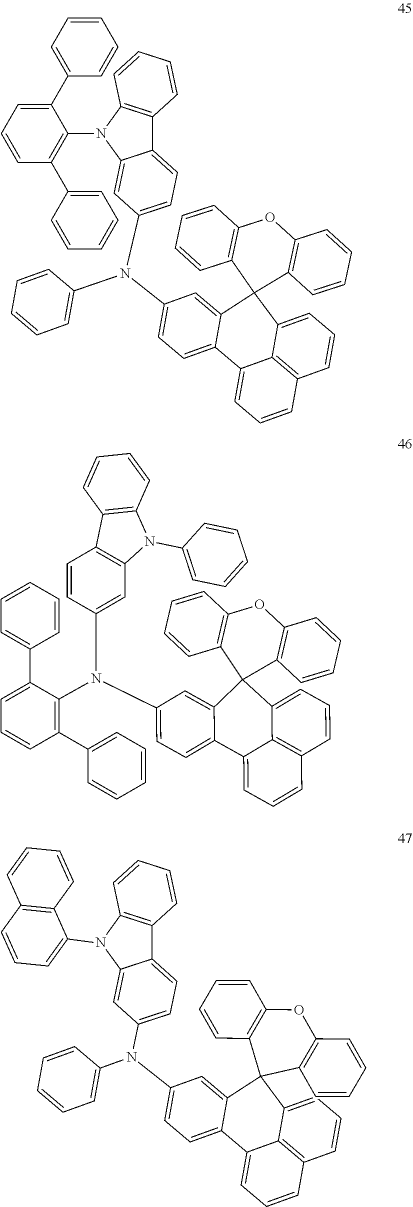 [SMILES: c1ccc(-c2cccc(-c3ccccc3)c2-n2c3ccccc3c3ccc(N(c4ccccc4)c4ccc5c(c4)C4(c6ccccc6Oc6ccccc64)c4cccc6cccc-5c46)cc32)cc1 ; c1ccc(-c2cccc(-c3ccccc3)c2N(c2ccc3c(c2)C2(c4ccccc4Oc4ccccc42)c2cccc4cccc-3c24)c2ccc3c4ccccc4n(-c4ccccc4)c3c2)cc1 ; c1ccc(N(c2ccc3c(c2)C2(c4ccccc4Oc4ccccc42)c2cccc4cccc-3c24)c2ccc3c4ccccc4n(-c4cccc5ccccc45)c3c2)cc1]

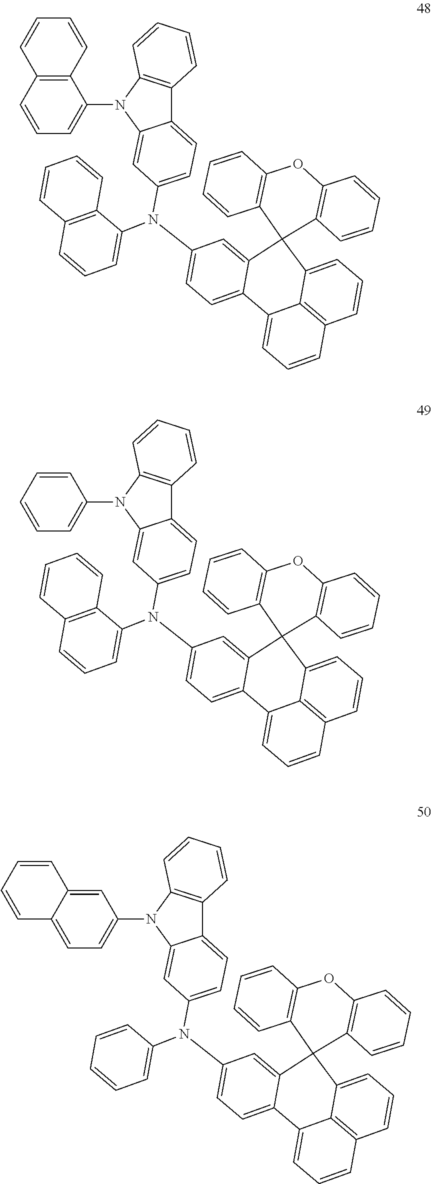 [SMILES: c1ccc2c(c1)Oc1ccccc1C21c2cc(N(c3ccc4c5ccccc5n(-c5cccc6ccccc56)c4c3)c3cccc4ccccc34)ccc2-c2cccc3cccc1c23 ; c1ccc(-n2c3ccccc3c3ccc(N(c4ccc5c(c4)C4(c6ccccc6Oc6ccccc64)c4cccc6cccc-5c46)c4cccc5ccccc45)cc32)cc1 ; c1ccc(N(c2ccc3c(c2)C2(c4ccccc4Oc4ccccc42)c2cccc4cccc-3c24)c2ccc3c4ccccc4n(-c4ccc5ccccc5c4)c3c2)cc1]

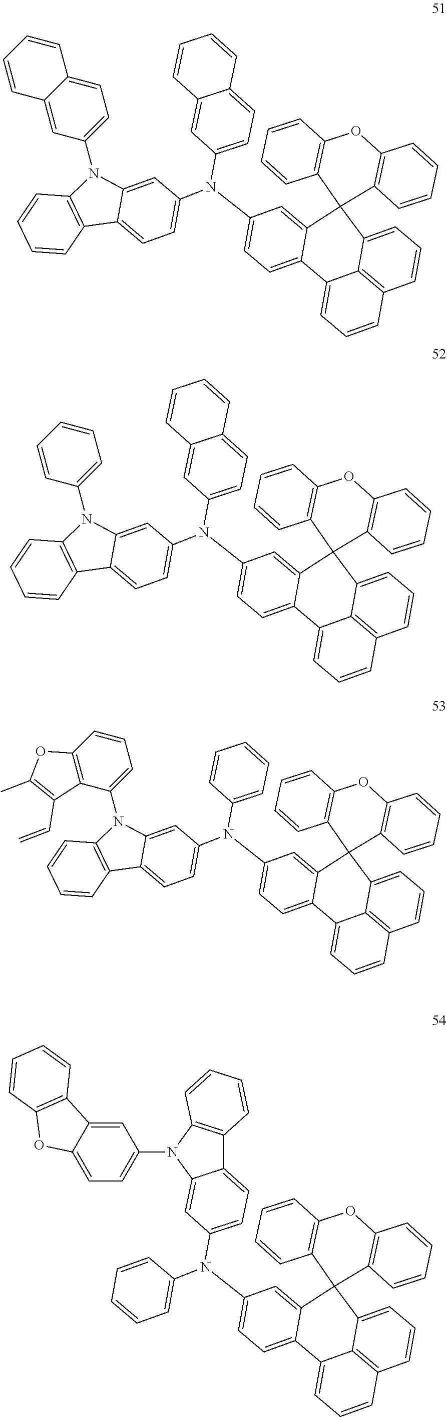 [SMILES: c1ccc2c(c1)Oc1ccccc1C21c2cc(N(c3ccc4ccccc4c3)c3ccc4c5ccccc5n(-c5ccc6ccccc6c5)c4c3)ccc2-c2cccc3cccc1c23 ; c1ccc(-n2c3ccccc3c3ccc(N(c4ccc5c(c4)C4(c6ccccc6Oc6ccccc64)c4cccc6cccc-5c46)c4ccc5ccccc5c4)cc32)cc1 ; C=Cc1c(C)oc2cccc(-n3c4ccccc4c4ccc(N(c5ccccc5)c5ccc6c(c5)C5(c7ccccc7Oc7ccccc75)c5cccc7cccc-6c57)cc43)c12 ; c1ccc(N(c2ccc3c(c2)C2(c4ccccc4Oc4ccccc42)c2cccc4cccc-3c24)c2ccc3c4ccccc4n(-c4ccc5oc6ccccc6c5c4)c3c2)cc1]

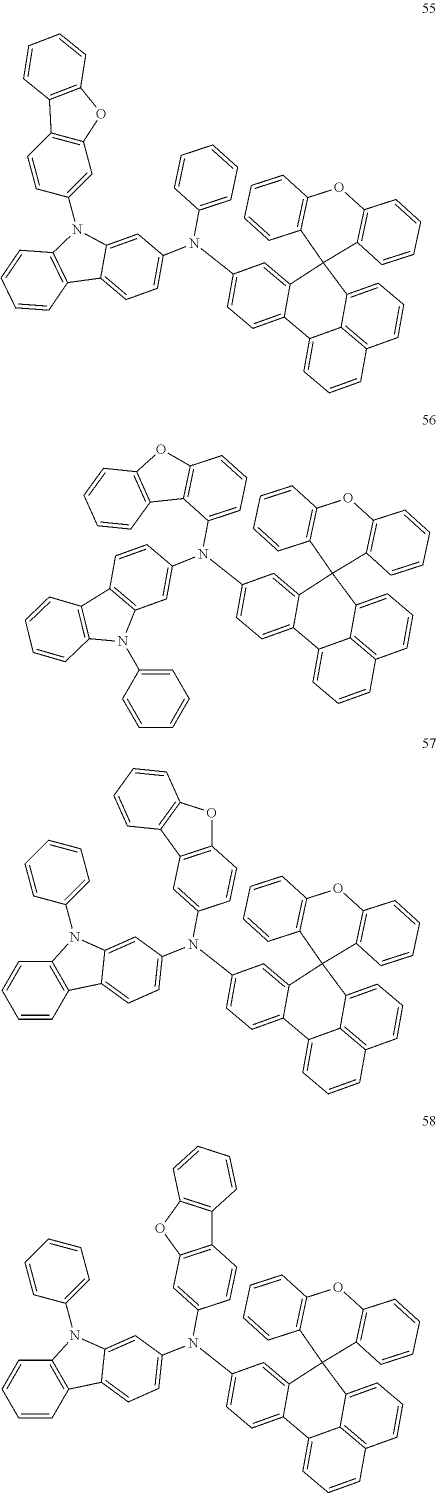

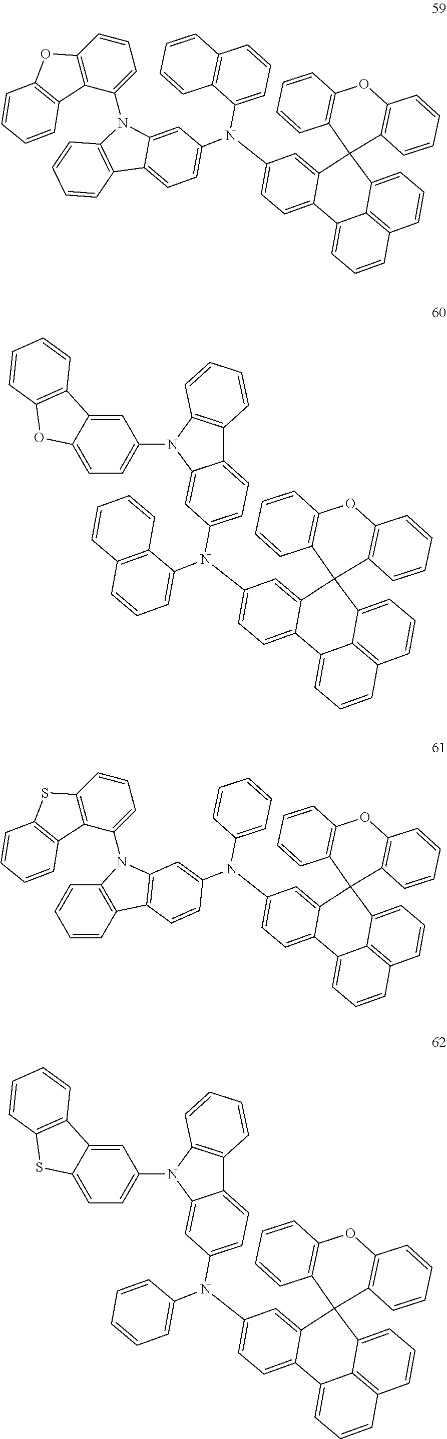 [SMILES: c1ccc2c(c1)Oc1ccccc1C21c2cc(N(c3ccc4c5ccccc5n(-c5cccc6oc7ccccc7c56)c4c3)c3cccc4ccccc34)ccc2-c2cccc3cccc1c23 ; c1ccc2c(c1)Oc1ccccc1C21c2cc(N(c3ccc4c5ccccc5n(-c5ccc6oc7ccccc7c6c5)c4c3)c3cccc4ccccc34)ccc2-c2cccc3cccc1c23 ; c1ccc(N(c2ccc3c(c2)C2(c4ccccc4Oc4ccccc42)c2cccc4cccc-3c24)c2ccc3c4ccccc4n(-c4cccc5sc6ccccc6c45)c3c2)cc1 ; c1ccc(N(c2ccc3c(c2)C2(c4ccccc4Oc4ccccc42)c2cccc4cccc-3c24)c2ccc3c4ccccc4n(-c4ccc5sc6ccccc6c5c4)c3c2)cc1]

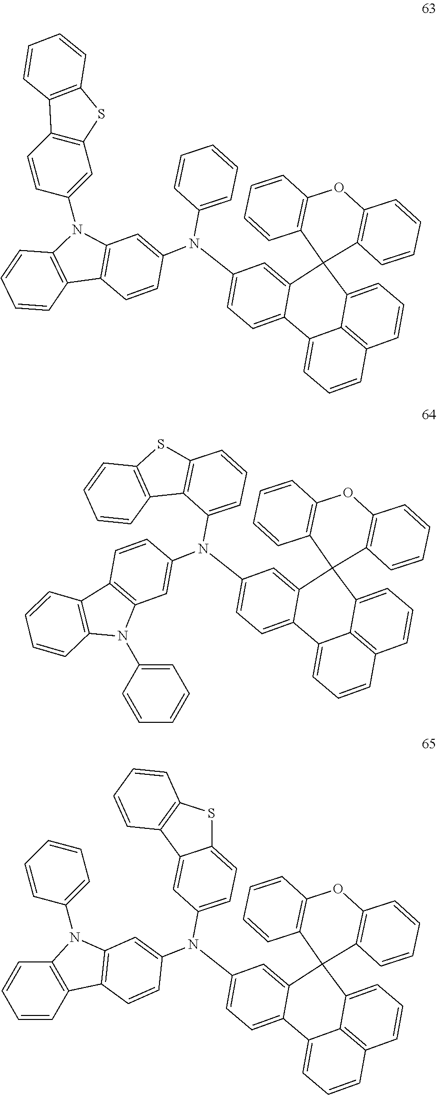 [SMILES: c1ccc(N(c2ccc3c(c2)C2(c4ccccc4Oc4ccccc42)c2cccc4cccc-3c24)c2ccc3c4ccccc4n(-c4ccc5c(c4)sc4ccccc45)c3c2)cc1 ; c1ccc(-n2c3ccccc3c3ccc(N(c4ccc5c(c4)C4(c6ccccc6Oc6ccccc64)c4cccc6cccc-5c46)c4cccc5sc6ccccc6c45)cc32)cc1 ; c1ccc(-n2c3ccccc3c3ccc(N(c4ccc5c(c4)C4(c6ccccc6Oc6ccccc64)c4cccc6cccc-5c46)c4ccc5sc6ccccc6c5c4)cc32)cc1]

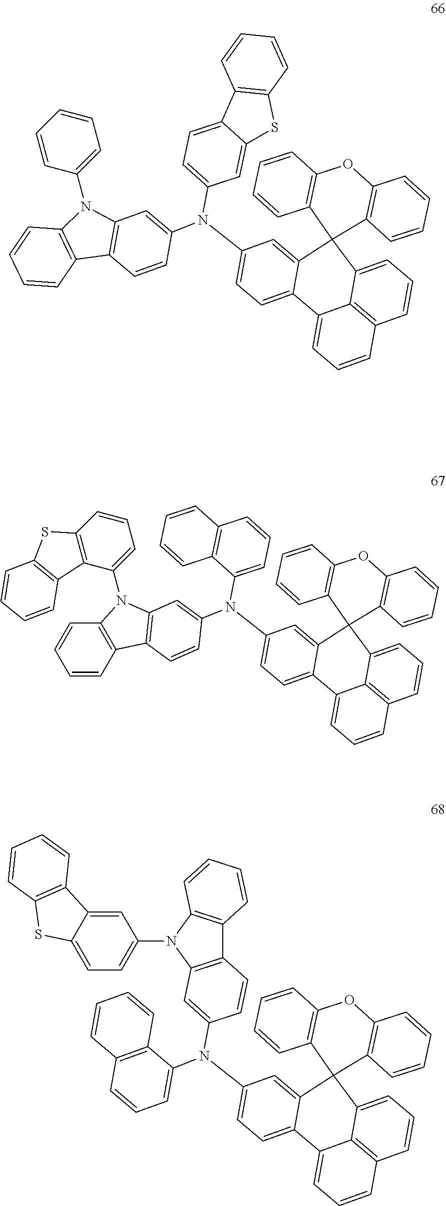 [SMILES: c1ccc(-n2c3ccccc3c3ccc(N(c4ccc5c(c4)C4(c6ccccc6Oc6ccccc64)c4cccc6cccc-5c46)c4ccc5c(c4)sc4ccccc45)cc32)cc1 ; c1ccc2c(c1)Oc1ccccc1C21c2cc(N(c3ccc4c5ccccc5n(-c5cccc6sc7ccccc7c56)c4c3)c3cccc4ccccc34)ccc2-c2cccc3cccc1c23 ; c1ccc2c(c1)Oc1ccccc1C21c2cc(N(c3ccc4c5ccccc5n(-c5ccc6sc7ccccc7c6c5)c4c3)c3cccc4ccccc34)ccc2-c2cccc3cccc1c23]

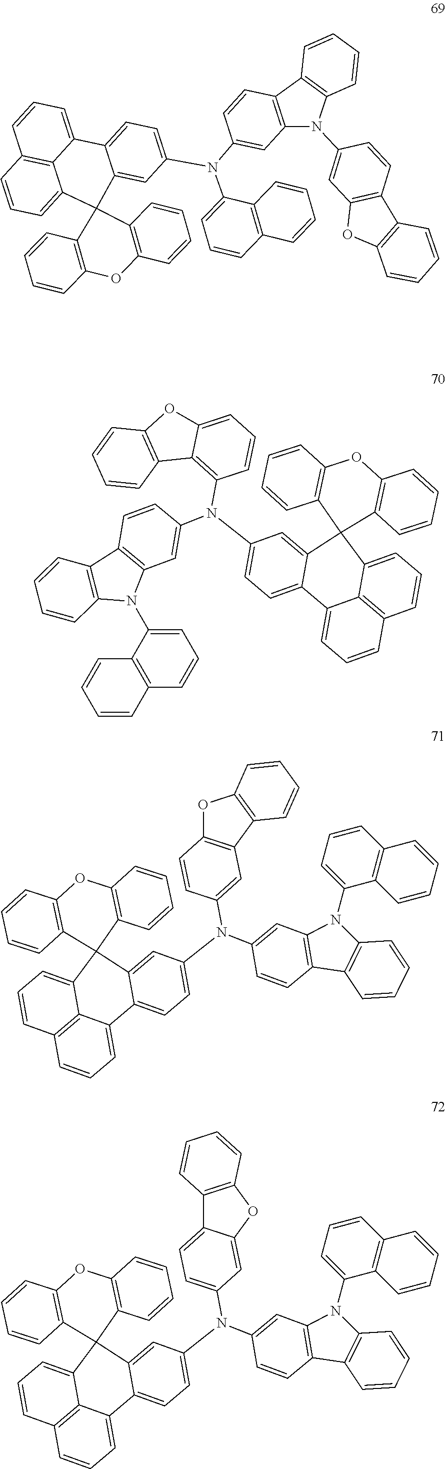 [SMILES: c1ccc2c(c1)Oc1ccccc1C21c2cc(N(c3ccc4c5ccccc5n(-c5ccc6c(c5)oc5ccccc56)c4c3)c3cccc4ccccc34)ccc2-c2cccc3cccc1c23 ; c1ccc2c(c1)Oc1ccccc1C21c2cc(N(c3ccc4c5ccccc5n(-c5cccc6ccccc56)c4c3)c3cccc4oc5ccccc5c34)ccc2-c2cccc3cccc1c23 ; c1ccc2c(c1)Oc1ccccc1C21c2cc(N(c3ccc4oc5ccccc5c4c3)c3ccc4c5ccccc5n(-c5cccc6ccccc56)c4c3)ccc2-c2cccc3cccc1c23 ; c1ccc2c(c1)Oc1ccccc1C21c2cc(N(c3ccc4c(c3)oc3ccccc34)c3ccc4c5ccccc5n(-c5cccc6ccccc56)c4c3)ccc2-c2cccc3cccc1c23]

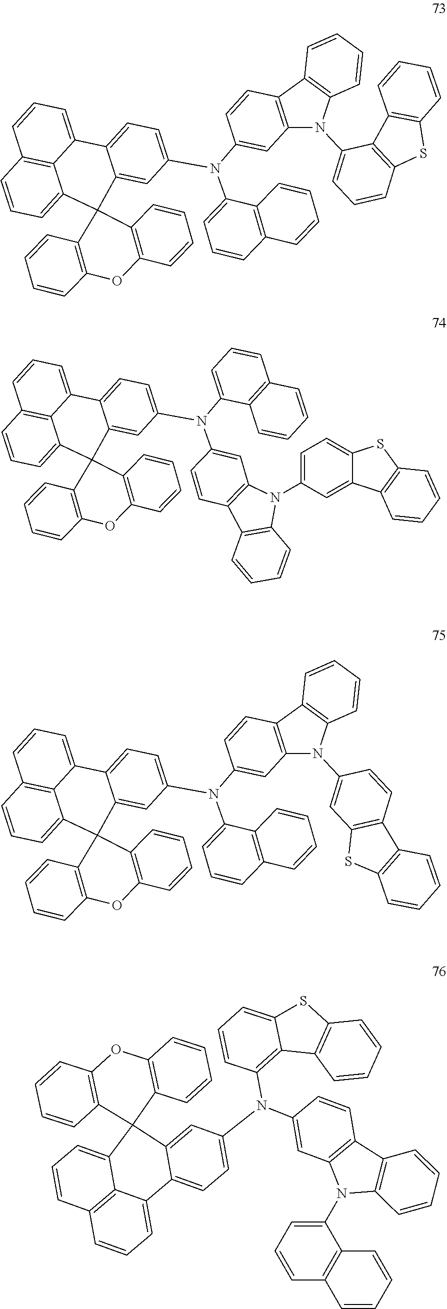

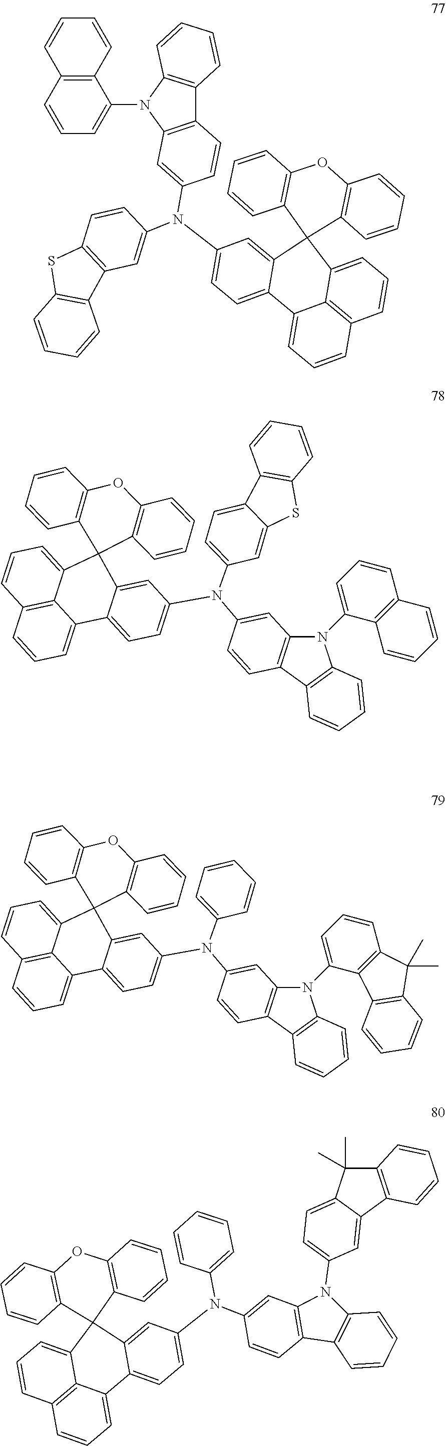 [SMILES: c1ccc2c(c1)Oc1ccccc1C21c2cc(N(c3ccc4sc5ccccc5c4c3)c3ccc4c5ccccc5n(-c5cccc6ccccc56)c4c3)ccc2-c2cccc3cccc1c23 ; c1ccc2c(c1)Oc1ccccc1C21c2cc(N(c3ccc4c(c3)sc3ccccc34)c3ccc4c5ccccc5n(-c5cccc6ccccc56)c4c3)ccc2-c2cccc3cccc1c23 ; CC1(C)c2ccccc2-c2c(-n3c4ccccc4c4ccc(N(c5ccccc5)c5ccc6c(c5)C5(c7ccccc7Oc7ccccc75)c5cccc7cccc-6c57)cc43)cccc21 ; CC1(C)c2ccccc2-c2cc(-n3c4ccccc4c4ccc(N(c5ccccc5)c5ccc6c(c5)C5(c7ccccc7Oc7ccccc75)c5cccc7cccc-6c57)cc43)ccc21]

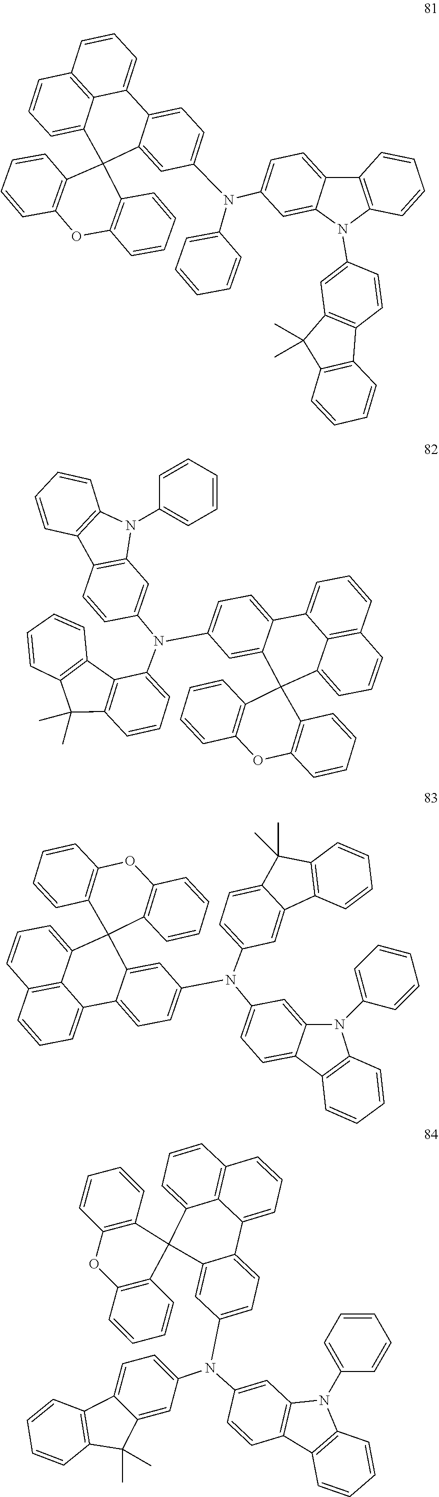 [SMILES: CC1(C)c2ccccc2-c2ccc(-n3c4ccccc4c4ccc(N(c5ccccc5)c5ccc6c(c5)C5(c7ccccc7Oc7ccccc75)c5cccc7cccc-6c57)cc43)cc21 ; CC1(C)c2ccccc2-c2c(N(c3ccc4c(c3)C3(c5ccccc5Oc5ccccc53)c3cccc5cccc-4c35)c3ccc4c5ccccc5n(-c5ccccc5)c4c3)cccc21 ; CC1(C)c2ccccc2-c2cc(N(c3ccc4c(c3)C3(c5ccccc5Oc5ccccc53)c3cccc5cccc-4c35)c3ccc4c5ccccc5n(-c5ccccc5)c4c3)ccc21 ; CC1(C)c2ccccc2-c2ccc(N(c3ccc4c(c3)C3(c5ccccc5Oc5ccccc53)c3cccc5cccc-4c35)c3ccc4c5ccccc5n(-c5ccccc5)c4c3)cc21]

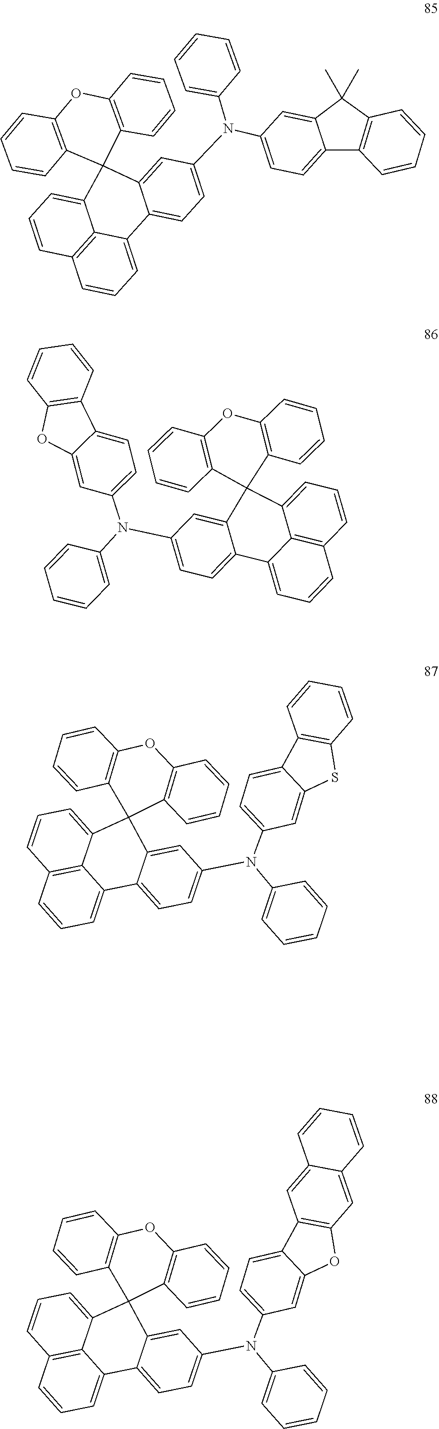 [SMILES: CC1(C)c2ccccc2-c2ccc(N(c3ccccc3)c3ccc4c(c3)C3(c5ccccc5Oc5ccccc53)c3cccc5cccc-4c35)cc21 ; c1ccc(N(c2ccc3c(c2)C2(c4ccccc4Oc4ccccc42)c2cccc4cccc-3c24)c2ccc3c(c2)oc2ccccc23)cc1 ; c1ccc(N(c2ccc3c(c2)C2(c4ccccc4Oc4ccccc42)c2cccc4cccc-3c24)c2ccc3c(c2)sc2ccccc23)cc1 ; c1ccc(N(c2ccc3c(c2)C2(c4ccccc4Oc4ccccc42)c2cccc4cccc-3c24)c2ccc3c(c2)oc2cc4ccccc4cc23)cc1]

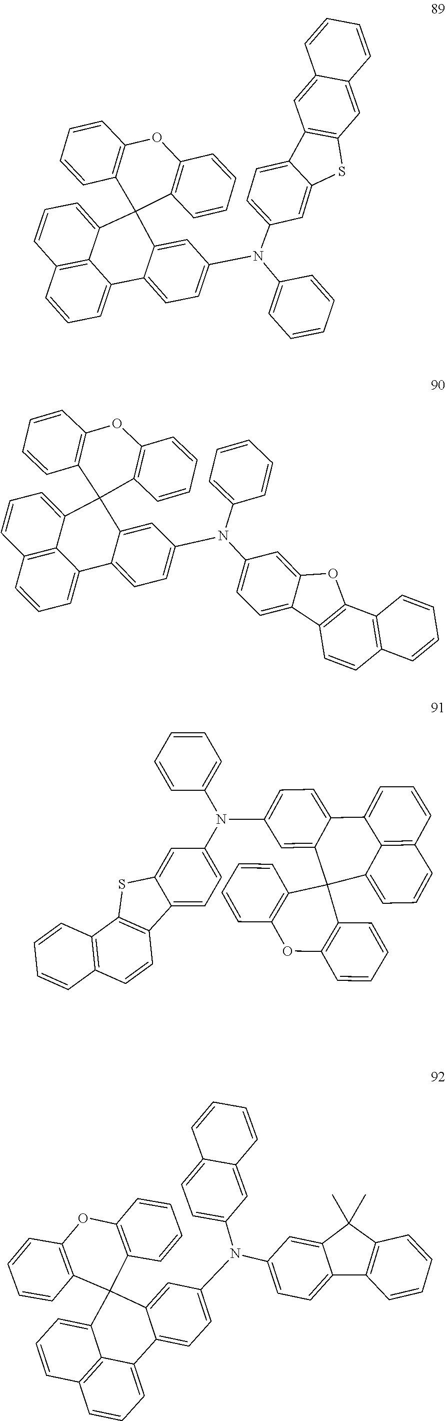 [SMILES: c1ccc(N(c2ccc3c(c2)C2(c4ccccc4Oc4ccccc42)c2cccc4cccc-3c24)c2ccc3c(c2)sc2cc4ccccc4cc23)cc1 ; c1ccc(N(c2ccc3c(c2)C2(c4ccccc4Oc4ccccc42)c2cccc4cccc-3c24)c2ccc3c(c2)oc2c4ccccc4ccc32)cc1 ; c1ccc(N(c2ccc3c(c2)C2(c4ccccc4Oc4ccccc42)c2cccc4cccc-3c24)c2ccc3c(c2)sc2c4ccccc4ccc32)cc1 ; CC1(C)c2ccccc2-c2ccc(N(c3ccc4c(c3)C3(c5ccccc5Oc5ccccc53)c3cccc5cccc-4c35)c3ccc4ccccc4c3)cc21]

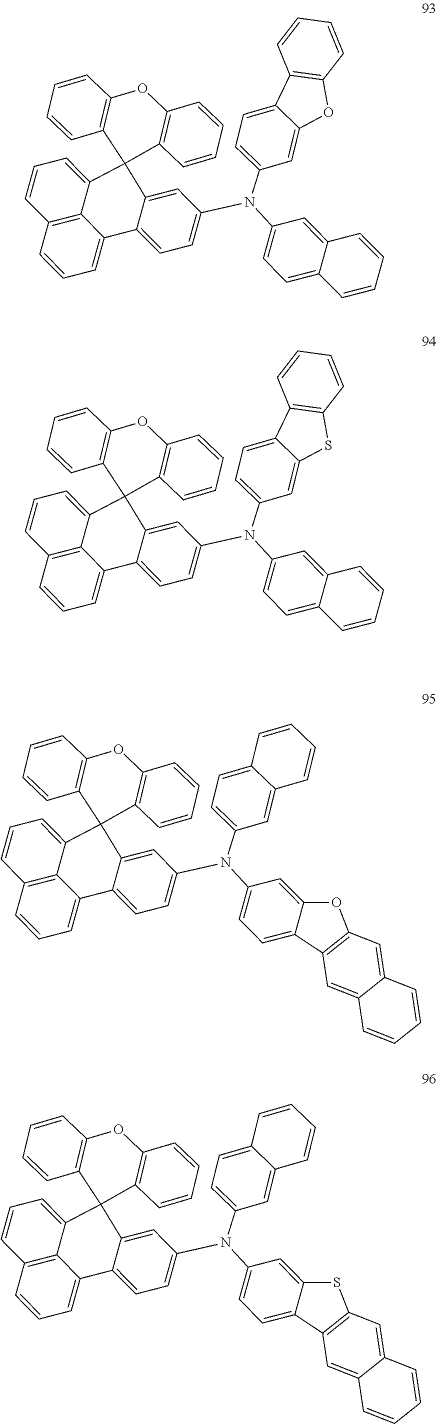 [SMILES: c1ccc2c(c1)Oc1ccccc1C21c2cc(N(c3ccc4ccccc4c3)c3ccc4c(c3)oc3ccccc34)ccc2-c2cccc3cccc1c23 ; c1ccc2c(c1)Oc1ccccc1C21c2cc(N(c3ccc4ccccc4c3)c3ccc4c(c3)sc3ccccc34)ccc2-c2cccc3cccc1c23 ; c1ccc2c(c1)Oc1ccccc1C21c2cc(N(c3ccc4ccccc4c3)c3ccc4c(c3)oc3cc5ccccc5cc34)ccc2-c2cccc3cccc1c23 ; c1ccc2c(c1)Oc1ccccc1C21c2cc(N(c3ccc4ccccc4c3)c3ccc4c(c3)sc3cc5ccccc5cc34)ccc2-c2cccc3cccc1c23]

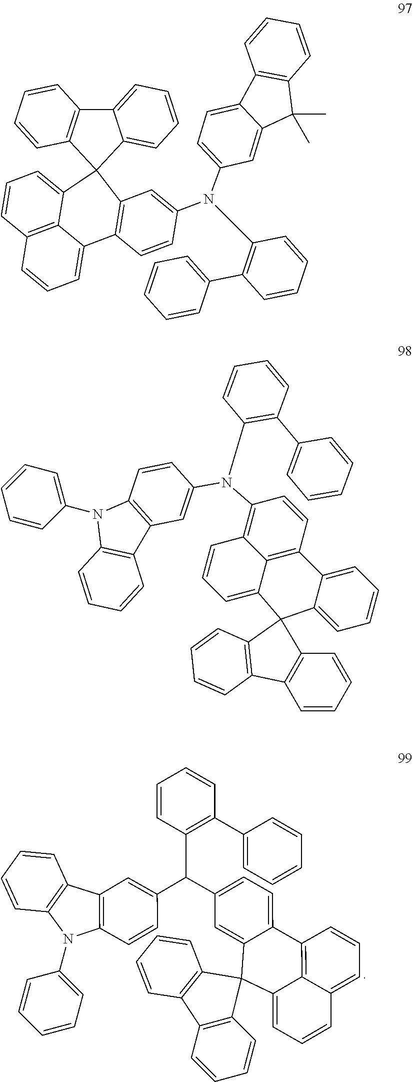 [SMILES: CC1(C)c2ccccc2-c2ccc(N(c3ccc4c(c3)C3(c5ccccc5-c5ccccc53)c3cccc5cccc-4c35)c3ccccc3-c3ccccc3)cc21 ; c1ccc(-c2ccccc2N(c2ccc3c(c2)c2ccccc2n3-c2ccccc2)c2ccc3c4c(cccc24)C2(c4ccccc4-c4ccccc42)c2ccccc2-3)cc1 ; c1ccc(-c2ccccc2C(c2ccc3c(c2)C2(c4ccccc4-c4ccccc42)c2cccc4cccc-3c24)c2ccc3c(c2)c2ccccc2n3-c2ccccc2)cc1]

18. An electronic apparatus comprising the light-emitting device of claim 1.

19. The electronic apparatus of claim 18, further comprising a thin-film transistor, wherein
the thin-film transistor comprises a source electrode and a drain electrode, and
the first electrode of the light-emitting device is electrically connected to at least one of the source electrode and the drain electrode of the thin-film transistor.

20. The electronic apparatus of claim 18, further comprising a color filter, a color conversion layer, a touch screen layer, a polarizing layer, or any combination thereof.
